(19) United States
(12) Patent Application Publication
(10) Pub. No.: US 2023/0399645 A1
(43) Pub. Date: Dec. 14, 2023

(54) OPTIMIZED SIRNA SCAFFOLDS

(71) Applicant: UNIVERSITY OF MASSACHUSETTS, Boston, MA (US)

(72) Inventors: Anastasia Khvorova, Westborough, MA (US); Daniel O'Reilly, Barnham (GB); Vignesh Narayan Hariharan, Natick, MA (US); Clemens Lochmann, Badem-Wuerttemberg (DE); David Cooper, Framingham, MA (US)

(21) Appl. No.: 18/197,948

(22) Filed: May 16, 2023

Related U.S. Application Data
(60) Provisional application No. 63/342,393, filed on May 16, 2022.

(51) Int. Cl. C12N 15/113; C12N 15/10

(52) U.S. Cl. CPC C12N 15/113; C12N 15/102; C12N 2310/315

(57) ABSTRACT

This disclosure relates to novel modified oligonucleotides with increased stability and extended in vivo mRNA silencing activity.

### CROSS REFERENCE TO RELATED APPLICATION

This application claims the benefit of U.S. Provisional Patent Application Ser. No. 63/342,393, filed May 16, 2022. The entire contents of the above-referenced patent application is incorporated by reference in their entirety herein.

### FIELD OF THE DISCLOSURE

This disclosure relates to the use of various chemical modifications on oligonucleotides to improve stability and extend in vivo silencing duration.

### BACKGROUND

Chemically modified siRNA are at the forefront of oligonucleotide therapeutics. Chemical modifications, such as 2′FRNA, 2′OMe, phosphorothioate, and vinyl phosphonate modifications, enhance efficacy and stability in vivo. However, numerous other modifications have not been translated to in vivo studies or the clinic.

Despite the general efficacy of the existing oligonucleotide modification patterns, long-term in vivo silencing of mRNA (e.g., 6-months or greater) remains elusive. Moreover, existing modification patterns may lead to excessive silencing of a target mRNA. Over silencing a target may lead to unwanted side effects, while under silencing the target may lead to no therapeutic benefit.

Accordingly, there exists a need in the art for optimized chemical modifications for oligonucleotides that achieve prolonged in vivo silencing activity while modulating the level of said silencing activity.

### SUMMARY

Provided herein are optimized oligonucleotide chemical modification patterns that may be used to achieve prolonged in vivo silencing (e.g., silencing of 6 months or greater). The optimized patterns may further be used to modulate the level of activity of said oligonucleotide, being capable of robust silencing (e.g., silencing of about 75% or greater) or modest silencing (e.g., silencing of about 25% to about 50%).

In one aspect, the disclosure provides an RNA molecule comprising a 5′ end and a 3′ end, wherein the RNA molecule comprises at least one alkyl modification within nucleotide positions 1-5 of one or both of the 5′ end and 3′ end.

In certain embodiments, the at least one alkyl modification comprises a C₁-C₁₀ alkyl. In certain embodiments, the at least one alkyl modification comprises a C₄ alkyl (i.e., butyl). In certain embodiments, the at least one alkyl modification comprises a C₁ alkyl. In certain embodiments, the at least one alkyl modification comprises a C₂ alkyl. In certain embodiments, the at least one alkyl modification comprises a C₃ alkyl. In certain embodiments, the at least one alkyl modification comprises a C₅ alkyl. In certain embodiments, the at least one alkyl modification comprises a C₆ alkyl. In certain embodiments, the at least one alkyl modification comprises a C₇ alkyl. In certain embodiments, the at least one alkyl modification comprises a C₈ alkyl. In certain embodiments, the at least one alkyl modification comprises a C₉ alkyl. In certain embodiments, the at least one alkyl modification comprises a C₁₀ alkyl.

In certain embodiments, the at least one alkyl modification is positioned between two adjacent nucleotides.

In certain embodiments, the at least one alkyl modification positioned between two adjacent nucleotides does not replace a nucleotide at a position within the RNA molecule relative to an RNA molecule that does not contain the at least one alkyl modification at the same position within the RNA molecule.

In certain embodiments, the at least one alkyl modification replaces a nucleotide at a position within the RNA molecule relative to an RNA molecule that does not contain the at least one alkyl modification at the same position within the RNA molecule.

In certain embodiments, the RNA molecule comprises a single stranded (ss) RNA or a double stranded (ds) RNA.

In certain embodiments, the dsRNA comprises an antisense strand and a sense strand, each strand comprising a 5′ end and a 3′ end.

In certain embodiments, the antisense strand comprises at least one alkyl modification within nucleotide positions 1-5 of one or both of the 5′ end and 3′ end.

In certain embodiments of the dsRNA, the at least one alkyl modification comprises a C₁-C₁₀ alkyl. In certain embodiments of the dsRNA, the at least one alkyl modification comprises a C₄ alkyl (i.e., butyl). In certain embodiments, the at least one alkyl modification comprises a C₁ alkyl. In certain embodiments, the at least one alkyl modification comprises a C₂ alkyl. In certain embodiments, the at least one alkyl modification comprises a C₃ alkyl. In certain embodiments, the at least one alkyl modification comprises a C₅ alkyl. In certain embodiments, the at least one alkyl modification comprises a C₆ alkyl. In certain embodiments, the at least one alkyl modification comprises a C₇ alkyl. In certain embodiments, the at least one alkyl modification comprises a C₈ alkyl. In certain embodiments, the at least one alkyl modification comprises a C₉ alkyl. In certain embodiments, the at least one alkyl modification comprises a Cm alkyl.

In certain embodiments of the dsRNA, the at least one alkyl modification is positioned between two adjacent nucleotides.

In certain embodiments of the dsRNA, the at least one alkyl modification positioned between two adjacent nucleotides does not replace a nucleotide at a position within the RNA molecule relative to an RNA molecule that does not contain the at least one alkyl modification at the same position within the RNA molecule.

In certain embodiments of the dsRNA, the at least one alkyl modification replaces a nucleotide at a position within the RNA molecule relative to an RNA molecule that does not contain the at least one alkyl modification at the same position within the RNA molecule.

In one aspect, the disclosure provides a double stranded (ds) RNA, comprising an antisense strand with a 5′ end and a 3′ end, and a sense strand with a 5′ end and a 3′ end, wherein the antisense strand comprises at least one alkyl modification.

In certain embodiments of the dsRNA, the antisense strand is between 15 and nucleotides in length. In certain embodiments of the dsRNA, the antisense strand is 18, 19, 21, 22, or 23 nucleotides in length. In certain embodiments of the dsRNA, the sense strand is between 15 and 25 nucleotides in length. In certain embodiments of the dsRNA, the sense strand is 14, 15, 16, or 17 nucleotides in length.

In certain embodiments of the dsRNA, the at least one alkyl modification is at any one of positions 1-25 from the 5′ end of the antisense strand.

In certain embodiments, the dsRNA further comprises at least one non-alkyl modified nucleotide. In certain embodiments, the at least one non-alkyl modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the dsRNA comprises at least one modified internucleotide linkage. In certain embodiments, the modified internucleotide linkage comprises a phosphorothioate internucleotide linkage. In certain embodiments, the dsRNA comprises 4-16 phosphorothioate internucleotide linkages. In certain embodiments, the dsRNA comprises 8-13 phosphorothioate internucleotide linkages.

In certain embodiments, the dsRNA comprises a blunt end.

In certain embodiments, the dsRNA comprises at least one single stranded nucleotide overhang. In certain embodiments, the dsRNA comprises about a 2-nucleotide to 5-nucleotide single stranded nucleotide overhang. In certain embodiments, the dsRNA comprises 2-nucleotide single stranded nucleotide overhang. In certain embodiments, the dsRNA comprises 5-nucleotide single stranded nucleotide overhang.

In certain embodiments, the single stranded nucleotide overhang comprises at least two alkyl modifications.

In certain embodiments, the single stranded nucleotide overhang comprises 2, 3, 4, or 5 alkyl modifications.

In certain embodiments, the dsRNA comprises an antisense strand with one of the following chemical modification patterns:

| | |
|---|---|
| P1_b1_as | P(but)#(fN)#(mN)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_b2_as | P(mN)#(but)#(mN)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_b3_as | P(mN)#(fN)#(but)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_b4_as | P(mN)#(fN)#(mN)(but)(fN)(fN)(mN)(fN)(mN)(fN)(mN)(fN)(mN)(fN)#(mN)# |
| | (fN)#(mN)#(mN)#(mN)#(fN)#(mN) |
| P1_b5_as | P(mN)#(fN)#(mN)(fN)(but)(fN)(mN)(fN)(mN)(fN)(mN)(fN)(mN)(fN)#(mN)# |
| | (fN)#(mN)#(mN)#(mN)#(fN)#(mN) |
| P1_b6_as | P(mN)#(fN)#(mN)(fN)(fN)(but)(mN)(fN)(mN)(fN)(mN)(fN)(mN)(fN)#(mN)# |
| | (fN)#(mN)#(mN)#(mN)#(fN)#(mN) |
| P1_b7_as | P(mN)#(fN)#(mN)(fN)(fN)(fN)(but)(fN)(mN)(fN)(mN)(fN)(mN)(fN)#(mN)# |
| | (fN)#(mN)#(mN)#(mN)#(fN)#(mN) |
| P1_b8_as | P(mN)#(fN)#(mN)(fN)(fN)(fN)(mN)(but)(mN)(fN)(mN)(fN)(mN)(fN)#(mN)# |
| | (fN)#(mN)#(mN)#(mN)#(fN)#(mN) |
| P1_b9_as | P(mN)#(fN)#(mN)(fN)(fN)(fN)(mN)(fN)(but)(fN)(mN)(fN)(mN)(fN)#(mN)# |
| | (fN)#(mN)#(mN)#(mN)#(fN)#(mN) |
| P1_b10_as | P(mN)#(fN)#(mN)(fN)(fN)(fN)(mN)(fN)(mN)(but)(mN)(fN)(mN)(fN)#(mN)# |
| | (fN)#(mN)#(mN)#(mN)#(fN)#(mN) |
| P1_b11_as | P(mN)#(fN)#(mN)(fN)(fN)(fN)(mN)(fN)(mN)(fN)(but)(fN)(mN)(fN)#(mN)# |
| | (fN)#(mN)#(mN)#(mN)#(fN)#(mN) |
| P1_b12_as | P(mN)#(fN)#(mN)(fN)(fN)(fN)(mN)(fN)(mN)(fN)(mN)(but)(mN)(fN)#(mN)# |
| | (fN)#(mN)#(mN)#(mN)#(fN)#(mN) |
| P1_b13_as | P(mN)#(fN)#(mN)(fN)(fN)(fN)(mN)(fN)(mN)(fN)(mN)(fN)(but)(fN)#(mN)# |
| | (fN)#(mN)#(mN)#(mN)#(fN)#(mN) |
| P1_b14_as | P(mN)#(fN)#(mN)(fN)(fN)(fN)(mN)(fN)(mN)(fN)(mN)(fN)(mN)(but)#(mN)# |
| | (fN)#(mN)#(mN)#(mN)#(fN)#(mN) |
| P1_b15_as | P(mN)#(fN)#(mN)(fN)(fN)(fN)(mN)(fN)(mN)(fN)(mN)(fN)(mN)(fN)#(but)# |
| | (fN)#(mN)#(mN)#(mN)#(fN)#(mN) |
| P1_b16_as | P(mN)#(fN)#(mN)(fN)(fN)(fN)(mN)(fN)(mN)(fN)(mN)(fN)(mN)(fN)#(mN)# |
| | (but)#(mN)#(mN)#(mN)#(fN)#(mN) |
| P1_b17_as | P(mN)#(fN)#(mN)(fN)(fN)(fN)(mN)(fN)(mN)(fN)(mN)(fN)(mN)(fN)#(mN)# |
| | (fN)#(but)#(mN)#(mN)#(fN)#(mN) |
| P1_b18_as | P(mN)#(fN)#(mN)(fN)(fN)(fN)(mN)(fN)(mN)(fN)(mN)(fN)(mN)(fN)#(mN)# |
| | (fN)#(mN)#(but)#(mN)#(fN)#(mN) |
| P1_b19_as | P(mN)#(fN)#(mN)(fN)(fN)(fN)(mN)(fN)(mN)(fN)(mN)(fN)(mN)(fN)#(mN)# |
| | (fN)#(mN)#(mN)#(but)#(fN)#(mN) |
| P1_b20_as | P(mN)#(fN)#(mN)(fN)(fN)(fN)(mN)(fN)(mN)(fN)(mN)(fN)(mN)(fN)#(mN)# |
| | (fN)#(mN)#(mN)#(mN)#(but)#(mN) |
| P1_b21_as | P(mN)#(fN)#(mN)(fN)(fN)(fN)(mN)(fN)(mN)(fN)(mN)(fN)(mN)(fN)#(mN)# |
| | (fN)#(mN)#(mN)#(mN)#(fN)#(but) |
| P2_b1_as | P(but)#(fN)#(mN)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b2_as | P(mN)#(but)#(mN)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b3_as | P(mN)#(fN)#(but)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b4_as | P(mN)#(fN)#(mN)(but)(mN)(fN)(mN)(mN)(mN)(mN)(mN)(mN)(mN)(fN)#(mN) |
| | #(fN)#(mN)#(mN)#(mN)#(fN)#(mN) |
| P2_b5_as | P(mN)#(fN)#(mN)(mN)(but)(fN)(mN)(mN)(mN)(mN)(mN)(mN)(mN)(fN)#(mN) |
| | #(fN)#(mN)#(mN)#(mN)#(fN)#(mN) |
| P2_b6_as | P(mN)#(fN)#(mN)(mN)(mN)(but)(mN)(mN)(mN)(mN)(mN)(mN)(mN)(fN)#(mN) |
| | #(fN)#(mN)#(mN)#(mN)#(fN)#(mN) |
| P2_b7_as | P(mN)#(fN)#(mN)(mN)(mN)(fN)(but)(mN)(mN)(mN)(mN)(mN)(mN)(fN)# |
| | (mN)#(fN)#(mN)#(mN)#(mN)#(fN)#(mN) |
| P2_b8_as | P(mN)#(fN)#(mN)(mN)(mN)(fN)(mN)(but)(mN)(mN)(mN)(mN)(mN)(fN)# |
| | (mN)#(fN)#(mN)#(mN)#(mN)#(fN)#(mN) |
| P2_b9_as | P(mN)#(fN)#(mN)(mN)(mN)(fN)(mN)(mN)(but)(mN)(mN)(mN)(mN)(fN)# |
| | (mN)#(fN)#(mN)#(mN)#(mN)#(fN)#(mN) |
| P2_b10_as | P(mN)#(fN)#(mN)(mN)(mN)(fN)(mN)(mN)(mN)(but)(mN)(mN)(mN)(fN)# |
| | (mN)#(fN)#(mN)#(mN)#(mN)#(fN)#(mN) |
| P2_b11_as | P(mN)#(fN)#(mN)(mN)(mN)(fN)(mN)(mN)(mN)(mN)(but)(mN)(mN)(fN)# |
| | (mN)#(fN)#(mN)#(mN)#(mN)#(fN)#(mN) |
| P2_b12_as | P(mN)#(fN)#(mN)(mN)(mN)(fN)(mN)(mN)(mN)(mN)(mN)(but)(mN)(fN)# |
| | (mN)#(fN)#(mN)#(mN)#(mN)#(fN)#(mN) |
| P2_b13_as | P(mN)#(fN)#(mN)(mN)(mN)(fN)(mN)(mN)(mN)(mN)(mN)(mN)(but)(fN)# |
| | (mN)#(fN)#(mN)#(mN)#(mN)#(fN)#(mN) |
| P2_b14_as | P(mN)#(fN)#(mN)(mN)(mN)(fN)(mN)(mN)(mN)(mN)(mN)(mN)(mN)(but)# |
| | (mN)#(fN)#(mN)#(mN)#(mN)#(fN)#(mN) |
| P2_b15_as | P(mN)#(fN)#(mN)(mN)(mN)(fN)(mN)(mN)(mN)(mN)(mN)(mN)(mN)(fN)# |
| | (but)#(fN)#(mN)#(mN)#(mN)#(fN)#(mN) |
| P2_b16_as | P(mN)#(fN)#(mN)(mN)(mN)(fN)(mN)(mN)(mN)(mN)(mN)(mN)(mN)(fN)# |
| | (mN)#(but)#(mN)#(mN)#(mN)#(fN)#(mN) |
| P2_b17_as | P(mN)#(fN)#(mN)(mN)(mN)(fN)(mN)(mN)(mN)(mN)(mN)(mN)(mN)(fN)# |
| | (mN)#(fN)#(but)#(mN)#(mN)#(fN)#(mN) |
| P2_b18_as | P(mN)#(fN)#(mN)(mN)(mN)(fN)(mN)(mN)(mN)(mN)(mN)(mN)(mN)(fN)# |
| | (mN)#(fN)#(mN)#(but)#(mN)#(fN)#(mN) |
| P2_b19_as | P(mN)#(fN)#(mN)(mN)(mN)(fN)(mN)(mN)(mN)(mN)(mN)(mN)(mN)(fN)# |
| | (mN)#(fN)#(mN)#(mN)#(but)#(fN)#(mN) |
| P2_b20_as | P(mN)#(fN)#(mN)(mN)(mN)(fN)(mN)(mN)(mN)(mN)(mN)(mN)(mN)(fN)# |
| | (mN)#(fN)#(mN)#(mN)#(mN)#(but)#(mN) |
| P2_b21_as | P(mN)#(fN)#(mN)(mN)(mN)(fN)(mN)(mN)(mN)(mN)(mN)(mN)(mN)(fN)# |
| | (mN)#(fN)#(mN)#(mN)#(mN)#(fN)#(but) |
| as_TW_ | P(mN)#(fN)#(mN)(mN)(mN)(fN)(mN)(mN)(mN)(mN)(mN)(mN)(mN)(fN)# |
| But1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But2 | (mN)#(fN)#(but)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But3 | (mN)#(fN)#(mN)#(but)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But4 | (mN)#(fN)#(mN)#(mN)#(but)#(mN)#(mN) |
| as_TW_ | P(mN)#(fN)#(mN)(mN)(mN)(fN)(mN)(mN)(mN)(mN)(mN)(mN)(mN)(fN)# |
| But5 | (mN)#(fN)#(mN)#(mN)#(mN)#(but)#(mN) |
| as_TW_ | P(mN)#(fN)#(mN)(mN)(mN)(fN)(mN)(mN)(mN)(mN)(mN)(mN)(mN)(fN)# |
| But6 | (mN)#(fN)#(mN)#(mN)#(mN)#(mN)#(but) |
| as_TW_ | P(mN)#(fN)#(mN)(mN)(mN)(fN)(mN)(mN)(mN)(mN)(mN)(mN)(mN)(fN)# |
| But7 | (mN)#(fN)#(but)#(but)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But8 | (mN)#(fN)#(mN)#(but)#(but)#(mN)#(mN) |
| as_TW_ | P(mN)#(fN)#(mN)(mN)(mN)(fN)(mN)(mN)(mN)(mN)(mN)(mN)(mN)(fN)# |
| But9 | (mN)#(fN)#(mN)#(mN)#(but)#(but)#(mN) |
| as_TW_ | P(mN)#(fN)#(mN)(mN)(mN)(fN)(mN)(mN)(mN)(mN)(mN)(mN)(mN)(fN)# |
| But10 | (mN)#(fN)#(mN)#(mN)#(mN)#(but)#(but) |
| as_TW_ | P(mN)#(fN)#(mN)(mN)(mN)(fN)(mN)(mN)(mN)(mN)(mN)(mN)(mN)(fN)# |
| But11 | (mN)#(fN)#(but)#(mN)#(but)#(mN)#(mN) |
| as_TW_ | P(mN)#(fN)#(mN)(mN)(mN)(fN)(mN)(mN)(mN)(mN)(mN)(mN)(mN)(fN)# |
| But12 | (mN)#(fN)#(mN)#(mN)#(but)#(mN)#(but) |
| as_TW_ | P(mN)#(fN)#(mN)(mN)(mN)(fN)(mN)(mN)(mN)(mN)(mN)(mN)(mN)(fN)# |
| But13 | (mN)#(fN)#(mN)#(but)#(mN)#(but)#(mN) |
| as_TW_ | P(mN)#(fN)#(mN)(mN)(mN)(fN)(mN)(mN)(mN)(mN)(mN)(mN)(mN)(fN)# |
| But14 | (mN)#(fN)#(but)#(but)#(but)#(mN)#(mN) |
| as_TW_ | P(mN)#(fN)#(mN)(mN)(mN)(fN)(mN)(mN)(mN)(mN)(mN)(mN)(mN)(fN)# |
| But15 | (mN)#(fN)#(mN)#(mN)#(but)#(but)#(but) |
| as_TW_ | P(mN)#(fN)#(mN)(mN)(mN)(fN)(mN)(mN)(mN)(mN)(mN)(mN)(mN)(fN)# |
| But16 | (mN)#(fN)#(but)#(but)#(but)#(but)#(but) |
| P1_ib1_as | P(ibut)(mN)#(fN)#(mN)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib2_as | P(mN)#(ibut)(fN)#(mN)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib3_as | P(mN)#(fN)#(ibut)(mN)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib4_as | P(mN)#(fN)#(mN)(ibut)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib5_as | P(mN)#(fN)#(mN)(fN)(ibut)(fN)(fN)(mN)(fN)(mN)(fN)(mN)(fN)(mN)(fN)# |
| | (mN)#(fN)#(mN)#(mN)#(mN)#(fN)#(mN) |
| P1_ib6_as | P(mN)#(fN)#(mN)(fN)(fN)(ibut)(fN)(mN)(fN)(mN)(fN)(mN)(fN)(mN)(fN)# |
| | (mN)#(fN)#(mN)#(mN)#(mN)#(fN)#(mN) |
| P1_ib7_as | P(mN)#(fN)#(mN)(fN)(fN)(fN)(ibut)(mN)(fN)(mN)(fN)(mN)(fN)(mN)(fN)# |
| | (mN)#(fN)#(mN)#(mN)#(mN)#(fN)#(mN) |
| P1_ib8_as | P(mN)#(fN)#(mN)(fN)(fN)(fN)(mN)(ibut)(fN)(mN)(fN)(mN)(fN)(mN)(fN)# |
| | (mN)#(fN)#(mN)#(mN)#(mN)#(fN)#(mN) |
| P1_ib9_as | P(mN)#(fN)#(mN)(fN)(fN)(fN)(mN)(fN)(ibut)(mN)(fN)(mN)(fN)(mN)(fN)# |
| | (mN)#(fN)#(mN)#(mN)#(mN)#(fN)#(mN) |
| P1_ib10_ | P(mN)#(fN)#(mN)(fN)(fN)(fN)(mN)(fN)(mN)(ibut)(fN)(mN)(fN)(mN)(fN)# |
| as | (mN)#(fN)#(mN)#(mN)#(mN)#(fN)#(mN) |
| P1_ib11_ | P(mN)#(fN)#(mN)(fN)(fN)(fN)(mN)(fN)(mN)(fN)(ibut)(mN)(fN)(mN)(fN)# |
| as | (mN)#(fN)#(mN)#(mN)#(mN)#(fN)#(mN) |
| P1_ib12_ | P(mN)#(fN)#(mN)(fN)(fN)(fN)(mN)(fN)(mN)(fN)(mN)(ibut)(fN)(mN)(fN)# |
| as | (mN)#(fN)#(mN)#(mN)#(mN)#(fN)#(mN) |
| P1_ib13_ | P(mN)#(fN)#(mN)(fN)(fN)(fN)(mN)(fN)(mN)(fN)(mN)(fN)(ibut)(mN)(fN)# |
| as | (mN)#(fN)#(mN)#(mN)#(mN)#(fN)#(mN) |
| P1_ib14_ | P(mN)#(fN)#(mN)(fN)(fN)(fN)(mN)(fN)(mN)(fN)(mN)(fN)(mN)(ibut)(fN)# |
| as | (mN)#(fN)#(mN)#(mN)#(mN)#(fN)#(mN) |
| P1_ib15_ | P(mN)#(fN)#(mN)(fN)(fN)(fN)(mN)(fN)(mN)(fN)(mN)(fN)(mN)(fN)#(ibut) |
| as | (mN)#(fN)#(mN)#(mN)#(mN)#(fN)#(mN) |
| P1_ib16_ | P(mN)#(fN)#(mN)(fN)(fN)(fN)(mN)(fN)(mN)(fN)(mN)(fN)(mN)(fN)#(mN)# |
| as | (ibut)(fN)#(mN)#(mN)#(mN)#(fN)#(mN) |
| P1_ib17_ | P(mN)#(fN)#(mN)(fN)(fN)(fN)(mN)(fN)(mN)(fN)(mN)(fN)(mN)(fN)#(mN)# |
| as | (fN)#(ibut)(mN)#(mN)#(mN)#(fN)#(mN) |
| P1_ib18_ | P(mN)#(fN)#(mN)(fN)(fN)(fN)(mN)(fN)(mN)(fN)(mN)(fN)(mN)(fN)#(mN)# |
| as | (fN)#(mN)#(ibut)(mN)#(mN)#(fN)#(mN) |
| P1_ib19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(ibut)(mN)#(fN)#(mN) |
| P1_ib20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(ibut)(fN)#(mN) |
| P1_ib21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(ibut)(mN) |
| P1_ib22_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(mN)(ibut) |
| P2_ib1_as | P(ibut)(mN)#(fN)#(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib2_as | P(mN)#(ibut)(fN)#(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib3_as | P(mN)#(fN)#(ibut)(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib4_as | P(mN)#(fN)#(mN)(ibut)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib5_as | P(mN)#(fN)#(mN)(mN)(ibut)(mN)(fN)(mN)(mN)(mN)(mN)(mN)(mN)(mN)(fN) |
| | #(mN)#(fN)#(mN)#(mN)#(mN)#(fN)#(mN) |
| P2_ib6_as | P(mN)#(fN)#(mN)(mN)(mN)(ibut)(fN)(mN)(mN)(mN)(mN)(mN)(mN)(mN)(fN) |
| | #(mN)#(fN)#(mN)#(mN)#(mN)#(fN)#(mN) |
| P2_ib7_as | P(mN)#(fN)#(mN)(mN)(mN)(fN)(ibut)(mN)(mN)(mN)(mN)(mN)(mN)(mN)(fN) |
| | #(mN)#(fN)#(mN)#(mN)#(mN)#(fN)#(mN) |
| P2_ib8_as | P(mN)#(fN)#(mN)(mN)(mN)(fN)(mN)(ibut)(mN)(mN)(mN)(mN)(mN)(mN)(fN) |
| | #(mN)#(fN)#(mN)#(mN)#(mN)#(fN)#(mN) |
| P2_ib9_as | P(mN)#(fN)#(mN)(mN)(mN)(fN)(mN)(mN)(ibut)(mN)(mN)(mN)(mN)(mN)(fN) |
| | #(mN)#(fN)#(mN)#(mN)#(mN)#(fN)#(mN) |
| P2_ib10_ | P(mN)#(fN)#(mN)(mN)(mN)(fN)(mN)(mN)(mN)(ibut)(mN)(mN)(mN)(mN)(fN) |
| as | #(mN)#(fN)#(mN)#(mN)#(mN)#(fN)#(mN) |
| P2_ib11_ | P(mN)#(fN)#(mN)(mN)(mN)(fN)(mN)(mN)(mN)(mN)(ibut)(mN)(mN)(mN)(fN) |
| as | #(mN)#(fN)#(mN)#(mN)#(mN)#(fN)#(mN) |
| P2_ib12_ | P(mN)#(fN)#(mN)(mN)(mN)(fN)(mN)(mN)(mN)(mN)(mN)(ibut)(mN)(mN)(fN) |
| as | #(mN)#(fN)#(mN)#(mN)#(mN)#(fN)#(mN) |
| P2_ib13_ | P(mN)#(fN)#(mN)(mN)(mN)(fN)(mN)(mN)(mN)(mN)(mN)(mN)(ibut)(mN)(fN) |
| as | #(mN)#(fN)#(mN)#(mN)#(mN)#(fN)#(mN) |
| P2_ib14_ | P(mN)#(fN)#(mN)(mN)(mN)(fN)(mN)(mN)(mN)(mN)(mN)(mN)(mN)(ibut)(fN) |
| as | #(mN)#(fN)#(mN)#(mN)#(mN)#(fN)#(mN) |
| P2_ib15_ | P(mN)#(fN)#(mN)(mN)(mN)(fN)(mN)(mN)(mN)(mN)(mN)(mN)(mN)(fN) |
| as | (ibut)#(mN)#(fN)#(mN)#(mN)#(mN)#(fN)#(mN) |
| P2_ib16_ | P(mN)#(fN)#(mN)(mN)(mN)(fN)(mN)(mN)(mN)(mN)(mN)(mN)(mN)(fN)# |
| as | (mN)(ibut)#(fN)#(mN)#(mN)#(mN)#(fN)#(mN) |
| P2_ib17_ | P(mN)#(fN)#(mN)(mN)(mN)(fN)(mN)(mN)(mN)(mN)(mN)(mN)(mN)(fN)# |
| as | (mN)#(fN)(ibut)#(mN)#(mN)#(mN)#(fN)#(mN) |
| P2_ib18_ | P(mN)#(fN)#(mN)(mN)(mN)(fN)(mN)(mN)(mN)(mN)(mN)(mN)(mN)(fN)# |
| as | (mN)#(fN)#(mN)(ibut)#(mN)#(mN)#(fN)#(mN) |
| P2_ib19_ | P(mN)#(fN)#(mN)(mN)(mN)(fN)(mN)(mN)(mN)(mN)(mN)(mN)(mN)(fN)# |
| as | (mN)#(fN)#(mN)#(mN)#(ibut)(mN)#(fN)#(mN) |
| P2_ib20_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(ibut)(fN)#(mN) |
| P2_ib21_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(ibut)(mN) |
| P2_ib22_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN)(ibut) |
| as_P5_ | P(mN)#(fN)#(mN)(mN)(mN)(fN)(mN)(mN)(mN)(mN)(mN)(mN)(mN)(fN)# |
| TW_iBut1 | (mN)#(fN)#(mN)#(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut2 | (mN)#(fN)#(ibut)(mN)#(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut3 | (mN)#(fN)#(mN)#(ibut)(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut4 | (mN)#(fN)#(mN)#(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut5 | (mN)#(fN)#(mN)#(mN)#(mN)#(ibut)(mN)#(mN) |
| as_P5_ | P(mN)#(fN)#(mN)(mN)(mN)(fN)(mN)(mN)(mN)(mN)(mN)(mN)(mN)(fN)# |
| TW_iBut6 | (mN)#(fN)#(mN)#(mN)#(mN)#(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut7 | (mN)#(fN)#(ibut)(mN)#(ibut)(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut8 | (mN)#(fN)#(mN)#(ibut)(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut9 | (mN)#(fN)#(mN)#(mN)#(ibut)(mN)#(ibut)(mN)#(mN) |
| as_P5_ | P(mN)#(fN)#(mN)(mN)(mN)(fN)(mN)(mN)(mN)(mN)(mN)(mN)(mN)(fN)# |
| TW_iBut10 | (mN)#(fN)#(mN)#(mN)#(mN)#(ibut)(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut11 | (mN)#(fN)#(ibut)(mN)#(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut12 | (mN)#(fN)#(mN)#(mN)#(ibut)(mN)#(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut13 | (mN)#(fN)#(mN)#(ibut)(mN)#(mN)#(ibut)(mN)#(mN) |
| as_P5_ | P(mN)#(fN)#(mN)(mN)(mN)(fN)(mN)(mN)(mN)(mN)(mN)(mN)(mN)(fN)# |
| TW_iBut14 | (mN)#(fN)#(ibut)(mN)#(ibut)(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut15 | (mN)#(fN)#(mN)#(mN)#(ibut)(mN)#(ibut)(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut16 | (mN)#(fN)#(ibut)(mN)#(ibut)(mN)#(ibut)(mN)#(ibut)(mN)#(ibut)(mN) |

In certain embodiments, the dsRNA comprises an sense strand with one of the following chemical modification patterns:

| | |
|---|---|
| P1_b1_s | (but)#(mN)#(mN)(fN)(mN)(fN)(mN)(fN)(mN)(fN)(mN)(mN)(mN)(fN)#(mN)# |
| | (mN) |
| P1_b2_s | (mN)#(but)#(mN)(fN)(mN)(fN)(mN)(fN)(mN)(fN)(mN)(mN)(mN)(fN)#(mN)# |
| | (mN) |
| P1_b3_s | (mN)#(mN)#(but)(fN)(mN)(fN)(mN)(fN)(mN)(fN)(mN)(mN)(mN)(fN)#(mN)# |
| | (mN) |
| P1_b4_s | (mN)#(mN)#(mN)(but)(mN)(fN)(mN)(fN)(mN)(fN)(mN)(mN)(mN)(fN)#(mN) |
| | #(mN) |
| P1_b5_s | (mN)#(mN)#(mN)(fN)(but)(fN)(mN)(fN)(mN)(fN)(mN)(mN)(mN)(fN)#(mN)# |
| | (mN) |
| P1_b6_s | (mN)#(mN)#(mN)(fN)(mN)(but)(mN)(fN)(mN)(fN)(mN)(mN)(mN)(fN)#(mN) |
| | #(mN) |
| P1_b7_s | (mN)#(mN)#(mN)(fN)(mN)(fN)(but)(fN)(mN)(fN)(mN)(mN)(mN)(fN)#(mN)# |
| | (mN) |
| P1_b8_s | (mN)#(mN)#(mN)(fN)(mN)(fN)(mN)(but)(mN)(fN)(mN)(mN)(mN)(fN)#(mN) |
| | #(mN) |
| P1_b9_s | (mN)#(mN)#(mN)(fN)(mN)(fN)(mN)(fN)(but)(fN)(mN)(mN)(mN)(fN)#(mN)# |
| | (mN) |
| P1_b10_ | #(mN)#(mN)#(mN)#(mN)(fN)(mN)(fN)(mN)(fN)(mN)(but)(mN)(mN)(mN)(fN) |
| s | #(mN) |
| P1_b11_ | (mN)#(mN)#(mN)(fN)(mN)(fN)(mN)(fN)(mN)(fN)(but)(mN)(mN)(fN)#(mN)# |
| s | (mN) |
| P1_b12_ | (mN)#(mN)#(mN)(fN)(mN)(fN)(mN)(fN)(mN)(fN)(mN)(but)(mN)(fN)#(mN)# |
| s | (mN) |
| P1_b13_ | (mN)#(mN)#(mN)(fN)(mN)(fN)(mN)(fN)(mN)(fN)(mN)(mN)(but)(fN)#(mN)# |
| s | (mN) |
| P1_b14_ | (mN)#(mN)#(mN)(fN)(mN)(fN)(mN)(fN)(mN)(fN)(mN)(mN)(mN)(but)#(mN) |
| s | #(mN) |
| P1_b15_ | (mN)#(mN)#(mN)(fN)(mN)(fN)(mN)(fN)(mN)(fN)(mN)(mN)(mN)(fN)#(but)# |
| s | (mN) |
| P1_b16_ | (mN)#(mN)#(mN)(fN)(mN)(fN)(mN)(fN)(mN)(fN)(mN)(mN)(mN)(fN)#(mN) |
| s | #(but) |
| P2_b1_s | (but)#(mN)#(mN)(mN)(mN)(fN)(fN)(fN)(mN)(fN)(mN)(mN)(mN)(mN)#(mN) |
| | #(mN) |
| P2_b2_s | (mN)#(but)#(mN)(mN)(mN)(fN)(fN)(fN)(mN)(fN)(mN)(mN)(mN)(mN)#(mN) |
| | #(mN) |
| P2_b3_s | (mN)#(mN)#(but)(mN)(mN)(fN)(fN)(fN)(mN)(fN)(mN)(mN)(mN)(mN)#(mN) |
| | #(mN) |
| P2_b4_s | (mN)#(mN)#(mN)(but)(mN)(fN)(fN)(fN)(mN)(fN)(mN)(mN)(mN)(mN)#(mN) |
| | #(mN) |
| P2_b5_s | (mN)#(mN)#(mN)(mN)(but)(fN)(fN)(fN)(mN)(fN)(mN)(mN)(mN)(mN)#(mN) |
| | #(mN) |
| P2_b6_s | (mN)#(mN)#(mN)(mN)(mN)(but)(fN)(fN)(mN)(fN)(mN)(mN)(mN)(mN)#(mN) |
| | #(mN) |
| P2_b7_s | (mN)#(mN)#(mN)(mN)(mN)(fN)(but)(fN)(mN)(fN)(mN)(mN)(mN)(mN)#(mN) |
| | #(mN) |
| P2_b8_s | (mN)#(mN)#(mN)(mN)(mN)(fN)(fN)(but)(mN)(fN)(mN)(mN)(mN)(mN)#(mN) |
| | #(mN) |
| P2_b9_s | (mN)#(mN)#(mN)(mN)(mN)(fN)(fN)(fN)(but)(fN)(mN)(mN)(mN)(mN)#(mN) |
| | #(mN) |
| P2_b10_ | (mN)#(mN)#(mN)(mN)(mN)(fN)(fN)(fN)(mN)(but)(mN)(mN)(mN)(mN)#(mN) |
| s | #(mN) |
| P2_b11_ | (mN)#(mN)#(mN)(mN)(mN)(fN)(fN)(fN)(mN)(fN)(but)(mN)(mN)(mN)#(mN) |
| s | #(mN) |
| P2_b12_ | (mN)#(mN)#(mN)(mN)(mN)(fN)(fN)(fN)(mN)(fN)(mN)(but)(mN)(mN)#(mN) |
| s | #(mN) |
| P2_b13_ | (mN)#(mN)#(mN)(mN)(mN)(fN)(fN)(fN)(mN)(fN)(mN)(mN)(but)(mN)#(mN) |
| s | #(mN) |
| P2_b14_ | (mN)#(mN)#(mN)(mN)(mN)(fN)(fN)(fN)(mN)(fN)(mN)(mN)(mN)(but)#(mN) |
| s | #(mN) |
| P2_b15_ | (mN)#(mN)#(mN)(mN)(mN)(fN)(fN)(fN)(mN)(fN)(mN)(mN)(mN)(mN)#(but) |
| s | #(mN) |
| P2_b16_ | (mN)#(mN)#(mN)(mN)(mN)(fN)(fN)(fN)(mN)(fN)(mN)(mN)(mN)(mN)#(mN) |
| s | #(but) |
| P1_ib1_ | (ibut)(mN)#(mN)#(mN)(fN)(mN)(fN)(mN)(fN)(mN)(fN)(mN)(mN)(mN)(fN)# |
| s | (mN)#(mN) |
| P1_ib2_ | (mN)#(ibut)(mN)#(mN)(fN)(mN)(fN)(mN)(fN)(mN)(fN)(mN)(mN)(mN)(fN)# |
| s | (mN)#(mN) |
| P1_ib3_ | (mN)#(mN)#(ibut)(mN)(fN)(mN)(fN)(mN)(fN)(mN)(fN)(mN)(mN)(mN)(fN)# |
| s | (mN)#(mN) |
| P1_ib4_ | (mN)#(mN)#(mN)(ibut)(fN)(mN)(fN)(mN)(fN)(mN)(fN)(mN)(mN)(mN)(fN)# |
| s | (mN)#(mN) |
| P1_ib5_ | (mN)#(mN)#(mN)(fN)(ibut)(mN)(fN)(mN)(fN)(mN)(fN)(mN)(mN)(mN)(fN)# |
| s | (mN)#(mN) |
| P1_ib6_ | (mN)#(mN)#(mN)(fN)(mN)(ibut)(fN)(mN)(fN)(mN)(fN)(mN)(mN)(mN)(fN)# |
| s | (mN)#(mN) |
| P1_ib7_ | (mN)#(mN)#(mN)(fN)(mN)(fN)(ibut)(mN)(fN)(mN)(fN)(mN)(mN)(mN)(fN)# |
| s | (mN)#(mN) |
| P1_ib8_ | (mN)#(mN)#(mN)(fN)(mN)(fN)(mN)(ibut)(fN)(mN)(fN)(mN)(mN)(mN)(fN)# |
| s | (mN)#(mN) |
| P1_ib9_ | (mN)#(mN)#(mN)(fN)(mN)(fN)(mN)(fN)(ibut)(mN)(fN)(mN)(mN)(mN)(fN)# |
| s | (mN)#(mN) |
| P1_ib10_ | (mN)#(mN)#(mN)(fN)(mN)(fN)(mN)(fN)(mN)(ibut)(fN)(mN)(mN)(mN)(fN)# |
| s | (mN)#(mN) |
| P1_ib11_ | (mN)#(mN)#(mN)(fN)(mN)(fN)(mN)(fN)(mN)(fN)(ibut)(mN)(mN)(mN)(fN)# |
| s | (mN)#(mN) |
| P1_ib12_ | (mN)#(mN)#(mN)(fN)(mN)(fN)(mN)(fN)(mN)(fN)(mN)(ibut)(mN)(mN)(fN)# |
| s | (mN)#(mN) |
| P1_ib13_ | (mN)#(mN)#(mN)(fN)(mN)(fN)(mN)(fN)(mN)(fN)(mN)(mN)(ibut)(mN)(fN)# |
| s | (mN)#(mN) |
| P1_ib14_ | (mN)#(mN)#(mN)(fN)(mN)(fN)(mN)(fN)(mN)(fN)(mN)(mN)(mN)(ibut)(fN)# |
| s | (mN)#(mN) |
| P1_ib15_ | (mN)#(mN)#(mN)(fN)(mN)(fN)(mN)(fN)(mN)(fN)(mN)(mN)(mN)(fN)#(ibut) |
| s | (mN)#(mN) |
| P1_ib16_ | (mN)#(mN)#(mN)(fN)(mN)(fN)(mN)(fN)(mN)(fN)(mN)(mN)(mN)(fN)#(mN) |
| s | #(ibut)(mN) |
| P1_ib17_ | (mN)#(mN)#(mN)(fN)(mN)(fN)(mN)(fN)(mN)(fN)(mN)(mN)(mN)(fN)#(mN) |
| s | #(mN)(ibut) |
| P2_ib1_ | (ibut)(mN)#(mN)#(mN)(mN)(mN)(fN)(fN)(fN)(mN)(fN)(mN)(mN)(mN)(mN) |
| s | #(mN)#(mN) |
| P2_ib2_ | (mN)(ibut)#(mN)#(mN)(mN)(mN)(fN)(fN)(fN)(mN)(fN)(mN)(mN)(mN)(mN) |
| s | #(mN)#(mN) |
| P2_ib3_ | (mN)#(mN)(ibut)#(mN)(mN)(mN)(fN)(fN)(fN)(mN)(fN)(mN)(mN)(mN)(mN) |
| s | #(mN)#(mN) |
| P2_ib4_ | (mN)#(mN)#(mN)(ibut)(mN)(mN)(fN)(fN)(fN)(mN)(fN)(mN)(mN)(mN)(mN) |
| s | #(mN)#(mN) |
| P2_ib5_ | (mN)#(mN)#(mN)(mN)(ibut)(mN)(fN)(fN)(fN)(mN)(fN)(mN)(mN)(mN)(mN) |
| s | #(mN)#(mN) |
| P2_ib6_ | (mN)#(mN)#(mN)(mN)(mN)(ibut)(fN)(fN)(fN)(mN)(fN)(mN)(mN)(mN)(mN) |
| s | #(mN)#(mN) |
| P2_ib7_ | (mN)#(mN)#(mN)(mN)(mN)(fN)(ibut)(fN)(fN)(mN)(fN)(mN)(mN)(mN)(mN) |
| s | #(mN)#(mN) |
| P2_ib8_ | (mN)#(mN)#(mN)(mN)(mN)(fN)(fN)(ibut)(fN)(mN)(fN)(mN)(mN)(mN)(mN) |
| s | #(mN)#(mN) |
| P2_ib9_ | (mN)#(mN)#(mN)(mN)(mN)(fN)(fN)(fN)(ibut)(mN)(fN)(mN)(mN)(mN)(mN) |
| s | #(mN)#(mN) |
| P2_ib10_ | (mN)#(mN)#(mN)(mN)(mN)(fN)(fN)(fN)(mN)(ibut)(fN)(mN)(mN)(mN)(mN) |
| s | #(mN)#(mN) |
| P2_ib11_ | (mN)#(mN)#(mN)(mN)(mN)(fN)(fN)(fN)(mN)(fN)(ibut)(mN)(mN)(mN)(mN) |
| s | #(mN)#(mN) |
| P2_ib12_ | (mN)#(mN)#(mN)(mN)(mN)(fN)(fN)(fN)(mN)(fN)(mN)(ibut)(mN)(mN)(mN) |
| s | #(mN)#(mN) |
| P2_ib13_ | (mN)#(mN)#(mN)(mN)(mN)(fN)(fN)(fN)(mN)(fN)(mN)(mN)(ibut)(mN)(mN) |
| s | #(mN)#(mN) |
| P2_ib14_ | (mN)#(mN)#(mN)(mN)(mN)(fN)(fN)(fN)(mN)(fN)(mN)(mN)(mN)(ibut)(mN) |
| s | #(mN)#(mN) |
| P2_ib15_ | (mN)#(mN)#(mN)(mN)(mN)(fN)(fN)(fN)(mN)(fN)(mN)(mN)(mN)(mN)(ibut) |
| s | #(mN)#(mN) |
| P2_ib16_ | (mN)#(mN)#(mN)(mN)(mN)(fN)(fN)(fN)(mN)(fN)(mN)(mN)(mN)(mN)#(mN) |
| s | #(ibut)(mN) |
| P2_ib17_ | (mN)#(mN)#(mN)(mN)(mN)(fN)(fN)(fN)(mN)(fN)(mN)(mN)(mN)(mN)#(mN) |
| s | #(mN)(ibut) |

In another aspect, the disclosure provides a double stranded (ds) RNA, comprising an antisense strand and a sense strand, each strand with a 5′ end and a 3′ end, and at least one single stranded nucleotide overhang of 2-5 nucleotides, wherein the single stranded nucleotide overhang comprises at least two nucleotide modifications selected from the group consisting of a 2′-deoxy modification, a 2′-MOE modification, an LNA modification, a UNA modification, and an alkyl modification.

In certain embodiments, the single stranded nucleotide overhang comprises 2, 3, 4, or 5 nucleotide modifications selected from the group consisting of a 2′-deoxy modification, a 2′-MOE modification, an LNA modification, a UNA modification, and an alkyl modification.

In certain embodiments, each nucleotide in the single stranded nucleotide overhang comprises the same nucleotide modification. In certain embodiments, the single stranded nucleotide overhang comprises at least two different nucleotide modifications.

In another aspect, the disclosure provides a double stranded (ds) RNA, comprising an antisense strand and a sense strand, each strand with a 5′ end and a 3′ end, wherein the antisense strand comprises a chemical modification pattern of any one of the chemical modification patterns provided in Tables 1-8.

In another aspect, the disclosure provides a double stranded (ds) RNA, comprising an antisense strand and a sense strand, each strand with a 5′ end and a 3′ end, wherein the sense strand comprises a chemical modification pattern of any one of the chemical modification patterns provided in Tables 1-8.

In another aspect, the disclosure provides a method for reducing the expression of a target mRNA in a subject, comprising administering to the subject the RNA molecule or the dsRNA described above, thereby reducing the expression of the target mRNA.

In certain embodiments, the expression of the target mRNA is reduced by at least about 20%, at least about 30%, at least about 40%, or at least about 50% over an expression level prior to administration of the RNA molecule or dsRNA.

In certain embodiments, the expression of the target mRNA is reduced for at least about 3 months, at least about 4 months, at least about 5 months, at least about 6 months, at least about 7 months, at least about 8 months, at least about 9 months, at least about 10 months, at least about 11 months, or at least about 12 months after administration of the RNA molecule or dsRNA.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts chemical modifications and two siRNA chemical modification patterns (Pattern 1 and Pattern 2) used in this disclosure as baseline modification patterns to which the alternative modifications are applied.

FIG. 2 depicts the alternative chemical modifications applied in this disclosure to Pattern 1 and Pattern 2.

FIG. 3 depicts relative MECP2 and HTT mRNA levels in cells incubated with various chemically modified siRNA. The siRNA contain a butane modification (replacement of whole nucleotide) or UNA modification at the recited positions in the antisense strand of Pattern 2. Cells were treated with 0.5 μM siRNA and mRNA levels were measured 72 hours later.

FIG. 4 depicts relative MECP2 and HTT mRNA levels in cells incubated with various chemically modified siRNA. The siRNA contain a butane modification (linked between two nucleotides) or a mismatch at the recited positions in the antisense strand of Pattern 2. Cells were treated with 0.5 μM siRNA and mRNA levels were measured 72 hours later.

FIG. 5 depicts relative MECP2 and HTT mRNA levels in cells incubated with various chemically modified siRNA. The siRNA contain an LNA modification or a 2′-MOE modification at the recited positions in the antisense strand of Pattern 2. Cells were treated with 0.5 μM siRNA and mRNA levels were measured 72 hours later.

FIG. 6 depicts relative MECP2 and HTT mRNA levels in cells incubated with various chemically modified siRNA. The siRNA contain a unmodified RNA or a DNA modification at the recited positions in the antisense strand of Pattern 2. Cells were treated with 0.5 μM siRNA and mRNA levels were measured 72 hours later.

FIG. 7 depicts relative MECP2 and HTT mRNA levels in cells incubated with various chemically modified siRNA. The siRNA contain a butane modification (replacement of whole nucleotide) or UNA modification at the recited positions in the sense strand of Pattern 2. Cells were treated with 0.5 μM siRNA and mRNA levels were measured 72 hours later.

FIG. 8 depicts relative MECP2 and HTT mRNA levels in cells incubated with various chemically modified siRNA. The siRNA contain a butane modification (linked between two nucleotides) or a mismatch at the recited positions in the sense strand of Pattern 2. Cells were treated with 0.5 μM siRNA and mRNA levels were measured 72 hours later.

FIG. 9 depicts relative MECP2 and HTT mRNA levels in cells incubated with various chemically modified siRNA. The siRNA contain an LNA modification or a 2′-MOE modification at the recited positions in the sense strand of Pattern 2. Cells were treated with 0.5 μM siRNA and mRNA levels were measured 72 hours later.

FIG. 10 depicts relative MECP2 and HTT mRNA levels in cells incubated with various chemically modified siRNA. The siRNA contain a DNA modification at the recited positions in the sense strand of Pattern 2. Cells were treated with 0.5 μM siRNA and mRNA levels were measured 72 hours later.

FIG. 11 depicts relative MECP2 and HTT mRNA levels in cells incubated with various chemically modified siRNA. The siRNA contain a UNA modification at the recited positions in the antisense strand of Pattern 1. Cells were treated with 0.5 μM siRNA and mRNA levels were measured 72 hours later.

FIG. 12 depicts relative MECP2 and HTT mRNA levels in cells incubated with various chemically modified siRNA. The siRNA contain a butane modification (linked between two nucleotides) or a mismatch at the recited positions in the antisense strand of Pattern 1. Cells were treated with 0.5 μM siRNA and mRNA levels were measured 72 hours later.

FIG. 13 depicts relative MECP2 and HTT mRNA levels in cells incubated with various chemically modified siRNA. The siRNA contain an LNA modification or a 2′-MOE modification at the recited positions in the antisense strand of Pattern 1. Cells were treated with 0.5 μM siRNA and mRNA levels were measured 72 hours later.

FIG. 14 depicts relative MECP2 and HTT mRNA levels in cells incubated with various chemically modified siRNA. The siRNA contain a unmodified RNA or a DNA modification at the recited positions in the antisense strand of Pattern 1. Cells were treated with 0.5 μM siRNA and mRNA levels were measured 72 hours later.

FIG. 15A-FIG. 15D depict relative MECP2 and HTT mRNA levels in cells incubated with various chemically modified siRNA at different doses. The siRNA contain a butane modification (replacement of whole nucleotide) or UNA modification at the recited positions in the sense strand of Pattern 1. Cells were treated with 0.5 μM siRNA (FIG. 15A) or otherwise indicated (FIG. 15B-FIG. 15D), and mRNA levels were measured 72 hours later.

FIG. 16A-FIG. 16I depict relative MECP2 and HTT mRNA levels in cells incubated with various chemically modified siRNA at different doses. The siRNA contain a butane modification (linked between two nucleotides) or a mismatch at the recited positions in the sense strand of Pattern 1. Cells were treated with 0.5 μM siRNA (FIG. 16A) or otherwise indicated (FIG. 16B-FIG. 16I), and mRNA levels were measured 72 hours later.

FIG. 17 depicts relative MECP2 and HTT mRNA levels in cells incubated with various chemically modified siRNA. The siRNA contain an LNA modification or a 2′-MOE modification at the recited positions in the sense strand of Pattern 1. Cells were treated with μM siRNA and mRNA levels were measured 72 hours later.

FIG. 18 depicts relative MECP2 and HTT mRNA levels in cells incubated with various chemically modified siRNA. The siRNA contain a DNA modification at the recited positions in the sense strand of Pattern 1. Cells were treated with 0.5 μM siRNA and mRNA levels were measured 72 hours later.

FIG. 19 depicts the alternative chemical modifications applied in this disclosure to siRNA tail region of Pattern 1T and Pattern 2T.

FIG. 20 depicts relative MECP2 and HTT mRNA levels in cells incubated with various chemically modified siRNA. The siRNA contain a butane modification (replacement of whole nucleotide) or UNA modification at the recited positions in the antisense strand tail of Pattern 2T. Cells were treated with 0.5 μM siRNA and mRNA levels were measured 72 hours later.

FIG. 21 depicts relative MECP2 and HTT mRNA levels in cells incubated with various chemically modified siRNA. The siRNA contain an LNA modification at the recited positions in the antisense strand tail of Pattern 2T. Cells were treated with 0.5 μM siRNA and mRNA levels were measured 72 hours later.

FIG. 22 depicts relative MECP2 and HTT mRNA levels in cells incubated with various chemically modified siRNA. The siRNA contain an unmodified RNA at the recited positions in the antisense strand tail of Pattern 2T. Cells were treated with 0.5 μM siRNA and mRNA levels were measured 72 hours later.

FIG. 23A and FIG. 23B depicts relative MECP2 and HTT mRNA levels in cells incubated with various chemically modified siRNA. The siRNA contain a butane (replacement of whole nucleotide), UNA, or 2′-F modification at the recited positions in the antisense strand tail of Pattern 1T. Cells were treated with 0.5 μM siRNA and mRNA levels were measured 72 hours later.

FIG. 24 depicts relative MECP2 and HTT mRNA levels in cells incubated with various chemically modified siRNA. The siRNA contain a butane modification (linked between two nucleotides) at the recited positions in the antisense strand tail of Pattern 1T. Cells were treated with 0.5 μM siRNA and mRNA levels were measured 72 hours later.

FIG. 25A and FIG. 25B depict relative MECP2 and HTT mRNA levels in cells incubated with various chemically modified siRNA. The siRNA contain an LNA, 2′-MOE, butane (replacement of whole nucleotide), or UNA modification at the recited positions in the antisense strand tail of Pattern 1T. Cells were treated with 0.5 μM siRNA and mRNA levels were measured 72 hours later.

FIG. 26 depicts relative MECP2 and HTT mRNA levels in cells incubated with various chemically modified siRNA. The siRNA contain an unmodified RNA at the recited positions in the antisense strand tail of Pattern 1T. Cells were treated with 0.5 μM siRNA and mRNA levels were measured 72 hours later.

FIG. 27 depicts relative HTT mRNA levels in cells incubated with various chemically modified siRNA. The siRNA contain a 2′-F at the recited positions in the antisense strand tail of Pattern 1T. Cells were treated with 0.5 μM siRNA and mRNA levels were measured 72 hours later.

FIG. 28A to FIG. 28D depicts relative MECP2 and HTT mRNA levels in cells incubated with various chemically modified siRNA at different doses. The siRNA contain a butane modification (replacement of whole nucleotide) at the recited positions in the sense strand of Pattern 1.

FIG. 29A to FIG. 29D depicts relative MECP2 and HTT mRNA levels in cells incubated with various chemically modified siRNA at different doses. The siRNA contain a 2′-MOE modification at the recited positions in the antisense strand tail of Pattern 1T.

FIG. 30 shows a schematic of an in vivo assay to measure the MECP2 and HTT mRNA levels and guide-strand tissue accumulations in mice injected with various chemically modified siRNA. Five (5) FVB/NJ female mice were injected subcutaneously with 10 mg/kg or 20 mg/kg of chemically modified siRNA conjugated with DCA and containing 2′-MOE, 2′-OMe, or butane (replacement of whole nucleotide) modifications.

FIG. 31A to FIG. 31D depict relative MECP2 and HTT mRNA levels and guide-strand tissue accumulations in mice injected with various chemically modified siRNA. Five (5) FVB/NJ female mice were injected subcutaneously with 10 mg/kg or 20 mg/kg of chemically modified siRNA. The siRNA were conjugated with DCA and contained 2′-MOE, 2′-OMe, or butane (replacement of whole nucleotide) modifications. The mRNA levels and siRNA accumulations were measured from heart, muscle, and lung tissues.

FIG. 32 depicts relative HTT mRNA levels in cells incubated with various chemically modified siRNA at different doses. The siRNA contain 1 to 5 butane modifications (replacement of whole nucleotide). The specific chemical modification patterns by Oligo ID are recited in Table 9.

FIG. 33 depicts relative HTT mRNA levels in cells incubated with various chemically modified siRNA at different doses. The siRNA contain a butane modification (replacement of whole nucleotide) or C2 modification. The specific chemical modification patterns by Oligo ID are recited in Table 9.

FIG. 34 depicts relative HTT mRNA levels in cells incubated with various chemically modified siRNA at different doses. The siRNA contain a butane modification (replacement of whole nucleotide) or C6 modification. The specific chemical modification patterns by Oligo ID are recited in Table 9.

FIG. 35 depicts relative HTT mRNA levels in cells incubated with various chemically modified siRNA at different doses. The siRNA contain a butane modification (replacement of whole nucleotide) or C3 modification. The specific chemical modification patterns by Oligo ID are recited in Table 9.

FIG. 36 depicts relative HTT mRNA levels in cells incubated with various chemically modified siRNA at different doses. The siRNA contain a butane modification (replacement of whole nucleotide) or C10 modification. The specific chemical modification patterns by Oligo ID are recited in Table 9.

### DETAILED DESCRIPTION OF CERTAIN EXEMPLARY EMBODIMENTS

Unless otherwise specified, nomenclature used in connection with cell and tissue culture, molecular biology, immunology, microbiology, genetics and protein and nucleic acid chemistry and hybridization described herein are those well-known and commonly used in the art. In addition, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by an unmodified phosphodiester or chemically-modified intersubunit linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. The siRNA of the disclosure may be single stranded (i.e., a single antisense strand), or double stranded (i.e., an antisense strand and sense strand annealed together to form a duplex). The double stranded siRNA of the disclosure comprise an antisense strand with sufficient complementarity to a target mRNA to mediate silencing of said mRNA, and a sense strand with sufficient complementarity to the antisense strand to form a duplex. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions, which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a polymer of nucleotides and/or nucleotide analogs. Oligonucleotides include, but are not limited to, siRNAs, antisense oligonucleotides, miRNAs, ribozymes, and mRNA.

The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Examples of RNA analogues include, but are not limited to, sugar- and/or backbone-modified ribonucleotides and/or deoxyribonucleotides. Such alterations or modifications can further include the addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which contains a “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence,” e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g. promoter or enhancer elements) of a target gene. In other embodiments, the target polynucleotide sequence is a target mRNA encoded by a target gene.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g. an orthologue or paralogue) of the target gene.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, and dual-function oligonucleotides as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

As used herein, the term “dual functional oligonucleotide” refers to a RNA silencing agent having the formula T-L-μ, wherein T is an mRNA targeting moiety, L is a linking moiety, and μ is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA). As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like between said nucleotides (or nucleotide analogs).

As used herein, the “5′ end,” as in the 5′ end of an oligonucleotide (e.g., an antisense strand or a sense strand of an siRNA), refers to the 5′ terminal nucleotides, e.g., between one and about five nucleotides at the 5′ terminus of an oligonucleotide. In certain embodiments, the 5′ end of an oligonucleotide corresponds to the first five nucleotides of the oligonucleotide. In certain embodiments, the 5′ end of an oligonucleotide is the first nucleotide. In certain embodiments, the 5′ end of an oligonucleotide is the first two consecutive nucleotides. In certain embodiments, the 5′ end of an oligonucleotide is the first three consecutive nucleotides. In certain embodiments, the 5′ end of an oligonucleotide is the first four consecutive nucleotides. In certain embodiments, the 5′ end of an oligonucleotide is the first five consecutive nucleotides.

As used herein, the “3′ end,” as in the 3′ end of an oligonucleotide (e.g., an antisense strand or a sense strand of an siRNA), refers to the 3′ terminal nucleotides, e.g., of between one and about five nucleotides at the 3′ terminus of an oligonucleotide. In certain embodiments, the 3′ end of an oligonucleotide corresponds to the last five nucleotides of the oligonucleotide. In certain embodiments, the 3′ end of an oligonucleotide is the last nucleotide. In certain embodiments, the 3′ end of an oligonucleotide is the last two consecutive nucleotides. In certain embodiments, the 3′ end of an oligonucleotide is the last three consecutive nucleotides. In certain embodiments, the 3′ end of an oligonucleotide is the last four consecutive nucleotides. In certain embodiments, the 3′ end of an oligonucleotide is the last five consecutive nucleotides.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g., in the antisense strand, mRNA targeting moiety or miRNA recruiting moiety) which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

As used herein, the term “alkyl” refers to a saturated hydrocarbon group that may be straight-chained or branched. The term “Cn-m alkyl,” refers to an alkyl group having n to m carbon atoms. An alkyl group formally corresponds to an alkane with one C H bond replaced by the point of attachment of the alkyl group to the remainder of the compound. In some embodiments, the alkyl group contains from 1 to 10 carbon atoms, from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “heteroalkyl” refers to optionally substituted alkyl radicals which have one or more skeletal chain atoms selected from an atom other than carbon, e.g., oxygen, nitrogen (e.g. NH or Nalkyl), sulfur, phosphorus, silicon, or combinations thereof. In some embodiments, heteroalkyl refers to an alkyl group in which one of the skeletal atoms of the alkyl is oxygen. In some embodiments, heteroalkyl refers to an alkyl group in which one of the skeletal atoms of the alkyl is NH or Nalkyl. In some embodiments, heteroalkyl refers to an alkyl group in which one of the skeletal atoms of the alkyl is O or S. Exemplary heteroalkyl groups include, but are not limited to, —(CH₂)ₙO—CH₃, —(CH₂)ₙOCH(CH₃)₂, —CH(CH₃)O—(CH₂)ₙ—CH₃, —C(CH₃)₂O—CH₃, —(CH₂)ₙS—CH₃, —(CH₂)ₙSCH(CH₃)₂, —CH(CH₃)S—(CH₂)ₙ—CH₃, —CH(CH₃)SO₂—(CH₂)ₙ—CH₃, —C(CH₃)₂SO₂—CH₃, —CH₂NH—(C₁-C₆alkyl), —C(CH₃)₂NH—(C₁-C₆alkyl), —CH(CH₃)NH—(C₁-C₆alkyl)₂, In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —O—CH₂—CH₂—N(CH₃)—CH₃, —CH₂—S—CH₂—CH₃, —CH₂—CH₂—S(O)—CH₃, —CH₂—CH₂—S(O)₂—CH₃, —O—CH₂—CH₂—S(O)₂—CH₃, —CH═CH—O—CH₃, —Si(CH₃)₃, —CH₂—CH═N—OCH₃, and —CH═CH—N(CH₃)—CH₃. In certain embodiments, where the heteroalkyl comprises a CH₃ group, the heteroalkyl is present at the 5′ end and/or 3′ end of the oligonucleotide.

As used herein, the term “alkoxy,” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like. In an embodiment, C₁-C₆ alkoxy groups are provided herein.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “hydroxy” alone or as part of another substituent means, unless otherwise stated, an alcohol moiety having the formula —OH.

Preparation of linkers can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety. Adjustments to the protecting groups and formation and cleavage methods described herein may be adjusted as necessary in light of the various substituents.

Various methodologies of the instant disclosure include step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and example are illustrative only and not intended to be limiting.

Various aspects of the disclosure are described in further detail in the following subsections.

### Nucleotide Modifications & Chemical Modification Patterns

Provided herein are a series of nucleotide modifications, any one or more of which that may be applied to an RNA molecule (e.g., a dsRNA) to prolong in vivo silencing activity, such as after a single administration. Also provided are RNA chemical modification patterns (e.g., antisense strand and sense strand chemical modification patterns) that achieve prolonged in vivo silencing.

In certain embodiments, the RNA molecule of the disclosure comprises one or more nucleotide modifications selected from the group consisting of an alkyl modification, a locked nucleic acid (LNA) modification, an unlocked nucleic acid (UNA) modification, a 2′-deoxy modification, and a 2′-MOE modification.

In certain embodiments, the RNA molecule comprises at least one alkyl modification within nucleotide positions 1-5 of one or both of the 5′ end and 3′ end.

In certain embodiments, the RNA molecule comprises an alkyl modification at nucleotide position 1 of the 5′ end. In certain embodiments, the RNA molecule comprises an alkyl modification at nucleotide position 2 of the 5′ end. In certain embodiments, the RNA molecule comprises an alkyl modification at nucleotide position 3 of the 5′ end. In certain embodiments, the RNA molecule comprises an alkyl modification at nucleotide position 4 of the 5′ end. In certain embodiments, the RNA molecule comprises an alkyl modification at nucleotide position 5 of the 5′ end. In certain embodiments, the RNA molecule comprises an alkyl modification at nucleotide position 1 and 2 of the 5′ end. In certain embodiments, the RNA molecule comprises an alkyl modification at nucleotide position 1-3 of the 5′ end. In certain embodiments, the RNA molecule comprises an alkyl modification at nucleotide position 1-4 of the 5′ end. In certain embodiments, the RNA molecule comprises an alkyl modification at nucleotide position 1-5 of the 5′ end.

In certain embodiments, the RNA molecule comprises an alkyl modification at nucleotide position 1 of the 3′ end. In certain embodiments, the RNA molecule comprises an alkyl modification at nucleotide position 2 of the 3′ end. In certain embodiments, the RNA molecule comprises an alkyl modification at nucleotide position 3 of the 3′ end. In certain embodiments, the RNA molecule comprises an alkyl modification at nucleotide position 4 of the 3′ end. In certain embodiments, the RNA molecule comprises an alkyl modification at nucleotide position 5 of the 3′ end. In certain embodiments, the RNA molecule comprises an alkyl modification at nucleotide position 1 and 2 of the 3′ end. In certain embodiments, the RNA molecule comprises an alkyl modification at nucleotide position 1-3 of the 3′ end. In certain embodiments, the RNA molecule comprises an alkyl modification at nucleotide position 1-4 of the 3′ end. In certain embodiments, the RNA molecule comprises an alkyl modification at nucleotide position 1-5 of the 3′ end.

In certain embodiments, the alkyl modification comprises a C₁-C₁₀ alkyl. In certain embodiments, the alkyl modification comprises a C₁ alkyl. In certain embodiments, the alkyl modification comprises a C₂ alkyl. In certain embodiments, the alkyl modification comprises a C₃ alkyl. In certain embodiments, the alkyl modification comprises a C₄ alkyl (i.e., butyl). In certain embodiments, the alkyl modification comprises a C₅ alkyl. In certain embodiments, the alkyl modification comprises a C₆ alkyl. In certain embodiments, the alkyl modification comprises a C₇ alkyl. In certain embodiments, the alkyl modification comprises a C₈ alkyl. In certain embodiments, the alkyl modification comprises a C₉ alkyl. In certain embodiments, the alkyl modification comprises a C₁₀ alkyl.

In certain embodiments, the alkyl modification comprises a branched C₃-C₁₀ alkyl. In certain embodiments, the alkyl modification comprises a branched C₃ alkyl. In certain embodiments, the alkyl modification comprises a branched C₄ alkyl. In certain embodiments, the alkyl modification comprises a branched C₅ alkyl. In certain embodiments, the alkyl modification comprises a branched C₆ alkyl. In certain embodiments, the alkyl modification comprises a branched C₇ alkyl. In certain embodiments, the alkyl modification comprises a branched C₈ alkyl. In certain embodiments, the alkyl modification comprises a branched C₉ alkyl. In certain embodiments, the alkyl modification comprises a branched Cm alkyl.

In certain embodiments, the branched alkyl is isopropyl. In certain embodiments, the branched alkyl is isobutyl. In certain embodiments, the branched alkyl is sec-butyl. In certain embodiments, the branched alkyl is tert-butyl.

In certain embodiments, the branch from the branched alkyl comprises a C₃-C₁₀ alkyl. In certain embodiments, the branch from the branched alkyl comprises a C₃ alkyl. In certain embodiments, the branch from the branched alkyl comprises a C₄ alkyl. In certain embodiments, the branch from the branched alkyl comprises a C₅ alkyl. In certain embodiments, the branch from the branched alkyl comprises a C₆ alkyl. In certain embodiments, the branch from the branched alkyl comprises a C₇ alkyl. In certain embodiments, the branch from the branched alkyl comprises a C₈ alkyl. In certain embodiments, the branch from the branched alkyl comprises a C₉ alkyl. In certain embodiments, the branch from the branched alkyl comprises a C₁₀ alkyl.

When an alkyl modification is employed, it may be inserted between two adjacent nucleotides or inserted in place of a nucleotide (i.e., replace the nucleotide). An RNA molecule of sequence ATGC will be used to exemplify the position of alkyl modifications. When an alkyl modification is inserted between two adjacent nucleotides, the exemplary sequence would be AT(ibut)GC, wherein “ibut” corresponds to an internal butyl modification. When an alkyl modification is inserted in place of a nucleotide, the exemplary sequence would be AT(but)C, wherein “but” corresponds to a butyl replacement modification.

In certain embodiments, the at least one alkyl modification is positioned between two adjacent nucleotides.

In certain embodiments, the at least one alkyl modification positioned between two adjacent nucleotides does not replace a nucleotide at a position within the RNA molecule relative to an RNA molecule that does not contain the at least one alkyl modification at the same position within the RNA molecule.

In certain embodiments, the at least one alkyl modification replaces a nucleotide at a position within the RNA molecule relative to an RNA molecule that does not contain the at least one alkyl modification at the same position within the RNA molecule.

In certain embodiments, the alkyl modification is linear (i.e., unbranched).

In other embodiments, the alkyl modification is branched.

In certain embodiments, the RNA molecule comprises a single stranded (ss) RNA or a double stranded (ds) RNA. The dsRNA of the disclosure comprises an antisense strand with complementarity to a target mRNA and a sense strand with complementarity to the antisense strand, each strand comprising a 5′ end and a 3′ end.

In certain embodiments, the antisense strand is between 15 and 25 nucleotides in length. In certain embodiments, the antisense strand is 18, 19, 20, 21, 22, or 23 nucleotides in length. In certain embodiments, the sense strand is between 15 and 25 nucleotides in length. In certain embodiments, the sense strand is 14, 15, 16, or 17 nucleotides in length.

In certain embodiments, the dsRNA comprises a double-stranded region of 15 base pairs to 20 base pairs. In certain embodiments, the dsRNA comprises a double-stranded region of 15 base pairs. In certain embodiments, the dsRNA comprises a double-stranded region of 16 base pairs. In certain embodiments, the dsRNA comprises a double-stranded region of 18 base pairs. In certain embodiments, the dsRNA comprises a double-stranded region of 20 base pairs.

In certain embodiments, the dsRNA comprises a blunt end. In certain embodiments, the dsRNA comprises at least one single stranded nucleotide overhang. In certain embodiments, the dsRNA comprises about a 2-nucleotide to 5-nucleotide single stranded nucleotide overhang. In certain embodiments, the dsRNA comprises 2-nucleotide single stranded nucleotide overhang. In certain embodiments, the dsRNA comprises 5-nucleotide single stranded nucleotide overhang.

The nucleotide modifications selected from the group consisting of an alkyl modification, a locked nucleic acid (LNA) modification, an unlocked nucleic acid (UNA) modification, a 2′-deoxy modification, and a 2′-MOE modification, may be applied to any one or more nucleotide positions within the antisense or sense strand.

In certain embodiments, the antisense strand comprises an alkyl modification at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In certain embodiments, the antisense strand comprises an LNA modification at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In certain embodiments, the antisense strand comprises an UNA modification at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In certain embodiments, the antisense strand comprises a 2′-deoxy modification at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In certain embodiments, the antisense strand comprises a 2′-MOE modification at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In certain embodiments, the antisense strand comprises an unmodified RNA nucleotide at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In certain embodiments, the sense strand comprises an alkyl modification at one or more of nucleotide positions 1, 2, 3, 4, 5, 6, 7, 8, 9, 10, 11, 12, 13, 14, 15, 16, 17, 18, 19, or counted from the 5′ end.

In certain embodiments, the sense strand comprises an LNA modification at one or more of nucleotide positions 1, 2, 3, 4, 5, 6, 7, 8, 9, 10, 11, 12, 13, 14, 15, 16, 17, 18, 19, or counted from the 5′ end.

In certain embodiments, the sense strand comprises an UNA modification at one or more of nucleotide positions 1, 2, 3, 4, 5, 6, 7, 8, 9, 10, 11, 12, 13, 14, 15, 16, 17, 18, 19, or counted from the 5′ end.

In certain embodiments, the sense strand comprises a 2′-deoxy modification at one or more of nucleotide positions 1, 2, 3, 4, 5, 6, 7, 8, 9, 10, 11, 12, 13, 14, 15, 16, 17, 18, 19, or 20, counted from the 5′ end.

In certain embodiments, the sense strand comprises a 2′-MOE modification at one or more of nucleotide positions 1, 2, 3, 4, 5, 6, 7, 8, 9, 10, 11, 12, 13, 14, 15, 16, 17, 18, 19, or 20, counted from the 5′ end.

In certain embodiments, the sense strand comprises an unmodified RNA nucleotide at one or more of nucleotide positions 1, 2, 3, 4, 5, 6, 7, 8, 9, 10, 11, 12, 13, 14, 15, 16, 17, 18, 19, 20, 21, 22, or 23, counted from the 5′ end.

In one aspect, the disclosure provides a double stranded (ds) RNA, comprising an antisense strand with a 5′ end and a 3′ end, and a sense strand with a 5′ end and a 3′ end, wherein the antisense strand comprises at least one alkyl modification.

In certain embodiments of the RNA molecule or dsRNA of the disclosure, the RNA molecule or dsRNA comprises at least one modified internucleotide linkage.

In certain embodiments, the modified internucleotide linkage comprises a phosphorothioate internucleotide linkage. In certain embodiments, the RNA molecule or dsRNA comprises 4-16 phosphorothioate internucleotide linkages. In certain embodiments, the RNA molecule or dsRNA comprises 8-13 phosphorothioate internucleotide linkages.

### Antisense Single-Stranded Overhang Modification

The instant disclosure further provides for antisense single-stranded overhang (i.e., tail) nucleotide modification. An antisense single-stranded overhang forms when the antisense strand is longer than the sense strand of a dsRNA. Single-stranded overhangs can be between 1 to 6 nucleotides in length.

In certain embodiments, the single-stranded overhang is 2 nucleotides long, 3 nucleotides long, 4 nucleotides long, or 5 nucleotides long.

In certain embodiments, the single-stranded overhang comprises an alkyl modification at one or more of nucleotide positions 1, 2, 3, 4, or 5, counted from the 5′ end.

In certain embodiments, the single-stranded overhang comprises an LNA modification at one or more of nucleotide positions 1, 2, 3, 4, or 5, counted from the 5′ end.

In certain embodiments, the single-stranded overhang comprises an UNA modification at one or more of nucleotide positions 1, 2, 3, 4, or 5, counted from the 5′ end.

In certain embodiments, the single-stranded overhang comprises a 2′-deoxy modification at one or more of nucleotide positions 1, 2, 3, 4, or 5, counted from the 5′ end.

In certain embodiments, the single-stranded overhang comprises a 2′-MOE modification at one or more of nucleotide positions 1, 2, 3, 4, or 5, counted from the 5′ end.

In certain embodiments, the single-stranded overhang comprises an unmodified RNA nucleotide at one or more of nucleotide positions 1, 2, 3, 4, or 5, counted from the 5′ end.

### Exemplary Chemical Modification Patterns

Provided below in Tables 1-8 are exemplary chemical modification patterns for antisense and sense strands.

TABLE 1
Butyl-containing chemical modification patterns

| | |
|---|---|
| P1_b1_as | P(but)#(fN)#(mN)(fN)(fN)(fN)(mN)(fN)(mN)(fN)(mN)(fN)(mN)(fN)#(mN)#(fN) |
| | #(mN)#(mN)#(mN)#(fN)#(mN) |
| P1_b2_as | P(mN)#(but)#(mN)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_b3_as | P(mN)#(fN)#(but)(fN)(fN)(fN)(mN)(fN)(mN)(fN)(mN)(fN)(mN)(fN)#(mN)#(fN) |
| | #(mN)#(mN)#(mN)#(fN)#(mN) |
| P1_b4_as | P(mN)#(fN)#(mN)(but)(fN)(fN)(mN)(fN)(mN)(fN)(mN)(fN)(mN)(fN)#(mN)# |
| | (fN)#(mN)#(mN)#(mN)#(fN)#(mN) |
| P1_b5_as | P(mN)#(fN)#(mN)(fN)(but)(fN)(mN)(fN)(mN)(fN)(mN)(fN)(mN)(fN)#(mN)# |
| | (fN)#(mN)#(mN)#(mN)#(fN)#(mN) |
| P1_b6_as | P(mN)#(fN)#(mN)(fN)(fN)(but)(mN)(fN)(mN)(fN)(mN)(fN)(mN)(fN)#(mN)# |
| | (fN)#(mN)#(mN)#(mN)#(fN)#(mN) |
| P1_b7_as | P(mN)#(fN)#(mN)(fN)(fN)(fN)(but)(fN)(mN)(fN)(mN)(fN)(mN)(fN)#(mN)#(fN) |
| | #(mN)#(mN)#(mN)#(fN)#(mN) |
| P1_b8_as | P(mN)#(fN)#(mN)(fN)(fN)(fN)(mN)(but)(mN)(fN)(mN)(fN)(mN)(fN)#(mN)# |
| | (fN)#(mN)#(mN)#(mN)#(fN)#(mN) |
| P1_b9_as | P(mN)#(fN)#(mN)(fN)(fN)(fN)(mN)(fN)(but)(fN)(mN)(fN)(mN)(fN)#(mN)#(fN) |
| | #(mN)#(mN)#(mN)#(fN)#(mN) |
| P1_b10_ | P(mN)#(fN)#(mN)(fN)(fN)(fN)(mN)(fN)(mN)(but)(mN)(fN)(mN)(fN)#(mN)# |
| as | (fN)#(mN)#(mN)#(mN)#(fN)#(mN) |
| P1_b11_ | P(mN)#(fN)#(mN)(fN)(fN)(fN)(mN)(fN)(mN)(fN)(but)(fN)(mN)(fN)#(mN)#(fN) |
| as | #(mN)#(mN)#(mN)#(fN)#(mN) |
| P1_b12_ | P(mN)#(fN)#(mN)(fN)(fN)(fN)(mN)(fN)(mN)(fN)(mN)(but)(mN)(fN)#(mN)# |
| as | (fN)#(mN)#(mN)#(mN)#(fN)#(mN) |
| P1_b13_ | P(mN)#(fN)#(mN)(fN)(fN)(fN)(mN)(fN)(mN)(fN)(mN)(fN)(but)(fN)#(mN)#(fN) |
| as | #(mN)#(mN)#(mN)#(fN)#(mN) |
| P1_b14_ | P(mN)#(fN)#(mN)(fN)(fN)(fN)(mN)(fN)(mN)(fN)(mN)(fN)(mN)(but)#(mN)# |
| as | (fN)#(mN)#(mN)#(mN)#(fN)#(mN) |
| P1_b15_ | P(mN)#(fN)#(mN)(fN)(fN)(fN)(mN)(fN)(mN)(fN)(mN)(fN)(mN)(fN)#(but)#(fN) |
| as | #(mN)#(mN)#(mN)#(fN)#(mN) |
| P1_b16_ | P(mN)#(fN)#(mN)(fN)(fN)(fN)(mN)(fN)(mN)(fN)(mN)(fN)(mN)(fN)#(mN)# |
| as | (but)#(mN)#(mN)#(mN)#(fN)#(mN) |
| P1_b17_ | P(mN)#(fN)#(mN)(fN)(fN)(fN)(mN)(fN)(mN)(fN)(mN)(fN)(mN)(fN)#(mN)#(fN) |
| as | #(but)#(mN)#(mN)#(fN)#(mN) |
| P1_b18_ | P(mN)#(fN)#(mN)(fN)(fN)(fN)(mN)(fN)(mN)(fN)(mN)(fN)(mN)(fN)#(mN)#(fN) |
| as | #(mN)#(but)#(mN)#(fN)#(mN) |
| P1_b19_ | P(mN)#(fN)#(mN)(fN)(fN)(fN)(mN)(fN)(mN)(fN)(mN)(fN)(mN)(fN)#(mN)#(fN) |
| as | #(mN)#(mN)#(but)#(fN)#(mN) |
| P1_b20_ | P(mN)#(fN)#(mN)(fN)(fN)(fN)(mN)(fN)(mN)(fN)(mN)(fN)(mN)(fN)#(mN)#(fN) |
| as | #(mN)#(mN)#(mN)#(but)#(mN) |
| P1_b21_ | P(mN)#(fN)#(mN)(fN)(fN)(fN)(mN)(fN)(mN)(fN)(mN)(fN)(mN)(fN)#(mN)#(fN) |
| as | #(mN)#(mN)#(mN)#(fN)#(but) |
| P2_b1_ | P(but)#(fN)#(mN)(mN)(mN)(fN)(mN)(mN)(mN)(mN)(mN)(mN)(mN)(fN)#(mN) |
| as | #(fN)#(mN)#(mN)#(mN)#(fN)#(mN) |
| P2_b2_as | P(mN)#(but)#(mN)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b3_as | P(mN)#(fN)#(but)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b4_as | P(mN)#(fN)#(mN)(but)(mN)(fN)(mN)(mN)(mN)(mN)(mN)(mN)(mN)(fN)# |
| | (mN)#(fN)#(mN)#(mN)#(mN)#(fN)#(mN) |
| P2_b5_as | P(mN)#(fN)#(mN)(mN)(but)(fN)(mN)(mN)(mN)(mN)(mN)(mN)(mN)(fN)# |
| | (mN)#(fN)#(mN)#(mN)#(mN)#(fN)#(mN) |
| P2_b6_as | P(mN)#(fN)#(mN)(mN)(mN)(but)(mN)(mN)(mN)(mN)(mN)(mN)(mN)(fN)# |
| | (mN)#(fN)#(mN)#(mN)#(mN)#(fN)#(mN) |
| P2_b7_as | P(mN)#(fN)#(mN)(mN)(mN)(fN)(but)(mN)(mN)(mN)(mN)(mN)(mN)(fN)# |
| | (mN)#(fN)#(mN)#(mN)#(mN)#(fN)#(mN) |
| P2_b8_as | P(mN)#(fN)#(mN)(mN)(mN)(fN)(mN)(but)(mN)(mN)(mN)(mN)(mN)(fN)# |
| | (mN)#(fN)#(mN)#(mN)#(mN)#(fN)#(mN) |
| P2_b9_as | P(mN)#(fN)#(mN)(mN)(mN)(fN)(mN)(mN)(but)(mN)(mN)(mN)(mN)(fN)# |
| | (mN)#(fN)#(mN)#(mN)#(mN)#(fN)#(mN) |
| P2_b10_ | P(mN)#(fN)#(mN)(mN)(mN)(fN)(mN)(mN)(mN)(but)(mN)(mN)(mN)(fN)# |
| as | (mN)#(fN)#(mN)#(mN)#(mN)#(fN)#(mN) |
| P2_b11_ | P(mN)#(fN)#(mN)(mN)(mN)(fN)(mN)(mN)(mN)(mN)(but)(mN)(mN)(fN)# |
| as | (mN)#(fN)#(mN)#(mN)#(mN)#(fN)#(mN) |
| P2_b12_ | P(mN)#(fN)#(mN)(mN)(mN)(fN)(mN)(mN)(mN)(mN)(mN)(but)(mN)(fN)# |
| as | (mN)#(fN)#(mN)#(mN)#(mN)#(fN)#(mN) |
| P2_b13_ | P(mN)#(fN)#(mN)(mN)(mN)(fN)(mN)(mN)(mN)(mN)(mN)(mN)(but)(fN)# |
| as | (mN)#(fN)#(mN)#(mN)#(mN)#(fN)#(mN) |
| P2_b14_ | P(mN)#(fN)#(mN)(mN)(mN)(fN)(mN)(mN)(mN)(mN)(mN)(mN)(mN)(but)# |
| as | (mN)#(fN)#(mN)#(mN)#(mN)#(fN)#(mN) |
| P2_b15_ | P(mN)#(fN)#(mN)(mN)(mN)(fN)(mN)(mN)(mN)(mN)(mN)(mN)(mN)(fN)# |
| as | (but)#(fN)#(mN)#(mN)#(mN)#(fN)#(mN) |
| P2_b16_ | P(mN)#(fN)#(mN)(mN)(mN)(fN)(mN)(mN)(mN)(mN)(mN)(mN)(mN)(fN)# |
| as | (mN)#(but)#(mN)#(mN)#(mN)#(fN)#(mN) |
| P2_b17_ | P(mN)#(fN)#(mN)(mN)(mN)(fN)(mN)(mN)(mN)(mN)(mN)(mN)(mN)(fN)# |
| as | (mN)#(fN)#(but)#(mN)#(mN)#(fN)#(mN) |
| P2_b18_ | P(mN)#(fN)#(mN)(mN)(mN)(fN)(mN)(mN)(mN)(mN)(mN)(mN)(mN)(fN)# |
| as | (mN)#(fN)#(mN)#(but)#(mN)#(fN)#(mN) |
| P2_b19_ | P(mN)#(fN)#(mN)(mN)(mN)(fN)(mN)(mN)(mN)(mN)(mN)(mN)(mN)(fN)# |
| as | (mN)#(fN)#(mN)#(mN)#(but)#(fN)#(mN) |
| P2_b20_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(but)#(mN) |
| P2_b21_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(but) |
| P1_b1_s | (but)#(mN)#(mN)(fN)(mN)(fN)(mN)(fN)(mN)(fN)(mN)(mN)(mN)(fN)#(mN) |
| | #(mN) |
| P1_b2_s | (mN)#(but)#(mN)(fN)(mN)(fN)(mN)(fN)(mN)(fN)(mN)(mN)(mN)(fN)#(mN) |
| | #(mN) |
| P1_b3_s | (mN)#(mN)#(but)(fN)(mN)(fN)(mN)(fN)(mN)(fN)(mN)(mN)(mN)(fN)#(mN) |
| | #(mN) |
| P1_b4_s | (mN)#(mN)#(mN)(but)(mN)(fN)(mN)(fN)(mN)(fN)(mN)(mN)(mN)(fN)#(mN) |
| | #(mN) |
| P1_b5_s | (mN)#(mN)#(mN)(fN)(but)(fN)(mN)(fN)(mN)(fN)(mN)(mN)(mN)(fN)#(mN) |
| | #(mN) |
| P1_b6_s | (mN)#(mN)#(mN)(fN)(mN)(but)(mN)(fN)(mN)(fN)(mN)(mN)(mN)(fN)#(mN) |
| | #(mN) |
| P1_b7_s | (mN)#(mN)#(mN)(fN)(mN)(fN)(but)(fN)(mN)(fN)(mN)(mN)(mN)(fN)#(mN) |
| | #(mN) |
| P1_b8_s | (mN)#(mN)#(mN)(fN)(mN)(fN)(mN)(but)(mN)(fN)(mN)(mN)(mN)(fN)#(mN) |
| | #(mN) |
| P1_b9_s | (mN)#(mN)#(mN)(fN)(mN)(fN)(mN)(fN)(but)(fN)(mN)(mN)(mN)(fN)#(mN) |
| | #(mN) |
| P1_b10_ | (mN)#(mN)#(mN)(fN)(mN)(fN)(mN)(fN)(mN)(but)(mN)(mN)(mN)(fN)#(mN) |
| s | #(mN) |
| P1_b11_ | (mN)#(mN)#(mN)(fN)(mN)(fN)(mN)(fN)(mN)(fN)(but)(mN)(mN)(fN)#(mN) |
| s | #(mN) |
| P1_b12_ | (mN)#(mN)#(mN)(fN)(mN)(fN)(mN)(fN)(mN)(fN)(mN)(but)(mN)(IN)#(mN) |
| s | #(mN) |
| P1_b13_ | (mN)#(mN)#(mN)(fN)(mN)(fN)(mN)(fN)(mN)(fN)(mN)(mN)(but)(fN)#(mN) |
| s | #(mN) |
| P1_b14_ | (mN)#(mN)#(mN)(fN)(mN)(fN)(mN)(fN)(mN)(fN)(mN)(mN)(mN)(but)#(mN) |
| s | #(mN) |
| P1_b15_ | (mN)#(mN)#(mN)(fN)(mN)(fN)(mN)(fN)(mN)(fN)(mN)(mN)(mN)(fN)#(but) |
| s | #(mN) |
| P1_b16_ | (mN)#(mN)#(mN)(fN)(mN)(fN)(mN)(fN)(mN)(fN)(mN)(mN)(mN)(fN)#(mN) |
| s | #(but) |
| P2_b1_s | (but)#(mN)#(mN)(mN)(mN)(fN)(fN)(fN)(mN)(fN)(mN)(mN)(mN)(mN)#(mN) |
| | #(mN) |
| P2_b2_s | (mN)#(but)#(mN)(mN)(mN)(fN)(fN)(fN)(mN)(fN)(mN)(mN)(mN)(mN)#(mN) |
| | #(mN) |
| P2_b3_s | (mN)#(mN)#(but)(mN)(mN)(fN)(fN)(fN)(mN)(fN)(mN)(mN)(mN)(mN)#(mN) |
| | #(mN) |
| P2_b4_s | (mN)#(mN)#(mN)(but)(mN)(fN)(fN)(fN)(mN)(fN)(mN)(mN)(mN)(mN)#(mN) |
| | #(mN) |
| P2_b5_s | (mN)#(mN)#(mN)(mN)(but)(fN)(fN)(fN)(mN)(fN)(mN)(mN)(mN)(mN)#(mN) |
| | #(mN) |
| P2_b6_s | (mN)#(mN)#(mN)(mN)(mN)(but)(fN)(fN)(mN)(fN)(mN)(mN)(mN)(mN)# |
| | (mN)#(mN) |
| P2_b7_s | (mN)#(mN)#(mN)(mN)(mN)(fN)(but)(fN)(mN)(fN)(mN)(mN)(mN)(mN)# |
| | (mN)#(mN) |
| P2_b8_s | (mN)#(mN)#(mN)(mN)(mN)(fN)(fN)(but)(mN)(fN)(mN)(mN)(mN)(mN)# |
| | (mN)#(mN) |
| P2_b9_s | (mN)#(mN)#(mN)(mN)(mN)(fN)(fN)(fN)(but)(fN)(mN)(mN)(mN)(mN)#(mN) |
| | #(mN) |
| P2_b10_ | (mN)#(mN)#(mN)(mN)(mN)(fN)(fN)(fN)(mN)(but)(mN)(mN)(mN)(mN)# |
| s | (mN)#(mN) |
| P2_b11_ | (mN)#(mN)#(mN)(mN)(mN)(fN)(fN)(fN)(mN)(fN)(but)(mN)(mN)(mN)#(mN) |
| s | #(mN) |
| P2_b12_ | (mN)#(mN)#(mN)(mN)(mN)(fN)(fN)(fN)(mN)(fN)(mN)(but)(mN)(mN)#(mN) |
| s | #(mN) |
| P2_b13_ | (mN)#(mN)#(mN)(mN)(mN)(fN)(fN)(fN)(mN)(fN)(mN)(mN)(but)(mN)#(mN) |
| s | #(mN) |
| P2_b14_ | (mN)#(mN)#(mN)(mN)(mN)(fN)(fN)(fN)(mN)(fN)(mN)(mN)(mN)(but)#(mN) |
| s | #(mN) |
| P2_b15_ | (mN)#(mN)#(mN)(mN)(mN)(fN)(IN)(fN)(mN)(fN)(mN)(mN)(mN)(mN)#(but) |
| s | #(mN) |
| P2_b16_ | (mN)#(mN)#(mN)(mN)(mN)(fN)(fN)(fN)(mN)(fN)(mN)(mN)(mN)(mN)#(mN) |
| s | #(but) |
| as_TW_ | P(mN)#(fN)#(mN)(mN)(mN)(fN)(mN)(mN)(mN)(mN)(mN)(mN)(mN)(fN)# |
| But1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But2 | (mN)#(fN)#(but)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But3 | (mN)#(fN)#(mN)#(but)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But4 | (mN)#(fN)#(mN)#(mN)#(but)#(mN)#(mN) |
| as_TW_ | P(mN)#(fN)#(mN)(mN)(mN)(fN)(mN)(mN)(mN)(mN)(mN)(mN)(mN)(fN)# |
| But5 | (mN)#(fN)#(mN)#(mN)#(mN)#(but)#(mN) |
| as_TW_ | P(mN)#(fN)#(mN)(mN)(mN)(fN)(mN)(mN)(mN)(mN)(mN)(mN)(mN)(fN)# |
| But6 | (mN)#(fN)#(mN)#(mN)#(mN)#(mN)#(but) |
| as_TW_ | P(mN)#(fN)#(mN)(mN)(mN)(fN)(mN)(mN)(mN)(mN)(mN)(mN)(mN)(fN)# |
| But7 | (mN)#(fN)#(but)#(but)#(mN)#(mN)#(mN) |
| as_TW_ | P(mN)#(fN)#(mN)(mN)(mN)(fN)(mN)(mN)(mN)(mN)(mN)(mN)(mN)(fN)# |
| But8 | (mN)#(fN)#(mN)#(but)#(but)#(mN)#(mN) |
| as_TW_ | P(mN)#(fN)#(mN)(mN)(mN)(fN)(mN)(mN)(mN)(mN)(mN)(mN)(mN)(fN)# |
| But9 | (mN)#(fN)#(mN)#(mN)#(but)#(but)#(mN) |
| as_TW_ | P(mN)#(fN)#(mN)(mN)(mN)(fN)(mN)(mN)(mN)(mN)(mN)(mN)(mN)(fN)# |
| But10 | (mN)#(fN)#(mN)#(mN)#(mN)#(but)#(but) |
| as_TW_ | P(mN)#(fN)#(mN)(mN)(mN)(fN)(mN)(mN)(mN)(mN)(mN)(mN)(mN)(fN)# |
| But11 | (mN)#(fN)#(but)#(mN)#(but)#(mN)#(mN) |
| as_TW_ | P(mN)#(fN)#(mN)(mN)(mN)(fN)(mN)(mN)(mN)(mN)(mN)(mN)(mN)(fN)# |
| But12 | (mN)#(fN)#(mN)#(mN)#(but)#(mN)#(but) |
| as_TW_ | P(mN)#(fN)#(mN)(mN)(mN)(fN)(mN)(mN)(mN)(mN)(mN)(mN)(mN)(fN)# |
| But13 | (mN)#(fN)#(mN)#(but)#(mN)#(but)#(mN) |
| as_TW_ | P(mN)#(fN)#(mN)(mN)(mN)(fN)(mN)(mN)(mN)(mN)(mN)(mN)(mN)(fN)# |
| But14 | (mN)#(fN)#(but)#(but)#(but)#(mN)#(mN) |
| as_TW_ | P(mN)#(fN)#(mN)(mN)(mN)(fN)(mN)(mN)(mN)(mN)(mN)(mN)(mN)(fN)# |
| But15 | (mN)#(fN)#(mN)#(mN)#(but)#(but)#(but) |
| as_TW_ | P(mN)#(fN)#(mN)(mN)(mN)(fN)(mN)(mN)(mN)(mN)(mN)(mN)(mN)(fN)# |
| But16 | (mN)#(fN)#(but)#(but)#(but)#(but)#(but) |

TABLE 2
Internal butyl-containing chemical modification patterns

| | |
|---|---|
| P1_ib1_as | P(ibut)(mN)#(fN)#(mN)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib2_as | P(mN)#(ibut)(fN)#(mN)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib3_as | P(mN)#(fN)#(ibut)(mN)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib4_as | P(mN)#(fN)#(mN)(ibut)(fN)(fN)(fN)(mN)(fN)(mN)(fN)(mN)(fN)(mN)(fN)# |
| | (mN)#(fN)#(mN)#(mN)#(mN)#(fN)#(mN) |
| P1_ib5_as | P(mN)#(fN)#(mN)(fN)(ibut)(fN)(fN)(mN)(fN)(mN)(fN)(mN)(fN)(mN)(fN)# |
| | (mN)#(fN)#(mN)#(mN)#(mN)#(fN)#(mN) |
| P1_ib6_as | P(mN)#(fN)#(mN)(fN)(fN)(ibut)(fN)(mN)(fN)(mN)(fN)(mN)(fN)(mN)(fN)# |
| | (mN)#(fN)#(mN)#(mN)#(mN)#(fN)#(mN) |
| P1_ib7_as | P(mN)#(fN)#(mN)(fN)(fN)(fN)(ibut)(mN)(fN)(mN)(fN)(mN)(fN)(mN)(fN)# |
| | (mN)#(fN)#(mN)#(mN)#(mN)#(fN)#(mN) |
| P1_ib8_as | P(mN)#(fN)#(mN)(fN)(fN)(fN)(mN)(ibut)(fN)(mN)(fN)(mN)(fN)(mN)(fN)# |
| | (mN)#(fN)#(mN)#(mN)#(mN)#(fN)#(mN) |
| P1_ib9_as | P(mN)#(fN)#(mN)(fN)(fN)(fN)(mN)(fN)(ibut)(mN)(fN)(mN)(fN)(mN)(fN)# |
| | (mN)#(fN)#(mN)#(mN)#(mN)#(fN)#(mN) |
| P1_ib10_ | P(mN)#(fN)#(mN)(fN)(fN)(fN)(mN)(fN)(mN)(ibut)(fN)(mN)(fN)(mN)(fN)# |
| as | (mN)#(fN)#(mN)#(mN)#(mN)#(fN)#(mN) |
| P1_ib11_ | P(mN)#(fN)#(mN)(fN)(fN)(fN)(mN)(fN)(mN)(fN)(ibut)(mN)(fN)(mN)(fN)# |
| as | (mN)#(fN)#(mN)#(mN)#(mN)#(fN)#(mN) |
| P1_ib12_ | P(mN)#(fN)#(mN)(fN)(fN)(fN)(mN)(fN)(mN)(fN)(mN)(ibut)(fN)(mN)(fN)# |
| as | (mN)#(fN)#(mN)#(mN)#(mN)#(fN)#(mN) |
| P1_ib13_ | P(mN)#(fN)#(mN)(fN)(fN)(fN)(mN)(fN)(mN)(fN)(mN)(fN)(ibut)(mN)(fN)# |
| as | (mN)#(fN)#(mN)#(mN)#(mN)#(fN)#(mN) |
| P1_ib14_ | P(mN)#(fN)#(mN)(fN)(fN)(fN)(mN)(fN)(mN)(fN)(mN)(fN)(mN)(ibut)(fN)# |
| as | (mN)#(fN)#(mN)#(mN)#(mN)#(fN)#(mN) |
| P1_ib15_ | P(mN)#(fN)#(mN)(fN)(fN)(fN)(mN)(fN)(mN)(fN)(mN)(fN)(mN)(fN)#(ibut) |
| as | (mN)#(fN)#(mN)#(mN)#(mN)#(fN)#(mN) |
| P1_ib16_ | P(mN)#(fN)#(mN)(fN)(fN)(fN)(mN)(fN)(mN)(fN)(mN)(fN)(mN)(fN)#(mN)# |
| as | (ibut)(fN)#(mN)#(mN)#(mN)#(fN)#(mN) |
| P1_ib17_ | P(mN)#(fN)#(mN)(fN)(fN)(fN)(mN)(fN)(mN)(fN)(mN)(fN)(mN)(fN)#(mN)# |
| as | (fN)#(ibut)(mN)#(mN)#(mN)#(fN)#(mN) |
| P1_ib18_ | P(mN)#(fN)#(mN)(fN)(fN)(fN)(mN)(fN)(mN)(fN)(mN)(fN)(mN)(fN)#(mN)# |
| as | (fN)#(mN)#(ibut)(mN)#(mN)#(fN)#(mN) |
| P1_ib19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(ibut)(mN)#(fN)#(mN) |
| P1_ib20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(ibut)(fN)#(mN) |
| P1_ib21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(ibut)(mN) |
| P1_ib22_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(mN)(ibut) |
| P2_ib1_as | P(ibut)(mN)#(fN)#(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib2_as | P(mN)#(ibut)(fN)#(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib3_as | P(mN)#(fN)#(ibut)(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib4_as | P(mN)#(fN)#(mN)(ibut)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib5_as | P(mN)#(fN)#(mN)(mN)(ibut)(mN)(fN)(mN)(mN)(mN)(mN)(mN)(mN)(mN)(fN) |
| | #(mN)#(fN)#(mN)#(mN)#(mN)#(fN)#(mN) |
| P2_ib6_as | P(mN)#(fN)#(mN)(mN)(mN)(ibut)(fN)(mN)(mN)(mN)(mN)(mN)(mN)(mN)(fN) |
| | #(mN)#(fN)#(mN)#(mN)#(mN)#(fN)#(mN) |
| P2_ib7_as | P(mN)#(fN)#(mN)(mN)(mN)(fN)(ibut)(mN)(mN)(mN)(mN)(mN)(mN)(mN)(fN) |
| | #(mN)#(fN)#(mN)#(mN)#(mN)#(fN)#(mN) |
| P2_ib8_as | P(mN)#(fN)#(mN)(mN)(mN)(fN)(mN)(ibut)(mN)(mN)(mN)(mN)(mN)(mN)(fN) |
| | #(mN)#(fN)#(mN)#(mN)#(mN)#(fN)#(mN) |
| P2_ib9_as | P(mN)#(fN)#(mN)(mN)(mN)(fN)(mN)(mN)(ibut)(mN)(mN)(mN)(mN)(mN)(fN) |
| | #(mN)#(fN)#(mN)#(mN)#(mN)#(fN)#(mN) |
| P2_ib10_ | P(mN)#(fN)#(mN)(mN)(mN)(fN)(mN)(mN)(mN)(ibut)(mN)(mN)(mN)(mN)(fN) |
| as | #(mN)#(fN)#(mN)#(mN)#(mN)#(fN)#(mN) |
| P2_ib11_ | P(mN)#(fN)#(mN)(mN)(mN)(fN)(mN)(mN)(mN)(mN)(ibut)(mN)(mN)(mN)(fN) |
| as | #(mN)#(fN)#(mN)#(mN)#(mN)#(fN)#(mN) |
| P2_ib12_ | P(mN)#(fN)#(mN)(mN)(mN)(fN)(mN)(mN)(mN)(mN)(mN)(ibut)(mN)(mN)(fN) |
| as | #(mN)#(fN)#(mN)#(mN)#(mN)#(fN)#(mN) |
| P2_ib13_ | P(mN)#(fN)#(mN)(mN)(mN)(fN)(mN)(mN)(mN)(mN)(mN)(mN)(ibut)(mN)(fN) |
| as | #(mN)#(fN)#(mN)#(mN)#(mN)#(fN)#(mN) |
| P2_ib14_ | P(mN)#(fN)#(mN)(mN)(mN)(fN)(mN)(mN)(mN)(mN)(mN)(mN)(mN)(ibut)(fN) |
| as | #(mN)#(fN)#(mN)#(mN)#(mN)#(fN)#(mN) |
| P2_ib15_ | P(mN)#(fN)#(mN)(mN)(mN)(fN)(mN)(mN)(mN)(mN)(mN)(mN)(mN)(fN) |
| as | (ibut)#(mN)#(fN)#(mN)#(mN)#(mN)#(fN)#(mN) |
| P2_ib16_ | P(mN)#(fN)#(mN)(mN)(mN)(fN)(mN)(mN)(mN)(mN)(mN)(mN)(mN)(fN)# |
| as | (mN)(ibut)#(fN)#(mN)#(mN)#(mN)#(fN)#(mN) |
| P2_ib17_ | P(mN)#(fN)#(mN)(mN)(mN)(fN)(mN)(mN)(mN)(mN)(mN)(mN)(mN)(fN)# |
| as | (mN)#(fN)(ibut)#(mN)#(mN)#(mN)#(fN)#(mN) |
| P2_ib18_ | P(mN)#(fN)#(mN)(mN)(mN)(fN)(mN)(mN)(mN)(mN)(mN)(mN)(mN)(fN)# |
| as | (mN)#(fN)#(mN)(ibut)#(mN)#(mN)#(fN)#(mN) |
| P2_ib19_ | P(mN)#(fN)#(mN)(mN)(mN)(fN)(mN)(mN)(mN)(mN)(mN)(mN)(mN)(fN)# |
| as | (mN)#(fN)#(mN)#(mN)#(ibut)(mN)#(fN)#(mN) |
| P2_ib20_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(ibut)(fN)#(mN) |
| P2_ib21_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(ibut)(mN) |
| P2_ib22_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN)(ibut) |
| P1_ib1_s | (ibut)(mN)#(mN)#(mN)(fN)(mN)(fN)(mN)(fN)(mN)(fN)(mN)(mN)(mN)(fN) |
| | #(mN)#(mN) |
| P1_ib2_s | (mN)#(ibut)(mN)#(mN)(fN)(mN)(fN)(mN)(fN)(mN)(fN)(mN)(mN)(mN)(fN) |
| | #(mN)#(mN) |
| P1_ib3_s | (mN)#(mN)#(ibut)(mN)(fN)(mN)(fN)(mN)(fN)(mN)(fN)(mN)(mN)(mN)(fN) |
| | #(mN)#(mN) |
| P1_ib4_s | (mN)#(mN)#(mN)(ibut)(fN)(mN)(fN)(mN)(fN)(mN)(fN)(mN)(mN)(mN)(fN) |
| | #(mN)#(mN) |
| P1_ib5_s | (mN)#(mN)#(mN)(fN)(ibut)(mN)(fN)(mN)(fN)(mN)(fN)(mN)(mN)(mN)(fN) |
| | #(mN)#(mN) |
| P1_ib6_s | (mN)#(mN)#(mN)(fN)(mN)(ibut)(fN)(mN)(fN)(mN)(fN)(mN)(mN)(mN)(fN) |
| | #(mN)#(mN) |
| P1_ib7_s | (mN)#(mN)#(mN)(fN)(mN)(fN)(ibut)(mN)(fN)(mN)(fN)(mN)(mN)(mN)(fN) |
| | #(mN)#(mN) |
| P1_ib8_s | (mN)#(mN)#(mN)(fN)(mN)(fN)(mN)(ibut)(fN)(mN)(fN)(mN)(mN)(mN)(fN) |
| | #(mN)#(mN) |
| P1_ib9_s | (mN)#(mN)#(mN)(fN)(mN)(fN)(mN)(fN)(ibut)(mN)(fN)(mN)(mN)(mN)(fN) |
| | #(mN)#(mN) |
| P1_ib10_ | (mN)#(mN)#(mN)(fN)(mN)(fN)(mN)(fN)(mN)(ibut)(fN)(mN)(mN)(mN)(fN) |
| s | #(mN)#(mN) |
| P1_ib11_ | (mN)#(mN)#(mN)(fN)(mN)(fN)(mN)(fN)(mN)(fN)(ibut)(mN)(mN)(mN)(fN) |
| s | #(mN)#(mN) |
| P1_ib12_ | (mN)#(mN)#(mN)(fN)(mN)(fN)(mN)(fN)(mN)(fN)(mN)(ibut)(mN)(mN)(fN) |
| s | #(mN)#(mN) |
| P1_ib13_ | (mN)#(mN)#(mN)(fN)(mN)(fN)(mN)(fN)(mN)(fN)(mN)(mN)(ibut)(mN)(fN) |
| s | #(mN)#(mN) |
| P1_ib14_ | (mN)#(mN)#(mN)(fN)(mN)(fN)(mN)(fN)(mN)(fN)(mN)(mN)(mN)(ibut)(fN) |
| s | #(mN)#(mN) |
| P1_ib15_ | (mN)#(mN)#(mN)(fN)(mN)(fN)(mN)(fN)(mN)(fN)(mN)(mN)(mN)(fN)#(ibut) |
| s | (mN)#(mN) |
| P1_ib16_ | (mN)#(mN)#(mN)(fN)(mN)(fN)(mN)(fN)(mN)(fN)(mN)(mN)(mN)(fN)#(mN) |
| s | #(ibut)(mN) |
| P1_ib17_ | (mN)#(mN)#(mN)(fN)(mN)(fN)(mN)(fN)(mN)(fN)(mN)(mN)(mN)(fN)#(mN) |
| s | #(mN)(ibut) |
| P2_ib1_s | (ibut)(mN)#(mN)#(mN)(mN)(mN)(fN)(fN)(fN)(mN)(fN)(mN)(mN)(mN)(mN) |
| | #(mN)#(mN) |
| P2_ib2_s | (mN)(ibut)#(mN)#(mN)(mN)(mN)(fN)(fN)(fN)(mN)(fN)(mN)(mN)(mN)(mN) |
| | #(mN)#(mN) |
| P2_ib3_s | (mN)#(mN)(ibut)#(mN)(mN)(mN)(fN)(fN)(fN)(mN)(fN)(mN)(mN)(mN)(mN) |
| | #(mN)#(mN) |
| P2_ib4_s | (mN)#(mN)#(mN)(ibut)(mN)(mN)(fN)(fN)(fN)(mN)(fN)(mN)(mN)(mN)(mN) |
| | #(mN)#(mN) |
| P2_ib5_s | (mN)#(mN)#(mN)(mN)(ibut)(mN)(fN)(fN)(fN)(mN)(fN)(mN)(mN)(mN)(mN) |
| | #(mN)#(mN) |
| P2_ib6_s | (mN)#(mN)#(mN)(mN)(mN)(ibut)(fN)(fN)(fN)(mN)(fN)(mN)(mN)(mN)(mN) |
| | #(mN)#(mN) |
| P2_ib7_s | (mN)#(mN)#(mN)(mN)(mN)(fN)(ibut)(fN)(fN)(mN)(fN)(mN)(mN)(mN)(mN) |
| | #(mN)#(mN) |
| P2_ib8_s | (mN)#(mN)#(mN)(mN)(mN)(fN)(fN)(ibut)(fN)(mN)(fN)(mN)(mN)(mN)(mN) |
| | #(mN)#(mN) |
| P2_ib9_s | (mN)#(mN)#(mN)(mN)(mN)(fN)(fN)(fN)(ibut)(mN)(fN)(mN)(mN)(mN)(mN) |
| | #(mN)#(mN) |
| P2_ib10_s | (mN)#(mN)#(mN)(mN)(mN)(fN)(fN)(fN)(mN)(ibut)(fN)(mN)(mN)(mN)(mN) |
| | #(mN)#(mN) |
| P2_ib11_ | (mN)#(mN)#(mN)(mN)(mN)(fN)(fN)(fN)(mN)(fN)(ibut)(mN)(mN)(mN)(mN) |
| s | #(mN)#(mN) |
| P2_ib12_ | (mN)#(mN)#(mN)(mN)(mN)(fN)(fN)(fN)(mN)(fN)(mN)(ibut)(mN)(mN)(mN) |
| s | #(mN)#(mN) |
| P2_ib13_ | (mN)#(mN)#(mN)(mN)(mN)(fN)(fN)(fN)(mN)(fN)(mN)(mN)(ibut)(mN)(mN) |
| s | #(mN)#(mN) |
| P2_ib14_ | (mN)#(mN)#(mN)(mN)(mN)(fN)(fN)(fN)(mN)(fN)(mN)(mN)(mN)(ibut)(mN) |
| s | #(mN)#(mN) |
| P2_ib15_ | (mN)#(mN)#(mN)(mN)(mN)(fN)(fN)(fN)(mN)(fN)(mN)(mN)(mN)(mN)(ibut) |
| s | #(mN)#(mN) |
| P2_ib16_ | (mN)#(mN)#(mN)(mN)(mN)(fN)(fN)(fN)(mN)(fN)(mN)(mN)(mN)(mN)# |
| s | (mN)#(ibut)(mN) |
| P2_ib17_ | (mN)#(mN)#(mN)(mN)(mN)(fN)(fN)(fN)(mN)(fN)(mN)(mN)(mN)(mN)# |
| s | (mN)#(mN)(ibut) |
| as_P5_ | P(mN)#(fN)#(mN)(mN)(mN)(fN)(mN)(mN)(mN)(mN)(mN)(mN)(mN)(fN)# |
| TW_iBut1 | (mN)#(fN)#(mN)#(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut2 | (mN)#(fN)#(ibut)(mN)#(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut3 | (mN)#(fN)#(mN)#(ibut)(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut4 | (mN)#(fN)#(mN)#(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut5 | (mN)#(fN)#(mN)#(mN)#(mN)#(ibut)(mN)#(mN) |
| as_P5_ | P(mN)#(fN)#(mN)(mN)(mN)(fN)(mN)(mN)(mN)(mN)(mN)(mN)(mN)(fN)# |
| TW_iBut6 | (mN)#(fN)#(mN)#(mN)#(mN)#(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut7 | (mN)#(fN)#(ibut)(mN)#(ibut)(mN)#(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut8 | (mN)#(fN)#(mN)#(ibut)(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut9 | (mN)#(fN)#(mN)#(mN)#(ibut)(mN)#(ibut)(mN)#(mN) |
| as_P5_ | P(mN)#(fN)#(mN)(mN)(mN)(fN)(mN)(mN)(mN)(mN)(mN)(mN)(mN)(fN)# |
| TW_iBut10 | (mN)#(fN)#(mN)#(mN)#(mN)#(ibut)(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut11 | (mN)#(fN)#(ibut)(mN)#(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut12 | (mN)#(fN)#(mN)#(mN)#(ibut)(mN)#(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut13 | (mN)#(fN)#(mN)#(ibut)(mN)#(mN)#(ibut)(mN)#(mN) |
| as_P5_ | P(mN)#(fN)#(mN)(mN)(mN)(fN)(mN)(mN)(mN)(mN)(mN)(mN)(mN)(fN)# |
| TW_iBut14 | (mN)#(fN)#(ibut)(mN)#(ibut)(mN)#(ibut)(mN)#(mN)#(mN) |
| as_P5_ | P(mN)#(fN)#(mN)(mN)(mN)(fN)(mN)(mN)(mN)(mN)(mN)(mN)(mN)(fN)# |
| TW_iBut15 | (mN)#(fN)#(mN)#(mN)#(ibut)(mN)#(ibut)(mN)#(ibut)(mN) |
| as_P5_ | P(mN)#(fN)#(mN)(mN)(mN)(fN)(mN)(mN)(mN)(mN)(mN)(mN)(mN)(fN)# |
| TW_iBut16 | (mN)#(fN)#(ibut)(mN)#(ibut)(mN)#(ibut)(mN)#(ibut)(mN)#(ibut)(mN) |

TABLE 3
LNA-containing chemical modification patterns

| | |
|---|---|
| P1_l1_as | P(lN)#(fN)#(mN)(fN)(fN)(fN)(mN)(fN)(mN)(fN)(mN)(fN)(mN)(fN)#(mN)#(fN) |
| s | #(mN)#(mN)#(mN)#(fN)#(mN) |
| P1_l2_as | P(mN)#(lN)#(mN)(fN)(fN)(fN)(mN)(fN)(mN)(fN)(mN)(fN)(mN)(fN)#(mN)# |
| s | (fN)#(mN)#(mN)#(mN)#(fN)#(mN) |
| P1_l3_as | P(mN)#(fN)#(lN)(fN)(fN)(fN)(mN)(fN)(mN)(fN)(mN)(fN)(mN)(fN)#(mN)# |
| s | (fN)#(mN)#(mN)#(mN)#(fN)#(mN) |
| P1_l4_as | P(mN)#(fN)#(mN)(lN)(fN)(fN)(mN)(fN)(mN)(fN)(mN)(fN)(mN)(fN)#(mN)# |
| s | (fN)#(mN)#(mN)#(mN)#(fN)#(mN) |
| P1_l5_as | P(mN)#(fN)#(mN)(fN)(lN)(fN)(mN)(fN)(mN)(fN)(mN)(fN)(mN)(fN)#(mN)# |
| s | (fN)#(mN)#(mN)#(mN)#(fN)#(mN) |
| P1_l6_as | P(mN)#(fN)#(mN)(fN)(fN)(lN)(mN)(fN)(mN)(fN)(mN)(fN)(mN)(fN)#(mN)# |
| s | (fN)#(mN)#(mN)#(mN)#(fN)#(mN) |
| P1_l7_as | P(mN)#(fN)#(mN)(fN)(fN)(fN)(lN)(fN)(mN)(fN)(mN)(fN)(mN)(fN)#(mN)#(fN) |
| s | #(mN)#(mN)#(mN)#(fN)#(mN) |
| P1_l8_as | P(mN)#(fN)#(mN)(fN)(fN)(fN)(mN)(lN)(mN)(fN)(mN)(fN)(mN)(fN)#(mN)# |
| s | (fN)#(mN)#(mN)#(mN)#(fN)#(mN) |
| P1_l9_as | P(mN)#(fN)#(mN)(fN)(fN)(fN)(mN)(fN)(lN)(fN)(mN)(fN)(mN)(fN)#(mN)#(fN) |
| s | #(mN)#(mN)#(mN)#(fN)#(mN) |
| P1_l10_ | P(mN)#(fN)#(mN)(fN)(fN)(fN)(mN)(fN)(mN)(lN)(mN)(fN)(mN)(fN)#(mN)# |
| as | (fN)#(mN)#(mN)#(mN)#(fN)#(mN) |
| P1_l11_ | P(mN)#(fN)#(mN)(fN)(fN)(fN)(mN)(fN)(mN)(fN)(lN)(fN)(mN)(fN)#(mN)#(fN) |
| as | #(mN)#(mN)#(mN)#(fN)#(mN) |
| P1_l12_ | P(mN)#(fN)#(mN)(fN)(fN)(fN)(mN)(fN)(mN)(fN)(mN)(lN)(mN)(fN)#(mN)# |
| as | (fN)#(mN)#(mN)#(mN)#(fN)#(mN) |
| P1_l13_ | P(mN)#(fN)#(mN)(fN)(fN)(fN)(mN)(fN)(mN)(fN)(mN)(fN)(lN)(fN)#(mN)#(fN) |
| as | #(mN)#(mN)#(mN)#(fN)#(mN) |
| P1_l14_ | P(mN)#(fN)#(mN)(fN)(fN)(fN)(mN)(fN)(mN)(fN)(mN)(fN)(mN)(lN)#(mN)# |
| as | (fN)#(mN)#(mN)#(mN)#(fN)#(mN) |
| P1_l15_ | P(mN)#(fN)#(mN)(fN)(fN)(fN)(mN)(fN)(mN)(fN)(mN)(fN)(mN)(fN)#(lN)#(fN) |
| as | #(mN)#(mN)#(mN)#(fN)#(mN) |
| P1_l16_ | P(mN)#(fN)#(mN)(fN)(fN)(fN)(mN)(fN)(mN)(fN)(mN)(fN)(mN)(fN)#(mN)# |
| as | (lN)#(mN)#(mN)#(mN)#(fN)#(mN) |
| P1_l17_ | P(mN)#(fN)#(mN)(fN)(fN)(fN)(mN)(fN)(mN)(fN)(mN)(fN)(mN)(fN)#(mN)# |
| as | (fN)#(lN)#(mN)#(mN)#(fN)#(mN) |
| P1_l18_ | P(mN)#(fN)#(mN)(fN)(fN)(fN)(mN)(fN)(mN)(fN)(mN)(fN)(mN)(fN)#(mN)# |
| as | (fN)#(mN)#(lN)#(mN)#(fN)#(mN) |
| P1_l19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(lN)#(fN)#(mN) |
| P1_l20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(lN)#(mN) |
| P1_l21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(lN) |
| P2_l1_as | P(lN)#(fN)#(mN)(mC)(mN)(fN)(mN)(mN)(mN)(mN)(mC)(mN)(mN)(fN)#(mN) |
| s | #(fN)#(mC)#(mN)#(mN)#(fN)#(mN) |
| P2_l2_as | P(mN)#(lN)#(mN)(mC)(mN)(fN)(mN)(mN)(mN)(mN)(mC)(mN)(mN)(fN)#(mN) |
| s | #(fN)#(mC)#(mN)#(mN)#(fN)#(mN) |
| P2_l3_as | P(mN)#(fN)#(lN)(mC)(mN)(fN)(mN)(mN)(mN)(mN)(mC)(mN)(mN)(fN)#(mN) |
| s | #(fN)#(mC)#(mN)#(mN)#(fN)#(mN) |
| P2_l4_as | P(mN)#(fN)#(mN)(lC)(mN)(fN)(mN)(mN)(mN)(mN)(mC)(mN)(mN)(fN)#(mN) |
| s | #(fN)#(mC)#(mN)#(mN)#(fN)#(mN) |
| P2_l5_as | P(mN)#(fN)#(mN)(mC)(lN)(fN)(mN)(mN)(mN)(mN)(mC)(mN)(mN)(fN)#(mN) |
| s | #(fN)#(mC)#(mN)#(mN)#(fN)#(mN) |
| P2_l6_as | P(mN)#(fN)#(mN)(mC)(mN)(lN)(mN)(mN)(mN)(mN)(mC)(mN)(mN)(fN)#(mN) |
| s | #(fN)#(mC)#(mN)#(mN)#(fN)#(mN) |
| P2_l7_as | P(mN)#(fN)#(mN)(mC)(mN)(fN)(lN)(mN)(mN)(mN)(mC)(mN)(mN)(fN)#(mN) |
| s | #(fN)#(mC)#(mN)#(mN)#(fN)#(mN) |
| P2_l8_as | P(mN)#(fN)#(mN)(mC)(mN)(fN)(mN)(lN)(mN)(mN)(mC)(mN)(mN)(fN)#(mN) |
| s | #(fN)#(mC)#(mN)#(mN)#(fN)#(mN) |
| P2_l9_as | P(mN)#(fN)#(mN)(mC)(mN)(fN)(mN)(mN)(lN)(mN)(mC)(mN)(mN)(fN)#(mN) |
| s | #(fN)#(mC)#(mN)#(mN)#(fN)#(mN) |
| P2_l10_ | P(mN)#(fN)#(mN)(mC)(mN)(fN)(mN)(mN)(mN)(lN)(mC)(mN)(mN)(fN)#(mN) |
| as | #(fN)#(mC)#(mN)#(mN)#(fN)#(mN) |
| P2_l11_ | P(mN)#(fN)#(mN)(mC)(mN)(fN)(mN)(mN)(mN)(mN)(lC)(mN)(mN)(fN)#(mN) |
| as | #(fN)#(mC)#(mN)#(mN)#(fN)#(mN) |
| P2_l12_ | P(mN)#(fN)#(mN)(mC)(mN)(fN)(mN)(mN)(mN)(mN)(mC)(lN)(mN)(fN)#(mN) |
| as | #(fN)#(mC)#(mN)#(mN)#(fN)#(mN) |
| P2_l13_ | P(mN)#(fN)#(mN)(mC)(mN)(fN)(mN)(mN)(mN)(mN)(mC)(mN)(lN)(fN)#(mN) |
| as | #(fN)#(mC)#(mN)#(mN)#(fN)#(mN) |
| P2_l14_ | P(mN)#(fN)#(mN)(mC)(mN)(fN)(mN)(mN)(mN)(mN)(mC)(mN)(mN)(lN)# |
| as | (mN)#(fN)#(mC)#(mN)#(mN)#(fN)#(mN) |
| P2_l15_ | P(mN)#(fN)#(mN)(mC)(mN)(fN)(mN)(mN)(mN)(mN)(mC)(mN)(mN)(fN)#(lN) |
| as | #(fN)#(mC)#(mN)#(mN)#(fN)#(mN) |
| P2_l16_ | P(mN)#(fN)#(mN)(mC)(mN)(fN)(mN)(mN)(mN)(mN)(mC)(mN)(mN)(fN)#(mN) |
| as | #(lN)#(mC)#(mN)#(mN)#(fN)#(mN) |
| P2_l17_ | P(mN)#(fN)#(mN)(mC)(mN)(fN)(mN)(mN)(mN)(mN)(mC)(mN)(mN)(fN)#(mN) |
| as | #(fN)#(lC)#(mN)#(mN)#(fN)#(mN) |
| P2_l18_ | P(mN)#(fN)#(mN)(mC)(mN)(fN)(mN)(mN)(mN)(mN)(mC)(mN)(mN)(fN)#(mN) |
| as | #(fN)#(mC)#(lN)#(mN)#(fN)#(mN) |
| P2_l19_ | P(mN)#(fN)#(mN)(mC)(mN)(fN)(mN)(mN)(mN)(mN)(mC)(mN)(mN)(fN)#(mN) |
| as | #(fN)#(mC)#(mN)#(lN)#(fN)#(mN) |
| P2_l20_ | P(mN)#(fN)#(mN)(mC)(mN)(fN)(mN)(mN)(mN)(mN)(mC)(mN)(mN)(fN)#(mN) |
| as | #(fN)#(mC)#(mN)#(mN)#(lN)#(mN) |
| P2_l21_ | P(mN)#(fN)#(mN)(mC)(mN)(fN)(mN)(mN)(mN)(mN)(mC)(mN)(mN)(fN)#(mN) |
| as | #(fN)#(mC)#(mN)#(mN)#(fN)#(lN) |
| P1_l1_s | (lN)#(mN)#(mN)(fN)(mN)(fN)(mN)(fN)(mN)(fN)(mN)(mN)(mN)(fN)#(mN)# |
| | (mN) |
| P1_l2_s | (mN)#(lN)#(mN)(fN)(mN)(fN)(mN)(fN)(mN)(fN)(mN)(mN)(mN)(fN)#(mN)# |
| | (mN) |
| P1_l3_s | (mN)#(mN)#(lN)(fN)(mN)(fN)(mN)(fN)(mN)(fN)(mN)(mN)(mN)(fN)#(mN)# |
| | (mN) |
| P1_l4_s | (mN)#(mN)#(mN)(lN)(mN)(fN)(mN)(fN)(mN)(fN)(mN)(mN)(mN)(fN)#(mN)# |
| | (mN) |
| P1_l5_s | (mN)#(mN)#(mN)(fN)(lN)(fN)(mN)(fN)(mN)(fN)(mN)(mN)(mN)(fN)#(mN)# |
| | (mN) |
| P1_l6_s | (mN)#(mN)#(mN)(fN)(mN)(lN)(mN)(fN)(mN)(fN)(mN)(mN)(mN)(fN)#(mN)# |
| | (mN) |
| P1_l7_s | (mN)#(mN)#(mN)(fN)(mN)(fN)(lN)(fN)(mN)(fN)(mN)(mN)(mN)(fN)#(mN)# |
| | (mN) |
| P1_l8_s | (mN)#(mN)#(mN)(fN)(mN)(fN)(mN)(lN)(mN)(fN)(mN)(mN)(mN)(fN)#(mN)# |
| | (mN) |
| P1_l9_s | (mN)#(mN)#(mN)(fN)(mN)(fN)(mN)(fN)(lN)(fN)(mN)(mN)(mN)(fN)#(mN)# |
| | (mN) |
| P1_l10_ | (mN)#(mN)#(mN)(fN)(mN)(fN)(mN)(fN)(mN)(lN)(mN)(mN)(mN)(fN)#(mN)# |
| s | (mN) |
| P1_l11_ | (mN)#(mN)#(mN)(fN)(mN)(fN)(mN)(fN)(mN)(fN)(lN)(mN)(mN)(fN)#(mN)# |
| s | (mN) |
| P1_l12_ | (mN)#(mN)#(mN)(fN)(mN)(fN)(mN)(fN)(mN)(fN)(mN)(lN)(mN)(fN)#(mN)# |
| s | (mN) |
| P1_l13_ | (mN)#(mN)#(mN)(fN)(mN)(fN)(mN)(fN)(mN)(fN)(mN)(mN)(lN)(fN)#(mN)# |
| s | (mN) |
| P1_l14_ | (mN)#(mN)#(mN)(fN)(mN)(fN)(mN)(fN)(mN)(fN)(mN)(mN)(mN)(lN)#(mN)# |
| s | (mN) |
| P1_l15_ | (mN)#(mN)#(mN)(fN)(mN)(fN)(mN)(fN)(mN)(fN)(mN)(mN)(mN)(fN)#(lN)# |
| s | (mN) |
| P1_l16_ | (mN)#(mN)#(mN)(fN)(mN)(fN)(mN)(fN)(mN)(fN)(mN)(mN)(mN)(fN)#(mN)# |
| s | (lN) |
| P2_l1_s | (lN)#(mN)#(mN)(mN)(mN)(fN)(fN)(fN)(mN)(fN)(mN)(mN)(mN)(mN)#(mN)# |
| | (mN) |
| P2_l2_s | (mN)#(lN)#(mN)(mN)(mN)(fN)(fN)(fN)(mN)(fN)(mN)(mN)(mN)(mN)#(mN)# |
| | (mN) |
| P2_l3_s | (mN)#(mN)#(lN)(mN)(mN)(fN)(fN)(fN)(mN)(fN)(mN)(mN)(mN)(mN)#(mN)# |
| | (mN) |
| P2_l4_s | (mN)#(mN)#(mN)(lN)(mN)(fN)(fN)(fN)(mN)(fN)(mN)(mN)(mN)(mN)#(mN)# |
| | (mN) |
| P2_l5_s | (mN)#(mN)#(mN)(mN)(lN)(fN)(fN)(fN)(mN)(fN)(mN)(mN)(mN)(mN)#(mN)# |
| | (mN) |
| P2_l6_s | (mN)#(mN)#(mN)(mN)(mN)(lN)(fN)(fN)(mN)(fN)(mN)(mN)(mN)(mN)#(mN) |
| | #(mN) |
| P2_l7_s | (mN)#(mN)#(mN)(mN)(mN)(fN)(lN)(fN)(mN)(fN)(mN)(mN)(mN)(mN)#(mN) |
| | #(mN) |
| P2_l8_s | (mN)#(mN)#(mN)(mN)(mN)(fN)(fN)(lN)(mN)(fN)(mN)(mN)(mN)(mN)#(mN) |
| | #(mN) |
| P2_l9_s | (mN)#(mN)#(mN)(mN)(mN)(fN)(fN)(fN)(lN)(fN)(mN)(mN)(mN)(mN)#(mN)# |
| | (mN) |
| P2_l10_ | (mN)#(mN)#(mN)(mN)(mN)(fN)(fN)(fN)(mN)(lN)(mN)(mN)(mN)(mN)#(mN) |
| s | #(mN) |
| P2_l11_ | (mN)#(mN)#(mN)(mN)(mN)(fN)(fN)(fN)(mN)(fN)(lN)(mN)(mN)(mN)#(mN)# |
| s | (mN) |
| P2_l12_ | (mN)#(mN)#(mN)(mN)(mN)(fN)(fN)(fN)(mN)(fN)(mN)(lN)(mN)(mN)#(mN)# |
| s | (mN) |
| P2_l13_ | (mN)#(mN)#(mN)(mN)(mN)(fN)(fN)(fN)(mN)(fN)(mN)(mN)(lN)(mN)#(mN)# |
| s | (mN) |
| P2_l14_ | (mN)#(mN)#(mN)(mN)(mN)(fN)(fN)(fN)(mN)(fN)(mN)(mN)(mN)(lN)#(mN)# |
| s | (mN) |
| P2_l15_ | (mN)#(mN)#(mN)(mN)(mN)(fN)(fN)(fN)(mN)(fN)(mN)(mN)(mN)(mN)#(lN)# |
| s | (mN) |
| P2_l16_ | (mN)#(mN)#(mN)(mN)(mN)(fN)(fN)(fN)(mN)(fN)(mN)(mN)(mN)(mN)#(mN) |
| s | #(lN) |
| as_TW_ | P(mN)#(fN)#(mN)(mN)(mN)(fN)(mN)(mN)(mN)(mN)(mN)(mN)(mN)(fN)# |
| L1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| L2 | (mN)#(fN)#(lN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| L3 | (mN)#(fN)#(mN)#(lN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| L4 | (mN)#(fN)#(mN)#(mN)#(lN)#(mN)#(mN) |
| as_TW_ | P(mN)#(fN)#(mN)(mN)(mN)(fN)(mN)(mN)(mN)(mN)(mN)(mN)(mN)(fN)# |
| L5 | (mN)#(fN)#(mN)#(mN)#(mN)#(lN)#(mN) |
| as_TW_ | P(mN)#(fN)#(mN)(mN)(mN)(fN)(mN)(mN)(mN)(mN)(mN)(mN)(mN)(fN)# |
| L6 | (mN)#(fN)#(mN)#(mN)#(mN)#(mN)#(lN) |
| as_TW_ | P(mN)#(fN)#(mN)(mN)(mN)(fN)(mN)(mN)(mN)(mN)(mN)(mN)(mN)(fN)# |
| L7 | (mN)#(fN)#(lN)#(lN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| L8 | (mN)#(fN)#(mN)#(lN)#(lN)#(mN)#(mN) |
| as_TW_ | P(mN)#(fN)#(mN)(mN)(mN)(fN)(mN)(mN)(mN)(mN)(mN)(mN)(mN)(fN)# |
| L9 | (mN)#(fN)#(mN)#(mN)#(lN)#(lN)#(mN) |
| as_TW_ | P(mN)#(fN)#(mN)(mN)(mN)(fN)(mN)(mN)(mN)(mN)(mN)(mN)(mN)(fN)# |
| L10 | (mN)#(fN)#(mN)#(mN)#(mN)#(lN)#(lN) |
| as_TW_ | P(mN)#(fN)#(mN)(mN)(mN)(fN)(mN)(mN)(mN)(mN)(mN)(mN)(mN)(fN)# |
| L11 | (mN)#(fN)#(lN)#(mN)#(lN)#(mN)#(mN) |
| as_TW_ | P(mN)#(fN)#(mN)(mN)(mN)(fN)(mN)(mN)(mN)(mN)(mN)(mN)(mN)(fN)# |
| L12 | (mN)#(fN)#(mN)#(mN)#(lN)#(mN)#(lN) |
| as_TW_ | P(mN)#(fN)#(mN)(mN)(mN)(fN)(mN)(mN)(mN)(mN)(mN)(mN)(mN)(fN)# |
| L13 | (mN)#(fN)#(mN)#(lN)#(mN)#(lN)#(mN) |
| as_TW_ | P(mN)#(fN)#(mN)(mN)(mN)(fN)(mN)(mN)(mN)(mN)(mN)(mN)(mN)(fN)# |
| L14 | (mN)#(fN)#(lN)#(lN)#(lN)#(mN)#(mN) |
| as_TW_ | P(mN)#(fN)#(mN)(mN)(mN)(fN)(mN)(mN)(mN)(mN)(mN)(mN)(mN)(fN)# |
| L15 | (mN)#(fN)#(mN)#(mN)#(lN)#(lN)#(lN) |
| as_TW_ | P(mN)#(fN)#(mN)(mN)(mN)(fN)(mN)(mN)(mN)(mN)(mN)(mN)(mN)(fN)# |
| L16 | (mN)#(fN)#(lN)#(lN)#(lN)#(lN)#(lN) |

TABLE 4
MOE-containing chemical modification patterns

| | |
|---|---|
| P1_e1_as | P(eN)#(fN)#(mN)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_e2_as | P(mN)#(eN)#(mN)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_e3_as | P(mN)#(fN)#(eN)(fN)(fN)(fN)(mN)(fN)(mN)(fN)(mN)(fN)(mN)(fN)#(mN)# |
| | (fN)#(mN)#(mN)#(mN)#(fN)#(mN) |
| P1_e4_as | P(mN)#(fN)#(mN)(eN)(fN)(fN)(mN)(fN)(mN)(fN)(mN)(fN)(mN)(fN)#(mN)# |
| | (fN)#(mN)#(mN)#(mN)#(fN)#(mN) |
| P1_e5_as | P(mN)#(fN)#(mN)(fN)(eN)(fN)(mN)(fN)(mN)(fN)(mN)(fN)(mN)(fN)#(mN)# |
| | (fN)#(mN)#(mN)#(mN)#(fN)#(mN) |
| P1_e6_as | P(mN)#(fN)#(mN)(fN)(fN)(eN)(mN)(fN)(mN)(fN)(mN)(fN)(mN)(fN)#(mN)# |
| | (fN)#(mN)#(mN)#(mN)#(fN)#(mN) |
| P1_e7_as | P(mN)#(fN)#(mN)(fN)(fN)(fN)(eN)(fN)(mN)(fN)(mN)(fN)(mN)(fN)#(mN)# |
| | (fN)#(mN)#(mN)#(mN)#(fN)#(mN) |
| P1_e8_as | P(mN)#(fN)#(mN)(fN)(fN)(fN)(mN)(eN)(mN)(fN)(mN)(fN)(mN)(fN)#(mN)# |
| | (fN)#(mN)#(mN)#(mN)#(fN)#(mN) |
| P1_e9_as | P(mN)#(fN)#(mN)(fN)(fN)(fN)(mN)(fN)(eN)(fN)(mN)(fN)(mN)(fN)#(mN)# |
| | (fN)#(mN)#(mN)#(mN)#(fN)#(mN) |
| P1_e10_ | P(mN)#(fN)#(mN)(fN)(fN)(fN)(mN)(fN)(mN)(eN)(mN)(fN)(mN)(fN)#(mN)# |
| as | (fN)#(mN)#(mN)#(mN)#(fN)#(mN) |
| P1_e11_ | P(mN)#(fN)#(mN)(fN)(fN)(fN)(mN)(fN)(mN)(fN)(eN)(fN)(mN)(fN)#(mN)# |
| as | (fN)#(mN)#(mN)#(mN)#(fN)#(mN) |
| P1_e12_ | P(mN)#(fN)#(mN)(fN)(fN)(fN)(mN)(fN)(mN)(fN)(mN)(eN)(mN)(fN)#(mN)# |
| as | (fN)#(mN)#(mN)#(mN)#(fN)#(mN) |
| P1_e13_ | P(mN)#(fN)#(mN)(fN)(fN)(fN)(mN)(fN)(mN)(fN)(mN)(fN)(eN)(fN)#(mN)# |
| as | (fN)#(mN)#(mN)#(mN)#(fN)#(mN) |
| P1_e14_ | P(mN)#(fN)#(mN)(fN)(fN)(fN)(mN)(fN)(mN)(fN)(mN)(fN)(mN)(eN)#(mN)# |
| as | (fN)#(mN)#(mN)#(mN)#(fN)#(mN) |
| P1_e15_ | P(mN)#(fN)#(mN)(fN)(fN)(fN)(mN)(fN)(mN)(fN)(mN)(fN)(mN)(fN)#(eN)# |
| as | (fN)#(mN)#(mN)#(mN)#(fN)#(mN) |
| P1_e16_ | P(mN)#(fN)#(mN)(fN)(fN)(fN)(mN)(fN)(mN)(fN)(mN)(fN)(mN)(fN)#(mN)# |
| as | (eN)#(mN)#(mN)#(mN)#(fN)#(mN) |
| P1_e17_ | P(mN)#(fN)#(mN)(fN)(fN)(fN)(mN)(fN)(mN)(fN)(mN)(fN)(mN)(fN)#(mN)# |
| as | (fN)#(eN)#(mN)#(mN)#(fN)#(mN) |
| P1_e18_ | P(mN)#(fN)#(mN)(fN)(fN)(fN)(mN)(fN)(mN)(fN)(mN)(fN)(mN)(fN)#(mN)# |
| as | (fN)#(mN)#(eN)#(mN)#(fN)#(mN) |
| P1_e19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(eN)#(fN)#(mN) |
| P1_e20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(eN)#(mN) |
| P1_e21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(eN) |
| P2_e1_ | P(eN)#(fN)#(mN)(mN)(mN)(fN)(mN)(mN)(mN)(mN)(mN)(mN)(mN)(fN)# |
| Ns | (mN)#(fN)#(mN)#(mN)#(mN)#(fN)#(mN) |
| P2_e2_ | P(mN)#(eN)#(mN)(mN)(mN)(fN)(mN)(mN)(mN)(mN)(mN)(mN)(mN)(fN)# |
| Ns | (mN)#(fN)#(mN)#(mN)#(mN)#(fN)#(mN) |
| P2_e3_ | P(mN)#(fN)#(eN)(mN)(mN)(fN)(mN)(mN)(mN)(mN)(mN)(mN)(mN)(fN)#(mN) |
| Ns | #(fN)#(mN)#(mN)#(mN)#(fN)#(mN) |
| P2_e4_ | P(mN)#(fN)#(mN)(eN)(mN)(fN)(mN)(mN)(mN)(mN)(mN)(mN)(mN)(fN)#(mN) |
| Ns | #(fN)#(mN)#(mN)#(mN)#(fN)#(mN) |
| P2_e5_ | P(mN)#(fN)#(mN)(mN)(eN)(fN)(mN)(mN)(mN)(mN)(mN)(mN)(mN)(fN)#(mN) |
| Ns | #(fN)#(mN)#(mN)#(mN)#(fN)#(mN) |
| P2_e6_ | P(mN)#(fN)#(mN)(mN)(mN)(eN)(mN)(mN)(mN)(mN)(mN)(mN)(mN)(fN)#(mN) |
| Ns | #(fN)#(mN)#(mN)#(mN)#(fN)#(mN) |
| P2_e7_ | P(mN)#(fN)#(mN)(mN)(mN)(fN)(eN)(mN)(mN)(mN)(mN)(mN)(mN)(fN)#(mN) |
| Ns | #(fN)#(mN)#(mN)#(mN)#(fN)#(mN) |
| P2_e8_ | P(mN)#(fN)#(mN)(mN)(mN)(fN)(mN)(eN)(mN)(mN)(mN)(mN)(mN)(fN)#(mN) |
| Ns | #(fN)#(mN)#(mN)#(mN)#(fN)#(mN) |
| P2_e9_ | P(mN)#(fN)#(mN)(mN)(mN)(fN)(mN)(mN)(eN)(mN)(mN)(mN)(mN)(fN)#(mN) |
| Ns | #(fN)#(mN)#(mN)#(mN)#(fN)#(mN) |
| P2_e10_ | P(mN)#(fN)#(mN)(mN)(mN)(fN)(mN)(mN)(mN)(eN)(mN)(mN)(mN)(fN)#(mN) |
| Ns | #(fN)#(mN)#(mN)#(mN)#(fN)#(mN) |
| P2_e11_ | P(mN)#(fN)#(mN)(mN)(mN)(fN)(mN)(mN)(mN)(mN)(eN)(mN)(mN)(fN)#(mN) |
| Ns | #(fN)#(mN)#(mN)#(mN)#(fN)#(mN) |
| P2_e12_ | P(mN)#(fN)#(mN)(mN)(mN)(fN)(mN)(mN)(mN)(mN)(mN)(eN)(mN)(fN)#(mN) |
| Ns | #(fN)#(mN)#(mN)#(mN)#(fN)#(mN) |
| P2_e13_ | P(mN)#(fN)#(mN)(mN)(mN)(fN)(mN)(mN)(mN)(mN)(mN)(mN)(eN)(fN)#(mN) |
| Ns | #(fN)#(mN)#(mN)#(mN)#(fN)#(mN) |
| P2_e14_ | P(mN)#(fN)#(mN)(mN)(mN)(fN)(mN)(mN)(mN)(mN)(mN)(mN)(mN)(eN)#(mN) |
| Ns | #(fN)#(mN)#(mN)#(mN)#(fN)#(mN) |
| P2_e15_ | P(mN)#(fN)#(mN)(mN)(mN)(fN)(mN)(mN)(mN)(mN)(mN)(mN)(mN)(fN)#(eN) |
| Ns | #(fN)#(mN)#(mN)#(mN)#(fN)#(mN) |
| P2_e16_ | P(mN)#(fN)#(mN)(mN)(mN)(fN)(mN)(mN)(mN)(mN)(mN)(mN)(mN)(fN)#(mN) |
| Ns | #(eN)#(mN)#(mN)#(mN)#(fN)#(mN) |
| P2_e17_ | P(mN)#(fN)#(mN)(mN)(mN)(fN)(mN)(mN)(mN)(mN)(mN)(mN)(mN)(fN)#(mN) |
| Ns | #(fN)#(eN)#(mN)#(mN)#(fN)#(mN) |
| P2_e18_ | P(mN)#(fN)#(mN)(mN)(mN)(fN)(mN)(mN)(mN)(mN)(mN)(mN)(mN)(fN)#(mN) |
| Ns | #(fN)#(mN)#(eN)#(mN)#(fN)#(mN) |
| P2_e19_ | P(mN)#(fN)#(mN)(mN)(mN)(fN)(mN)(mN)(mN)(mN)(mN)(mN)(mN)(fN)#(mN) |
| Ns | #(fN)#(mN)#(mN)#(eN)#(fN)#(mN) |
| P2_e20_ | P(mN)#(fN)#(mN)(mN)(mN)(fN)(mN)(mN)(mN)(mN)(mN)(mN)(mN)(fN)#(mN) |
| Ns | #(fN)#(mN)#(mN)#(mN)#(eN)#(mN) |
| P2_e21_ | P(mN)#(fN)#(mN)(mN)(mN)(fN)(mN)(mN)(mN)(mN)(mN)(mN)(mN)(fN)#(mN) |
| Ns | #(fN)#(mN)#(mN)#(mN)#(fN)#(eN) |
| P1_e1_s | (eN)#(mN)#(mN)(fN)(mN)(fN)(mN)(fN)(mN)(fN)(mN)(mN)(mN)(fN)#(mN)# |
| | (mN) |
| P1_e2_s | (mN)#(eN)#(mN)(fN)(mN)(fN)(mN)(fN)(mN)(fN)(mN)(mN)(mN)(fN)#(mN)# |
| | (mN) |
| P1_e3_s | (mN)#(mN)#(eN)(fN)(mN)(fN)(mN)(fN)(mN)(fN)(mN)(mN)(mN)(fN)#(mN)# |
| | (mN) |
| P1_e4_s | (mN)#(mN)#(mN)(eN)(mN)(fN)(mN)(fN)(mN)(fN)(mN)(mN)(mN)(fN)#(mN) |
| | #(mN) |
| P1_e5_s | (mN)#(mN)#(mN)(fN)(eN)(fN)(mN)(fN)(mN)(fN)(mN)(mN)(mN)(fN)#(mN)# |
| | (mN) |
| P1_e6_s | (mN)#(mN)#(mN)(fN)(mN)(eN)(mN)(fN)(mN)(fN)(mN)(mN)(mN)(fN)#(mN) |
| | #(mN) |
| P1_e7_s | (mN)#(mN)#(mN)(fN)(mN)(fN)(eN)(fN)(mN)(fN)(mN)(mN)(mN)(fN)#(mN)# |
| | (mN) |
| P1_e8_s | (mN)#(mN)#(mN)(fN)(mN)(fN)(mN)(eN)(mN)(fN)(mN)(mN)(mN)(fN)#(mN) |
| | #(mN) |
| P1_e9_s | (mN)#(mN)#(mN)(fN)(mN)(fN)(mN)(fN)(eN)(fN)(mN)(mN)(mN)(fN)#(mN)# |
| | (mN) |
| P1_e10_ | (mN)#(mN)#(mN)(fN)(mN)(fN)(mN)(fN)(mN)(eN)(mN)(mN)(mN)(fN)#(mN) |
| s | #(mN) |
| P1_e11_ | (mN)#(mN)#(mN)(fN)(mN)(fN)(mN)(fN)(mN)(fN)(eN)(mN)(mN)(fN)#(mN)# |
| s | (mN) |
| P1_e12_ | (mN)#(mN)#(mN)(fN)(mN)(fN)(mN)(fN)(mN)(fN)(mN)(eN)(mN)(fN)#(mN)# |
| s | (mN) |
| P1_e13_ | (mN)#(mN)#(mN)(fN)(mN)(fN)(mN)(fN)(mN)(fN)(mN)(mN)(eN)(fN)#(mN)# |
| s | (mN) |
| P1_e14_ | (mN)#(mN)#(mN)(fN)(mN)(fN)(mN)(fN)(mN)(fN)(mN)(mN)(mN)(eN)#(mN) |
| s | #(mN) |
| P1_e15_ | (mN)#(mN)#(mN)(fN)(mN)(fN)(mN)(fN)(mN)(fN)(mN)(mN)(mN)(fN)#(eN)# |
| s | (mN) |
| P1_e16_ | (mN)#(mN)#(mN)(fN)(mN)(fN)(mN)(fN)(mN)(fN)(mN)(mN)(mN)(fN)#(mN)# |
| s | (eN) |
| P2_e1_s | (eN)#(mN)#(mN)(mN)(mN)(fN)(fN)(fN)(mN)(fN)(mN)(mN)(mN)(mN)#(mN) |
| | #(mN) |
| P2_e2_s | (mN)#(eN)#(mN)(mN)(mN)(fN)(fN)(fN)(mN)(fN)(mN)(mN)(mN)(mN)#(mN) |
| | #(mN) |
| P2_e3_s | (mN)#(mN)#(eN)(mN)(mN)(fN)(fN)(fN)(mN)(fN)(mN)(mN)(mN)(mN)#(mN) |
| | #(mN) |
| P2_e4_s | (mN)#(mN)#(mN)(eN)(mN)(fN)(fN)(fN)(mN)(fN)(mN)(mN)(mN)(mN)#(mN) |
| | #(mN) |
| P2_e5_s | (mN)#(mN)#(mN)(mN)(eN)(fN)(fN)(fN)(mN)(fN)(mN)(mN)(mN)(mN)#(mN) |
| | #(mN) |
| P2_e6_s | (mN)#(mN)#(mN)(mN)(mN)(eN)(fN)(fN)(mN)(fN)(mN)(mN)(mN)(mN)#(mN) |
| | #(mN) |
| P2_e7_s | (mN)#(mN)#(mN)(mN)(mN)(fN)(eN)(fN)(mN)(fN)(mN)(mN)(mN)(mN)#(mN) |
| | #(mN) |
| P2_e8_s | (mN)#(mN)#(mN)(mN)(mN)(fN)(fN)(eN)(mN)(fN)(mN)(mN)(mN)(mN)#(mN) |
| | #(mN) |
| P2_e9_s | (mN)#(mN)#(mN)(mN)(mN)(fN)(fN)(fN)(eN)(fN)(mN)(mN)(mN)(mN)#(mN) |
| | #(mN) |
| P2_e10_ | (mN)#(mN)#(mN)(mN)(mN)(fN)(fN)(fN)(mN)(eN)(mN)(mN)(mN)(mN)#(mN) |
| s | #(mN) |
| P2_e11_ | (mN)#(mN)#(mN)(mN)(mN)(fN)(fN)(fN)(mN)(fN)(eN)(mN)(mN)(mN)#(mN) |
| s | #(mN) |
| P2_e12_ | (mN)#(mN)#(mN)(mN)(mN)(fN)(fN)(fN)(mN)(fN)(mN)(eN)(mN)(mN)#(mN) |
| s | #(mN) |
| P2_e13_ | (mN)#(mN)#(mN)(mN)(mN)(fN)(fN)(fN)(mN)(fN)(mN)(mN)(eN)(mN)#(mN) |
| s | #(mN) |
| P2_e14_ | (mN)#(mN)#(mN)(mN)(mN)(fN)(fN)(fN)(mN)(fN)(mN)(mN)(mN)(eN)#(mN) |
| s | #(mN) |
| P2_e15_ | (mN)#(mN)#(mN)(mN)(mN)(fN)(fN)(fN)(mN)(fN)(mN)(mN)(mN)(mN)#(eN) |
| s | #(mN) |
| P2_e16_ | (mN)#(mN)#(mN)(mN)(mN)(fN)(fN)(fN)(mN)(fN)(mN)(mN)(mN)(mN)#(mN) |
| s | #(eN) |
| as_TW_ | P(mN)#(fN)#(mN)(mN)(mN)(fN)(mN)(mN)(mN)(mN)(mN)(mN)(mN)(fN)# |
| M1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| M2 | (mN)#(fN)#(eN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| M3 | (mN)#(fN)#(mN)#(eN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| M4 | (mN)#(fN)#(mN)#(mN)#(eN)#(mN)#(mN) |
| as_TW_ | P(mN)#(fN)#(mN)(mN)(mN)(fN)(mN)(mN)(mN)(mN)(mN)(mN)(mN)(fN)#(mN) |
| M5 | #(fN)#(mN)#(mN)#(mN)#(eN)#(mN) |
| as_TW_ | P(mN)#(fN)#(mN)(mN)(mN)(fN)(mN)(mN)(mN)(mN)(mN)(mN)(mN)(fN)#(mN) |
| M6 | #(fN)#(mN)#(mN)#(mN)#(mN)#(eN) |
| as_TW_ | P(mN)#(fN)#(mN)(mN)(mN)(fN)(mN)(mN)(mN)(mN)(mN)(mN)(mN)(fN)#(mN) |
| M7 | #(fN)#(eN)#(eN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| M8 | #(fN)#(mN)#(eN)#(eN)#(mN)#(mN) |
| as_TW_ | P(mN)#(fN)#(mN)(mN)(mN)(fN)(mN)(mN)(mN)(mN)(mN)(mN)(mN)(fN)#(mN) |
| M9 | #(fN)#(mN)#(mN)#(eN)#(eN)#(mN) |
| as_TW_ | P(mN)#(fN)#(mN)(mN)(mN)(fN)(mN)(mN)(mN)(mN)(mN)(mN)(mN)(fN)#(mN) |
| M10 | #(fN)#(mN)#(mN)#(mN)#(eN)#(eN) |
| as_TW_ | P(mN)#(fN)#(mN)(mN)(mN)(fN)(mN)(mN)(mN)(mN)(mN)(mN)(mN)(fN)#(mN) |
| M11 | #(fN)#(eN)#(mN)#(eN)#(mN)#(mN) |
| as_TW_ | P(mN)#(fN)#(mN)(mN)(mN)(fN)(mN)(mN)(mN)(mN)(mN)(mN)(mN)(fN)#(mN) |
| M12 | #(fN)#(mN)#(mN)#(eN)#(mN)#(eN) |
| as_TW_ | P(mN)#(fN)#(mN)(mN)(mN)(fN)(mN)(mN)(mN)(mN)(mN)(mN)(mN)(fN)#(mN) |
| M13 | #(fN)#(mN)#(eN)#(mN)#(eN)#(mN) |
| as_TW_ | P(mN)#(fN)#(mN)(mN)(mN)(fN)(mN)(mN)(mN)(mN)(mN)(mN)(mN)(fN)#(mN) |
| M14 | #(fN)#(eN)#(eN)#(eN)#(mN)#(mN) |
| as_TW_ | P(mN)#(fN)#(mN)(mN)(mN)(fN)(mN)(mN)(mN)(mN)(mN)(mN)(mN)(fN)#(mN) |
| M15 | #(fN)#(mN)#(mN)#(eN)#(eN)#(eN) |
| as_TW_ | P(mN)#(fN)#(mN)(mN)(mN)(fN)(mN)(mN)(mN)(mN)(mN)(mN)(mN)(fN)#(mN) |
| M16 | #(fN)#(eN)#(eN)#(eN)#(eN)#(eN) |

TABLE 5
DNA-containing chemical modification patterns

| | |
|---|---|
| P1_d1_ | P(dN)#(fN)#(mN)(fN)(fN)(fN)(mN)(fN)(mN)(fN)(mN)(fN)(mN)(fN)#(mN)#(fN) |
| as | #(mN)#(mN)#(mN)#(fN)#(mN) |
| P1_d2_ | P(mN)#(dN)#(mN)(fN)(fN)(fN)(mN)(fN)(mN)(fN)(mN)(fN)(mN)(fN)#(mN)# |
| as | (fN)#(mN)#(mN)#(mN)#(fN)#(mN) |
| P1_d3_ | P(mN)#(fN)#(dN)(fN)(fN)(fN)(mN)(fN)(mN)(fN)(mN)(fN)(mN)(fN)#(mN)# |
| as | (fN)#(mN)#(mN)#(mN)#(fN)#(mN) |
| P1_d4_ | P(mN)#(fN)#(mN)(dN)(fN)(fN)(mN)(fN)(mN)(fN)(mN)(fN)(mN)(fN)#(mN)# |
| as | (fN)#(mN)#(mN)#(mN)#(fN)#(mN) |
| P1_d5_ | P(mN)#(fN)#(mN)(fN)(dN)(fN)(mN)(fN)(mN)(fN)(mN)(fN)(mN)(fN)#(mN)# |
| as | (fN)#(mN)#(mN)#(mN)#(fN)#(mN) |
| P1_d6_ | P(mN)#(fN)#(mN)(fN)(fN)(dN)(mN)(fN)(mN)(fN)(mN)(fN)(mN)(fN)#(mN)# |
| as | (fN)#(mN)#(mN)#(mN)#(fN)#(mN) |
| P1_d7_ | P(mN)#(fN)#(mN)(fN)(fN)(fN)(dN)(fN)(mN)(fN)(mN)(fN)(mN)(fN)#(mN)# |
| as | (fN)#(mN)#(mN)#(mN)#(fN)#(mN) |
| P1_d8_ | P(mN)#(fN)#(mN)(fN)(fN)(fN)(mN)(dN)(mN)(fN)(mN)(fN)(mN)(fN)#(mN)# |
| as | (fN)#(mN)#(mN)#(mN)#(fN)#(mN) |
| P1_d9_ | P(mN)#(fN)#(mN)(fN)(fN)(fN)(mN)(fN)(dN)(fN)(mN)(fN)(mN)(fN)#(mN)# |
| as | (fN)#(mN)#(mN)#(mN)#(fN)#(mN) |
| P1_d10_ | P(mN)#(fN)#(mN)(fN)(fN)(fN)(mN)(fN)(mN)(dN)(mN)(fN)(mN)(fN)#(mN)# |
| as | (fN)#(mN)#(mN)#(mN)#(fN)#(mN) |
| P1_d11_ | P(mN)#(fN)#(mN)(fN)(fN)(fN)(mN)(fN)(mN)(fN)(dN)(fN)(mN)(fN)#(mN)# |
| as | (fN)#(mN)#(mN)#(mN)#(fN)#(mN) |
| P1_d12_ | P(mN)#(fN)#(mN)(fN)(fN)(fN)(mN)(fN)(mN)(fN)(mN)(dN)(mN)(fN)#(mN)# |
| as | (fN)#(mN)#(mN)#(mN)#(fN)#(mN) |
| P1_d13_ | P(mN)#(fN)#(mN)(fN)(fN)(fN)(mN)(fN)(mN)(fN)(mN)(fN)(dN)(fN)#(mN)# |
| as | (fN)#(mN)#(mN)#(mN)#(fN)#(mN) |
| P1_d14_ | P(mN)#(fN)#(mN)(fN)(fN)(fN)(mN)(fN)(mN)(fN)(mN)(fN)(mN)(dN)#(mN)# |
| as | (fN)#(mN)#(mN)#(mN)#(fN)#(mN) |
| P1_d15_ | P(mN)#(fN)#(mN)(fN)(fN)(fN)(mN)(fN)(mN)(fN)(mN)(fN)(mN)(fN)#(dN)# |
| as | (fN)#(mN)#(mN)#(mN)#(fN)#(mN) |
| P1_d16_ | P(mN)#(fN)#(mN)(fN)(fN)(fN)(mN)(fN)(mN)(fN)(mN)(fN)(mN)(fN)#(mN)# |
| as | (dN)#(mN)#(mN)#(mN)#(fN)#(mN) |
| P1_d17_ | P(mN)#(fN)#(mN)(fN)(fN)(fN)(mN)(fN)(mN)(fN)(mN)(fN)(mN)(fN)#(mN)# |
| as | (fN)#(dN)#(mN)#(mN)#(fN)#(mN) |
| P1_d18_ | P(mN)#(fN)#(mN)(fN)(fN)(fN)(mN)(fN)(mN)(fN)(mN)(fN)(mN)(fN)#(mN)# |
| as | (fN)#(mN)#(dN)#(mN)#(fN)#(mN) |
| P1_d19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(dN)#(fN)#(mN) |
| P1_d20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(dN)#(mN) |
| P1_d21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(dN) |
| P2_d1_ | P(dN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_d2_ | P(mN)#(dN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_d3_ | P(mN)#(fN)#(dN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_d4_ | P(mN)#(fN)#(mN)(dN)(mN)(fN)(mN)(mN)(mN)(mN)(mN)(mN)(mN)(fN)# |
| as | (mN)#(fN)#(mN)#(mN)#(mN)#(fN)#(mN) |
| P2_d5_ | P(mN)#(fN)#(mN)(mN)(dN)(fN)(mN)(mN)(mN)(mN)(mN)(mN)(mN)(fN)# |
| as | (mN)#(fN)#(mN)#(mN)#(mN)#(fN)#(mN) |
| P2_d6_ | P(mN)#(fN)#(mN)(mN)(mN)(dN)(mN)(mN)(mN)(mN)(mN)(mN)(mN)(fN)# |
| as | (mN)#(fN)#(mN)#(mN)#(mN)#(fN)#(mN) |
| P2_d7_ | P(mN)#(fN)#(mN)(mN)(mN)(fN)(dN)(mN)(mN)(mN)(mN)(mN)(mN)(fN)# |
| as | (mN)#(fN)#(mN)#(mN)#(mN)#(fN)#(mN) |
| P2_d8_ | P(mN)#(fN)#(mN)(mN)(mN)(fN)(mN)(dN)(mN)(mN)(mN)(mN)(mN)(fN)# |
| as | (mN)#(fN)#(mN)#(mN)#(mN)#(fN)#(mN) |
| P2_d9_ | P(mN)#(fN)#(mN)(mN)(mN)(fN)(mN)(mN)(dN)(mN)(mN)(mN)(mN)(fN)# |
| as | (mN)#(fN)#(mN)#(mN)#(mN)#(fN)#(mN) |
| P2_d10_ | P(mN)#(fN)#(mN)(mN)(mN)(fN)(mN)(mN)(mN)(dN)(mN)(mN)(mN)(fN)# |
| as | (mN)#(fN)#(mN)#(mN)#(mN)#(fN)#(mN) |
| P2_d11_ | P(mN)#(fN)#(mN)(mN)(mN)(fN)(mN)(mN)(mN)(mN)(dN)(mN)(mN)(fN)# |
| as | (mN)#(fN)#(mN)#(mN)#(mN)#(fN)#(mN) |
| P2_d12_ | P(mN)#(fN)#(mN)(mN)(mN)(fN)(mN)(mN)(mN)(mN)(mN)(dN)(mN)(fN)# |
| as | (mN)#(fN)#(mN)#(mN)#(mN)#(fN)#(mN) |
| P2_d13_ | P(mN)#(fN)#(mN)(mN)(mN)(fN)(mN)(mN)(mN)(mN)(mN)(mN)(dN)(fN)# |
| as | (mN)#(fN)#(mN)#(mN)#(mN)#(fN)#(mN) |
| P2_d14_ | P(mN)#(fN)#(mN)(mN)(mN)(fN)(mN)(mN)(mN)(mN)(mN)(mN)(mN)(dN)# |
| as | (mN)#(fN)#(mN)#(mN)#(mN)#(fN)#(mN) |
| P2_d15_ | P(mN)#(fN)#(mN)(mN)(mN)(fN)(mN)(mN)(mN)(mN)(mN)(mN)(mN)(fN)# |
| as | (dN)#(fN)#(mN)#(mN)#(mN)#(fN)#(mN) |
| P2_d16_ | P(mN)#(fN)#(mN)(mN)(mN)(fN)(mN)(mN)(mN)(mN)(mN)(mN)(mN)(fN)# |
| as | (mN)#(dN)#(mN)#(mN)#(mN)#(fN)#(mN) |
| P2_d17_ | P(mN)#(fN)#(mN)(mN)(mN)(fN)(mN)(mN)(mN)(mN)(mN)(mN)(mN)(fN)# |
| as | (mN)#(fN)#(dN)#(mN)#(mN)#(fN)#(mN) |
| P2_d18_ | P(mN)#(fN)#(mN)(mN)(mN)(fN)(mN)(mN)(mN)(mN)(mN)(mN)(mN)(fN)# |
| as | (mN)#(fN)#(mN)#(dN)#(mN)#(fN)#(mN) |
| P2_d19_ | P(mN)#(fN)#(mN)(mN)(mN)(fN)(mN)(mN)(mN)(mN)(mN)(mN)(mN)(fN)# |
| as | (mN)#(fN)#(mN)#(mN)#(dN)#(fN)#(mN) |
| P2_d20_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(dN)#(mN) |
| P2_d21_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(dN) |
| P1_d1_ | (dN)#(mN)#(mN)(fN)(mN)(fN)(mN)(fN)(mN)(fN)(mN)(mN)(mN)(fN)#(mN)# |
| s | (mN) |
| P1_d2_ | (mN)#(dN)#(mN)(fN)(mN)(fN)(mN)(fN)(mN)(fN)(mN)(mN)(mN)(fN)#(mN)# |
| s | (mN) |
| P1_d3_ | (mN)#(mN)#(dN)(fN)(mN)(fN)(mN)(fN)(mN)(fN)(mN)(mN)(mN)(fN)#(mN)# |
| s | (mN) |
| P1_d4_ | (mN)#(mN)#(mN)(dN)(mN)(fN)(mN)(fN)(mN)(fN)(mN)(mN)(mN)(fN)#(mN) |
| s | #(mN) |
| P1_d5_ | (mN)#(mN)#(mN)(fN)(dN)(fN)(mN)(fN)(mN)(fN)(mN)(mN)(mN)(fN)#(mN)# |
| s | (mN) |
| P1_d6_ | (mN)#(mN)#(mN)(fN)(mN)(dN)(mN)(fN)(mN)(fN)(mN)(mN)(mN)(fN)#(mN) |
| s | #(mN) |
| P1_d7_ | (mN)#(mN)#(mN)(fN)(mN)(fN)(dN)(fN)(mN)(fN)(mN)(mN)(mN)(fN)#(mN)# |
| s | (mN) |
| P1_d8_ | (mN)#(mN)#(mN)(fN)(mN)(fN)(mN)(dN)(mN)(fN)(mN)(mN)(mN)(fN)#(mN) |
| s | #(mN) |
| P1_d9_ | (mN)#(mN)#(mN)(fN)(mN)(fN)(mN)(fN)(dN)(fN)(mN)(mN)(mN)(fN)#(mN)# |
| s | (mN) |
| P1_d10_ | (mN)#(mN)#(mN)(fN)(mN)(fN)(mN)(fN)(mN)(dN)(mN)(mN)(mN)(fN)#(mN) |
| s | #(mN) |
| P1_d11_ | (mN)#(mN)#(mN)(fN)(mN)(fN)(mN)(fN)(mN)(fN)(dN)(mN)(mN)(fN)#(mN)# |
| s | (mN) |
| P1_d12_ | (mN)#(mN)#(mN)(fN)(mN)(fN)(mN)(fN)(mN)(fN)(mN)(dN)(mN)(fN)#(mN)# |
| s | (mN) |
| P1_d13_ | (mN)#(mN)#(mN)(fN)(mN)(fN)(mN)(fN)(mN)(fN)(mN)(mN)(dN)(fN)#(mN)# |
| s | (mN) |
| P1_d14_ | (mN)#(mN)#(mN)(fN)(mN)(fN)(mN)(fN)(mN)(fN)(mN)(mN)(mN)(dN)#(mN) |
| s | #(mN) |
| P1_d15_ | (mN)#(mN)#(mN)(fN)(mN)(fN)(mN)(fN)(mN)(fN)(mN)(mN)(mN)(fN)#(dN)# |
| s | (mN) |
| P1_d16_ | (mN)#(mN)#(mN)(fN)(mN)(fN)(mN)(fN)(mN)(fN)(mN)(mN)(mN)(fN)#(mN)# |
| s | (dN) |
| P2_d1_ | (dN)#(mN)#(mN)(mN)(mN)(fN)(fN)(fN)(mN)(fN)(mN)(mN)(mN)(mN)#(mN) |
| s | #(mN) |
| P2_d2_ | (mN)#(dN)#(mN)(mN)(mN)(fN)(fN)(fN)(mN)(fN)(mN)(mN)(mN)(mN)#(mN) |
| s | #(mN) |
| P2_d3_ | (mN)#(mN)#(dN)(mN)(mN)(fN)(fN)(fN)(mN)(fN)(mN)(mN)(mN)(mN)#(mN) |
| s | #(mN) |
| P2_d4_ | (mN)#(mN)#(mN)(dN)(mN)(fN)(fN)(fN)(mN)(fN)(mN)(mN)(mN)(mN)#(mN) |
| s | #(mN) |
| P2_d5_ | (mN)#(mN)#(mN)(mN)(dN)(fN)(fN)(fN)(mN)(fN)(mN)(mN)(mN)(mN)#(mN) |
| s | #(mN) |
| P2_d6_ | (mN)#(mN)#(mN)(mN)(mN)(dN)(fN)(fN)(mN)(fN)(mN)(mN)(mN)(mN)#(mN) |
| s | #(mN) |
| P2_d7_ | (mN)#(mN)#(mN)(mN)(mN)(fN)(dN)(fN)(mN)(fN)(mN)(mN)(mN)(mN)#(mN) |
| s | #(mN) |
| P2_d8_ | (mN)#(mN)#(mN)(mN)(mN)(fN)(fN)(dN)(mN)(fN)(mN)(mN)(mN)(mN)#(mN) |
| s | #(mN) |
| P2_d9_ | (mN)#(mN)#(mN)(mN)(mN)(fN)(fN)(fN)(dN)(fN)(mN)(mN)(mN)(mN)#(mN) |
| s | #(mN) |
| P2_d10_ | (mN)#(mN)#(mN)(mN)(mN)(fN)(fN)(fN)(mN)(dN)(mN)(mN)(mN)(mN)#(mN) |
| s | #(mN) |
| P2_d11_ | (mN)#(mN)#(mN)(mN)(mN)(fN)(fN)(fN)(mN)(fN)(dN)(mN)(mN)(mN)#(mN) |
| s | #(mN) |
| P2_d12_ | (mN)#(mN)#(mN)(mN)(mN)(fN)(fN)(fN)(mN)(fN)(mN)(dN)(mN)(mN)#(mN) |
| s | #(mN) |
| P2_d13_ | (mN)#(mN)#(mN)(mN)(mN)(fN)(fN)(fN)(mN)(fN)(mN)(mN)(dN)(mN)#(mN) |
| s | #(mN) |
| P2_d14_ | (mN)#(mN)#(mN)(mN)(mN)(fN)(fN)(fN)(mN)(fN)(mN)(mN)(mN)(dN)#(mN) |
| s | #(mN) |
| P2_d15_ | (mN)#(mN)#(mN)(mN)(mN)(fN)(fN)(fN)(mN)(fN)(mN)(mN)(mN)(mN)#(dN) |
| s | #(mN) |
| P2_d16_ | (mN)#(mN)#(mN)(mN)(mN)(fN)(fN)(fN)(mN)(fN)(mN)(mN)(mN)(mN)#(mN) |
| s | #(dN) |
| as_TW_ | P(mN)#(fN)#(mN)(mN)(mN)(fN)(mN)(mN)(mN)(mN)(mN)(mN)(mN)(fN)# |
| D1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| D2 | (mN)#(fN)#(dN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| D3 | (mN)#(fN)#(mN)#(dN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| D4 | (mN)#(fN)#(mN)#(mN)#(dN)#(mN)#(mN) |
| as_TW_ | P(mN)#(fN)#(mN)(mN)(mN)(fN)(mN)(mN)(mN)(mN)(mN)(mN)(mN)(fN)# |
| D5 | (mN)#(fN)#(mN)#(mN)#(mN)#(dN)#(mN) |
| as_TW_ | P(mN)#(fN)#(mN)(mN)(mN)(fN)(mN)(mN)(mN)(mN)(mN)(mN)(mN)(fN)# |
| D6 | (mN)#(fN)#(mN)#(mN)#(mN)#(mN)#(dN) |
| as_TW_ | P(mN)#(fN)#(mN)(mN)(mN)(fN)(mN)(mN)(mN)(mN)(mN)(mN)(mN)(fN)# |
| D7 | (mN)#(fN)#(dN)#(dN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| D8 | (mN)#(fN)#(mN)#(dN)#(dN)#(mN)#(mN) |
| as_TW_ | P(mN)#(fN)#(mN)(mN)(mN)(fN)(mN)(mN)(mN)(mN)(mN)(mN)(mN)(fN)# |
| D9 | (mN)#(fN)#(mN)#(mN)#(dN)#(dN)#(mN) |
| as_TW_ | P(mN)#(fN)#(mN)(mN)(mN)(fN)(mN)(mN)(mN)(mN)(mN)(mN)(mN)(fN)# |
| D10 | (mN)#(fN)#(mN)#(mN)#(mN)#(dN)#(dN) |
| as_TW_ | P(mN)#(fN)#(mN)(mN)(mN)(fN)(mN)(mN)(mN)(mN)(mN)(mN)(mN)(fN)# |
| D11 | (mN)#(fN)#(dN)#(mN)#(dN)#(mN)#(mN) |
| as_TW_ | P(mN)#(fN)#(mN)(mN)(mN)(fN)(mN)(mN)(mN)(mN)(mN)(mN)(mN)(fN)# |
| D12 | (mN)#(fN)#(mN)#(mN)#(dN)#(mN)#(dN) |
| as_TW_ | P(mN)#(fN)#(mN)(mN)(mN)(fN)(mN)(mN)(mN)(mN)(mN)(mN)(mN)(fN)# |
| D13 | (mN)#(fN)#(mN)#(dN)#(mN)#(dN)#(mN) |
| as_TW_ | P(mN)#(fN)#(mN)(mN)(mN)(fN)(mN)(mN)(mN)(mN)(mN)(mN)(mN)(fN)# |
| D14 | (mN)#(fN)#(dN)#(dN)#(dN)#(mN)#(mN) |
| as_TW_ | P(mN)#(fN)#(mN)(mN)(mN)(fN)(mN)(mN)(mN)(mN)(mN)(mN)(mN)(fN)# |
| D15 | (mN)#(fN)#(mN)#(mN)#(dN)#(dN)#(dN) |
| as_TW_ | P(mN)#(fN)#(mN)(mN)(mN)(fN)(mN)(mN)(mN)(mN)(mN)(mN)(mN)(fN)# |
| D16 | (mN)#(fN)#(dN)#(dN)#(dN)#(dN)#(dN) |

TABLE 6
UNA-containing chemical modification patterns

| | |
|---|---|
| P1_u1_ | P(uN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(mN) |
| P1_u2_ | P(mN)#(uN)#(mN)(fN)(fN)(fN)(mN)(fN)(mN)(fN)(mN)(fN)(mN)(fN)#(mN)#(fN) |
| as | #(mN)#(mN)#(mN)#(fN)#(mN) |
| P1_u3_ | P(mN)#(fN)#(uN)(fN)(fN)(fN)(mN)(fN)(mN)(fN)(mN)(fN)(mN)(fN)#(mN)#(fN) |
| as | #(mN)#(mN)#(mN)#(fN)#(mN) |
| P1_u4_ | P(mN)#(fN)#(mN)(uN)(fN)(fN)(mN)(fN)(mN)(fN)(mN)(fN)(mN)(fN)#(mN)#(fN) |
| as | #(mN)#(mN)#(mN)#(fN)#(mN) |
| P1_u5_ | P(mN)#(fN)#(mN)(fN)(uN)(fN)(mN)(fN)(mN)(fN)(mN)(fN)(mN)(fN)#(mN)# |
| as | (fN)#(mN)#(mN)#(mN)#(fN)#(mN) |
| P1_u6_ | P(mN)#(fN)#(mN)(fN)(fN)(uN)(mN)(fN)(mN)(fN)(mN)(fN)(mN)(fN)#(mN)# |
| as | (fN)#(mN)#(mN)#(mN)#(fN)#(mN) |
| P1_u7_ | P(mN)#(fN)#(mN)(fN)(fN)(fN)(uN)(fN)(mN)(fN)(mN)(fN)(mN)(fN)#(mN)# |
| as | (fN)#(mN)#(mN)#(mN)#(fN)#(mN) |
| P1_u8_ | P(mN)#(fN)#(mN)(fN)(fN)(fN)(mN)(uN)(mN)(fN)(mN)(fN)(mN)(fN)#(mN)# |
| as | (fN)#(mN)#(mN)#(mN)#(fN)#(mN) |
| P1_u9_ | P(mN)#(fN)#(mN)(fN)(fN)(fN)(mN)(fN)(uN)(fN)(mN)(fN)(mN)(fN)#(mN)# |
| as | (fN)#(mN)#(mN)#(mN)#(fN)#(mN) |
| P1_u10_ | P(mN)#(fN)#(mN)(fN)(fN)(fN)(mN)(fN)(mN)(uN)(mN)(fN)(mN)(fN)#(mN)# |
| as | (fN)#(mN)#(mN)#(mN)#(fN)#(mN) |
| P1_u11_ | P(mN)#(fN)#(mN)(fN)(fN)(fN)(mN)(fN)(mN)(fN)(uN)(fN)(mN)(fN)#(mN)# |
| as | (fN)#(mN)#(mN)#(mN)#(fN)#(mN) |
| P1_u12_ | P(mN)#(fN)#(mN)(fN)(fN)(fN)(mN)(fN)(mN)(fN)(mN)(uN)(mN)(fN)#(mN)# |
| as | (fN)#(mN)#(mN)#(mN)#(fN)#(mN) |
| P1_u13_ | P(mN)#(fN)#(mN)(fN)(fN)(fN)(mN)(fN)(mN)(fN)(mN)(fN)(uN)(fN)#(mN)# |
| as | (fN)#(mN)#(mN)#(mN)#(fN)#(mN) |
| P1_u14_ | P(mN)#(fN)#(mN)(fN)(fN)(fN)(mN)(fN)(mN)(fN)(mN)(fN)(mN)(uN)#(mN)# |
| as | (fN)#(mN)#(mN)#(mN)#(fN)#(mN) |
| P1_u15_ | P(mN)#(fN)#(mN)(fN)(fN)(fN)(mN)(fN)(mN)(fN)(mN)(fN)(mN)(fN)#(uN)# |
| as | (fN)#(mN)#(mN)#(mN)#(fN)#(mN) |
| P1_u16_ | P(mN)#(fN)#(mN)(fN)(fN)(fN)(mN)(fN)(mN)(fN)(mN)(fN)(mN)(fN)#(mN)# |
| as | (uN)#(mN)#(mN)#(mN)#(fN)#(mN) |
| P1_u17_ | P(mN)#(fN)#(mN)(fN)(fN)(fN)(mN)(fN)(mN)(fN)(mN)(fN)(mN)(fN)#(mN)# |
| as | (fN)#(uN)#(mN)#(mN)#(fN)#(mN) |
| P1_u18_ | P(mN)#(fN)#(mN)(fN)(fN)(fN)(mN)(fN)(mN)(fN)(mN)(fN)(mN)(fN)#(mN)# |
| as | (fN)#(mN)#(uN)#(mN)#(fN)#(mN) |
| P1_u19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(uN)#(fN)#(mN) |
| P1_u20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(uN)#(mN) |
| P1_u21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(uN) |
| P2_u1_ | P(uU)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_u2_ | P(mN)#(uU)#(mN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_u3_ | P(mN)#(fN)#(uU)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_u4_ | P(mN)#(fN)#(mN)(uU)(mN)(fN)(mN)(mN)(mN)(mN)(mN)(mN)(mN)(fN)# |
| as | (mN)#(fN)#(mN)#(mN)#(mN)#(fN)#(mN) |
| P2_u5_ | P(mN)#(fN)#(mN)(mN)(uU)(fN)(mN)(mN)(mN)(mN)(mN)(mN)(mN)(fN)# |
| as | (mN)#(fN)#(mN)#(mN)#(mN)#(fN)#(mN) |
| P2_u6_ | P(mN)#(fN)#(mN)(mN)(mN)(uU)(mN)(mN)(mN)(mN)(mN)(mN)(mN)(fN)# |
| as | (mN)#(fN)#(mN)#(mN)#(mN)#(fN)#(mN) |
| P2_u7_ | P(mN)#(fN)#(mN)(mN)(mN)(fN)(uU)(mN)(mN)(mN)(mN)(mN)(mN)(fN)# |
| as | (mN)#(fN)#(mN)#(mN)#(mN)#(fN)#(mN) |
| P2_u8_ | P(mN)#(fN)#(mN)(mN)(mN)(fN)(mN)(uU)(mN)(mN)(mN)(mN)(mN)(fN)# |
| as | (mN)#(fN)#(mN)#(mN)#(mN)#(fN)#(mN) |
| P2_u9_ | P(mN)#(fN)#(mN)(mN)(mN)(fN)(mN)(mN)(uU)(mN)(mN)(mN)(mN)(fN)# |
| as | (mN)#(fN)#(mN)#(mN)#(mN)#(fN)#(mN) |
| P2_u10_ | P(mN)#(fN)#(mN)(mN)(mN)(fN)(mN)(mN)(mN)(uU)(mN)(mN)(mN)(fN)# |
| as | (mN)#(fN)#(mN)#(mN)#(mN)#(fN)#(mN) |
| P2_u11_ | P(mN)#(fN)#(mN)(mN)(mN)(fN)(mN)(mN)(mN)(mN)(uU)(mN)(mN)(fN)# |
| as | (mN)#(fN)#(mN)#(mN)#(mN)#(fN)#(mN) |
| P2_u12_ | P(mN)#(fN)#(mN)(mN)(mN)(fN)(mN)(mN)(mN)(mN)(mN)(uU)(mN)(fN)# |
| as | (mN)#(fN)#(mN)#(mN)#(mN)#(fN)#(mN) |
| P2_u13_ | P(mN)#(fN)#(mN)(mN)(mN)(fN)(mN)(mN)(mN)(mN)(mN)(mN)(uU)(fN)# |
| as | (mN)#(fN)#(mN)#(mN)#(mN)#(fN)#(mN) |
| P2_u14_ | P(mN)#(fN)#(mN)(mN)(mN)(fN)(mN)(mN)(mN)(mN)(mN)(mN)(mN)(uU)# |
| as | (mN)#(fN)#(mN)#(mN)#(mN)#(fN)#(mN) |
| P2_u15_ | P(mN)#(fN)#(mN)(mN)(mN)(fN)(mN)(mN)(mN)(mN)(mN)(mN)(mN)(fN)# |
| as | (uU)#(fN)#(mN)#(mN)#(mN)#(fN)#(mN) |
| P2_u16_ | P(mN)#(fN)#(mN)(mN)(mN)(fN)(mN)(mN)(mN)(mN)(mN)(mN)(mN)(fN)# |
| as | (mN)#(uU)#(mN)#(mN)#(mN)#(fN)#(mN) |
| P2_u17_ | P(mN)#(fN)#(mN)(mN)(mN)(fN)(mN)(mN)(mN)(mN)(mN)(mN)(mN)(fN)# |
| as | (mN)#(fN)#(uUJ)#(mN)#(mN)#(fN)#(mN) |
| P2_u18_ | P(mN)#(fN)#(mN)(mN)(mN)(fN)(mN)(mN)(mN)(mN)(mN)(mN)(mN)(fN)# |
| as | (mN)#(fN)#(mN)#(uU)#(mN)#(fN)#(mN) |
| P2_u19_ | P(mN)#(fN)#(mN)(mN)(mN)(fN)(mN)(mN)(mN)(mN)(mN)(mN)(mN)(fN)# |
| as | (mN)#(fN)#(mN)#(mN)#(uU)#(fN)#(mN) |
| P2_u20_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(uU)#(mN) |
| P2_u21_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(uU) |
| P1_u1_ | (uN)#(mN)#(mN)(fN)(mN)(fN)(mN)(fN)(mN)(fN)(mN)(mN)(mN)(fN)#(mN)# |
| s | (mN) |
| P1_u2_ | (mN)#(uN)#(mN)(fN)(mN)(fN)(mN)(fN)(mN)(fN)(mN)(mN)(mN)(fN)#(mN)# |
| s | (mN) |
| P1_u3_ | (mN)#(mN)#(uN)(fN)(mN)(fN)(mN)(fN)(mN)(fN)(mN)(mN)(mN)(fN)#(mN)# |
| s | (mN) |
| P1_u4_ | (mN)#(mN)#(mN)(uN)(mN)(fN)(mN)(fN)(mN)(fN)(mN)(mN)(mN)(fN)#(mN) |
| s | #(mN) |
| P1_u5_ | (mN)#(mN)#(mN)(fN)(uN)(fN)(mN)(fN)(mN)(fN)(mN)(mN)(mN)(fN)#(mN)# |
| s | (mN) |
| P1_u6_ | (mN)#(mN)#(mN)(fN)(mN)(uN)(mN)(fN)(mN)(fN)(mN)(mN)(mN)(fN)#(mN) |
| s | #(mN) |
| P1_u7_ | (mN)#(mN)#(mN)(fN)(mN)(fN)(uN)(fN)(mN)(fN)(mN)(mN)(mN)(fN)#(mN)# |
| s | (mN) |
| P1_u8_ | (mN)#(mN)#(mN)(fN)(mN)(fN)(mN)(uN)(mN)(fN)(mN)(mN)(mN)(fN)#(mN) |
| s | #(mN) |
| P1_u9_ | (mN)#(mN)#(mN)(fN)(mN)(fN)(mN)(fN)(uN)(fN)(mN)(mN)(mN)(fN)#(mN)# |
| s | (mN) |
| P1_u10_ | (mN)#(mN)#(mN)(fN)(mN)(fN)(mN)(fN)(mN)(uN)(mN)(mN)(mN)(fN)#(mN) |
| s | #(mN) |
| P1_u11_ | (mN)#(mN)#(mN)(fN)(mN)(fN)(mN)(fN)(mN)(fN)(uN)(mN)(mN)(fN)#(mN)# |
| s | (mN) |
| P1_u12_ | (mN)#(mN)#(mN)(fN)(mN)(fN)(mN)(fN)(mN)(fN)(mN)(uN)(mN)(fN)#(mN)# |
| s | (mN) |
| P1_u13_ | (mN)#(mN)#(mN)(fN)(mN)(fN)(mN)(fN)(mN)(fN)(mN)(mN)(uN)(fN)#(mN)# |
| s | (mN) |
| P1_u14_ | (mN)#(mN)#(mN)(fN)(mN)(fN)(mN)(fN)(mN)(fN)(mN)(mN)(mN)(uN)#(mN) |
| s | #(mN) |
| P1_u15_ | (mN)#(mN)#(mN)(fN)(mN)(fN)(mN)(fN)(mN)(fN)(mN)(mN)(mN)(fN)#(uN)# |
| s | (mN) |
| P1_u16_ | (mN)#(mN)#(mN)(fN)(mN)(fN)(mN)(fN)(mN)(fN)(mN)(mN)(mN)(fN)#(mN)# |
| s | (UN) |
| P2_u1_ | (uN)#(mN)#(mN)(mN)(mN)(fN)(fN)(fN)(mN)(fN)(mN)(mN)(mN)(mN)#(mN) |
| s | #(mN) |
| P2_u2_ | (mN)#(uN)#(mN)(mN)(mN)(fN)(fN)(fN)(mN)(fN)(mN)(mN)(mN)(mN)#(mN) |
| s | #(mN) |
| P2_u3_ | (mN)#(mN)#(uN)(mN)(mN)(fN)(fN)(fN)(mN)(fN)(mN)(mN)(mN)(mN)#(mN) |
| s | #(mN) |
| P2_u4_ | (mN)#(mN)#(mN)(uN)(mN)(fN)(fN)(fN)(mN)(fN)(mN)(mN)(mN)(mN)#(mN) |
| s | #(mN) |
| P2_u5_ | (mN)#(mN)#(mN)(mN)(uN)(fN)(fN)(fN)(mN)(fN)(mN)(mN)(mN)(mN)#(mN) |
| s | #(mN) |
| P2_u6_ | (mN)#(mN)#(mN)(mN)(mN)(uN)(fN)(fN)(mN)(fN)(mN)(mN)(mN)(mN)#(mN) |
| s | #(mN) |
| P2_u7_ | (mN)#(mN)#(mN)(mN)(mN)(fN)(uN)(fN)(mN)(fN)(mN)(mN)(mN)(mN)#(mN) |
| s | #(mN) |
| P2_u8_ | (mN)#(mN)#(mN)(mN)(mN)(fN)(fN)(uN)(mN)(fN)(mN)(mN)(mN)(mN)#(mN) |
| s | #(mN) |
| P2_u9_ | (mN)#(mN)#(mN)(mN)(mN)(fN)(fN)(fN)(uN)(fN)(mN)(mN)(mN)(mN)#(mN) |
| s | #(mN) |
| P2_u10_ | (mN)#(mN)#(mN)(mN)(mN)(fN)(fN)(fN)(mN)(uN)(mN)(mN)(mN)(mN)#(mN) |
| s | #(mN) |
| P2_u11_ | (mN)#(mN)#(mN)(mN)(mN)(fN)(fN)(fN)(mN)(fN)(uN)(mN)(mN)(mN)#(mN) |
| s | #(mN) |
| P2_u12_ | (mN)#(mN)#(mN)(mN)(mN)(fN)(fN)(fN)(mN)(fN)(mN)(uN)(mN)(mN)#(mN) |
| s | #(mN) |
| P2_u13_ | (mN)#(mN)#(mN)(mN)(mN)(fN)(fN)(fN)(mN)(fN)(mN)(mN)(uN)(mN)#(mN) |
| s | #(mN) |
| P2_u14_ | (mN)#(mN)#(mN)(mN)(mN)(fN)(fN)(fN)(mN)(fN)(mN)(mN)(mN)(uN)#(mN) |
| s | #(mN) |
| P2_u15_ | (mN)#(mN)#(mN)(mN)(mN)(fN)(fN)(fN)(mN)(fN)(mN)(mN)(mN)(mN)#(uN) |
| s | #(mN) |
| P2_u16_ | (mN)#(mN)#(mN)(mN)(mN)(fN)(fN)(fN)(mN)(fN)(mN)(mN)(mN)(mN)#(mN) |
| s | #(UN) |
| as_TW_ | P(mN)#(fN)#(mN)(mN)(mN)(fN)(mN)(mN)(mN)(mN)(mN)(mN)(mN)(fN)# |
| U1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| U2 | (mN)#(fN)#(uN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| U3 | (mN)#(fN)#(mN)#(uN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| U4 | (mN)#(fN)#(mN)#(mN)#(uN)#(mN)#(mN) |
| as_TW_ | P(mN)#(fN)#(mN)(mN)(mN)(fN)(mN)(mN)(mN)(mN)(mN)(mN)(mN)(fN)# |
| U5 | (mN)#(fN)#(mN)#(mN)#(mN)#(uN)#(mN) |
| as_TW_ | P(mN)#(fN)#(mN)(mN)(mN)(fN)(mN)(mN)(mN)(mN)(mN)(mN)(mN)(fN)# |
| U6 | (mN)#(fN)#(mN)#(mN)#(mN)#(mN)#(uN) |
| as_TW_ | P(mN)#(fN)#(mN)(mN)(mN)(fN)(mN)(mN)(mN)(mN)(mN)(mN)(mN)(fN)# |
| U7 | (mN)#(fN)#(uN)#(uN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| U8 | (mN)#(fN)#(mN)#(uN)#(uN)#(mN)#(mN) |
| as_TW_ | P(mN)#(fN)#(mN)(mN)(mN)(fN)(mN)(mN)(mN)(mN)(mN)(mN)(mN)(fN)# |
| U9 | (mN)#(fN)#(mN)#(mN)#(uN)#(uN)#(mN) |
| as_TW_ | P(mN)#(fN)#(mN)(mN)(mN)(fN)(mN)(mN)(mN)(mN)(mN)(mN)(mN)(fN)# |
| U10 | (mN)#(fN)#(mN)#(mN)#(mN)#(uN)#(uN) |
| as_TW_ | P(mN)#(fN)#(mN)(mN)(mN)(fN)(mN)(mN)(mN)(mN)(mN)(mN)(mN)(fN)# |
| U11 | (mN)#(fN)#(uN)#(mN)#(uN)#(mN)#(mN) |
| as_TW_ | P(mN)#(fN)#(mN)(mN)(mN)(fN)(mN)(mN)(mN)(mN)(mN)(mN)(mN)(fN)# |
| U12 | (mN)#(fN)#(mN)#(mN)#(uN)#(mN)#(uN) |
| as_TW_ | P(mN)#(fN)#(mN)(mN)(mN)(fN)(mN)(mN)(mN)(mN)(mN)(mN)(mN)(fN)# |
| U13 | (mN)#(fN)#(mN)#(uN)#(mN)#(uN)#(mN) |
| as_TW_ | P(mN)#(fN)#(mN)(mN)(mN)(fN)(mN)(mN)(mN)(mN)(mN)(mN)(mN)(fN)# |
| U14 | (mN)#(fN)#(uN)#(uN)#(uN)#(mN)#(mN) |
| as_TW_ | P(mN)#(fN)#(mN)(mN)(mN)(fN)(mN)(mN)(mN)(mN)(mN)(mN)(mN)(fN)# |
| U15 | (mN)#(fN)#(mN)#(mN)#(uN)#(uN)#(uN) |
| as_TW_ | P(mN)#(fN)#(mN)(mN)(mN)(fN)(mN)(mN)(mN)(mN)(mN)(mN)(mN)(fN)# |
| U16 | (mN)#(fN)#(uN)#(uN)#(uN)#(uN)#(uN) |

TABLE 7
RNA-containing chemical modification patterns

| | |
|---|---|
| P1_r1_ | P(rN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(mN) |
| P1_r2_ | P(mN)#(rN)#(mN)(fN)(fN)(fN)(mN)(fN)(mN)(fN)(mN)(fN)(mN)(fN)#(mN)# |
| as | (fN)#(mN)#(mN)#(mN)#(fN)#(mN) |
| P1_r3_ | P(mN)#(fN)#(rN)(fN)(fN)(fN)(mN)(fN)(mN)(fN)(mN)(fN)(mN)(fN)#(mN)# |
| as | (fN)#(mN)#(mN)#(mN)#(fN)#(mN) |
| P1_r4_ | P(mN)#(fN)#(mN)(rN)(fN)(fN)(mN)(fN)(mN)(fN)(mN)(fN)(mN)(fN)#(mN)# |
| as | (fN)#(mN)#(mN)#(mN)#(fN)#(mN) |
| P1_r5_ | P(mN)#(fN)#(mN)(fN)(rN)(fN)(mN)(fN)(mN)(fN)(mN)(fN)(mN)(fN)#(mN)#(fN) |
| as | #(mN)#(mN)#(mN)#(fN)#(mN) |
| P1_r6_ | P(mN)#(fN)#(mN)(fN)(fN)(rN)(mN)(fN)(mN)(fN)(mN)(fN)(mN)(fN)#(mN)#(fN) |
| as | #(mN)#(mN)#(mN)#(fN)#(mN) |
| P1_r7_ | P(mN)#(fN)#(mN)(fN)(fN)(fN)(rN)(fN)(mN)(fN)(mN)(fN)(mN)(fN)#(mN)#(fN) |
| as | #(mN)#(mN)#(mN)#(fN)#(mN) |
| P1_r8_ | P(mN)#(fN)#(mN)(fN)(fN)(fN)(mN)(rN)(mN)(fN)(mN)(fN)(mN)(fN)#(mN)# |
| as | (fN)#(mN)#(mN)#(mN)#(fN)#(mN) |
| P1_r9_ | P(mN)#(fN)#(mN)(fN)(fN)(fN)(mN)(fN)(rN)(fN)(mN)(fN)(mN)(fN)#(mN)# |
| as | (fN)#(mN)#(mN)#(mN)#(fN)#(mN) |
| P1_r10_ | P(mN)#(fN)#(mN)(fN)(fN)(fN)(mN)(fN)(mN)(rN)(mN)(fN)(mN)(fN)#(mN)# |
| as | (fN)#(mN)#(mN)#(mN)#(fN)#(mN) |
| P1_r11_ | P(mN)#(fN)#(mN)(fN)(fN)(fN)(mN)(fN)(mN)(fN)(rN)(fN)(mN)(fN)#(mN)# |
| as | (fN)#(mN)#(mN)#(mN)#(fN)#(mN) |
| P1_r12_ | P(mN)#(fN)#(mN)(fN)(fN)(fN)(mN)(fN)(mN)(fN)(mN)(rN)(mN)(fN)#(mN)# |
| as | (fN)#(mN)#(mN)#(mN)#(fN)#(mN) |
| P1_r13_ | P(mN)#(fN)#(mN)(fN)(fN)(fN)(mN)(fN)(mN)(fN)(mN)(fN)(rN)(fN)#(mN)# |
| as | (fN)#(mN)#(mN)#(mN)#(fN)#(mN) |
| P1_r14_ | P(mN)#(fN)#(mN)(fN)(fN)(fN)(mN)(fN)(mN)(fN)(mN)(fN)(mN)(rN)#(mN)# |
| as | (fN)#(mN)#(mN)#(mN)#(fN)#(mN) |
| P1_r15_ | P(mN)#(fN)#(mN)(fN)(fN)(fN)(mN)(fN)(mN)(fN)(mN)(fN)(mN)(fN)#(rN)# |
| as | (fN)#(mN)#(mN)#(mN)#(fN)#(mN) |
| P1_r16_ | P(mN)#(fN)#(mN)(fN)(fN)(fN)(mN)(fN)(mN)(fN)(mN)(fN)(mN)(fN)#(mN)# |
| as | (rN)#(mN)#(mN)#(mN)#(fN)#(mN) |
| P1_r17_ | P(mN)#(fN)#(mN)(fN)(fN)(fN)(mN)(fN)(mN)(fN)(mN)(fN)(mN)(fN)#(mN)# |
| as | (fN)#(rN)#(mN)#(mN)#(fN)#(mN) |
| P1_r18_ | P(mN)#(fN)#(mN)(fN)(fN)(fN)(mN)(fN)(mN)(fN)(mN)(fN)(mN)(fN)#(mN)# |
| as | (fN)#(mN)#(rN)#(mN)#(fN)#(mN) |
| P1_r19_ | P(mN)#(fN)#(mN)(fN)(fN)(fN)(mN)(fN)(mN)(fN)(mN)(fN)(mN)(fN)#(mN)# |
| as | (fN)#(mN)#(mN)#(rN)#(fN)#(mN) |
| P1_r20_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(rN)#(mN) |
| P1_r21_ | P(mN)#(fN)#(mN)(fN)(fN)(fN)(mN)(fN)(mN)(fN)(mN)(fN)(mN)(fN)#(mN)# |
| as | (fN)#(mN)#(mN)#(mN)#(fN)#(rN) |
| P2_r1_ | P(IN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_r2_ | P(mN)#(rN)#(mN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_r3_ | P(mN)#(fN)#(rN)(mN)(mN)(fN)(mN)(mN)(mN)(mN)(mN)(mN)(mN)(fN)# |
| as | (mN)#(fN)#(mN)#(mN)#(mN)#(fN)#(mN) |
| P2_r4_ | P(mN)#(fN)#(mN)(rN)(mN)(fN)(mN)(mN)(mN)(mN)(mN)(mN)(mN)(fN)# |
| as | (mN)#(fN)#(mN)#(mN)#(mN)#(fN)#(mN) |
| P2_r5_ | P(mN)#(fN)#(mN)(mN)(rN)(fN)(mN)(mN)(mN)(mN)(mN)(mN)(mN)(fN)# |
| as | (mN)#(fN)#(mN)#(mN)#(mN)#(fN)#(mN) |
| P2_r6_ | P(mN)#(fN)#(mN)(mN)(mN)(rN)(mN)(mN)(mN)(mN)(mN)(mN)(mN)(fN)# |
| as | (mN)#(fN)#(mN)#(mN)#(mN)#(fN)#(mN) |
| P2_r7_ | P(mN)#(fN)#(mN)(mN)(mN)(fN)(rN)(mN)(mN)(mN)(mN)(mN)(mN)(fN)# |
| as | (mN)#(fN)#(mN)#(mN)#(mN)#(fN)#(mN) |
| P2_r8_ | P(mN)#(fN)#(mN)(mN)(mN)(fN)(mN)(rN)(mN)(mN)(mN)(mN)(mN)(fN)# |
| as | (mN)#(fN)#(mN)#(mN)#(mN)#(fN)#(mN) |
| P2_r9_ | P(mN)#(fN)#(mN)(mN)(mN)(fN)(mN)(mN)(rN)(mN)(mN)(mN)(mN)(fN)# |
| as | (mN)#(fN)#(mN)#(mN)#(mN)#(fN)#(mN) |
| P2_r10_ | P(mN)#(fN)#(mN)(mN)(mN)(fN)(mN)(mN)(mN)(rN)(mN)(mN)(mN)(fN)# |
| as | (mN)#(fN)#(mN)#(mN)#(mN)#(fN)#(mN) |
| P2_r11_ | P(mN)#(fN)#(mN)(mN)(mN)(fN)(mN)(mN)(mN)(mN)(rN)(mN)(mN)(fN)# |
| as | (mN)#(fN)#(mN)#(mN)#(mN)#(fN)#(mN) |
| P2_r12_ | P(mN)#(fN)#(mN)(mN)(mN)(fN)(mN)(mN)(mN)(mN)(mN)(rN)(mN)(fN)# |
| as | (mN)#(fN)#(mN)#(mN)#(mN)#(fN)#(mN) |
| P2_r13_ | P(mN)#(fN)#(mN)(mN)(mN)(fN)(mN)(mN)(mN)(mN)(mN)(mN)(rN)(fN)# |
| as | (mN)#(fN)#(mN)#(mN)#(mN)#(fN)#(mN) |
| P2_r14_ | P(mN)#(fN)#(mN)(mN)(mN)(fN)(mN)(mN)(mN)(mN)(mN)(mN)(mN)(rN)# |
| as | (mN)#(fN)#(mN)#(mN)#(mN)#(fN)#(mN) |
| P2_r15_ | P(mN)#(fN)#(mN)(mN)(mN)(fN)(mN)(mN)(mN)(mN)(mN)(mN)(mN)(fN)# |
| as | (rN)#(fN)#(mN)#(mN)#(mN)#(fN)#(mN) |
| P2_r16_ | P(mN)#(fN)#(mN)(mN)(mN)(fN)(mN)(mN)(mN)(mN)(mN)(mN)(mN)(fN)# |
| as | (mN)#(rN)#(mN)#(mN)#(mN)#(fN)#(mN) |
| P2_r17_ | P(mN)#(fN)#(mN)(mN)(mN)(fN)(mN)(mN)(mN)(mN)(mN)(mN)(mN)(fN)# |
| as | (mN)#(fN)#(rN)#(mN)#(mN)#(fN)#(mN) |
| P2_r18_ | P(mN)#(fN)#(mN)(mN)(mN)(fN)(mN)(mN)(mN)(mN)(mN)(mN)(mN)(fN)# |
| as | (mN)#(fN)#(mN)#(rN)#(mN)#(fN)#(mN) |
| P2_r19_ | P(mN)#(fN)#(mN)(mN)(mN)(fN)(mN)(mN)(mN)(mN)(mN)(mN)(mN)(fN)# |
| as | (mN)#(fN)#(mN)#(mN)#(rN)#(fN)#(mN) |
| P2_r20_ | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(rN)#(mN) |
| P2_r21 | P(mN)#(fN)#(mN)(mN)(mN)(fN)(mN)(mN)(mN)(mN)(mN)(mN)(mN)(fN)# |
| as | (mN)#(fN)#(mN)#(mN)#(mN)#(fN)#(rN) |
| P1_r1_s | (rN)#(mN)#(mN)(fN)(mN)(fN)(mN)(fN)(mN)(fN)(mN)(mN)(mN)(fN)#(mN)# |
| | (mN) |
| P1_r2_s | (mN)#(rN)#(mN)(fN)(mN)(fN)(mN)(fN)(mN)(fN)(mN)(mN)(mN)(fN)#(mN)# |
| | (mN) |
| P1_r3_s | (mN)#(mN)#(rN)(fN)(mN)(fN)(mN)(fN)(mN)(fN)(mN)(mN)(mN)(fN)#(mN)# |
| | (mN) |
| P1_r4_s | (mN)#(mN)#(mN)(rN)(mN)(fN)(mN)(fN)(mN)(fN)(mN)(mN)(mN)(fN)#(mN)# |
| | (mN) |
| P1_r5_s | (mN)#(mN)#(mN)(fN)(rN)(fN)(mN)(fN)(mN)(fN)(mN)(mN)(mN)(fN)#(mN)# |
| | (mN) |
| P1_r6_s | (mN)#(mN)#(mN)(fN)(mN)(rN)(mN)(fN)(mN)(fN)(mN)(mN)(mN)(fN)#(mN)# |
| | (mN) |
| P1_r7_s | (mN)#(mN)#(mN)(fN)(mN)(fN)(rN)(fN)(mN)(fN)(mN)(mN)(mN)(fN)#(mN)# |
| | (mN) |
| P1_r8_s | (mN)#(mN)#(mN)(fN)(mN)(fN)(mN)(rN)(mN)(fN)(mN)(mN)(mN)(fN)#(mN)# |
| | (mN) |
| P1_r9_s | (mN)#(mN)#(mN)(fN)(mN)(fN)(mN)(fN)(rN)(fN)(mN)(mN)(mN)(fN)#(mN)# |
| | (mN) |
| P1_r10_ | (mN)#(mN)#(mN)(fN)(mN)(fN)(mN)(fN)(mN)(rN)(mN)(mN)(mN)(fN)#(mN)# |
| s | (mN) |
| P1_r11_ | (mN)#(mN)#(mN)(fN)(mN)(fN)(mN)(fN)(mN)(fN)(rN)(mN)(mN)(fN)#(mN)# |
| s | (mN) |
| P1_r12_ | (mN)#(mN)#(mN)(fN)(mN)(fN)(mN)(fN)(mN)(fN)(mN)(rN)(mN)(fN)#(mN)# |
| s | (mN) |
| P1_r13_ | (mN)#(mN)#(mN)(fN)(mN)(fN)(mN)(fN)(mN)(fN)(mN)(mN)(rN)(fN)#(mN)# |
| s | (mN) |
| P1_r14_ | (mN)#(mN)#(mN)(fN)(mN)(fN)(mN)(fN)(mN)(fN)(mN)(mN)(mN)(rN)#(mN)# |
| s | (mN) |
| P1_r15_ | (mN)#(mN)#(mN)(fN)(mN)(fN)(mN)(fN)(mN)(fN)(mN)(mN)(mN)(fN)#(rN)# |
| s | (mN) |
| P1_r16_ | (mN)#(mN)#(mN)(fN)(mN)(fN)(mN)(fN)(mN)(fN)(mN)(mN)(mN)(fN)#(mN)# |
| s | (rN) |
| P2_r1_s | (rN)#(mN)#(mN)(mN)(mN)(fN)(fN)(fN)(mN)(fN)(mN)(mN)(mN)(mN)#(mN)# |
| | (mN) |
| P2_r2_s | (mN)#(rN)#(mN)(mN)(mN)(fN)(fN)(fN)(mN)(fN)(mN)(mN)(mN)(mN)#(mN)# |
| | (mN) |
| P2_r3_s | (mN)#(mN)#(rN)(mN)(mN)(fN)(fN)(fN)(mN)(fN)(mN)(mN)(mN)(mN)#(mN)# |
| | (mN) |
| P2_r4_s | (mN)#(mN)#(mN)(rN)(mN)(fN)(fN)(fN)(mN)(fN)(mN)(mN)(mN)(mN)#(mN)# |
| | (mN) |
| P2_r5_s | (mN)#(mN)#(mN)(mN)(rN)(fN)(fN)(fN)(mN)(fN)(mN)(mN)(mN)(mN)#(mN)# |
| | (mN) |
| P2_r6_s | (mN)#(mN)#(mN)(mN)(mN)(IN)(fN)(fN)(mN)(fN)(mN)(mN)(mN)(mN)#(mN) |
| | #(mN) |
| P2_r7_s | (mN)#(mN)#(mN)(mN)(mN)(fN)(rN)(fN)(mN)(fN)(mN)(mN)(mN)(mN)#(mN) |
| | #(mN) |
| P2_r8_s | (mN)#(mN)#(mN)(mN)(mN)(fN)(fN)(rN)(mN)(fN)(mN)(mN)(mN)(mN)#(mN) |
| | #(mN) |
| P2_r9_s | (mN)#(mN)#(mN)(mN)(mN)(fN)(fN)(fN)(rN)(fN)(mN)(mN)(mN)(mN)#(mN)# |
| | (mN) |
| P2_r10_ | (mN)#(mN)#(mN)(mN)(mN)(fN)(fN)(fN)(mN)(rN)(mN)(mN)(mN)(mN)#(mN) |
| s | #(mN) |
| P2_r11_ | (mN)#(mN)#(mN)(mN)(mN)(fN)(fN)(fN)(mN)(fN)(rN)(mN)(mN)(mN)#(mN)# |
| s | (mN) |
| P2_r12_ | (mN)#(mN)#(mN)(mN)(mN)(fN)(fN)(fN)(mN)(fN)(mN)(rN)(mN)(mN)#(mN)# |
| s | (mN) |
| P2_r13_ | (mN)#(mN)#(mN)(mN)(mN)(fN)(fN)(fN)(mN)(fN)(mN)(mN)(rN)(mN)#(mN)# |
| s | (mN) |
| P2_r14_ | (mN)#(mN)#(mN)(mN)(mN)(fN)(fN)(fN)(mN)(fN)(mN)(mN)(mN)(rN)#(mN)# |
| s | (mN) |
| P2_r15_ | (mN)#(mN)#(mN)(mN)(mN)(fN)(fN)(fN)(mN)(fN)(mN)(mN)(mN)(mN)#(rN)# |
| s | (mN) |
| P2_r16_ | (mN)#(mN)#(mN)(mN)(mN)(fN)(fN)(fN)(mN)(fN)(mN)(mN)(mN)(mN)#(mN) |
| s | #(rN) |
| as_TW_ | P(mN)#(fN)#(mN)(mN)(mN)(fN)(mN)(mN)(mN)(mN)(mN)(mN)(mN)(fN)# |
| R1 | (mN)#(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| R2 | (mN)#(fN)#(rN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| R3 | (mN)#(fN)#(mN)#(rN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| R4 | (mN)#(fN)#(mN)#(mN)#(rN)#(mN)#(mN) |
| as_TW_ | P(mN)#(fN)#(mN)(mN)(mN)(fN)(mN)(mN)(mN)(mN)(mN)(mN)(mN)(fN)# |
| R5 | (mN)#(fN)#(mN)#(mN)#(mN)#(rN)#(mN) |
| as_TW_ | P(mN)#(fN)#(mN)(mN)(mN)(fN)(mN)(mN)(mN)(mN)(mN)(mN)(mN)(fN)# |
| R6 | (mN)#(fN)#(mN)#(mN)#(mN)#(mN)#(rN) |
| as_TW_ | P(mN)#(fN)#(mN)(mN)(mN)(fN)(mN)(mN)(mN)(mN)(mN)(mN)(mN)(fN)# |
| R7 | (mN)#(fN)#(rN)#(rN)#(mN)#(mN)#(mN) |
| as_TW_ | P(mN)#(fN)#(mN)(mN)(mN)(fN)(mN)(mN)(mN)(mN)(mN)(mN)(mN)(fN)# |
| R8 | (mN)#(fN)#(mN)#(rN)#(rN)#(mN)#(mN) |
| as_TW_ | P(mN)#(fN)#(mN)(mN)(mN)(fN)(mN)(mN)(mN)(mN)(mN)(mN)(mN)(fN)# |
| R9 | (mN)#(fN)#(mN)#(mN)#(rN)#(rN)#(mN) |
| as_TW_ | P(mN)#(fN)#(mN)(mN)(mN)(fN)(mN)(mN)(mN)(mN)(mN)(mN)(mN)(fN)# |
| R10 | (mN)#(fN)#(mN)#(mN)#(mN)#(rN)#(rN) |
| as_TW_ | P(mN)#(fN)#(mN)(mN)(mN)(fN)(mN)(mN)(mN)(mN)(mN)(mN)(mN)(fN)# |
| R11 | (mN)#(fN)#(rN)#(mN)#(rN)#(mN)#(mN) |
| as_TW_ | P(mN)#(fN)#(mN)(mN)(mN)(fN)(mN)(mN)(mN)(mN)(mN)(mN)(mN)(fN)# |
| R12 | (mN)#(fN)#(mN)#(mN)#(rN)#(mN)#(rN) |
| as_TW_ | P(mN)#(fN)#(mN)(mN)(mN)(fN)(mN)(mN)(mN)(mN)(mN)(mN)(mN)(fN)# |
| R13 | (mN)#(fN)#(mN)#(rN)#(mN)#(rN)#(mN) |
| as_TW_ | P(mN)#(fN)#(mN)(mN)(mN)(fN)(mN)(mN)(mN)(mN)(mN)(mN)(mN)(fN)# |
| R14 | (mN)#(fN)#(rN)#(rN)#(rN)#(mN)#(mN) |
| as_TW_ | P(mN)#(fN)#(mN)(mN)(mN)(fN)(mN)(mN)(mN)(mN)(mN)(mN)(mN)(fN)# |
| R15 | (mN)#(fN)#(mN)#(mN)#(rN)#(rN)#(rN) |
| as_TW_ | P(mN)#(fN)#(mN)(mN)(mN)(fN)(mN)(mN)(mN)(mN)(mN)(mN)(mN)(fN)# |
| R16 | (mN)#(fN)#(rN)#(rN)#(rN)#(rN)#(rN) |

TABLE 8
2′-F-containing chemical modification patterns in the antisense strand tail

| | |
|---|---|
| as_TW_ | P(mN)#(fN)#(mN)(mN)(mN)(fN)(mN)(mN)(mN)(mN)(mN)(mN)(mN)(fN)#(mN) |
| F1 | #(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F2 | #(fN)#(fN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F3 | #(fN)#(mN)#(fN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F4 | #(fN)#(mN)#(mN)#(fN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F5 | #(fN)#(mN)#(mN)#(mN)#(fN)#(mN) |
| as_TW_ | P(mN)#(fN)#(mN)(mN)(mN)(fN)(mN)(mN)(mN)(mN)(mN)(mN)(mN)(fN)#(mN) |
| F6 | #(fN)#(mN)#(mN)#(mN)#(mN)#(fN) |
| as_TW_ | P(mN)#(fN)#(mN)(mN)(mN)(fN)(mN)(mN)(mN)(mN)(mN)(mN)(mN)(fN)#(mN) |
| F7 | #(fN)#(fN)#(fN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F8 | #(fN)#(mN)#(fN)#(fN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F9 | #(fN)#(mN)#(mN)#(fN)#(fN)#(mN) |
| as_TW_ | P(mN)#(fN)#(mN)(mN)(mN)(fN)(mN)(mN)(mN)(mN)(mN)(mN)(mN)(fN)#(mN) |
| F10 | #(fN)#(mN)#(mN)#(mN)#(fN)#(fN) |
| as_TW_ | P(mN)#(fN)#(mN)(mN)(mN)(fN)(mN)(mN)(mN)(mN)(mN)(mN)(mN)(fN)#(mN) |
| F11 | #(fN)#(fN)#(mN)#(fN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F12 | #(fN)#(mN)#(mN)#(fN)#(mN)#(fN) |
| as_TW_ | P(mN)#(fN)#(mN)(mN)(mN)(fN)(mN)(mN)(mN)(mN)(mN)(mN)(mN)(fN)# |
| F13 | (mN)#(fN)#(mN)#(fN)#(mN)#(fN)#(mN) |
| as_TW_ | P(mN)#(fN)#(mN)(mN)(mN)(fN)(mN)(mN)(mN)(mN)(mN)(mN)(mN)(fN)# |
| F14 | (mN)#(fN)#(fN)#(fN)#(fN)#(mN)#(mN) |
| as_TW_ | P(mN)#(fN)#(mN)(mN)(mN)(fN)(mN)(mN)(mN)(mN)(mN)(mN)(mN)(fN)#(mN) |
| F15 | #(fN)#(mN)#(mN)#(fN)#(fN)#(fN) |
| as_TW_ | P(mN)#(fN)#(mN)(mN)(mN)(fN)(mN)(mN)(mN)(mN)(mN)(mN)(mN)(fN)#(mN) |
| F16 | #(fN)#(fN)#(fN)#(fN)#(fN)#(fN) |

For the chemical modification patterns recited above in Tables 1-8, the following abbreviations are used: “as” corresponds to an siRNA antisense strand; “s” corresponds to an siRNA sense strand; “m” corresponds to a 2′-O-methyl (2′-OMe) chemical modification; “f” corresponds to a 2′-fluoro (2′-F) chemical modification; “but” corresponds to a butane chemical modification (replacement of a nucleotide with a butane); “ibut” corresponds to an internal butane chemical modification (butane linked between two nucleotides); “1” corresponds to an LNA chemical modification; “e” corresponds to a 2′-O-methoxyethyl (MOE) chemical modification; “d” corresponds to a 2′-deoxy (DNA) chemical modification; “u” corresponds to an unlocked (UNA) chemical modification; “r” corresponds to an unmodified ribonucleotide; and “#” corresponds to a phosphorothioate internucleotide linkage.

siRNA Design

In some embodiments, siRNAs are designed as follows. First, a portion of a target gene is selected. Cleavage of mRNA at these sites should eliminate translation of corresponding protein. Antisense strands were designed based on the target sequence and sense strands were designed to be complementary to the antisense strand. Hybridization of the antisense and sense strands forms the siRNA duplex. The antisense strand includes about 19 to 25 nucleotides, e.g., 19, 20, 21, 22, 23, 24 or 25 nucleotides. In other embodiments, the antisense strand includes 20, 21, 22 or 23 nucleotides. The sense strand includes about 14 to 25 nucleotides, e.g., 14, 15, 16, 17, 18, 19, 20, 21, 22, 23, 24 or 25 nucleotides. In other embodiments, the sense strand is 15 nucleotides. In other embodiments, the sense strand is 16 nucleotides. In other embodiments, the sense strand is 17 nucleotides. In other embodiments, the sense strand is 18 nucleotides. In other embodiments, the sense strand is 19 nucleotides. In other embodiments, the sense strand is 20 nucleotides. The skilled artisan will appreciate, however, that siRNAs having antisense strands with a length of less than 19 nucleotides or greater than 25 nucleotides can also function to mediate RNAi. Accordingly, siRNAs of such length are also within the scope of the instant disclosure, provided that they retain the ability to mediate RNAi. Longer RNAi agents have been demonstrated to elicit an interferon or PKR response in certain mammalian cells, which may be undesirable. In certain embodiments, the RNAi agents of the disclosure do not elicit a PKR response (i.e., are of a sufficiently short length). However, longer RNAi agents may be useful, for example, in cell types incapable of generating a PKR response or in situations where the PKR response has been down-regulated or dampened by alternative means.

The sense strand sequence can be designed such that the target sequence is essentially in the middle of the strand. Moving the target sequence to an off-center position can, in some instances, reduce efficiency of cleavage by the siRNA. Such compositions, i.e., less efficient compositions, may be desirable for use if off-silencing of the wild-type mRNA is detected.

The antisense strand can be the same length as the sense strand and includes complementary nucleotides. In one embodiment, the strands are fully complementary, i.e., the strands are blunt-ended when aligned or annealed. In another embodiment, the strands align or anneal such that 1-, 2-, 3-, 4-, 5-, 6-, 7-, or 8-nucleotide overhangs are generated, i.e., the 3′ end of the sense strand extends 1, 2, 3, 4, 5, 6, 7, or 8 nucleotides further than the 5′ end of the antisense strand and/or the 3′ end of the antisense strand extends 1, 2, 3, 4, 5, 6, 7, or 8 nucleotides further than the 5′ end of the sense strand. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced, as described in detail in U.S. Pat. Nos. 7,459,547, 7,772,203 and 7,732,593, entitled “Methods and Compositions for Controlling Efficacy of RNA Silencing” (filed Jun. 2, 2003) and U.S. Pat. Nos. 8,309,704, 7,750,144, 8,304,530, 8,329,892 and 8,309,705, entitled “Methods and Compositions for Enhancing the Efficacy and Specificity of RNAi” (filed Jun. 2, 2003), the contents of which are incorporated in their entirety by this reference. In one embodiment of these aspects of the disclosure, the base-pair strength is less due to fewer G:C base pairs between the 5′ end of the first or antisense strand and the 3′ end of the second or sense strand than between the 3′ end of the first or antisense strand and the 5′ end of the second or sense strand. In another embodiment, the base pair strength is less due to at least one mismatched base pair between the 5′ end of the first or antisense strand and the 3′ end of the second or sense strand. In certain exemplary embodiments, the mismatched base pair is selected from the group consisting of G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair comprising a rare nucleotide, e.g., inosine (I). In certain exemplary embodiments, the base pair is selected from the group consisting of an I:A, I:U and I:C. In yet another embodiment, the base pair strength is less due to at least one base pair comprising a modified nucleotide. In certain exemplary embodiments, the modified nucleotide is selected from the group consisting of 2-amino-G, 2-amino-A, 2,6-diamino-G, and 2,6-diamino-A.

To validate the effectiveness by which siRNAs destroy mRNAs (e.g., mRNA expressed from a target gene), the siRNA can be incubated with cDNA (e.g., cDNA derived from a target gene) in a Drosophila-based in vitro mRNA expression system. Radiolabeled with ³²P, newly synthesized mRNAs (e.g., target mRNA) are detected autoradiographically on an agarose gel. The presence of cleaved mRNA indicates mRNA nuclease activity. Suitable controls include omission of siRNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence. Sites of siRNA-mRNA complementation are selected which result in optimal mRNA specificity and maximal mRNA cleavage.

### RNA Silencing Agent Features

In certain embodiments, the RNA silencing agent comprises at least 80% chemically modified nucleotides. In certain embodiments, the RNA silencing agent is fully chemically modified, i.e., 100% of the nucleotides are chemically modified.

In certain embodiments, the RNA silencing agent is 2′-O-methyl rich, i.e., comprises greater than 50% 2′-O-methyl content. In certain embodiments, the RNA silencing agent comprises at least about 55%, 60%, 65%, 70%, 75%, 80%, 85%, 90%, 95%, or 100% 2′-O-methyl nucleotide content. In certain embodiments, the RNA silencing agent comprises at least about 70% 2′-O-methyl nucleotide modifications. In certain embodiments, the RNA silencing agent comprises between about 70% and about 90% 2′-O-methyl nucleotide modifications. In certain embodiments, the RNA silencing agent is a dsRNA comprising an antisense strand and sense strand. In certain embodiments, the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich RNA silencing agents and specific chemical modification patterns are further described in U.S. Ser. No. 16/550,076 (filed Aug. 23, 2019) and U.S. Ser. No. 62/891,185 (filed Aug. 23, 2019), each of which is incorporated herein by reference.

### Conjugated Functional Moieties

In other embodiments, RNA silencing agents may be modified with one or more functional moieties. A functional moiety is a molecule that confers one or more additional activities to the RNA silencing agent. In certain embodiments, the functional moieties enhance cellular uptake by target cells (e.g., neuronal cells). Thus, the disclosure includes RNA silencing agents which are conjugated or unconjugated (e.g., at its 5′ and/or 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Sunni. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA silencing agent of disclosure is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., 0-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP. In certain embodiments, the ligand is GalNAc or a derivative thereof.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid based ligand binds HSA. A lipid-based ligand can bind HSA with a sufficient affinity such that the conjugate will be distributed to a non-kidney tissue. However, it is contemplated that the affinity not be so strong that the HSA-ligand binding cannot be reversed. In another embodiment, the lipid based ligand binds HSA weakly or not at all, such that the conjugate will be distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

In certain embodiments, the functional moiety is linked to the 5′ end and/or 3′ end of the RNA silencing agent of the disclosure. In certain embodiments, the functional moiety is linked to the 5′ end and/or 3′ end of an antisense strand of the RNA silencing agent of the disclosure. In certain embodiments, the functional moiety is linked to the 5′ end and/or 3′ end of a sense strand of the RNA silencing agent of the disclosure. In certain embodiments, the functional moiety is linked to the 3′ end of a sense strand of the RNA silencing agent of the disclosure.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker comprises a divalent or trivalent linker. In certain embodiments, the linker comprises an ethylene glycol chain, an alkyl chain, a peptide, RNA, DNA, a phosphodiester, a phosphorothioate, a phosphoramidate, an amide, a carbamate, or a combination thereof. In certain embodiments, the divalent or trivalent linker is selected from:

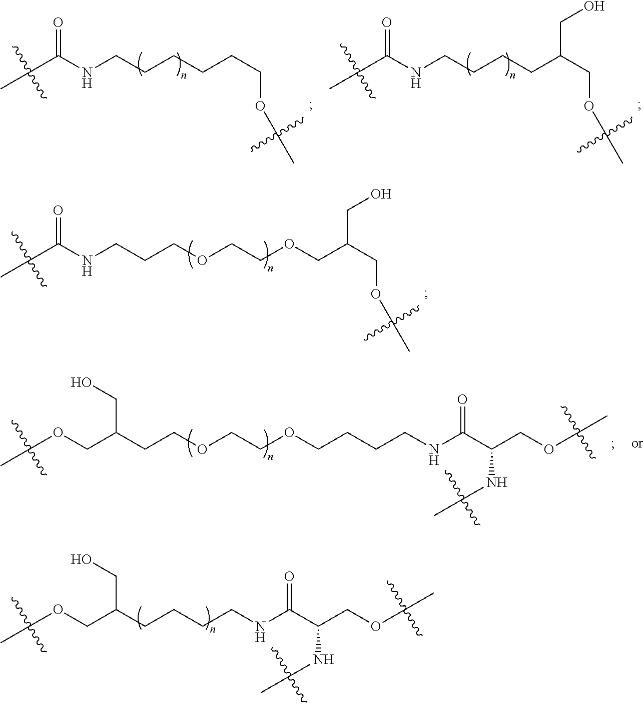

wherein n is 1, 2, 3, 4, or 5.

In certain embodiments, the linker further comprises a phosphodiester or phosphodiester derivative. In certain embodiments, the phosphodiester or phosphodiester derivative is selected from the group consisting of:

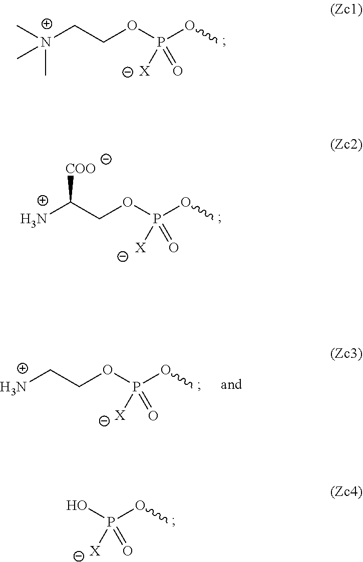

wherein X is O, S or BH₃.

The various functional moieties of the disclosure and means to conjugate them to RNA silencing agents are described in further detail in WO2017/030973A1 and WO2018/031933A2, incorporated herein by reference.

### Branched Oligonucleotides

Two or more RNA silencing agents as disclosed supra, for example oligonucleotide constructs such as siRNAs, may be connected to one another by one or more moieties independently selected from a linker, a spacer and a branching point, to form a branched oligonucleotide RNA silencing agent. In certain embodiments, the branched oligonucleotide RNA silencing agent consists of two siRNAs to form a di-branched siRNA (“di-siRNA”) scaffolding for delivering two siRNAs. In representative embodiments, the nucleic acids of the branched oligonucleotide each comprise an antisense strand (or portions thereof), wherein the antisense strand has sufficient complementarity to a target mRNA to mediate an RNA-mediated silencing mechanism (e.g. RNAi).

In exemplary embodiments, the branched oligonucleotides may have two to eight RNA silencing agents attached through a linker. The linker may be hydrophobic. In an embodiment, branched oligonucleotides of the present application have two to three oligonucleotides. In an embodiment, the oligonucleotides independently have substantial chemical stabilization (e.g., at least 40% of the constituent bases are chemically-modified). In an exemplary embodiment, the oligonucleotides have full chemical stabilization (i.e., all the constituent bases are chemically-modified). In some embodiments, branched oligonucleotides comprise one or more single-stranded phosphorothioated tails, each independently having two to twenty nucleotides. In a non-limiting embodiment, each single-stranded tail has two to ten nucleotides.

In certain embodiments, branched oligonucleotides are characterized by three properties: (1) a branched structure, (2) full metabolic stabilization, and (3) the presence of a single-stranded tail comprising phosphorothioate linkers. In certain embodiments, branched oligonucleotides have 2 or 3 branches. It is believed that the increased overall size of the branched structures promotes increased uptake. Also, without being bound by a particular theory of activity, multiple adjacent branches (e.g., 2 or 3) are believed to allow each branch to act cooperatively and thus dramatically enhance rates of internalization, trafficking and release.

Branched oligonucleotides are provided in various structurally diverse embodiments. In some embodiments nucleic acids attached at the branching points are single stranded or double stranded and consist of miRNA inhibitors, gapmers, mixmers, SSOs, PMOs, or PNAs. These single strands can be attached at their 3′ or 5′ end. Combinations of siRNA and single stranded oligonucleotides could also be used for dual function. In another embodiment, short nucleic acids complementary to the gapmers, mixmers, miRNA inhibitors, SSOs, PMOs, and PNAs are used to carry these active single-stranded nucleic acids and enhance distribution and cellular internalization. The short duplex region has a low melting temperature (Tm˜37° C.) for fast dissociation upon internalization of the branched structure into the cell.

The Di-siRNA branched oligonucleotides may comprise chemically diverse conjugates, such as the functional moieties described above. Conjugated bioactive ligands may be used to enhance cellular specificity and to promote membrane association, internalization, and serum protein binding. Examples of bioactive moieties to be used for conjugation include DHA, GalNAc, and cholesterol. These moieties can be attached to Di-siRNA either through the connecting linker or spacer, or added via an additional linker or spacer attached to another free siRNA end.

The presence of a branched structure improves the level of tissue retention in the brain more than 100-fold compared to non-branched compounds of identical chemical composition, suggesting a new mechanism of cellular retention and distribution. Branched oligonucleotides have unexpectedly uniform distribution throughout the spinal cord and brain. Moreover, branched oligonucleotides exhibit unexpectedly efficient systemic delivery to a variety of tissues, and very high levels of tissue accumulation.

Branched oligonucleotides comprise a variety of therapeutic nucleic acids, including siRNAs, ASOs, miRNAs, miRNA inhibitors, splice switching, PMOs, PNAs. In some embodiments, branched oligonucleotides further comprise conjugated hydrophobic moieties and exhibit unprecedented silencing and efficacy in vitro and in vivo.

Linkers

In an embodiment of the branched oligonucleotide, each linker is independently selected from an ethylene glycol chain, an alkyl chain, a peptide, RNA, DNA, a phosphate, a phosphonate, a phosphoramidate, an ester, an amide, a triazole, and combinations thereof; wherein any carbon or oxygen atom of the linker is optionally replaced with a nitrogen atom, bears a hydroxyl substituent, or bears an oxo substituent. In one embodiment, each linker is an ethylene glycol chain. In another embodiment, each linker is an alkyl chain. In another embodiment, each linker is a peptide. In another embodiment, each linker is RNA. In another embodiment, each linker is DNA. In another embodiment, each linker is a phosphate. In another embodiment, each linker is a phosphonate. In another embodiment, each linker is a phosphoramidate. In another embodiment, each linker is an ester. In another embodiment, each linker is an amide. In another embodiment, each linker is a triazole.

Branched oligonucleotides, including synthesis and methods of use, are described in greater detail in WO2017/132669, incorporated herein by reference.

### Methods of Introducing RNA Silencing Agents

RNA silencing agents of the disclosure may be directly introduced into the cell (e.g., a neural cell) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the disclosure can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or otherwise increase inhibition of the target gene.

Physical methods of introducing nucleic acids include injection of a solution containing the RNA, bombardment by particles covered by the RNA, soaking the cell or organism in a solution of the RNA, or electroporation of cell membranes in the presence of the RNA. Other methods known in the art for introducing nucleic acids to cells may be used, such as lipid-mediated carrier transport, chemical-mediated transport, such as calcium phosphate, and the like. Thus, the RNA may be introduced along with components that perform one or more of the following activities: enhance RNA uptake by the cell, inhibit annealing of single strands, stabilize the single strands, or otherwise increase inhibition of the target gene.

RNA may be directly introduced into the cell (i.e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the RNA may be introduced.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadioImmunoAssay (RIA), other immunoassays, and Fluorescence Activated Cell Sorting (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Depending on the assay, quantitation of the amount of gene expression allows one to determine a degree of inhibition which is greater than 10%, 33%, 50%, 90%, 95% or 99% as compared to a cell not treated according to the present disclosure. Lower doses of injected material and longer times after administration of RNAi agent may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In an exemplary aspect, the efficacy of an RNAi agent of the disclosure (e.g., an siRNA targeting a target sequence of interest) is tested for its ability to specifically degrade mutant mRNA (e.g., target mRNA and/or the production of target protein) in cells, in particular, in neurons (e.g., striatal or cortical neuronal clonal lines and/or primary neurons). Also suitable for cell-based validation assays are other readily transfectable cells, for example, HeLa cells or COS cells. Cells are transfected with human wild type or mutant cDNAs (e.g., human wild type or mutant target cDNA). Standard siRNA, modified siRNA or vectors able to produce siRNA from U-looped mRNA are co-transfected. Selective reduction in target mRNA and/or target protein is measured. Reduction of target mRNA or protein can be compared to levels of target mRNA or protein in the absence of an RNAi agent or in the presence of an RNAi agent that does not target the target mRNA. Exogenously-introduced mRNA or protein (or endogenous mRNA or protein) can be assayed for comparison purposes. When utilizing neuronal cells, which are known to be somewhat resistant to standard transfection techniques, it may be desirable to introduce RNAi agents (e.g., siRNAs) by passive uptake.

### Methods of Treatment

“Treatment,” or “treating,” as used herein, is defined as the application or administration of a therapeutic agent (e.g., a RNA agent) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosure provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the disclosure pertains to methods treating subjects therapeutically, i.e., alter onset of symptoms of the disease or disorder.

With regards to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype,” or “drug response genotype”). Thus, another aspect of the disclosure provides methods for tailoring an individual's prophylactic or therapeutic treatment with either the target gene molecules of the present disclosure or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in an appropriate animal model. For example, an RNAi agent (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

### Pharmaceutical Compositions and Methods of Administration

The disclosure pertains to uses of the above-described agents for prophylactic and/or therapeutic treatments as described infra. Accordingly, the modulators (e.g., RNAi agents) of the present disclosure can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. In certain exemplary embodiments, the pharmaceutical composition of the disclosure is administered intravenously and is capable of crossing the blood brain barrier to enter the central nervous system In certain exemplary embodiments, a pharmaceutical composition of the disclosure is delivered to the cerebrospinal fluid (CSF) by a route of administration that includes, but is not limited to, intrastriatal (IS) administration, intracerebroventricular (ICV) administration and intrathecal (IT) administration (e.g., via a pump, an infusion or the like).

In certain embodiments, a composition that includes a compound of the disclosure can be delivered to the nervous system of a subject by a variety of routes. Exemplary routes include intrathecal, parenchymal (e.g., in the brain), nasal, and ocular delivery. The composition can also be delivered systemically, e.g., by intravenous, subcutaneous or intramuscular injection. One route of delivery is directly to the brain, e.g., into the ventricles or the hypothalamus of the brain, or into the lateral or dorsal areas of the brain. The compounds for neural cell delivery can be incorporated into pharmaceutical compositions suitable for administration.

For example, compositions can include one or more species of a compound of the disclosure and a pharmaceutically acceptable carrier. The pharmaceutical compositions of the present disclosure may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, intrathecal, or intraventricular (e.g., intracerebroventricular) administration. In certain exemplary embodiments, an RNA silencing agent of the disclosure is delivered across the Blood-Brain-Barrier (BBB) suing a variety of suitable compositions and methods described herein.

The route of delivery can be dependent on the disorder of the patient. In addition to a compound of the disclosure, a patient can be administered a second therapy, e.g., a palliative therapy and/or disease-specific therapy. The secondary therapy can be, for example, symptomatic (e.g., for alleviating symptoms), neuroprotective (e.g., for slowing or halting disease progression), or restorative (e.g., for reversing the disease process). Other therapies can include psychotherapy, physiotherapy, speech therapy, communicative and memory aids, social support services, and dietary advice.

A compound of the disclosure can be delivered to neural cells of the brain. In certain embodiments, the compounds of the disclosure may be delivered to the brain without direct administration to the central nervous system, i.e., the compounds may be delivered intravenously and cross the blood brain barrier to enter the brain. Delivery methods that do not require passage of the composition across the blood-brain barrier can be utilized. For example, a pharmaceutical composition containing a compound of the disclosure can be delivered to the patient by injection directly into the area containing the disease-affected cells. For example, the pharmaceutical composition can be delivered by injection directly into the brain. The injection can be by stereotactic injection into a particular region of the brain (e.g., the substantia nigra, cortex, hippocampus, striatum, or globus pallidus). The compound can be delivered into multiple regions of the central nervous system (e.g., into multiple regions of the brain, and/or into the spinal cord). The compound can be delivered into diffuse regions of the brain (e.g., diffuse delivery to the cortex of the brain).

In one embodiment, the compound can be delivered by way of a cannula or other delivery device having one end implanted in a tissue, e.g., the brain, e.g., the substantia nigra, cortex, hippocampus, striatum or globus pallidus of the brain. The cannula can be connected to a reservoir containing the compound. The flow or delivery can be mediated by a pump, e.g., an osmotic pump or minipump, such as an Alzet pump (Durect, Cupertino, CA). In one embodiment, a pump and reservoir are implanted in an area distant from the tissue, e.g., in the abdomen, and delivery is effected by a conduit leading from the pump or reservoir to the site of release. Devices for delivery to the brain are described, for example, in U.S. Pat. Nos. 6,093,180, and 5,814,014.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and is not intended to be limiting.

### Example 1. Screen of siRNA Chemical Modifications

The instant disclosure described numerous novel chemical modification patterns to enhance long term siRNA silencing activity while tailoring an appropriate level of target knock down. The siRNA utilized in the following chemical modification screen have a 21-nucleotide antisense and a 16-nucleotide sense strand. Modification patterns with only 2′-F and 2′-OMe have been successfully applied in vivo (see, e.g., US20160319278, US20200087663, and US20210115442, each of which is incorporated herein by reference). However, current siRNA scaffolds only last up to six months in vivo. Moreover, these prior modification patterns were capable of high-level silencing of targets by 80% or more. However, in certain contexts, it is desirable to tune the level target silencing such that a large amount of the target remains (i.e., knock down of about 50%). To those ends, this disclosure sought to identified nucleotide chemical modifications that can prolong the duration of in vivo silencing and tune silencing efficacy.

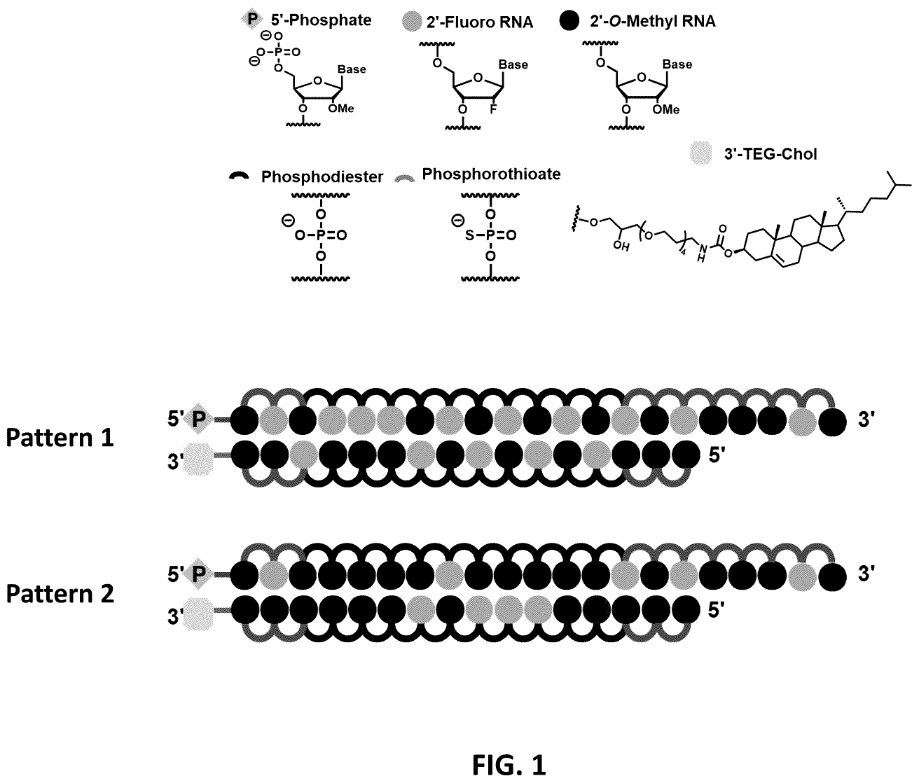

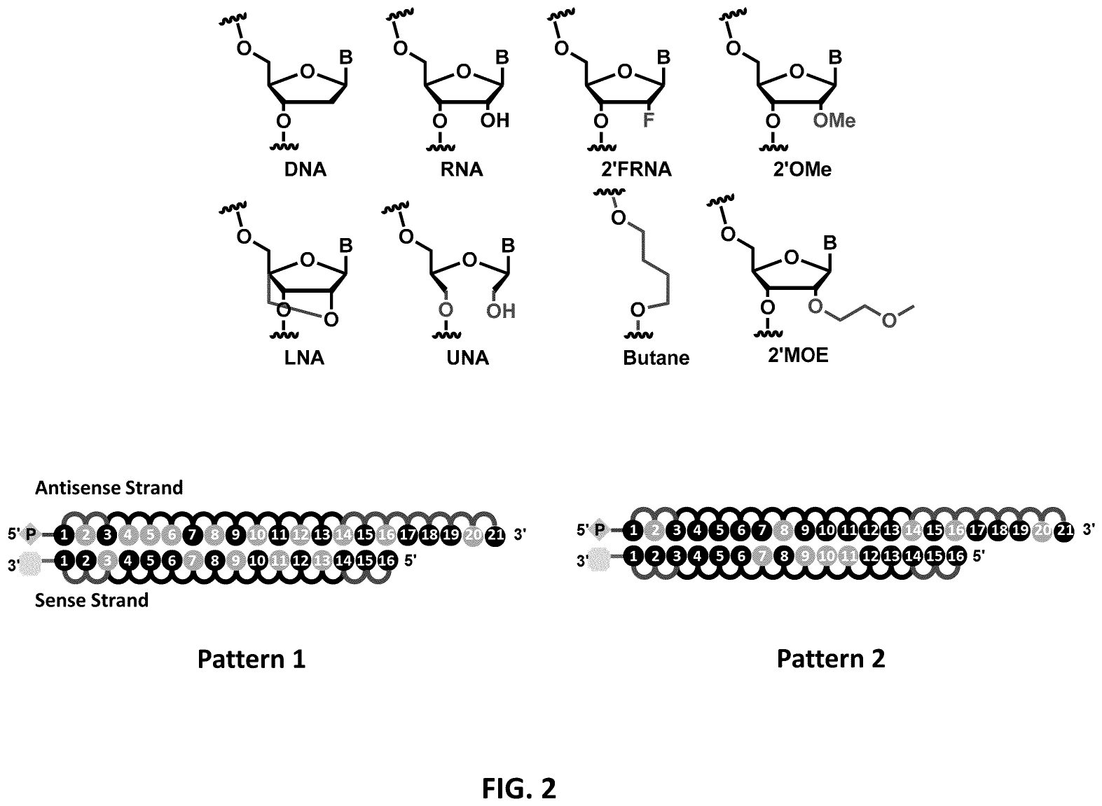

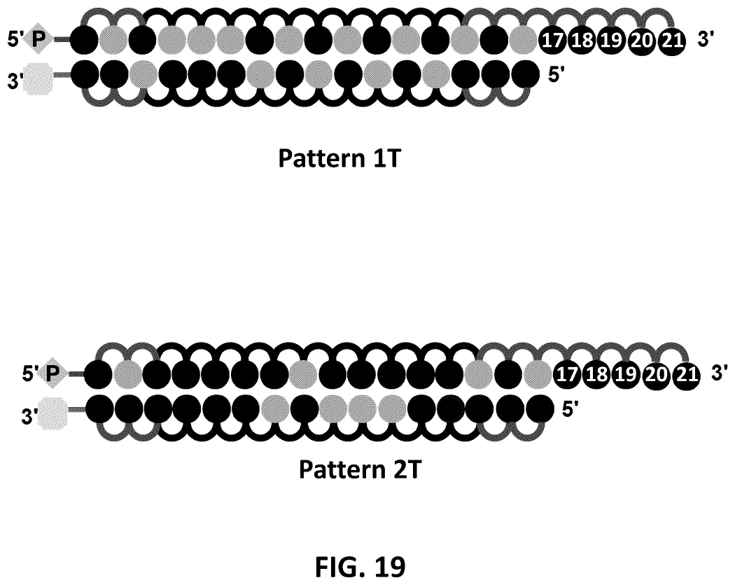

In this screen, certain modifications were placed at every position within the antisense and sense strand. Two parental chemical modification patterns were employed, Pattern 1 (P1) and Pattern 2 (P2), shown below (see also FIG. 1, FIG. 2, and FIG. 19):

P1 Antisense (5′ to 3′):
P(mN)#(fN)#(mN)(fN)(fN)(fN)(mN)(fN)(mN)(fN)(mN)
(fN)(mN)(fN)#(mN)#(fN)#(mN)#(mN)#(mN)#(fN)#(mN)
P1 Sense (5′ to 3′):
(mN)#(mN)#(mN)(fN)(mN)(fN)(mN)(fN)(mN)(fN)(mN)
(mN)(mN)(fN)#(mN)#(mN)-TegChol
P2 Antisense (5′ to 3′):
P(mN)#(fN)#(mN)(mN)(mN)(fN)(mN)(mN)(mN)(mN)(mN)
(mN)(mN)(fN)#(mN)#(fN)#(mN)#(mN)#(mN)#(fN)#(mN)
P2 Sense (5′ to 3′):
(mN)#(mN)#(mN)(mN)(mN)(fN)(fN)(fN)(mN)(fN)(mN)
(mN)(mN)(mN)#(mN)#(mN)-TegChol

In addition to using two different parental patterns, two different targets were used, one against HTT mRNA and one against MECP2 mRNA. The modifications utilized were: 2′-MOE, locked nucleic acid (LNA), unlocked nucleic acid (UNA), a butyl group (both in between two adjacent nucleotides and as an entire replacement of one nucleotide), 2′-deoxy, an unmodified ribonucleotide, and a base mismatch.

To perform the screen, cells were incubated with 0.5 μM siRNA and mRNA levels were measured 72 hours later using the QuantiGene SinglePlex assay.

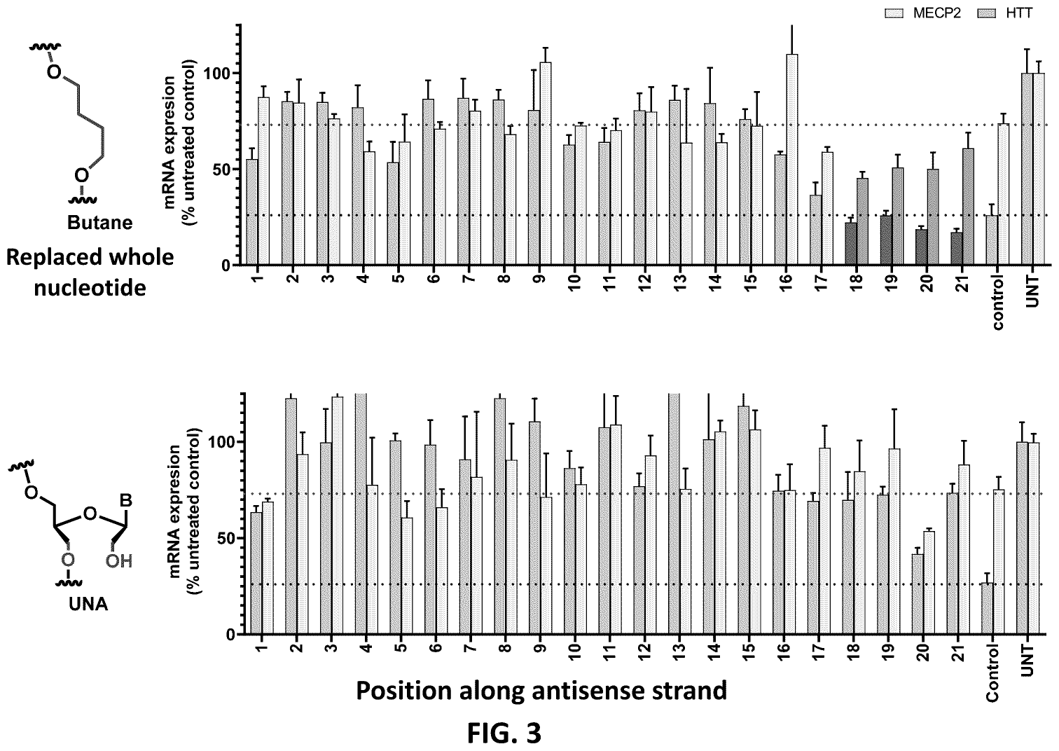

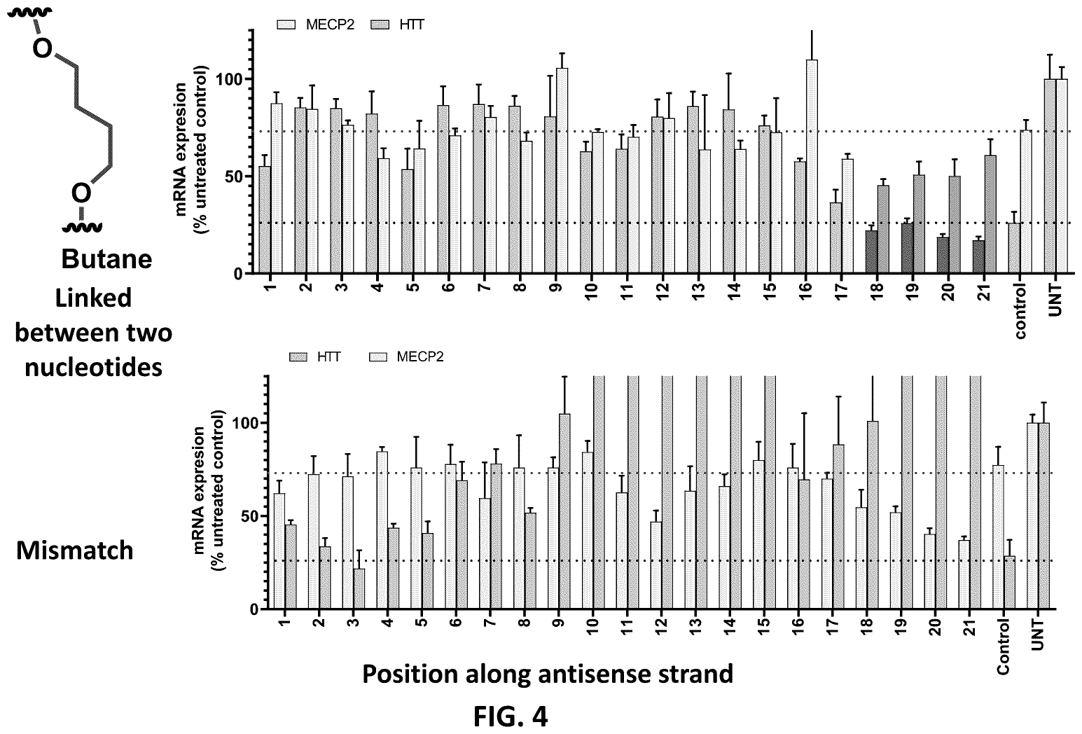

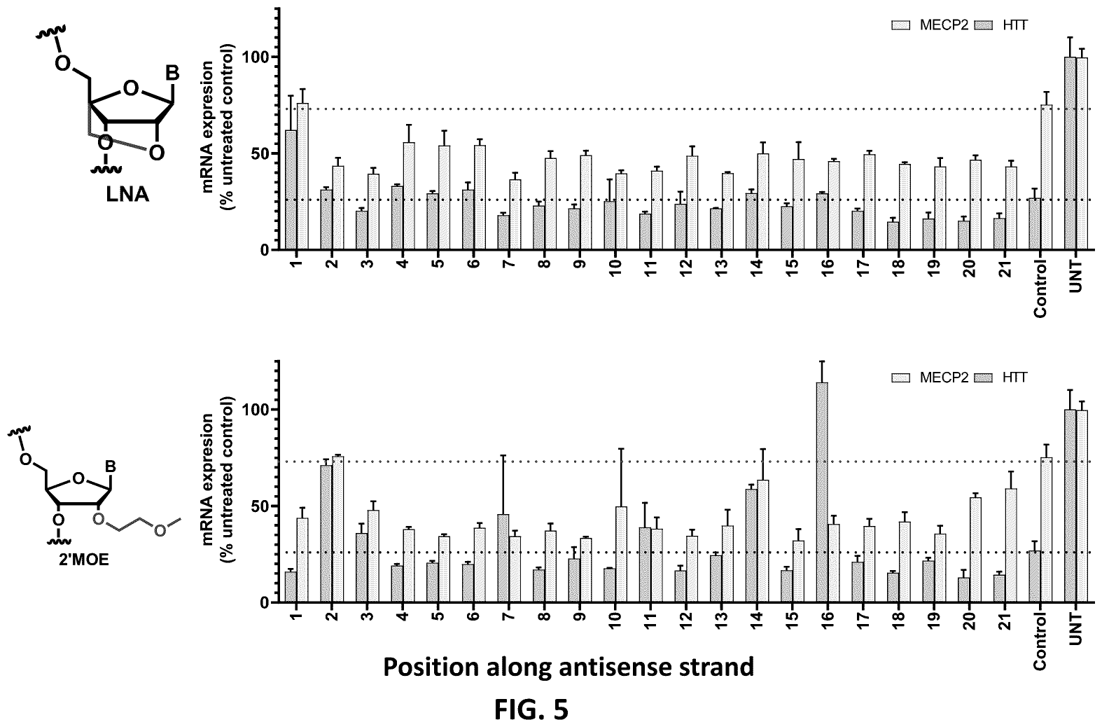

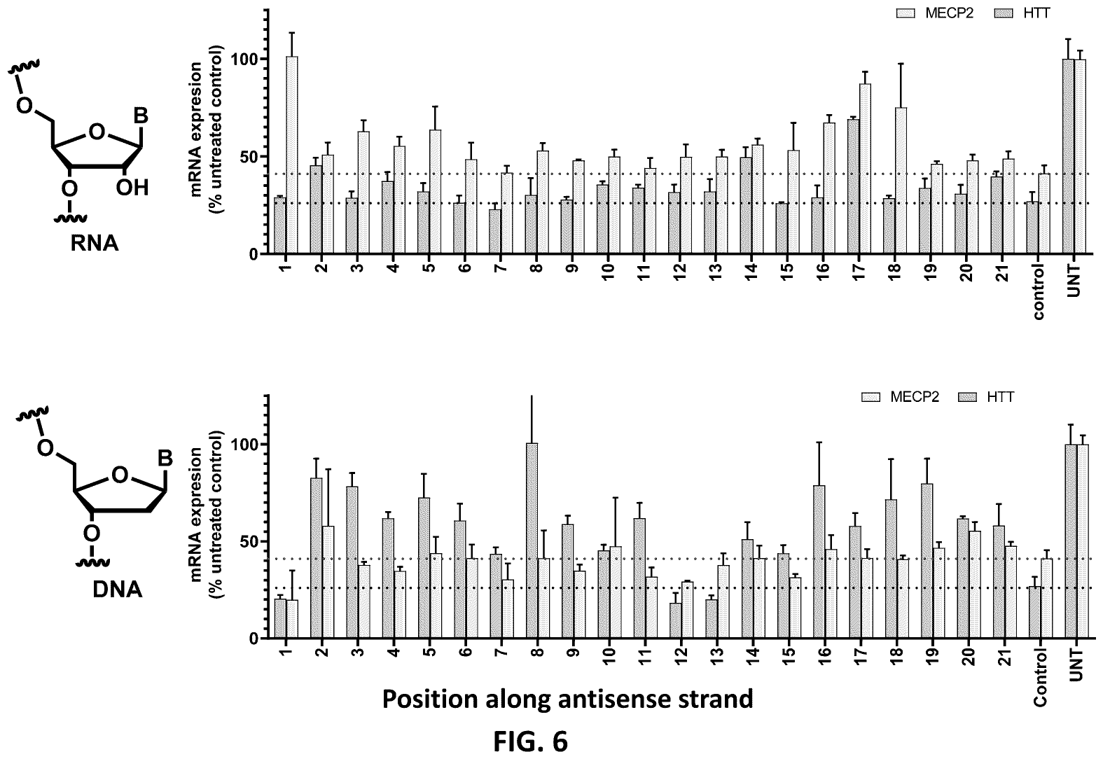

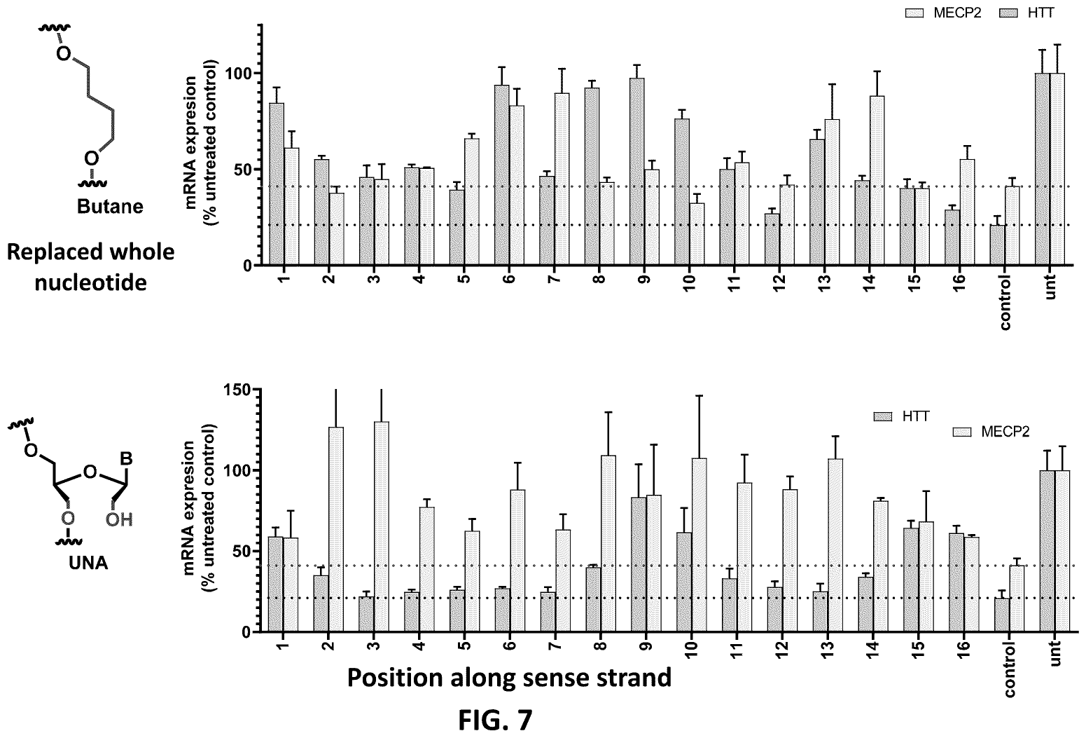

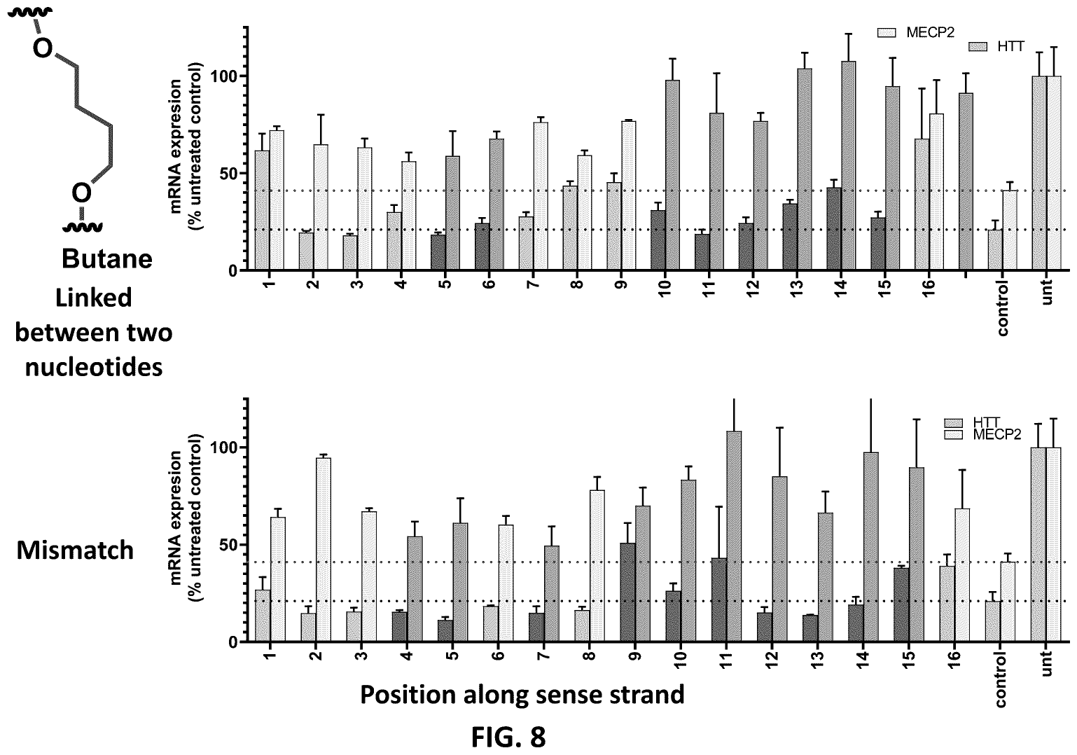

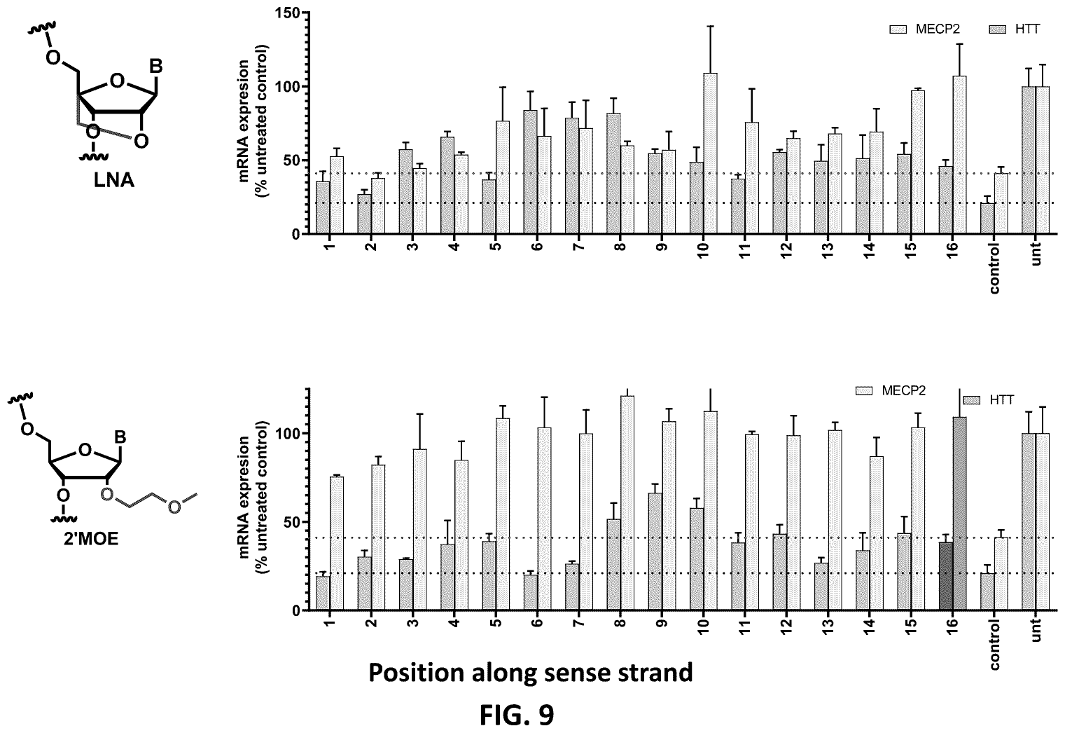

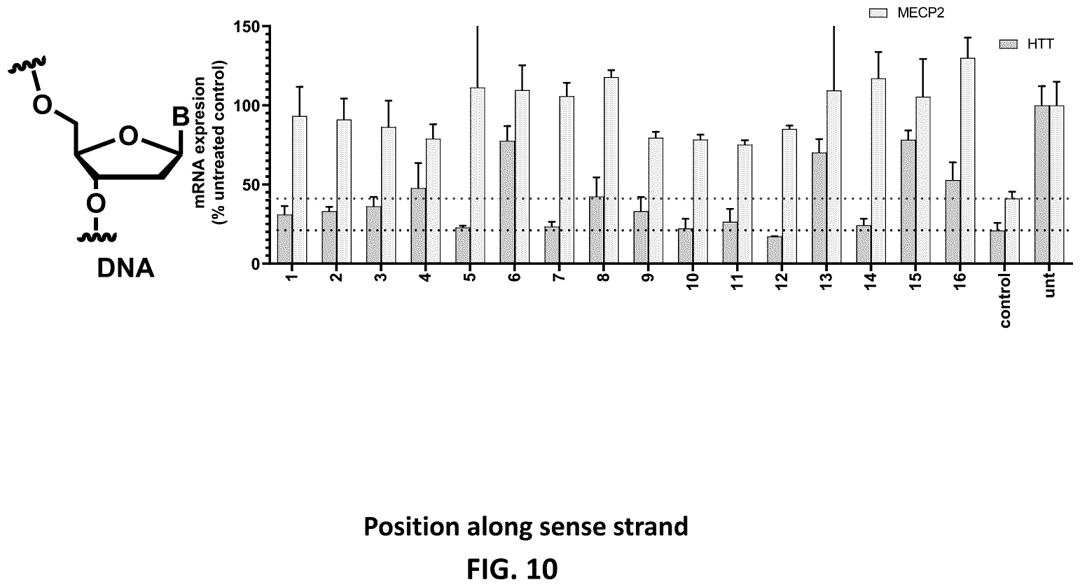

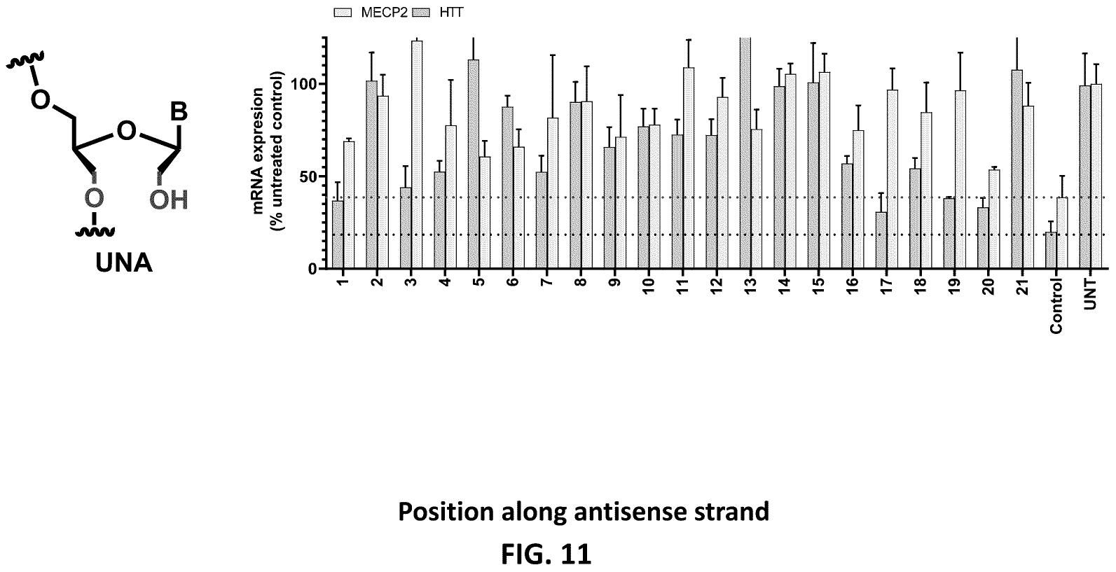

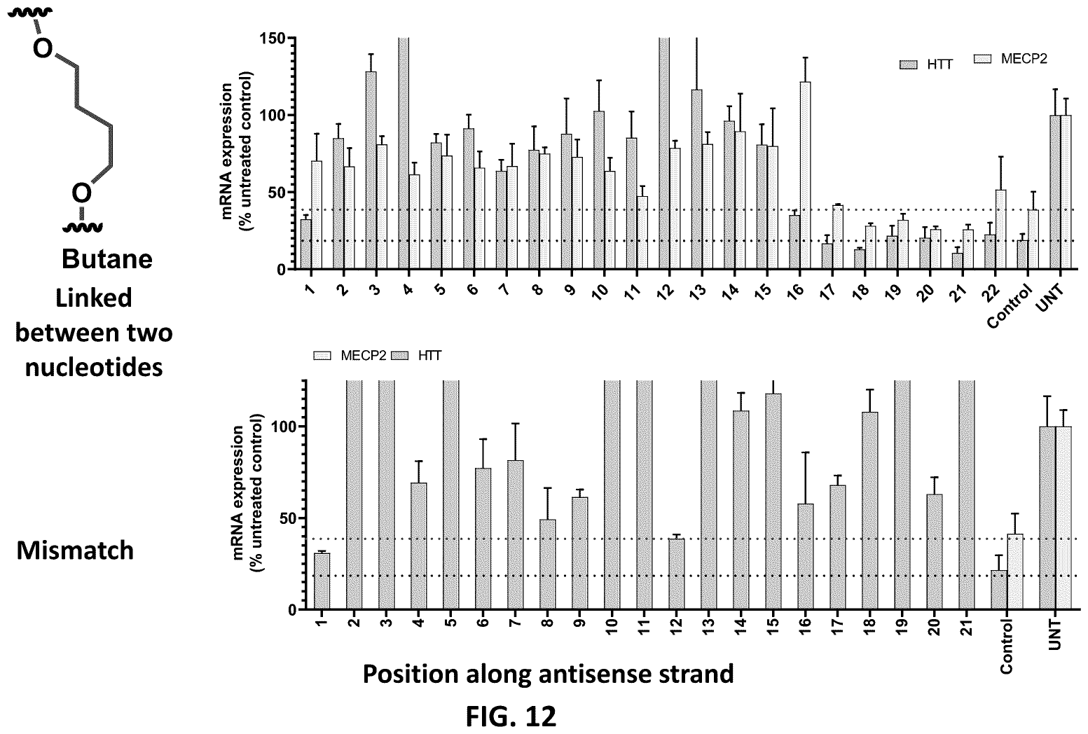

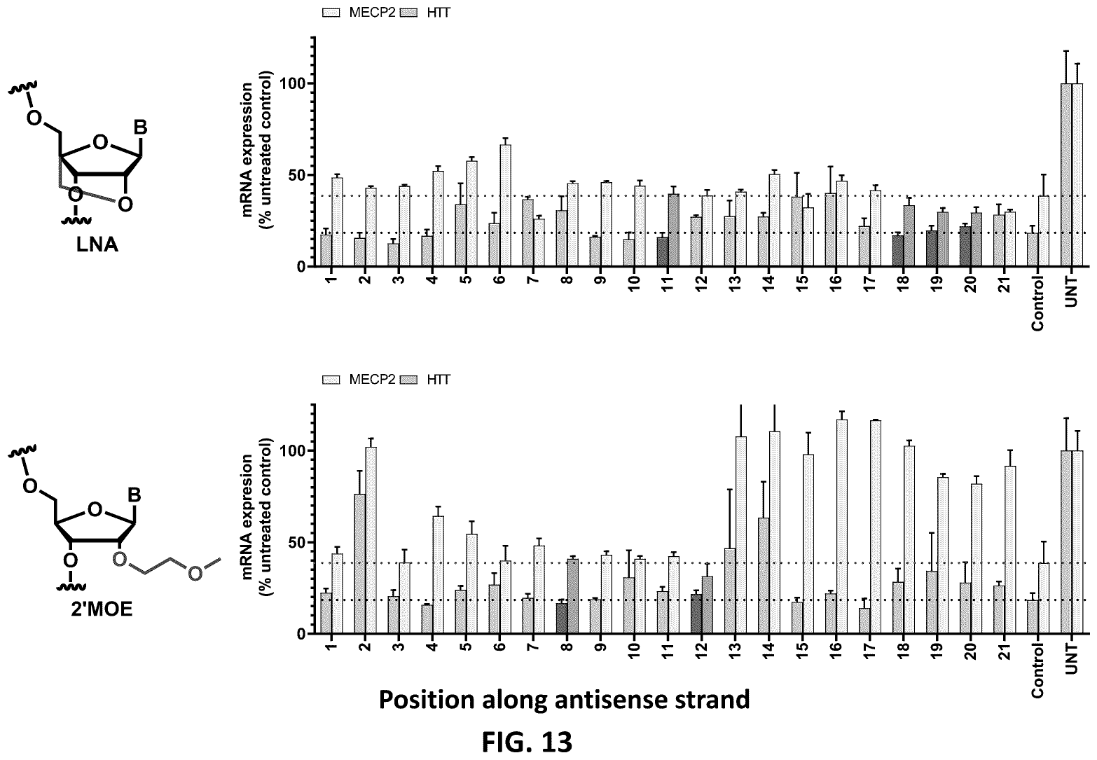

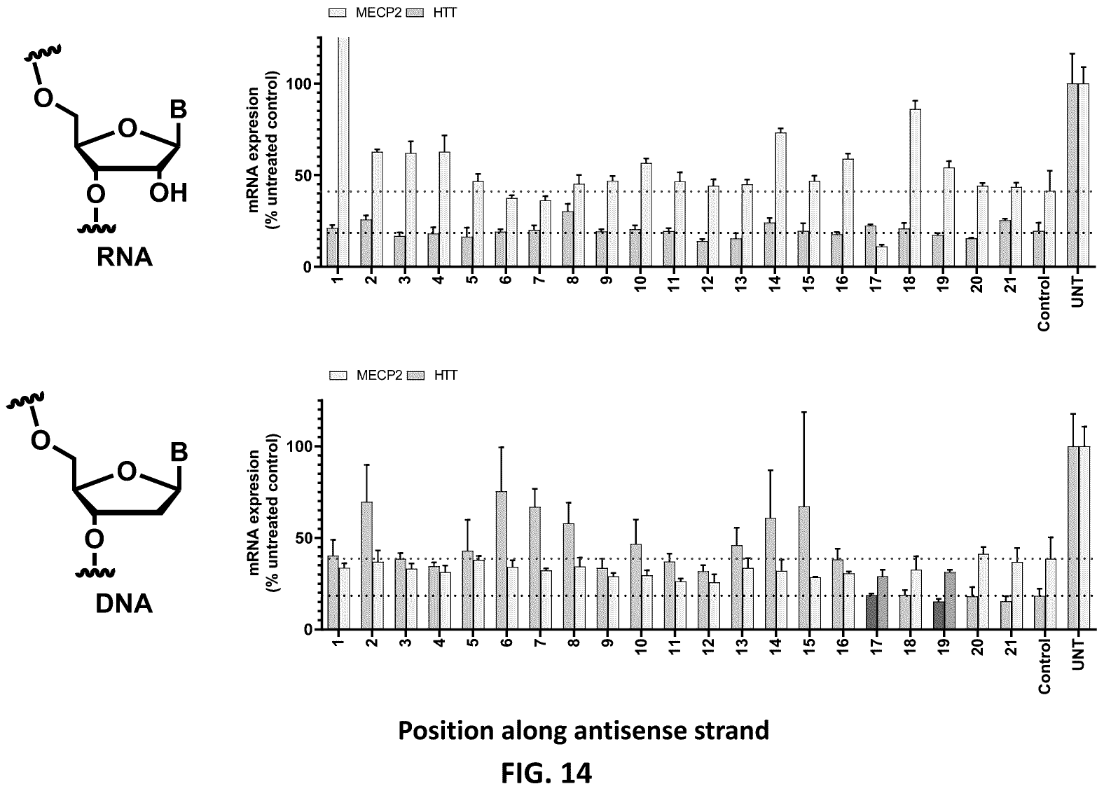

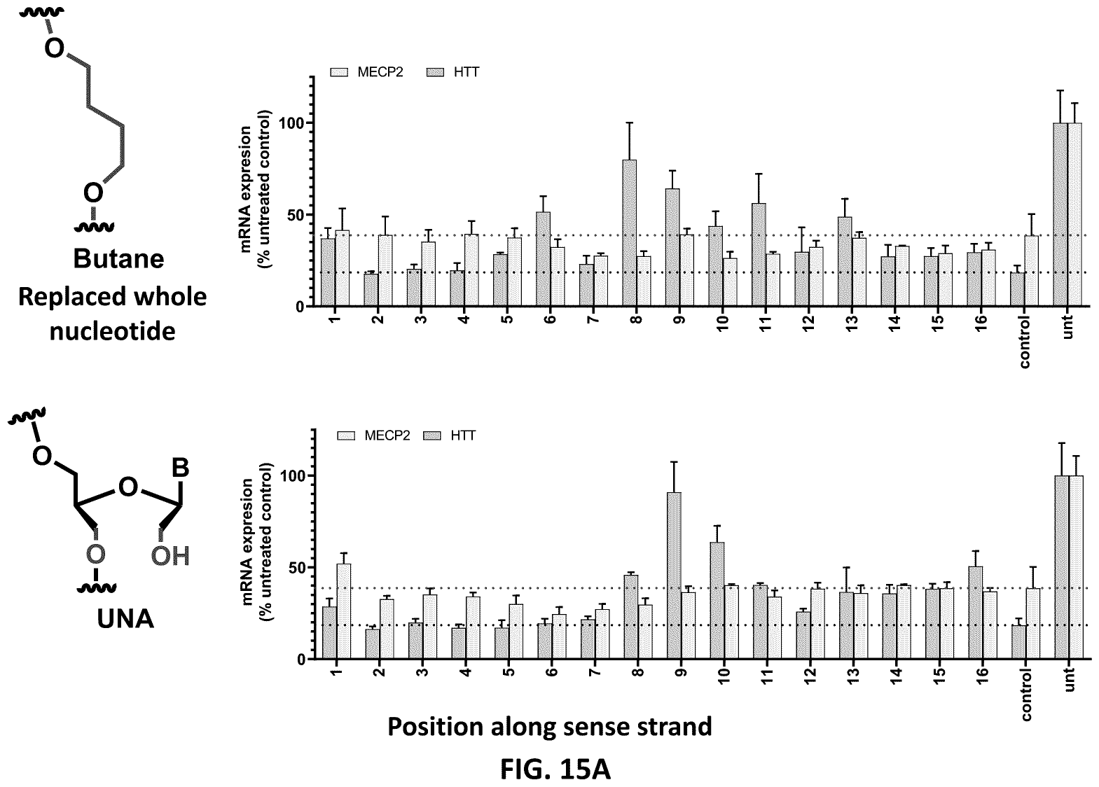

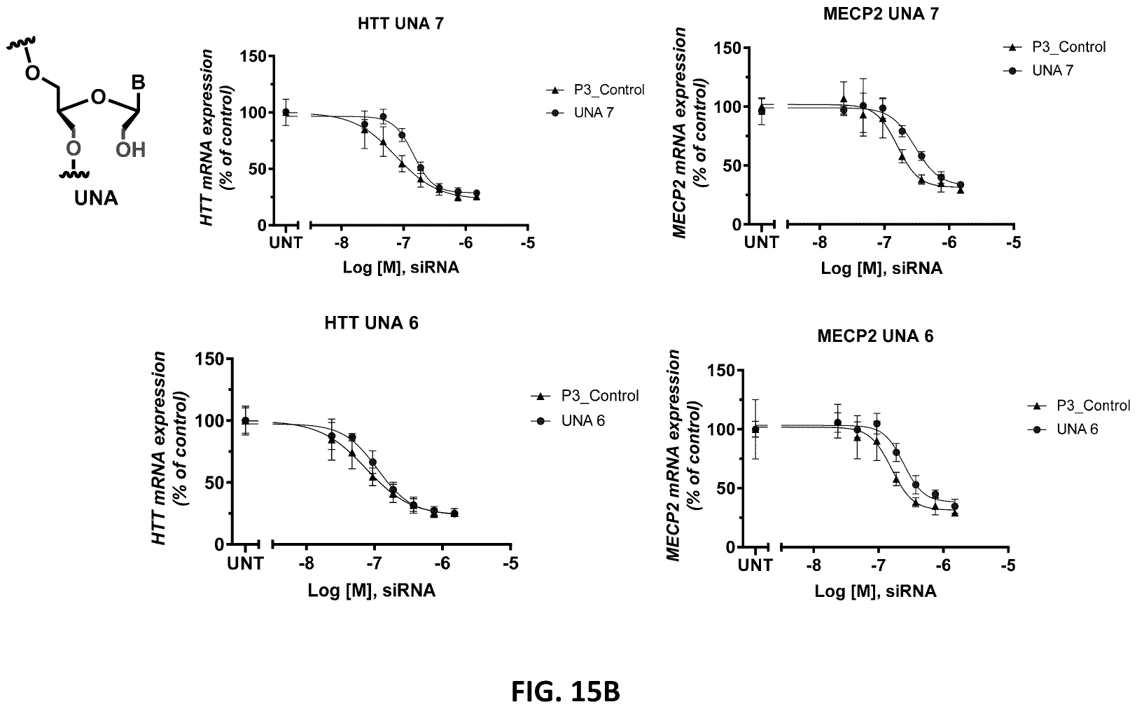

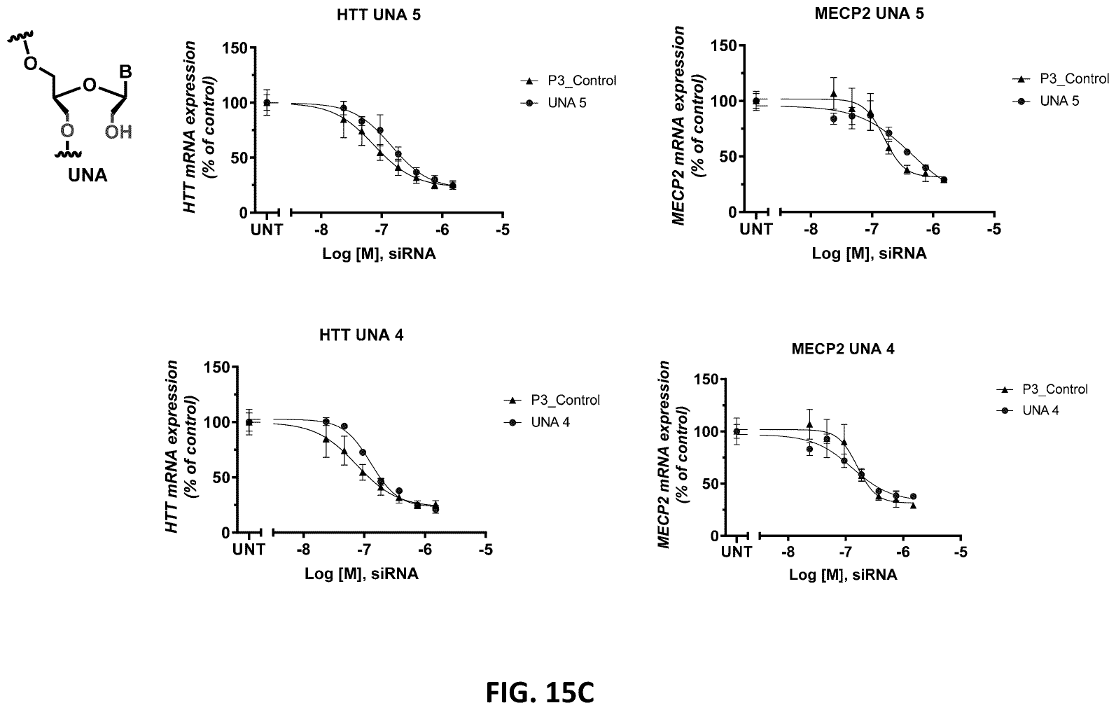

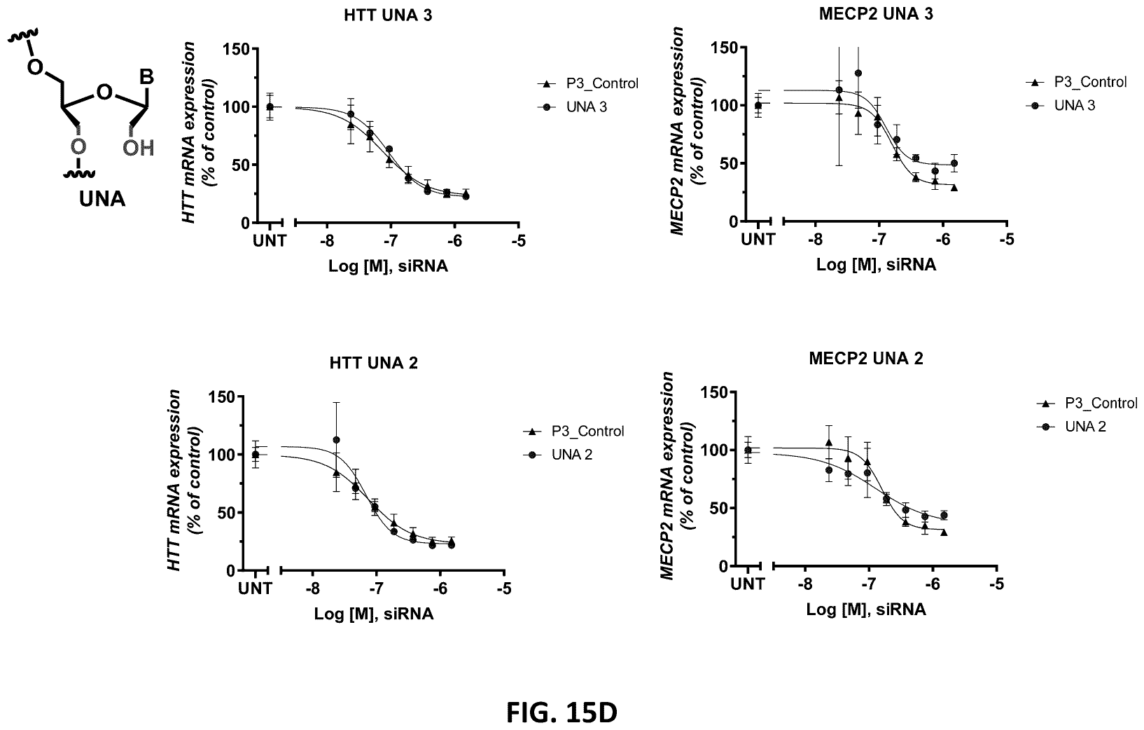

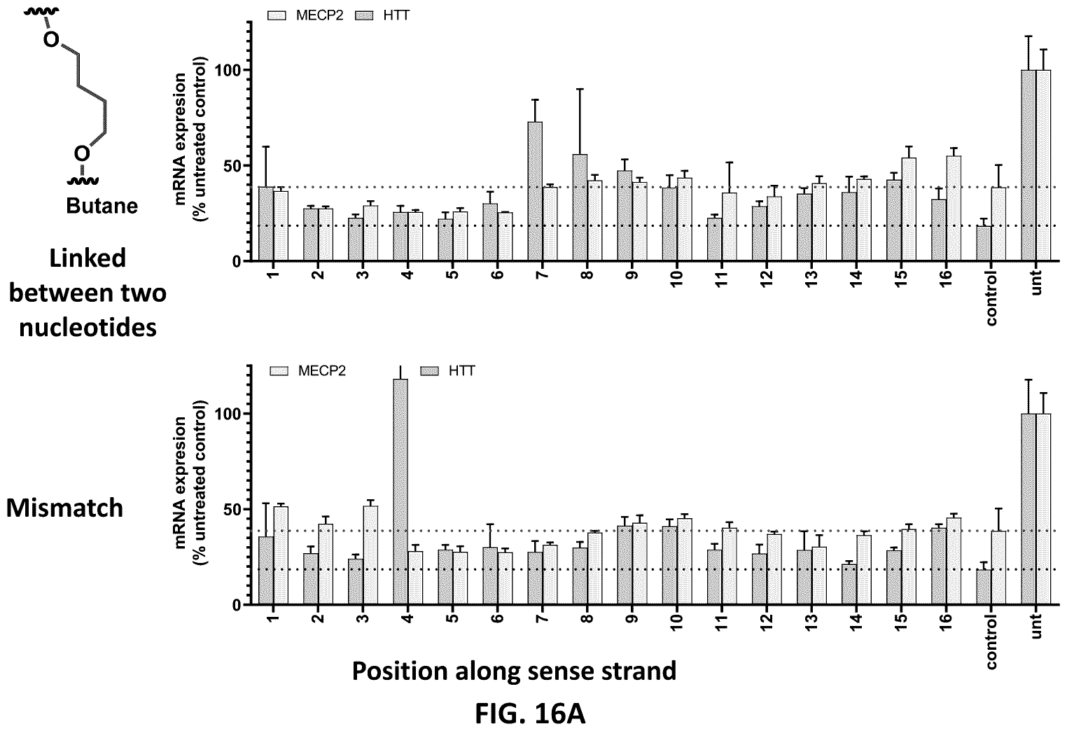

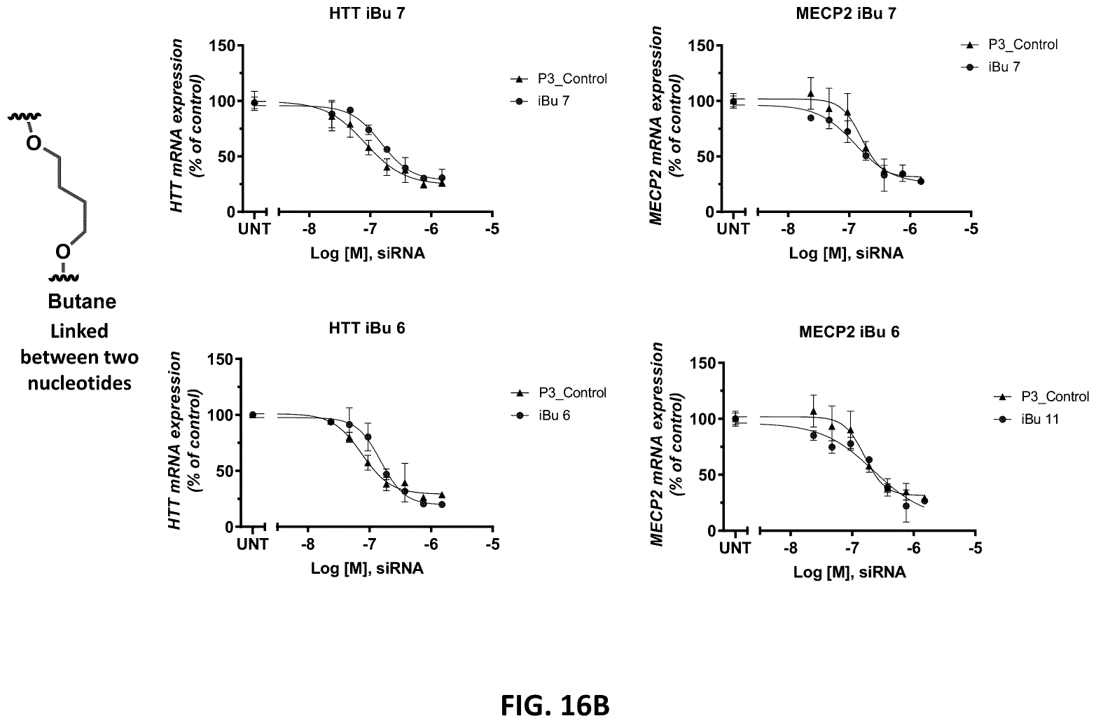

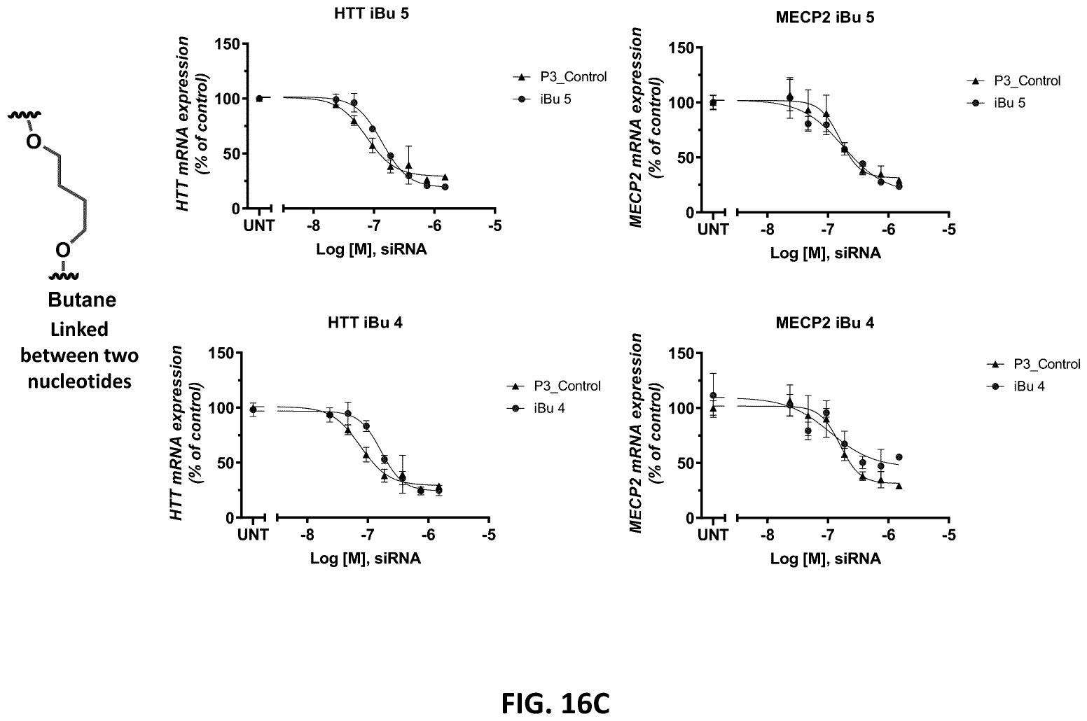

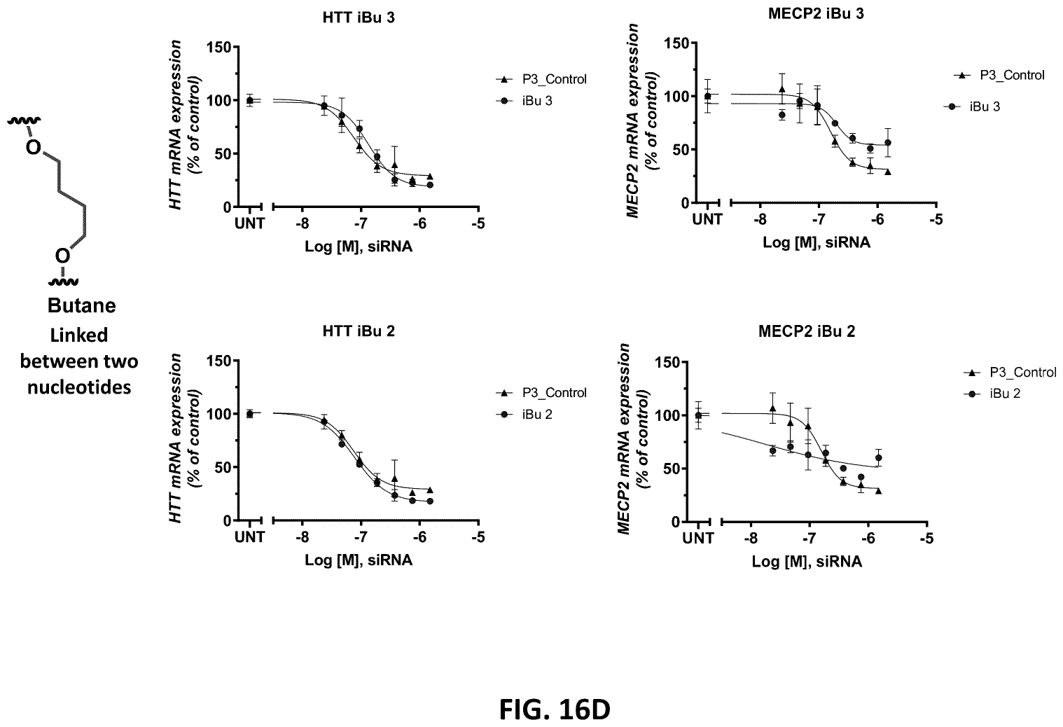

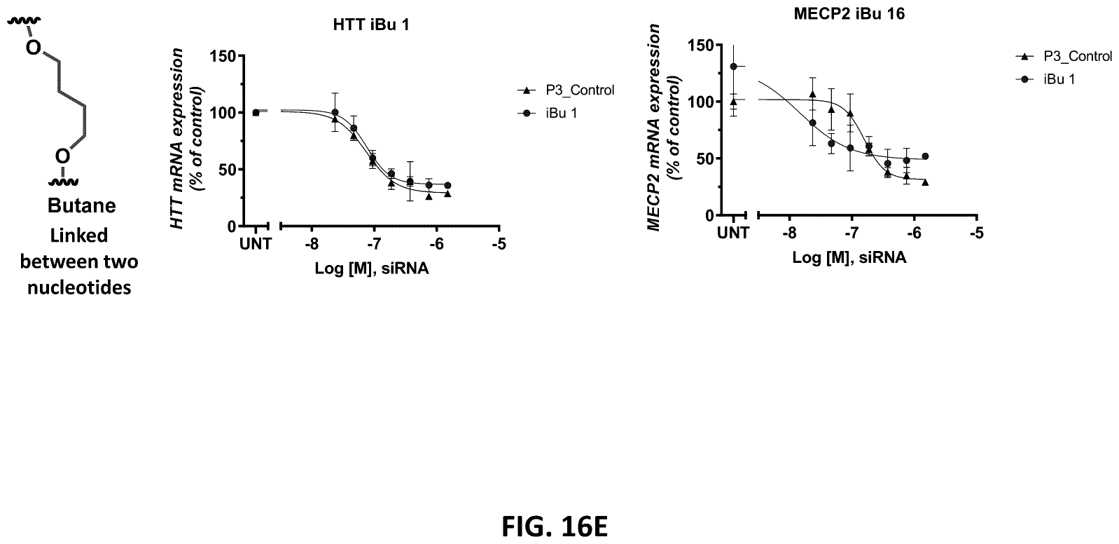

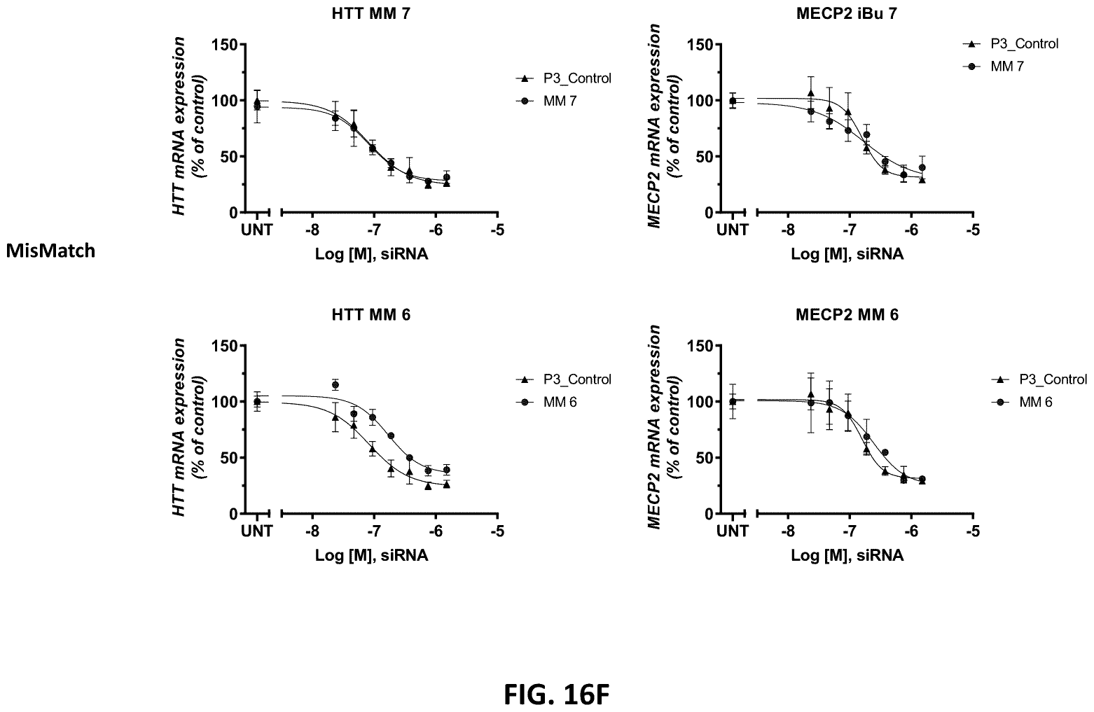

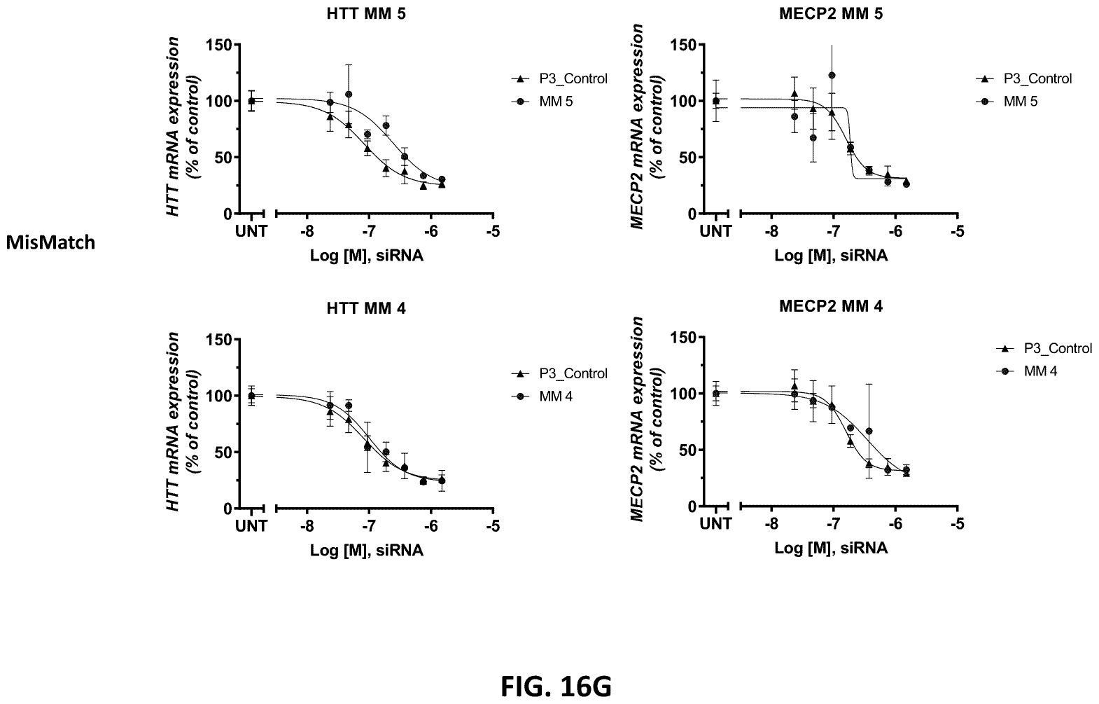

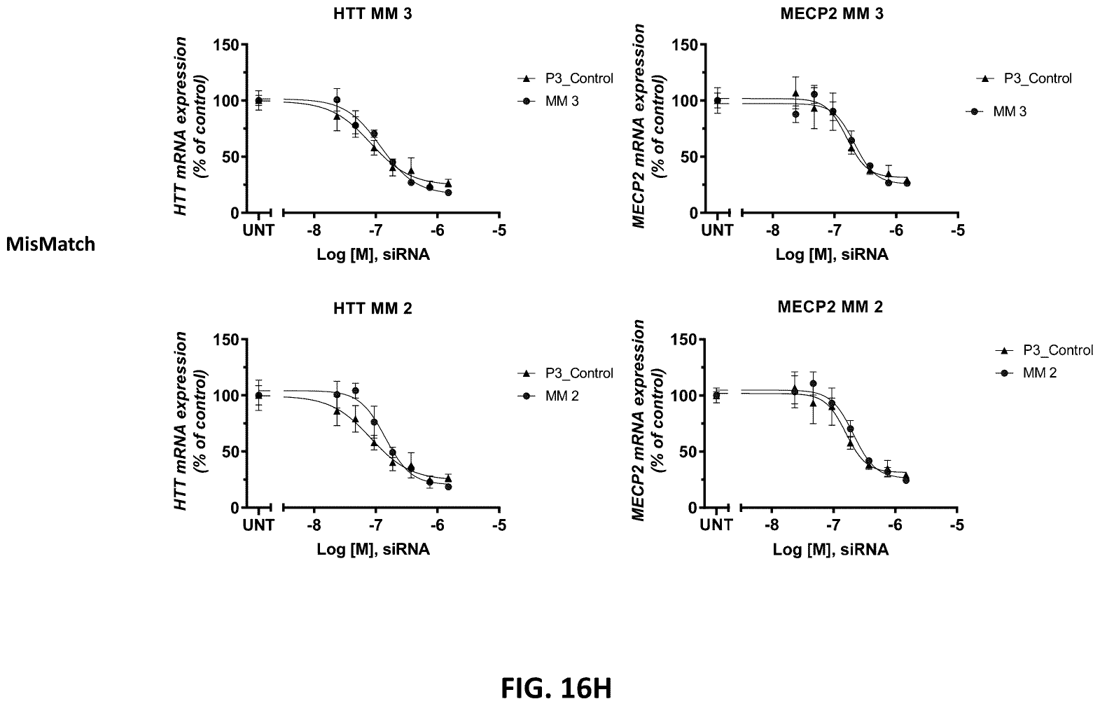

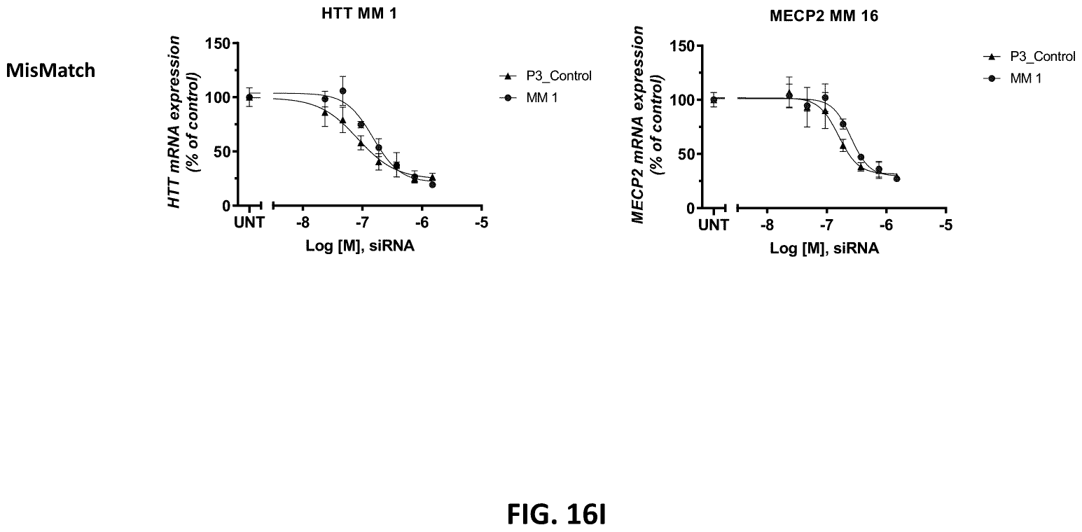

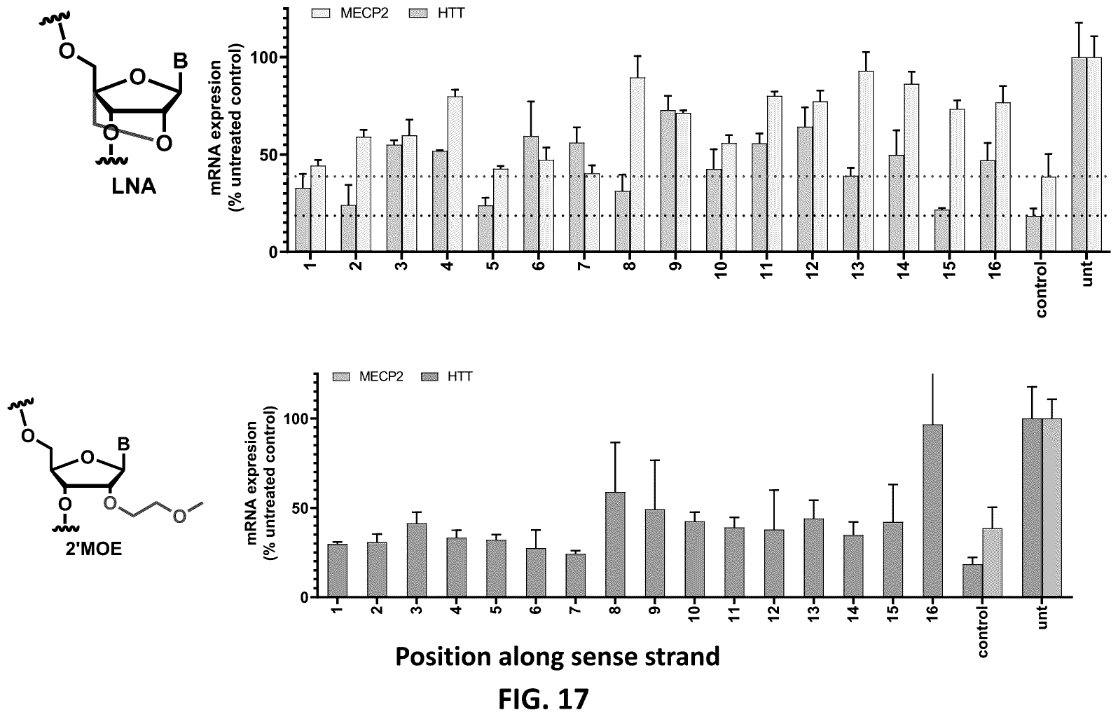

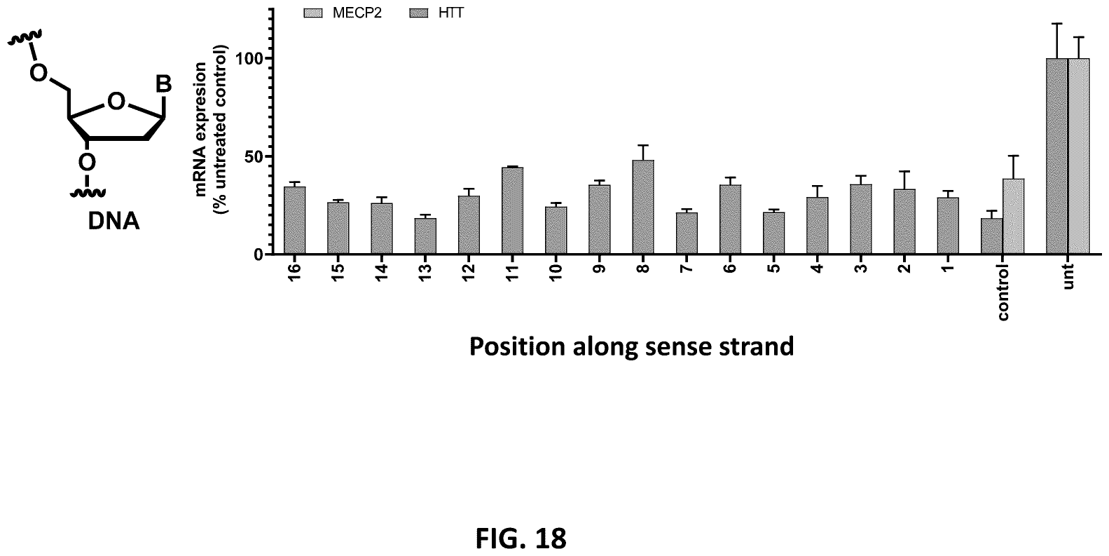

As shown in FIG. 3-18, the tested chemical modifications were capable of robust silencing at each of the positions within the antisense and sense strand. This was also true across the two different modification patterns and two different target mRNA.

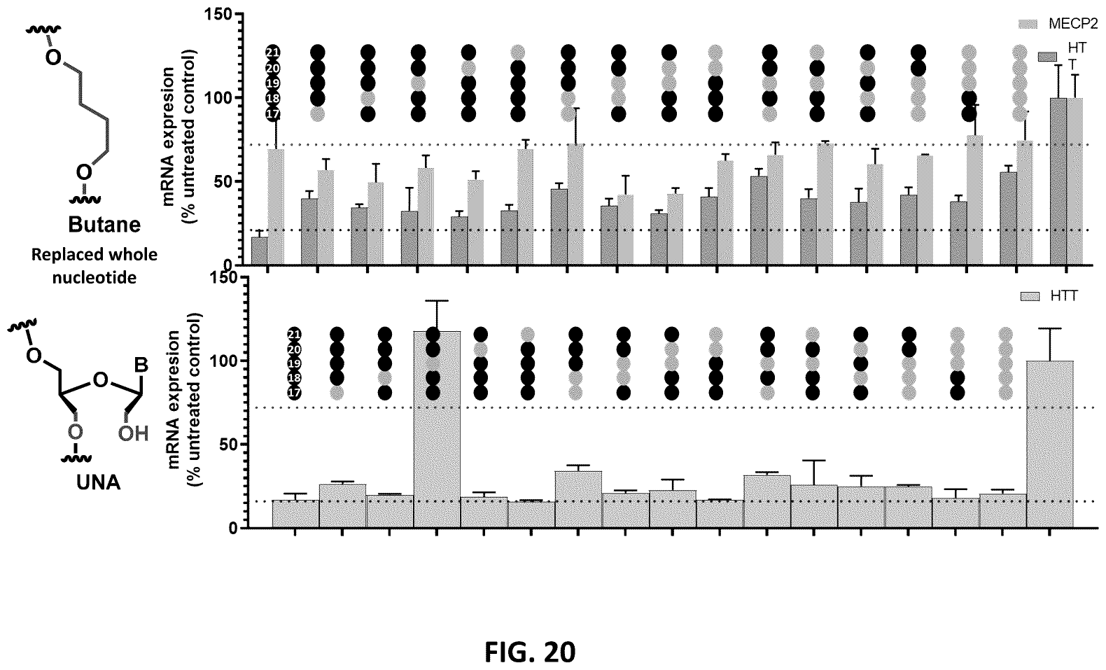

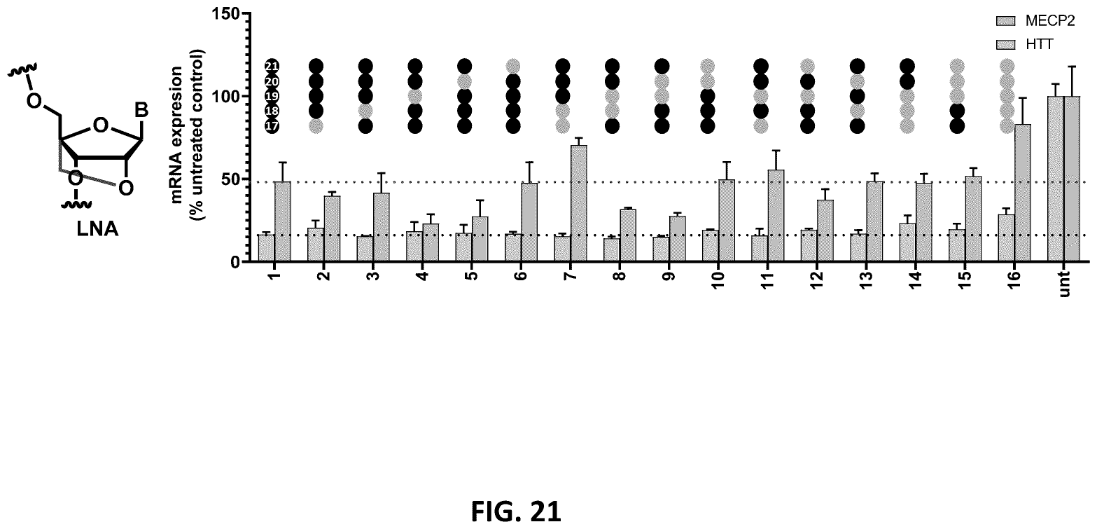

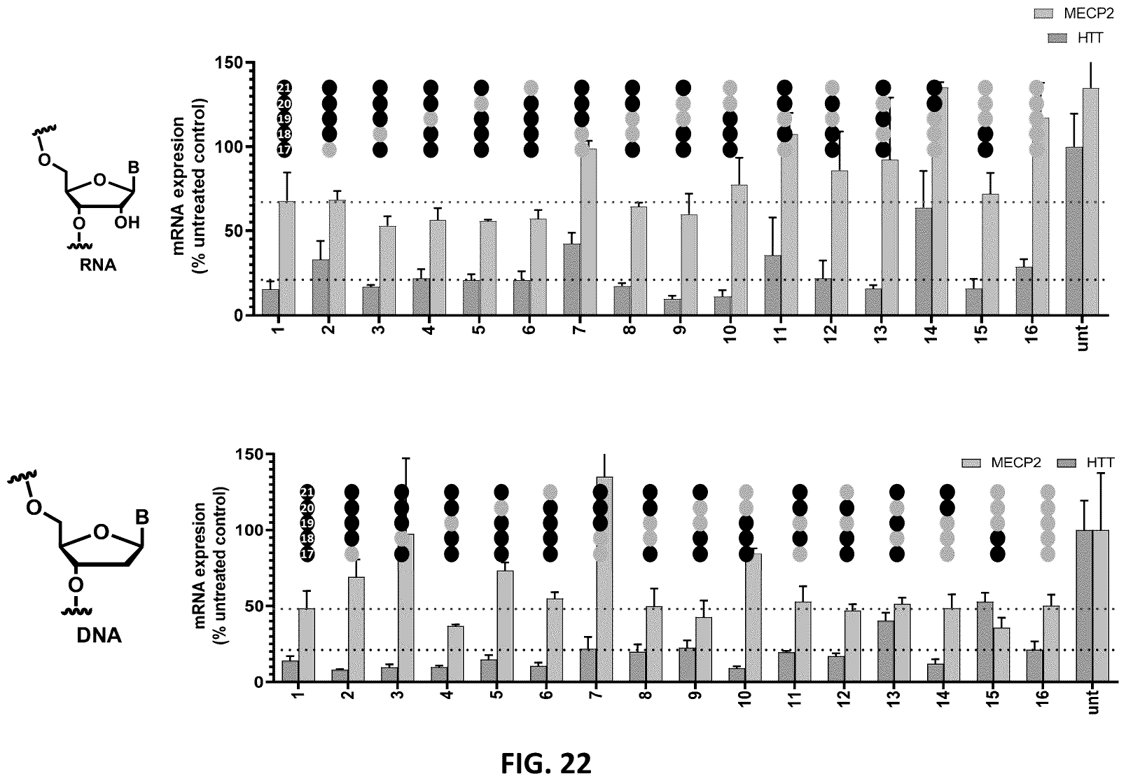

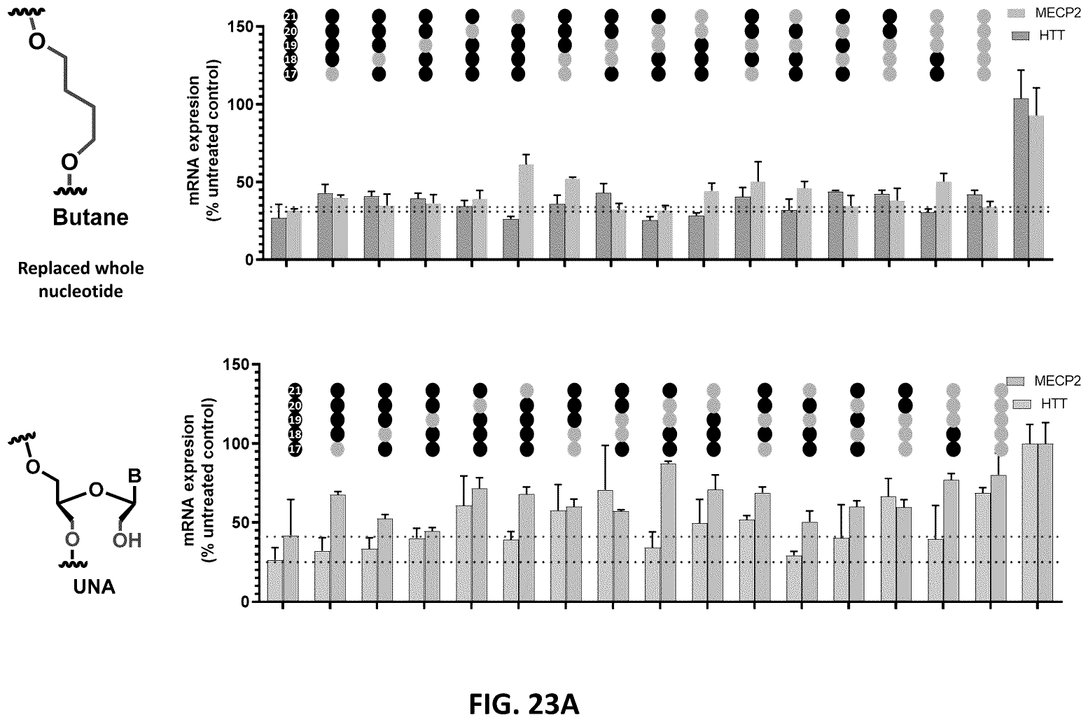

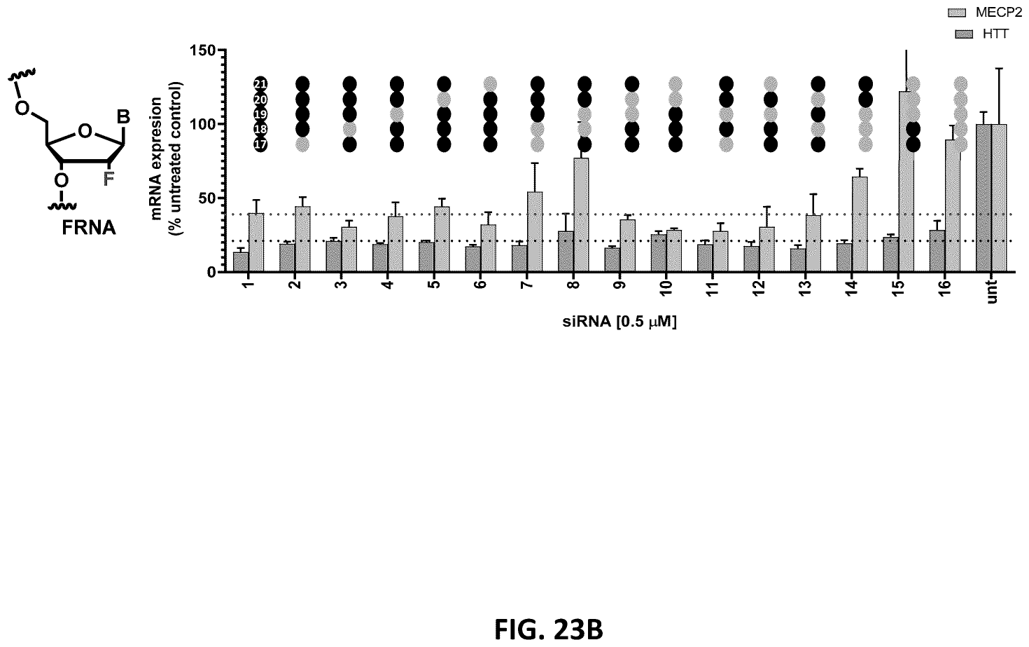

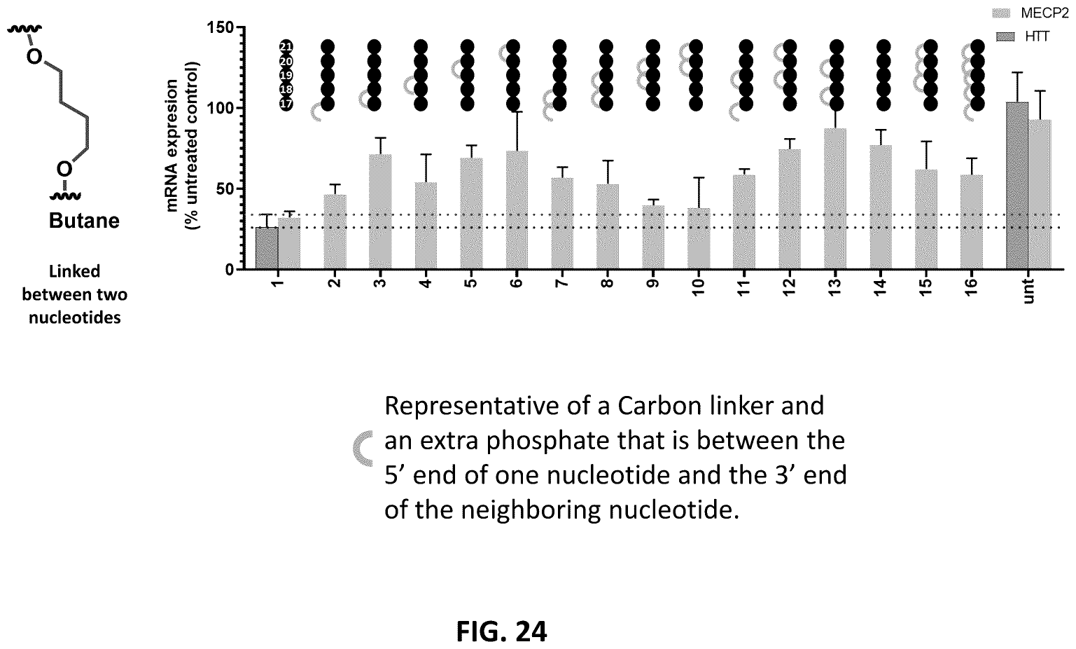

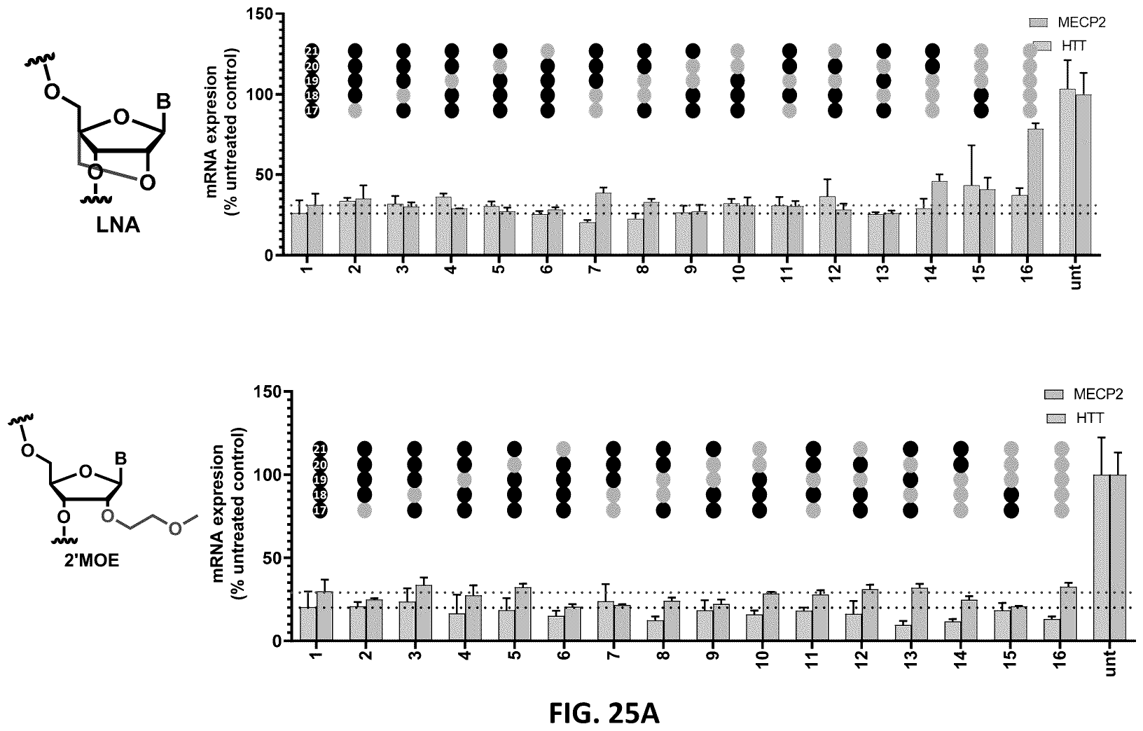

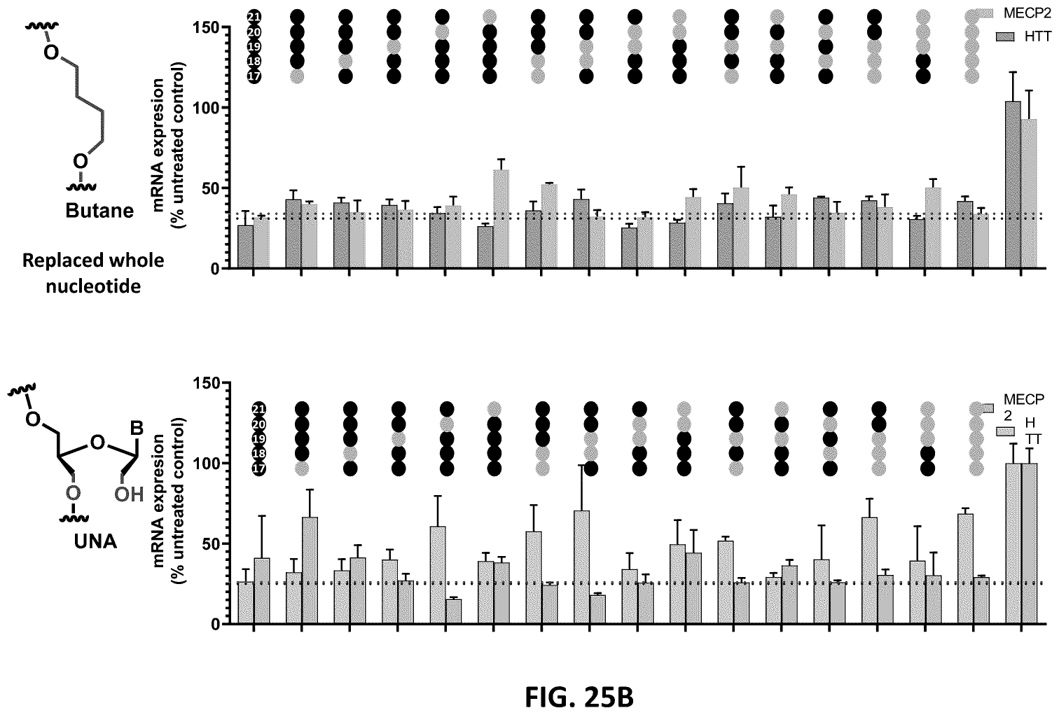

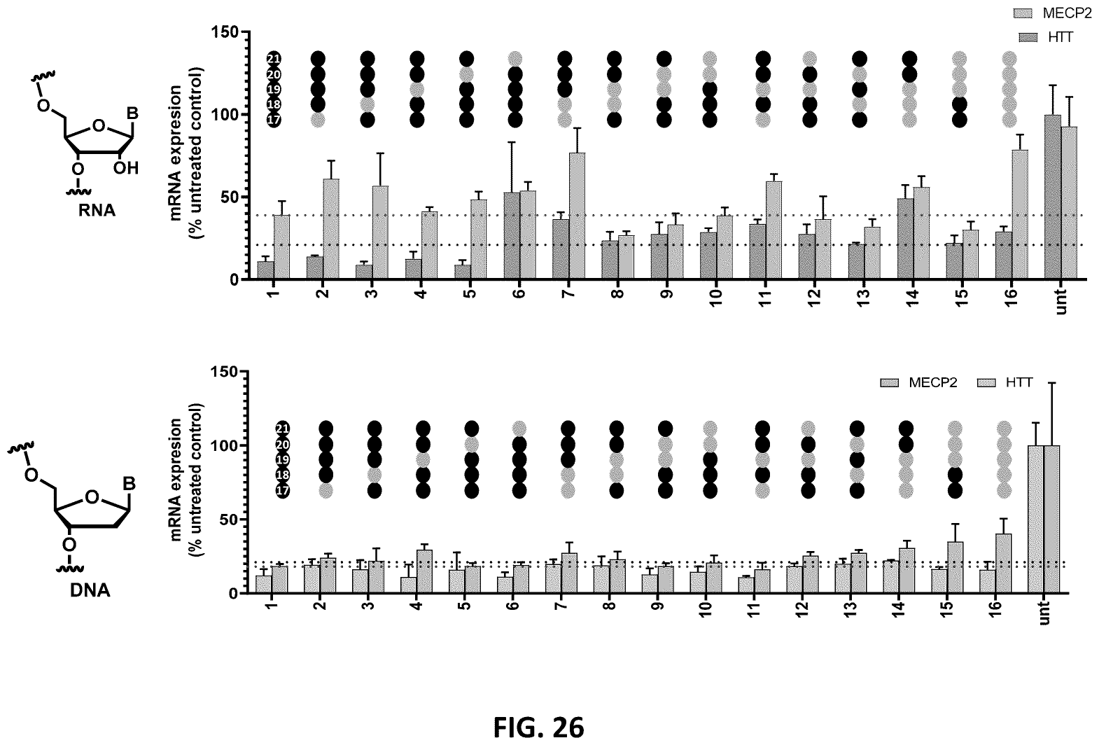

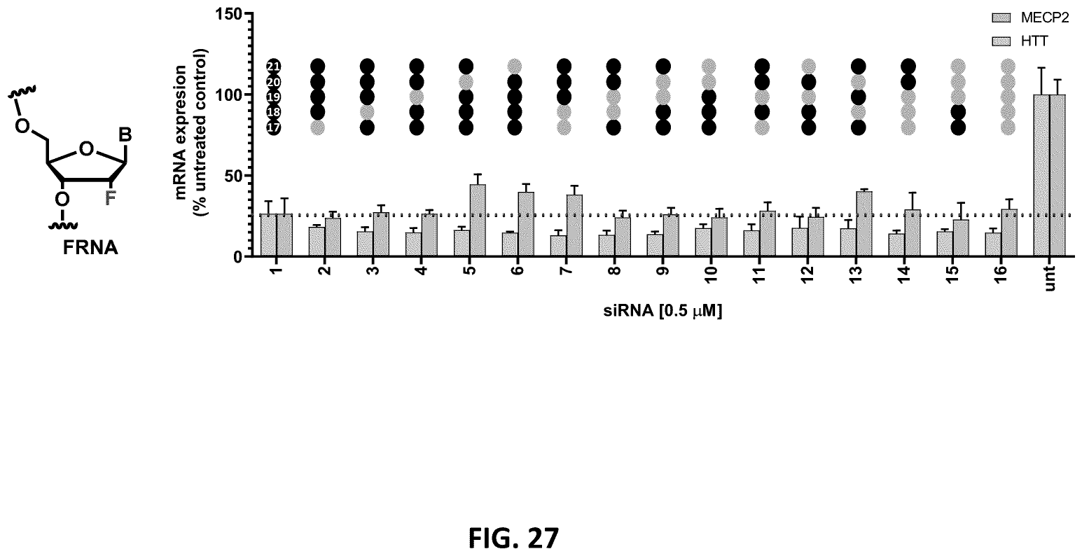

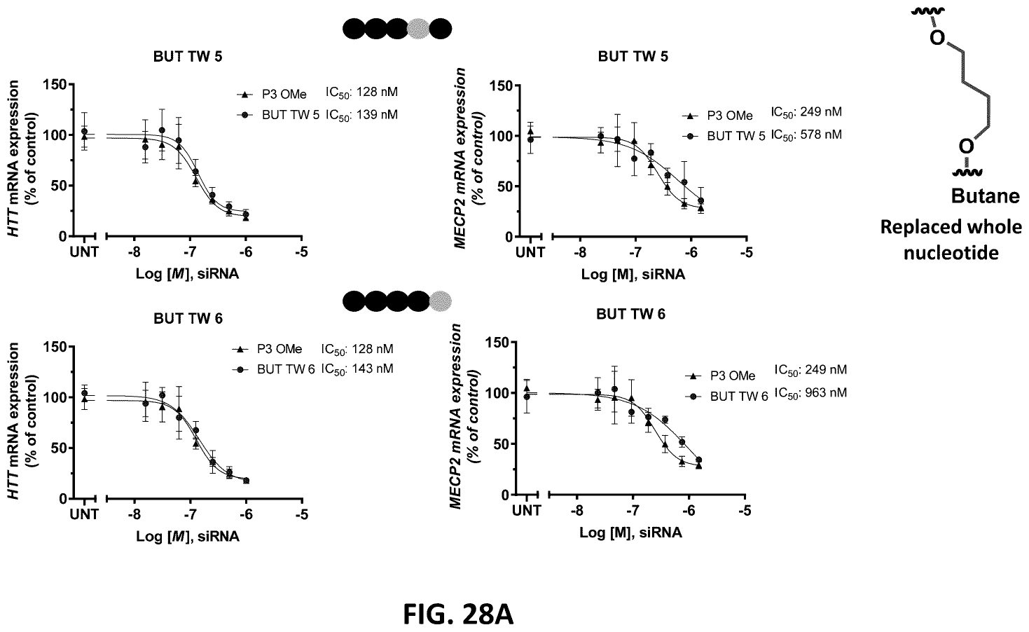

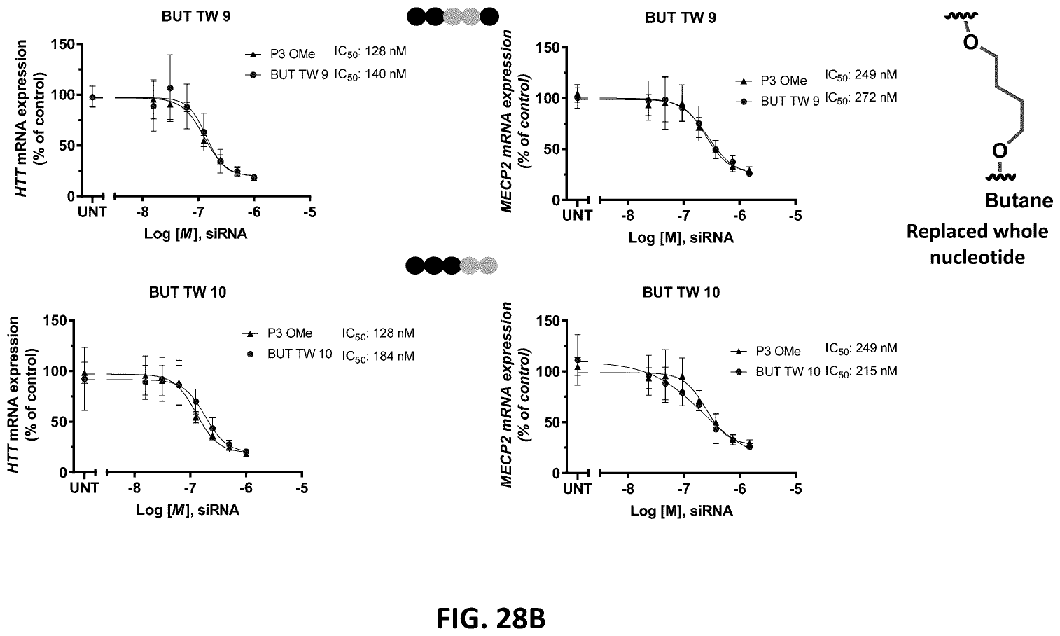

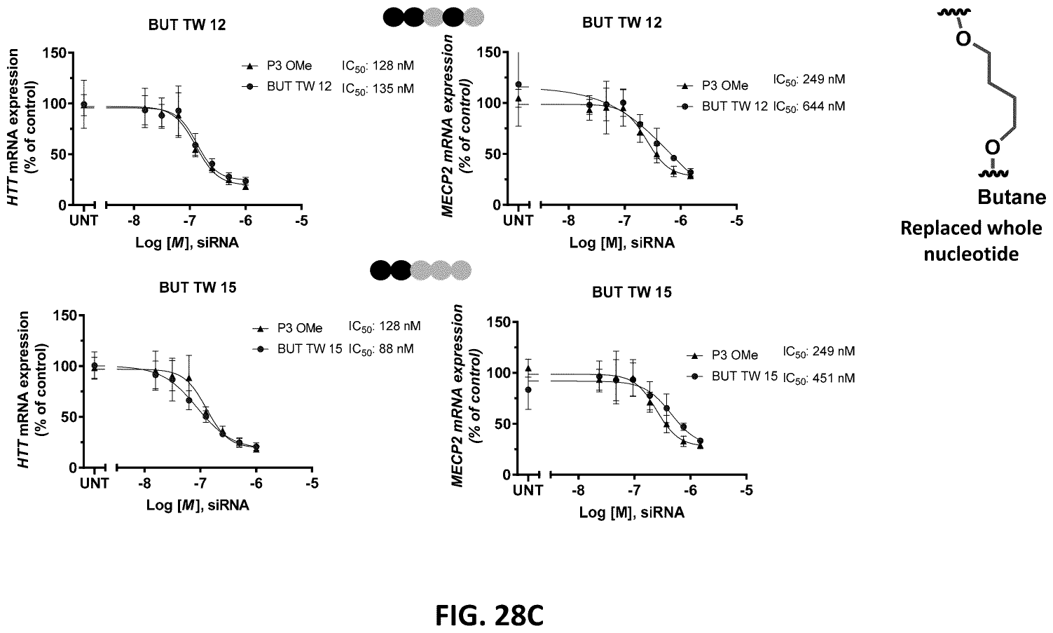

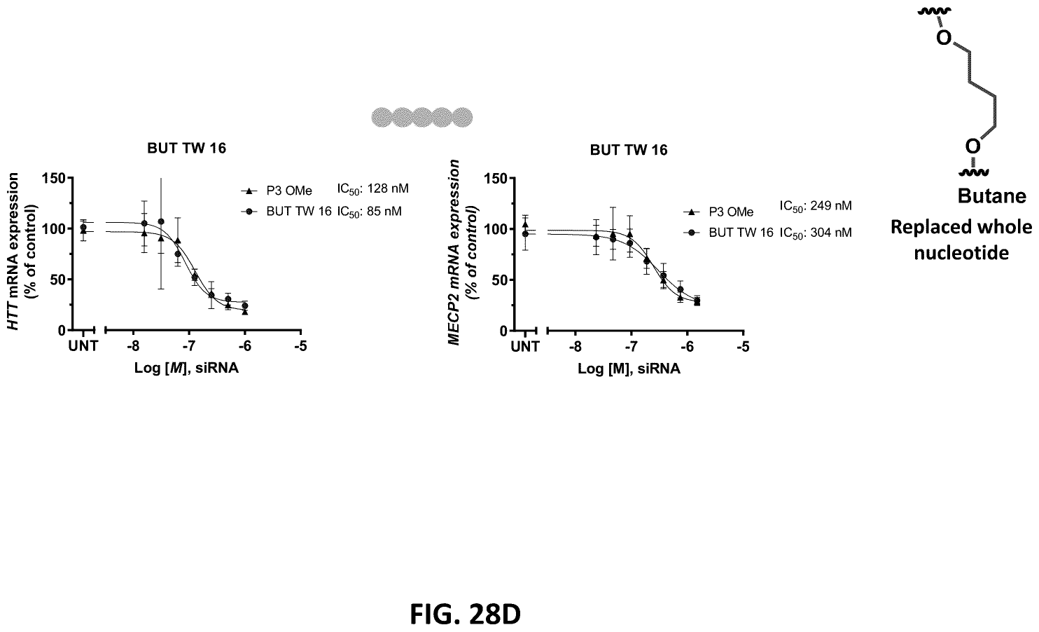

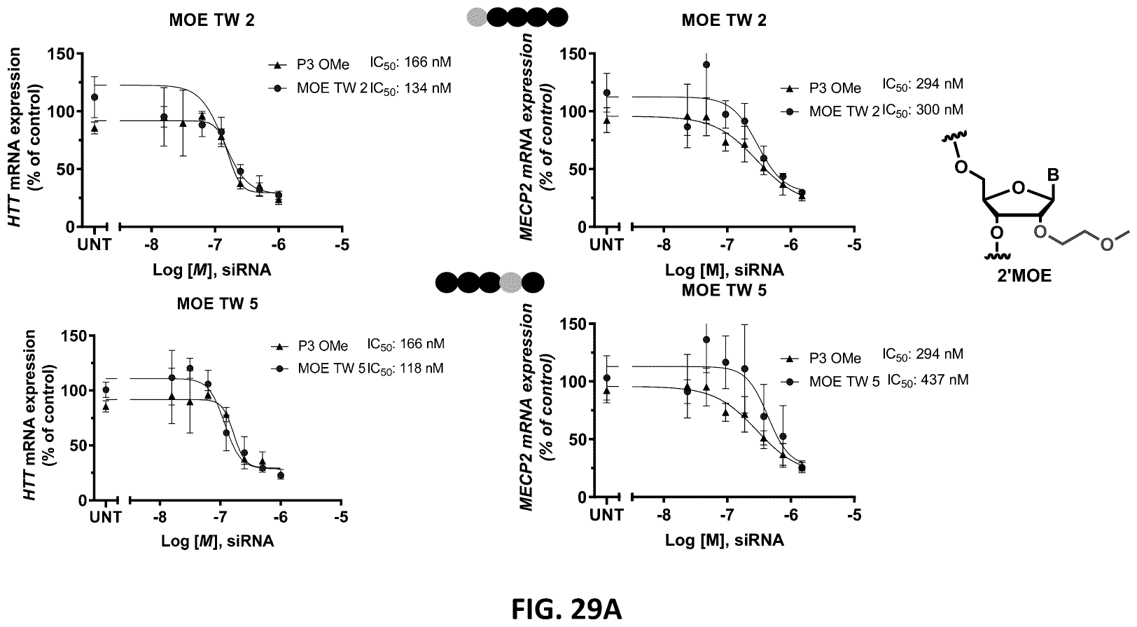

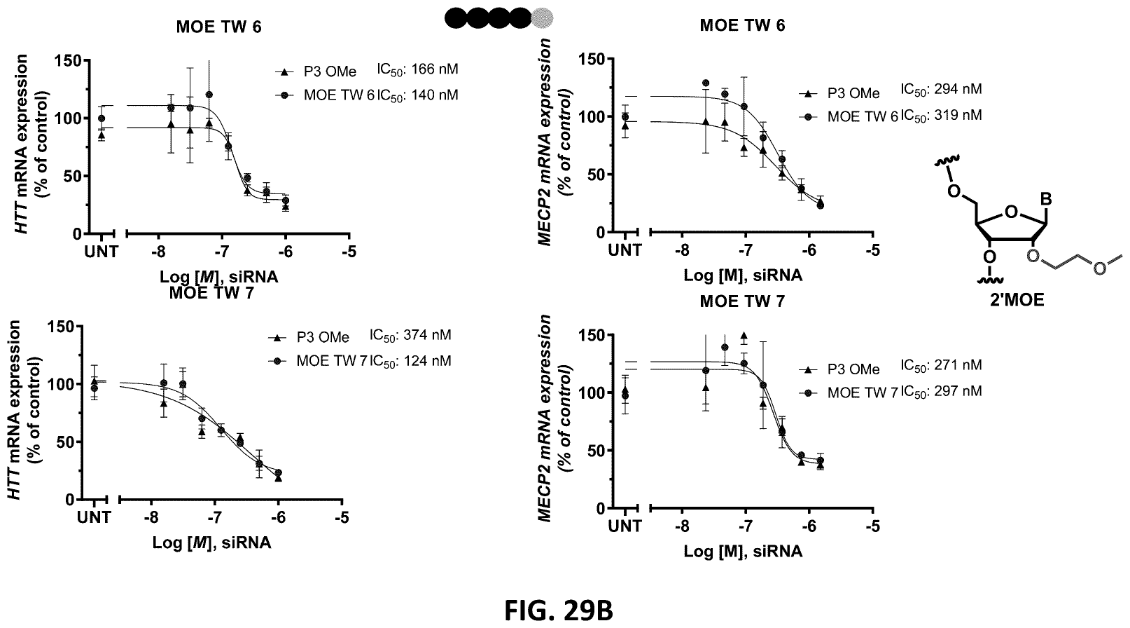

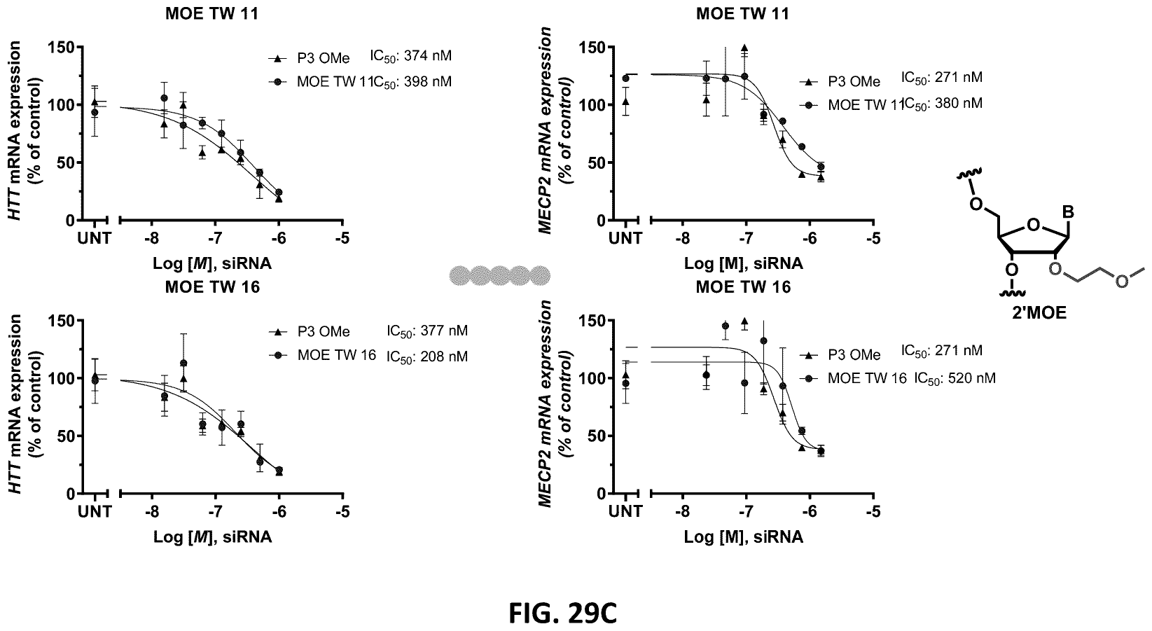

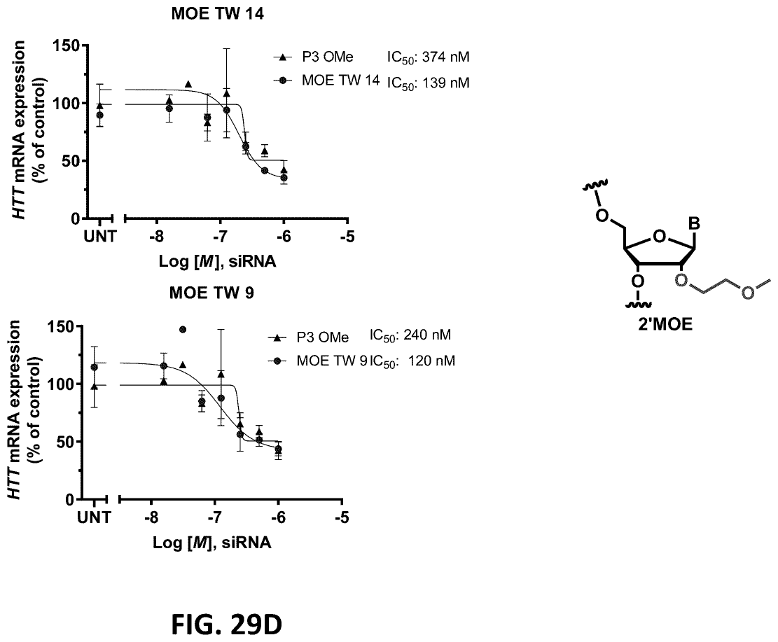

The effect of these nucleotide modifications was next tested in the antisense tail region. The siRNAs used in the screen have an asymmetric structure, with a 21-nucleotide antisense strand and a 16-nucleotide sense strand. This leads to a 5-nucleotide single stranded antisense strand overhang. Each position with the 5-nucleotide tail was modified with each of the modifications described above, including full modification of all 5 nucleotides. As shown in FIG. 19-29, the tested chemical modifications were capable of robust silencing at each of the positions within the antisense strand overhang.

### Example 2. Screen of siRNA Chemical Modifications In Vivo

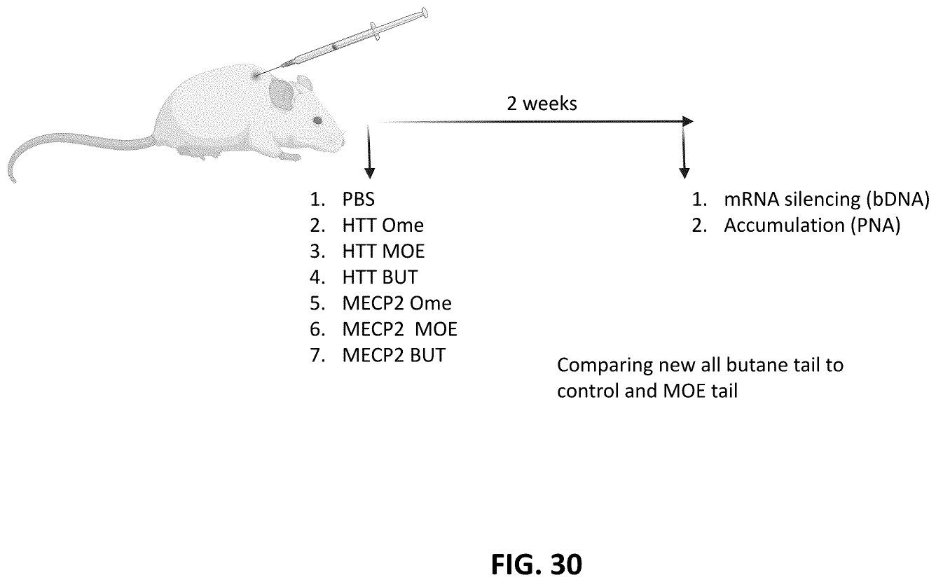

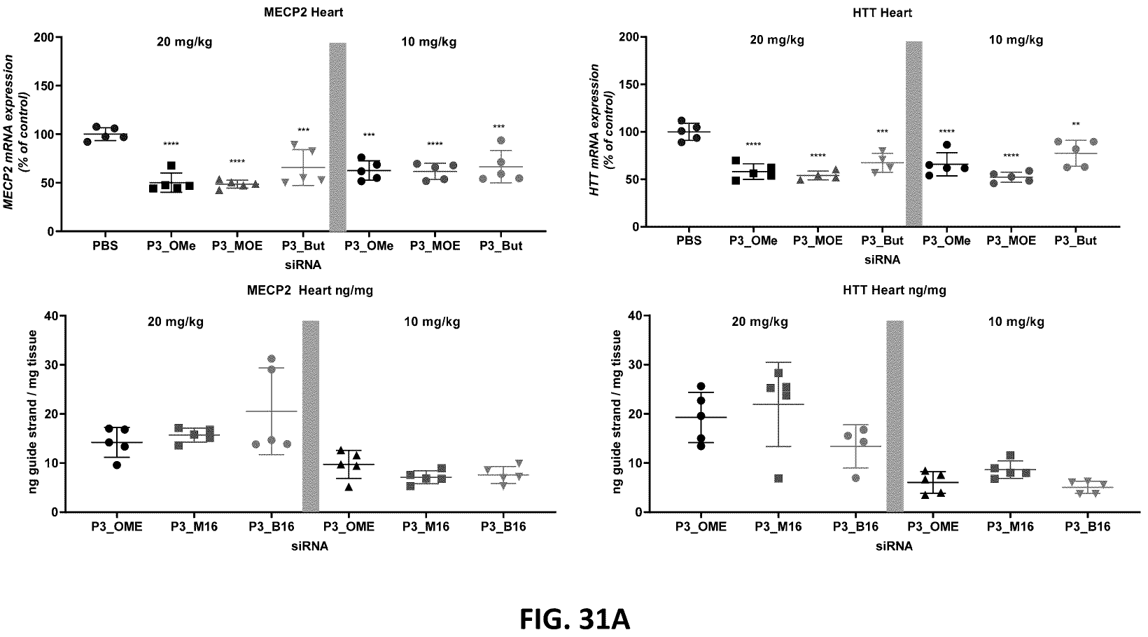

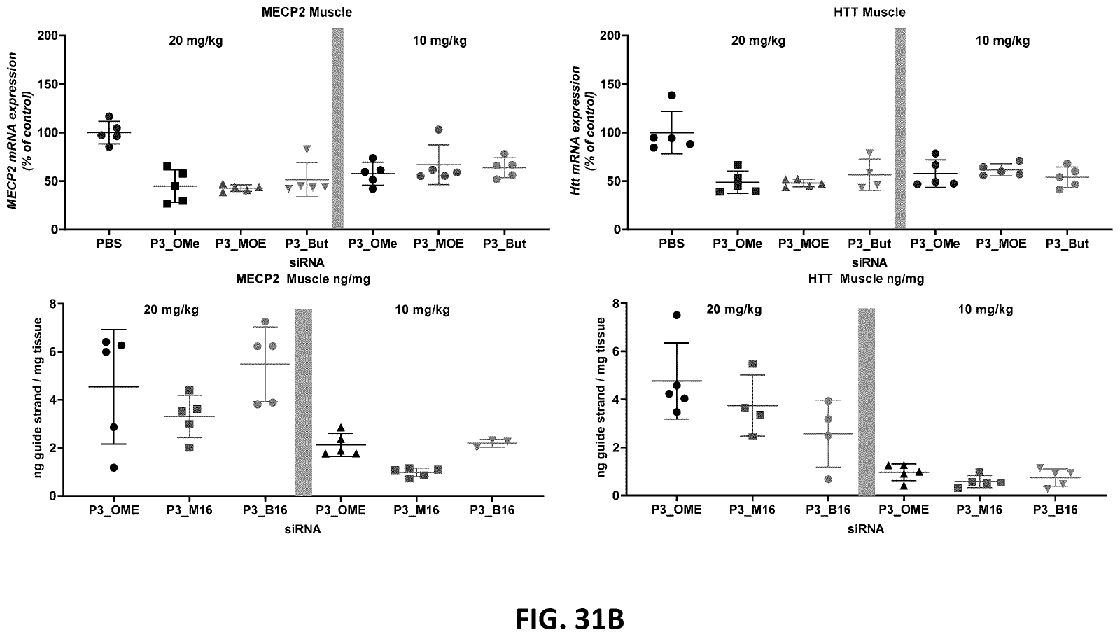

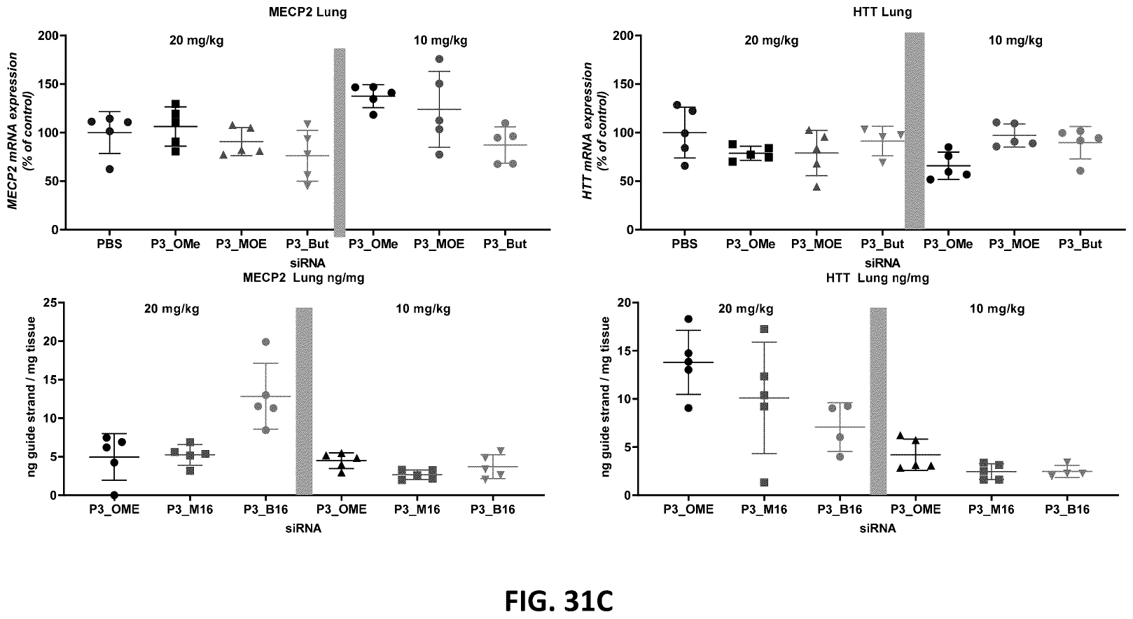

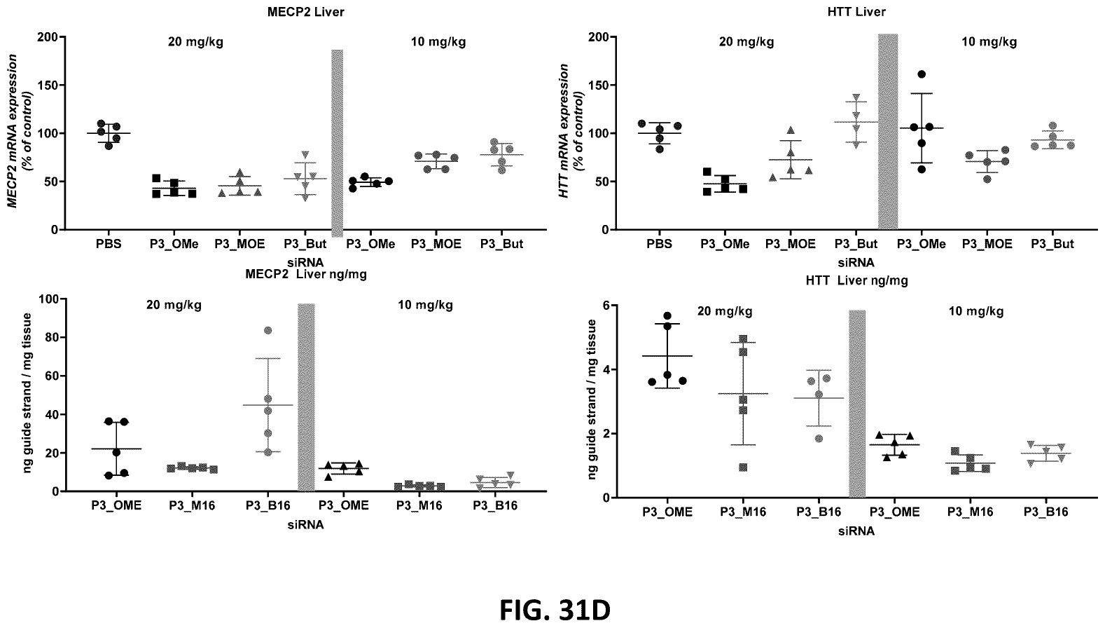

The above recited siRNA chemical modification patterns were tested in vivo in mouse models (FIG. 30 and FIGS. 31A-31D). The MECP2 and HTT mRNA levels and guide-strand tissue accumulations in mice injected with various chemically modified siRNA were assessed. FVB/NJ female mice were injected subcutaneously with 10 mg/kg or 20 mg/kg of chemically modified siRNA. The siRNA were conjugated with DCA and contained 2′-MOE, 2′-OMe, or butane (replacement of whole nucleotide) modifications.

### Example 3. Testing of Additional Alkyl Modifications in siRNA

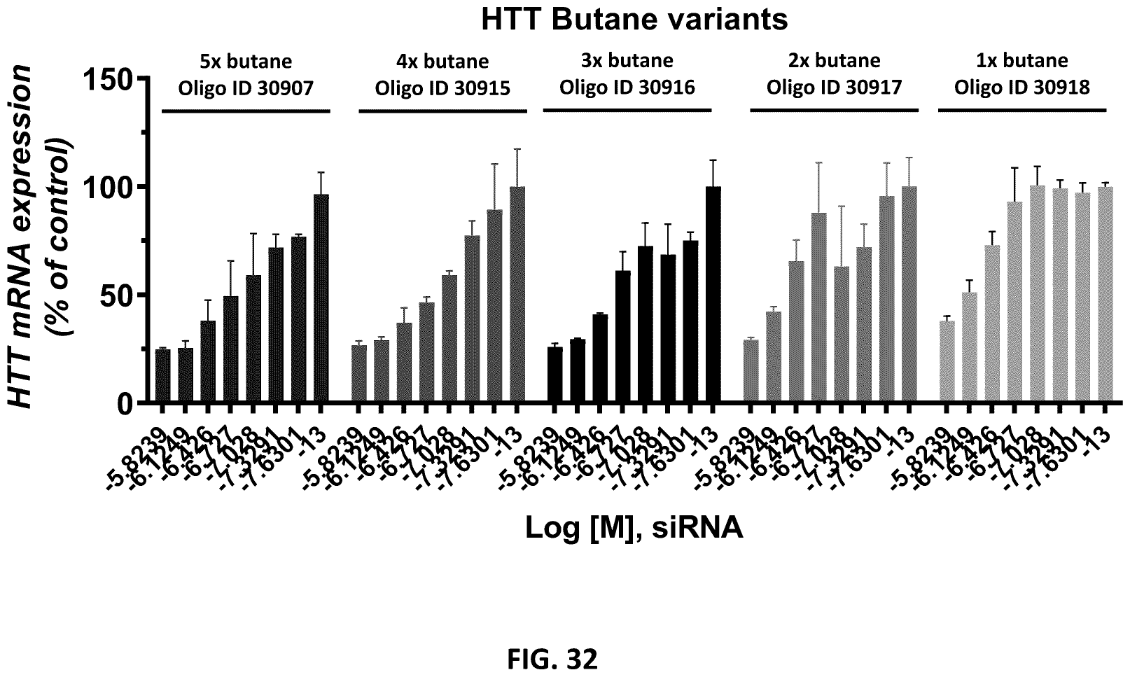

Numerous butyl modifications were tested in siRNA, as described in Examples 1 and 2. In particular, FIG. 32 describes the presence of multiple butyl modifications (1 to 5) at the 3′ end of the antisense strand in the silencing of an exemplary target mRNA. Any number from 1 to 5 butyl modifications was effective at maintaining target silencing.

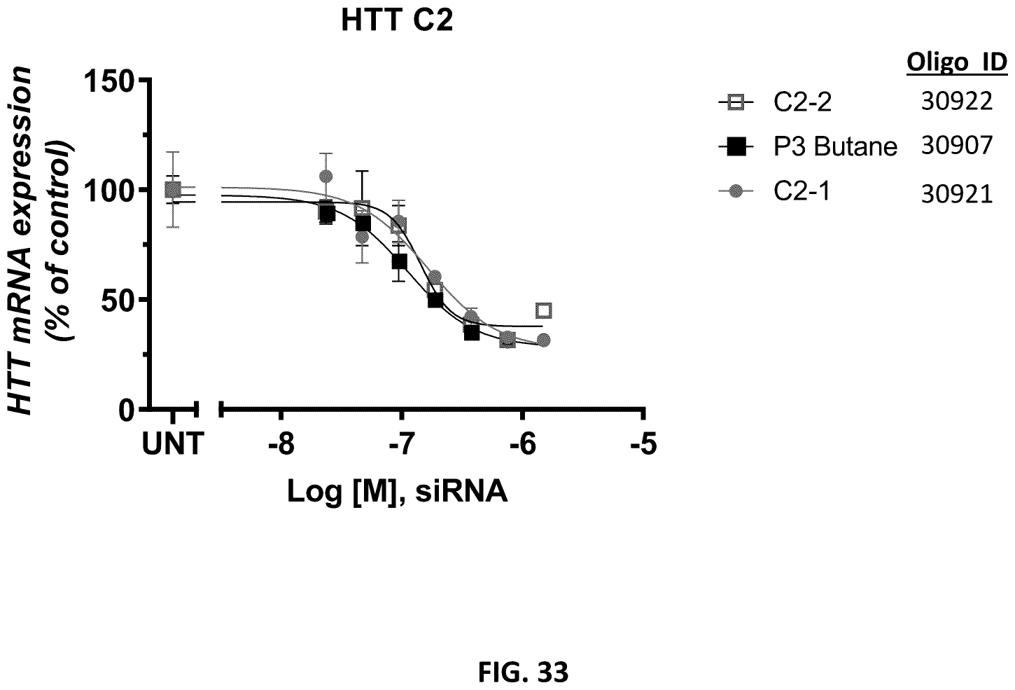

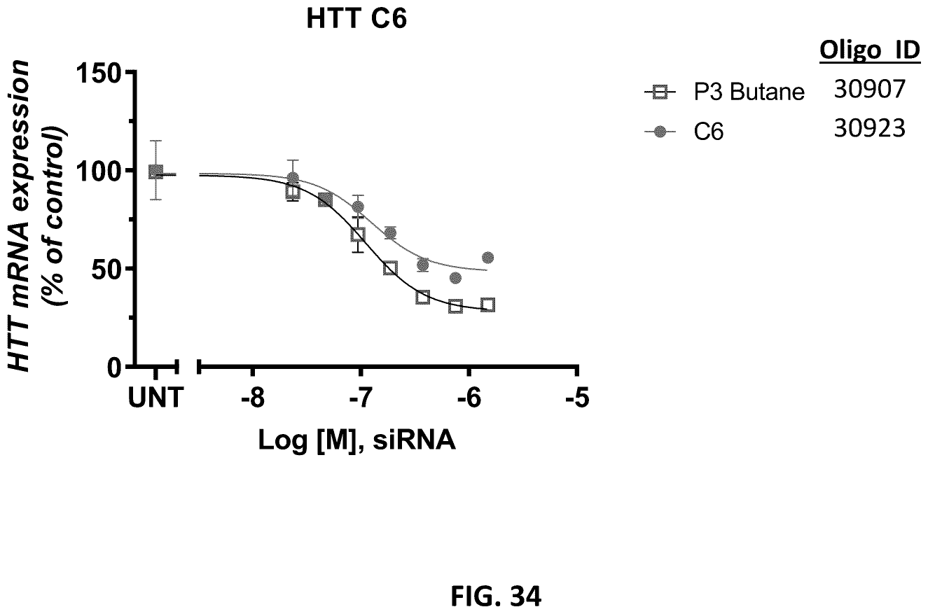

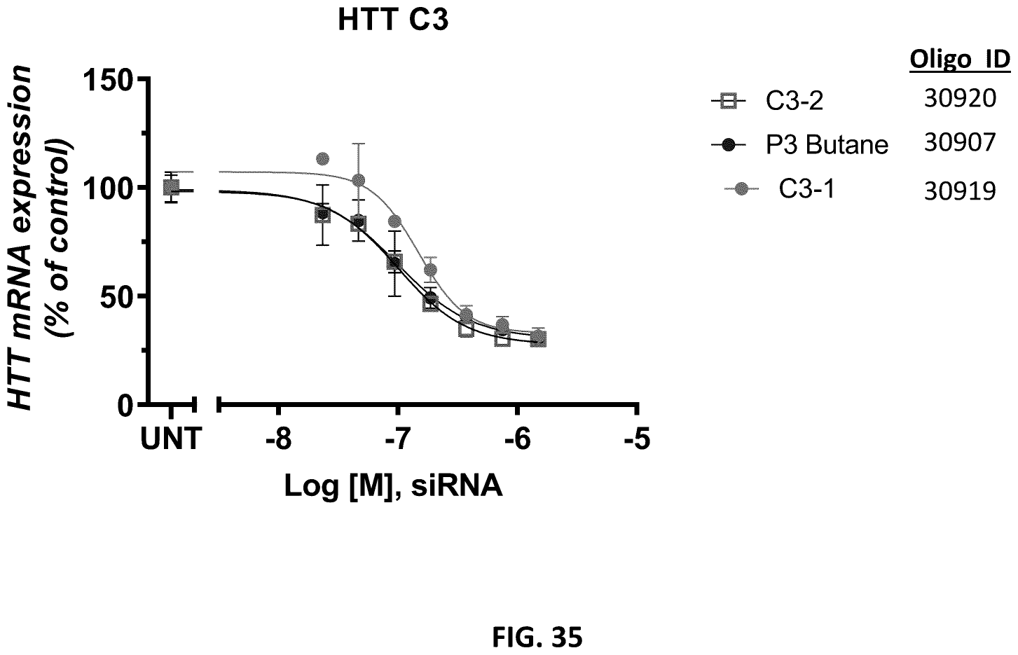

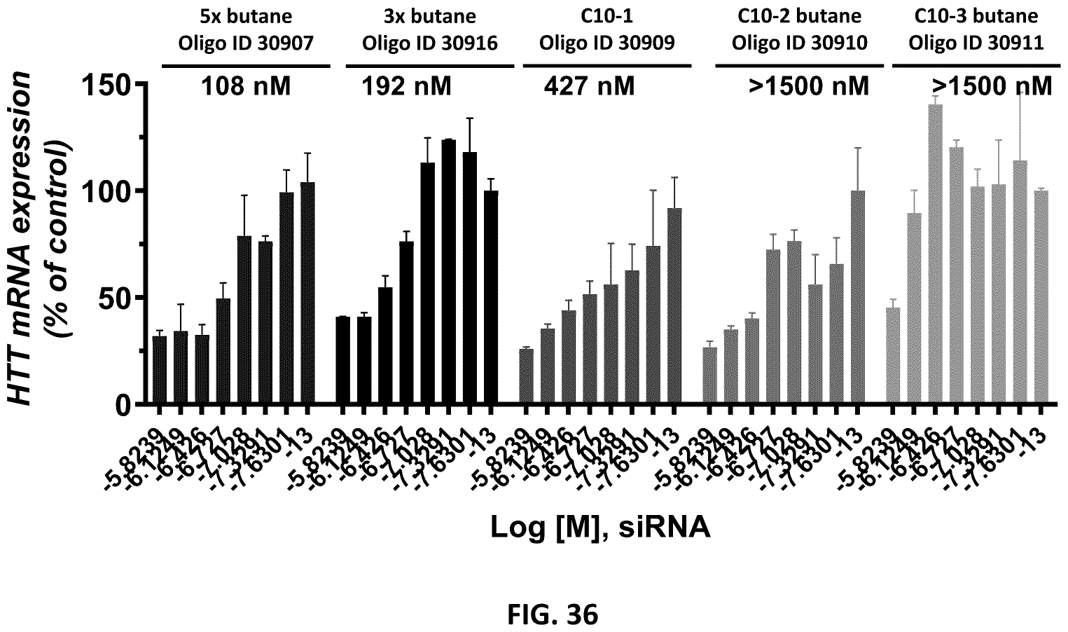

Alkyl modifications of different lengths (other than the 4 carbons of butyl) were tested. A C2 alkyl (FIG. 33), C6 alkyl (FIG. 34), C3 alkyl (FIG. 35), and C10 alkyl (FIG. 36), were tested. The chemical modification patterns used are described in Table 9. In each instance, the alkyl modification did not negatively affect siRNA silencing of the target mRNA.

Provided below in Table 9 are exemplary siRNA with various chemical modifications.

TABLE 9
Chemically modified siRNA.

| Oligo | | |
|---|---|---|
| ID | Description | Sequence |
| 30907 | HTT_as_P3_ | P(mU)#(fU)#(mA)(fA)(fU)(fC)(mU)(fC)(mU)(fU)(mU)(fA)(mC) |
| | 5x Butane | (fU)#(mG)#(fA)#(but)#(but)#(but)#(but)#(but) |
| 30915 | HTT_as_P3_ | P(mU)#(fU)#(mA)(fA)(fU)(fC)(mU)(fC)(mU)(fU)(mU)(fA)(mC) |
| | 4x Butane | (fU)#(mG)#(fA)#(but)#(but)#(but)#(but) |
| 30916 | HTT_as_P3_ | P(mU)#(fU)#(mA)(fA)(fU)(fC)(mU)(fC)(mU)(fU)(mU)(fA)(mC) |
| | TW_3x | (fU)#(mG)#(fA)#(but)#(but)#(but) |
| | Butane | |
| 30917 | HTT_as_P3_ | P(mU)#(fU)#(mA)(fA)(fU)(fC)(mU)(fC)(mU)(fU)(mU)(fA)(mC) |
| | TW_2x | (fU)#(mG)#(fA)#(but)#(but) |
| | Butane | |
| 30918 | HTT_as_P3_ | P(mU)#(fU)#(mA)(fA)(fU)(fC)(mU)(fC)(mU)(fU)(mU)(fA)(mC) |
| | TW_1x | (fU)#(mG)#(fA)#(but) |
| | Butane | |
| 30921 | HTT_as_P3_ | P(mU)#(fU)#(mA)(fA)(fU)(fC)(mU)(fC)(mU)(fU)(mU)(fA)(mC) |
| | TW_C2_1 | (fU)#(mG)#(fA)#(C2)#(C2)#(C2)#(C2)#(C2) |
| 30922 | HTT_as_P3_ | P(mU)#(fU)#(mA)(fA)(fU)(fC)(mU)(fC)(mU)(fU)(mU)(fA)(mC) |
| | TW_C2_2 | (fU)#(mG)#(fA)#(C2)#(C2)#(C2)#(C2)#(C2)#(C2)#(C2)#(C2)#(C2)# |
| | | (C2) |
| 30923 | HTT_as_P3_ | P(mU)#(fU)#(mA)(fA)(fU)(fC)(mU)(fC)(mU)(fU)(mU)(fA)(mC) |
| | TW_C6 | (fU)#(mG)#(fA)#(C6)#(C6)#(C6)#(C6)#(C6) |
| 30919 | HTT_as_P3_ | P(mU)#(fU)#(mA)(fA)(fU)(fC)(mU)(fC)(mU)(fU)(mU)(fA)(mC) |
| | TW_C3_1 | (fU)#(mG)#(fA)#(C3)#(C3)#(C3)#(C3)#(C3) |
| 30920 | HTT_as_P3_ | P(mU)#(fU)#(mA)(fA)(fU)(fC)(mU)(fC)(mU)(fU)(mU)(fA)(mC) |
| | TW_C3_2 | (fU)#(mG)#(fA)#(C3)#(C3)#(C3)#(C3)#(C3)#(C3)#(C3) |
| 30909 | HTT_as_P3_ | P(mU)#(fU)#(mA)(fA)(fU)(fC)(mU)(fC)(mU)(fU)(mU)(fA)(mC) |
| | TW_C10_1 | (fU)#(mG)#(fA)#(C10)#(but)#(but) |
| 30910 | HTT_as_P3_ | P(mU)#(fU)#(mA)(fA)(fU)(fC)(mU)(fC)(mU)(fU)(mU)(fA)(mC) |
| | TW_C10_2 | (fU)#(mG)#(fA)#(but)#(but)#(C10) |
| 30911 | HTT_as_P3_ | P(mU)#(fU)#(mA)(fA)(fU)(fC)(mU)(fC)(mU)(fU)(mU)(fA)(mC) |
| | TW_C10_3 | (fU)#(mG)#(fA)#(C10)#(C10) |

In Table 9, “P” corresponds to a 5′ phosphate, “m” corresponds to a 2′-OMe modification, “f” corresponds to a 2′-fluoro modification, “#” corresponds to a phosphorothioate internucleotide linkage, “but” corresponds to a butyl modification, “C2” corresponds to a C₂ alkyl modification, “C3” corresponds to a C₃ alkyl modification, “C6” corresponds to a C₆ alkyl modification, and “C10” corresponds to a C₁₀ alkyl modification.

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Atwell et al. J. Mol. Biol. 1997, 270: 26-35;
Ausubel et al. (eds.), CURRENT PROTOCOLS IN MOLECULAR BIOLOGY, John Wiley &Sons, N Y (1993);
Ausubel, F. M. et al. eds., SHORT PROTOCOLS IN MOLECULAR BIOLOGY (4th Ed. 1999) John Wiley & Sons, NY. (ISBN 0-471-32938-X);
CONTROLLED DRUG BIOAVAILABILITY, DRUG PRODUCT DESIGN AND PERFORMANCE, Smolen and Ball (eds.), Wiley, New York (1984);
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, New York, (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, U.S. Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).
Kriegler, Gene Transfer and Expression, A Laboratory Manual, Stockton Press, N Y (1990);
Lu and Weiner eds., CLONING AND EXPRESSION VECTORS FOR GENE FUNCTION ANALYSIS (2001) BioTechniques Press. Westborough, MA. 298 pp. (ISBN 1-881299-21-X).
MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION TO GENETIC ENGINEERING (3d Ed. 1985) Blackwell Scientific Publications, Boston. Studies in Microbiology; V. 2:409 pp. (ISBN 0-632-01318-4).
Sambrook, J. et al. eds., MOLECULAR CLONING: A LABORATORY MANUAL (2d Ed. 1989) Cold Spring Harbor Laboratory Press, NY. Vols. 1-3. (ISBN 0-87969-309-6).
SUSTAINED AND CONTROLLED RELEASE DRUG DELIVERY SYSTEMS, J. R. Robinson, ed., Marcel Dekker, Inc., New York, 1978
Winnacker, E. L. FROM GENES TO CLONES: INTRODUCTION TO GENE TECHNOLOGY (1987) VCH Publishers, NY (translated by Horst Ibelgaufts). 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. An RNA molecule comprising a 5′ end and a 3′ end, wherein the RNA molecule comprises at least one alkyl modification within nucleotide positions 1-5 of one or both of the 5′ end and 3′ end.

2. The RNA molecule of claim 1, wherein the at least one alkyl modification comprises a C₁-C₁₀ alkyl.

3. The RNA molecule of claim 1, wherein the at least one alkyl modification comprises a C₄ alkyl.

4. The RNA molecule of claim 1, wherein:
the at least one alkyl modification is positioned between two adjacent nucleotides, optionally wherein the at least one alkyl modification positioned between two adjacent nucleotides does not replace a nucleotide at a position within the RNA molecule relative to an RNA molecule that does not contain the at least one alkyl modification at the same position within the RNA molecule; or
the at least one alkyl modification replaces a nucleotide at a position within the RNA molecule relative to an RNA molecule that does not contain the at least one alkyl modification at the same position within the RNA molecule.

5-6. (canceled)

7. The RNA molecule of claim 1, wherein the RNA molecule comprises a single stranded (ss) RNA or a double stranded (ds) RNA, optionally wherein:
the RNA molecule comprises a dsRNA and the dsRNA comprises an antisense strand with a 5′ end and a 3′ end, and a sense strand with a 5′ end and a 3′ end, optionally wherein the antisense strand comprises at least one alkyl modification within nucleotide positions 1-5 of one or both of the 5′ end and 3′ end, optionally wherein;
the at least one alkyl modification comprises a C₁-C₁₀ alkyl;
the at least one alkyl modification comprises a C₄ alkyl;
the at least one alkyl modification is positioned between two adjacent nucleotides;
the at least one alkyl modification positioned between two adjacent nucleotides does not replace a nucleotide at a position within the RNA molecule relative to an RNA molecule that does not contain the at least one alkyl modification at the same position within the RNA molecule; and/or
the at least one alkyl modification replaces a nucleotide at a position within the RNA molecule relative to an RNA molecule that does not contain the at least one alkyl modification at the same position within the RNA molecule.

8-14. (canceled)

15. A double stranded (ds) RNA, comprising an antisense strand with a 5′ end and a 3′ end, and a sense strand with a 5′ end and a 3′ end, wherein the antisense strand comprises at least one alkyl modification.

16. The dsRNA of claim 15, wherein the antisense strand is between 15 and 25 nucleotides in length, optionally wherein the antisense strand is 18, 19, 20, 21, 22, or 23 nucleotides in length.

17. (canceled)

18. The dsRNA of claim 15, wherein the sense strand is between 15 and 25 nucleotides in length, optionally wherein the sense strand is 14, 15, 16, or 17 nucleotides in length.

19. (canceled)

20. The dsRNA of claim 16, wherein the at least one alkyl modification is at any one of positions 1-25 from the 5′ end of the antisense strand.

21. The dsRNA of claim 15, further comprising at least one non-alkyl modified nucleotide, optionally wherein the at least one non-alkyl modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

22. (canceled)

23. The dsRNA of claim 15, wherein the dsRNA comprises at least one modified internucleotide linkage, optionally wherein said modified internucleotide linkage comprises a phosphorothioate internucleotide linkage.

24. (canceled)

25. The dsRNA of claim 15, wherein:
the dsRNA comprises 4-16 phosphorothioate internucleotide linkages;
the dsRNA comprises 8-13 phosphorothioate internucleotide linkages; and/or
the dsRNA comprises a blunt end.

26-27. (canceled)

28. The dsRNA of claim 15, wherein said dsRNA comprises at least one single stranded nucleotide overhang, optionally wherein the dsRNA comprises about a 2-nucleotide to 5-nucleotide single stranded nucleotide overhang, 2-nucleotide single stranded nucleotide overhang, or 5-nucleotide single stranded nucleotide overhang; and/or
the single stranded nucleotide overhang comprises at least two alkyl modifications or 2, 3, 4, or 5 alkyl modifications.

29-33. (canceled)

34. The dsRNA of claim 15, comprising an antisense strand with one of the following chemical modification patterns:

| | |
|---|---|
| P1_b1_as | P(but)#(fN)#(mN)(fN)(fN)(fN)(mN)(fN)(mN)(fN)(mN)(fN)(mN)(fN)#(mN)#(fN) |
| | #(mN)#(mN)#(mN)#(fN)#(mN) |
| P1_b2_as | P(mN)#(but)#(mN)(fN)(fN)(fN)(mN)(fN)(mN)(fN)(mN)(fN)(mN)(fN)#(mN)#(fN) |
| | #(mN)#(mN)#(mN)#(fN)#(mN) |
| P1_b3_as | P(mN)#(fN)#(but)(fN)(fN)(fN)(mN)(fN)(mN)(fN)(mN)(fN)(mN)(fN)#(mN)#(fN) |
| | #(mN)#(mN)#(mN)#(fN)#(mN) |
| P1_b4_as | P(mN)#(fN)#(mN)(but)(fN)(fN)(mN)(fN)(mN)(fN)(mN)(fN)(mN)(fN)#(mN)#(fN) |
| | #(mN)#(mN)#(mN)#(fN)#(mN) |
| P1_b5_as | P(mN)#(fN)#(mN)(fN)(but)(fN)(mN)(fN)(mN)(fN)(mN)(fN)(mN)(fN)#(mN)#(fN) |
| | #(mN)#(mN)#(mN)#(fN)#(mN) |
| P1_b6_as | P(mN)#(fN)#(mN)(fN)(fN)(but)(mN)(fN)(mN)(fN)(mN)(fN)(mN)(fN)#(mN)#(fN) |
| | #(mN)#(mN)#(mN)#(fN)#(mN) |
| P1_b7_as | P(mN)#(fN)#(mN)(fN)(fN)(fN)(but)(fN)(mN)(fN)(mN)(fN)(mN)(fN)#(mN)#(fN) |
| | #(mN)#(mN)#(mN)#(fN)#(mN) |
| P1_b8_as | P(mN)#(fN)#(mN)(fN)(fN)(fN)(mN)(but)(mN)(fN)(mN)(fN)(mN)(fN)#(mN)#(fN) |
| | #(mN)#(mN)#(mN)#(fN)#(mN) |
| P1_b9_as | P(mN)#(fN)#(mN)(fN)(fN)(fN)(mN)(fN)(but)(fN)(mN)(fN)(mN)(fN)#(mN)#(fN) |
| | #(mN)#(mN)#(mN)#(fN)#(mN) |
| P1_b10_as | P(mN)#(fN)#(mN)(fN)(fN)(fN)(mN)(fN)(mN)(but)(mN)(fN)(mN)(fN)#(mN)#(fN) |
| | #(mN)#(mN)#(mN)#(fN)#(mN) |
| P1_b11_as | P(mN)#(fN)#(mN)(fN)(fN)(fN)(mN)(fN)(mN)(fN)(but)(fN)(mN)(fN)#(mN)#(fN) |
| | #(mN)#(mN)#(mN)#(fN)#(mN) |
| P1_b12_as | P(mN)#(fN)#(mN)(fN)(fN)(fN)(mN)(fN)(mN)(fN)(mN)(but)(mN)(fN)#(mN)#(fN) |
| | #(mN)#(mN)#(mN)#(fN)#(mN) |
| P1_b13_as | P(mN)#(fN)#(mN)(fN)(fN)(fN)(mN)(fN)(mN)(fN)(mN)(fN)(but)(fN)#(mN)#(fN) |
| | #(mN)#(mN)#(mN)#(fN)#(mN) |
| P1_b14_as | P(mN)#(fN)#(mN)(fN)(fN)(fN)(mN)(fN)(mN)(fN)(mN)(fN)(mN)(but)#(mN)#(fN) |
| | #(mN)#(mN)#(mN)#(fN)#(mN) |
| P1_b15_as | P(mN)#(fN)#(mN)(fN)(fN)(fN)(mN)(fN)(mN)(fN)(mN)(fN)(mN)(fN)#(but)#(fN) |
| | #(mN)#(mN)#(mN)#(fN)#(mN) |
| P1_b16_as | P(mN)#(fN)#(mN)(fN)(fN)(fN)(mN)(fN)(mN)(fN)(mN)(fN)(mN)(fN)#(mN)#(but) |
| | #(mN)#(mN)#(mN)#(fN)#(mN) |
| P1_b17_as | P(mN)#(fN)#(mN)(fN)(fN)(fN)(mN)(fN)(mN)(fN)(mN)(fN)(mN)(fN)#(mN)#(fN) |
| | #(but)#(mN)#(mN)#(fN)#(mN) |
| P1_b18_as | P(mN)#(fN)#(mN)(fN)(fN)(fN)(mN)(fN)(mN)(fN)(mN)(fN)(mN)(fN)#(mN)#(fN) |
| | #(mN)#(but)#(mN)#(fN)#(mN) |
| P1_b19_as | P(mN)#(fN)#(mN)(fN)(fN)(fN)(mN)(fN)(mN)(fN)(mN)(fN)(mN)(fN)#(mN)#(fN) |
| | #(mN)#(mN)#(but)#(fN)#(mN) |
| P1_b20_as | P(mN)#(fN)#(mN)(fN)(fN)(fN)(mN)(fN)(mN)(fN)(mN)(fN)(mN)(fN)#(mN)#(fN) |
| | #(mN)#(mN)#(mN)#(but)#(mN) |
| P1_b21_as | P(mN)#(fN)#(mN)(fN)(fN)(fN)(mN)(fN)(mN)(fN)(mN)(fN)(mN)(fN)#(mN)#(fN) |
| | #(mN)#(mN)#(mN)#(fN)#(but) |
| P2_b1_as | P(but)#(fN)#(mN)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b2_as | P(mN)#(but)#(mN)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b3_as | P(mN)#(fN)#(but)(mN)(mN)(fN)(mN)(mN)(mN)(mN)(mN)(mN)(mN)(fN)#(mN) |
| | #(fN)#(mN)#(mN)#(mN)#(fN)#(mN) |
| P2_b4_as | P(mN)#(fN)#(mN)(but)(mN)(fN)(mN)(mN)(mN)(mN)(mN)(mN)(mN)(fN)#(mN) |
| | #(fN)#(mN)#(mN)#(mN)#(fN)#(mN) |
| P2_b5_as | P(mN)#(fN)#(mN)(mN)(but)(fN)(mN)(mN)(mN)(mN)(mN)(mN)(mN)(fN)#(mN) |
| | #(fN)#(mN)#(mN)#(mN)#(fN)#(mN) |
| P2_b6_as | P(mN)#(fN)#(mN)(mN)(mN)(but)(mN)(mN)(mN)(mN)(mN)(mN)(mN)(fN)#(mN) |
| | #(fN)#(mN)#(mN)#(mN)#(fN)#(mN) |
| P2_b7_as | P(mN)#(fN)#(mN)(mN)(mN)(fN)(but)(mN)(mN)(mN)(mN)(mN)(mN)(fN)#(mN) |
| | #(fN)#(mN)#(mN)#(mN)#(fN)#(mN) |
| P2_b8_as | P(mN)#(fN)#(mN)(mN)(mN)(fN)(mN)(but)(mN)(mN)(mN)(mN)(mN)(fN)#(mN) |
| | #(fN)#(mN)#(mN)#(mN)#(fN)#(mN) |
| P2_b9_as | P(mN)#(fN)#(mN)(mN)(mN)(fN)(mN)(mN)(but)(mN)(mN)(mN)(mN)(fN)#(mN) |
| | #(fN)#(mN)#(mN)#(mN)#(fN)#(mN) |
| P2_b10_as | P(mN)#(fN)#(mN)(mN)(mN)(fN)(mN)(mN)(mN)(but)(mN)(mN)(mN)(fN)#(mN) |
| | #(fN)#(mN)#(mN)#(mN)#(fN)#(mN) |
| P2_b11_as | P(mN)#(fN)#(mN)(mN)(mN)(fN)(mN)(mN)(mN)(mN)(but)(mN)(mN)(fN)#(mN) |
| | #(fN)#(mN)#(mN)#(mN)#(fN)#(mN) |
| P2_b12_as | P(mN)#(fN)#(mN)(mN)(mN)(fN)(mN)(mN)(mN)(mN)(mN)(but)(mN)(fN)#(mN) |
| | #(fN)#(mN)#(mN)#(mN)#(fN)#(mN) |
| P2_b13_as | P(mN)#(fN)#(mN)(mN)(mN)(fN)(mN)(mN)(mN)(mN)(mN)(mN)(but)(fN)#(mN) |
| | #(fN)#(mN)#(mN)#(mN)#(fN)#(mN) |
| P2_b14_as | P(mN)#(fN)#(mN)(mN)(mN)(fN)(mN)(mN)(mN)(mN)(mN)(mN)(mN)(but)#(mN) |
| | #(fN)#(mN)#(mN)#(mN)#(fN)#(mN) |
| P2_b15_as | P(mN)#(fN)#(mN)(mN)(mN)(fN)(mN)(mN)(mN)(mN)(mN)(mN)(mN)(fN)#(but) |
| | #(fN)#(mN)#(mN)#(mN)#(fN)#(mN) |
| P2_b16_as | P(mN)#(fN)#(mN)(mN)(mN)(fN)(mN)(mN)(mN)(mN)(mN)(mN)(mN)(fN)#(mN) |
| | #(but)#(mN)#(mN)#(mN)#(fN)#(mN) |
| P2_b17_ | P(mN)#(fN)#(mN)(mN)(mN)(fN)(mN)(mN)(mN)(mN)(mN)(mN)(mN)(fN)#(mN) |
| as | #(fN)#(but)#(mN)#(mN)#(fN)#(mN) |
| P2_b18_as | P(mN)#(fN)#(mN)(mN)(mN)(fN)(mN)(mN)(mN)(mN)(mN)(mN)(mN)(fN)#(mN) |
| | #(fN)#(mN)#(but)#(mN)#(fN)#(mN) |
| P2_b19_as | P(mN)#(fN)#(mN)(mN)(mN)(fN)(mN)(mN)(mN)(mN)(mN)(mN)(mN)(fN)#(mN) |
| | #(fN)#(mN)#(mN)#(but)#(fN)#(mN) |
| P2_b20_as | P(mN)#(fN)#(mN)(mN)(mN)(fN)(mN)(mN)(mN)(mN)(mN)(mN)(mN)(fN)#(mN) |
| | #(fN)#(mN)#(mN)#(mN)#(but)#(mN) |
| P2_b21_as | P(mN)#(fN)#(mN)(mN)(mN)(fN)(mN)(mN)(mN)(mN)(mN)(mN)(mN)(fN)#(mN) |
| | #(fN)#(mN)#(mN)#(mN)#(fN)#(but) |
| as_TW_ | P(mN)#(fN)#(mN)(mN)(mN)(fN)(mN)(mN)(mN)(mN)(mN)(mN)(mN)(fN)#(mN) |
| But1 | #(fN)#(mN)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| But2 | #(fN)#(but)#(mN)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| But3 | #(fN)#(mN)#(but)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| But4 | #(fN)#(mN)#(mN)#(but)#(mN)#(mN) |
| as_TW_ | P(mN)#(fN)#(mN)(mN)(mN)(fN)(mN)(mN)(mN)(mN)(mN)(mN)(mN)(fN)#(mN) |
| But5 | #(fN)#(mN)#(mN)#(mN)#(but)#(mN) |
| as_TW_ | P(mN)#(fN)#(mN)(mN)(mN)(fN)(mN)(mN)(mN)(mN)(mN)(mN)(mN)(fN)#(mN) |
| But6 | #(fN)#(mN)#(mN)#(mN)#(mN)#(but) |
| as_TW_ | P(mN)#(fN)#(mN)(mN)(mN)(fN)(mN)(mN)(mN)(mN)(mN)(mN)(mN)(fN)#(mN) |
| But7 | #(fN)#(but)#(but)#(mN)#(mN)#(mN) |
| as_TW_ | P(mN)#(fN)#(mN)(mN)(mN)(fN)(mN)(mN)(mN)(mN)(mN)(mN)(mN)(fN)#(mN) |
| But8 | #(fN)#(mN)#(but)#(but)#(mN)#(mN) |
| as_TW_ | P(mN)#(fN)#(mN)(mN)(mN)(fN)(mN)(mN)(mN)(mN)(mN)(mN)(mN)(fN)#(mN) |
| But9 | #(fN)#(mN)#(mN)#(but)#(but)#(mN) |
| as_TW_ | P(mN)#(fN)#(mN)(mN)(mN)(fN)(mN)(mN)(mN)(mN)(mN)(mN)(mN)(fN)#(mN) |
| But10 | #(fN)#(mN)#(mN)#(mN)#(but)#(but) |
| as_TW_ | P(mN)#(fN)#(mN)(mN)(mN)(fN)(mN)(mN)(mN)(mN)(mN)(mN)(mN)(fN)#(mN) |
| But11 | #(fN)#(but)#(mN)#(but)#(mN)#(mN) |
| as_TW_ | P(mN)#(fN)#(mN)(mN)(mN)(fN)(mN)(mN)(mN)(mN)(mN)(mN)(mN)(fN)#(mN) |
| But12 | #(fN)#(mN)#(mN)#(but)#(mN)#(but) |
| as_TW_ | P(mN)#(fN)#(mN)(mN)(mN)(fN)(mN)(mN)(mN)(mN)(mN)(mN)(mN)(fN)#(mN) |
| But13 | #(fN)#(mN)#(but)#(mN)#(but)#(mN) |
| as_TW_ | P(mN)#(fN)#(mN)(mN)(mN)(fN)(mN)(mN)(mN)(mN)(mN)(mN)(mN)(fN)#(mN) |
| But14 | #(fN)#(but)#(but)#(but)#(mN)#(mN) |
| as_TW_ | P(mN)#(fN)#(mN)(mN)(mN)(fN)(mN)(mN)(mN)(mN)(mN)(mN)(mN)(fN)#(mN) |
| But15 | #(fN)#(mN)#(mN)#(but)#(but)#(but) |
| as_TW_ | P(mN)#(fN)#(mN)(mN)(mN)(fN)(mN)(mN)(mN)(mN)(mN)(mN)(mN)(fN)#(mN) |
| But16 | #(fN)#(but)#(but)#(but)#(but)#(but) |
| P1_ib1_as | P(ibut)(mN)#(fN)#(mN)(fN)(fN)(fN)(mN)(fN)(mN)(fN)(mN)(fN)(mN)(fN)#(mN) |
| | #(fN)#(mN)#(mN)#(mN)#(fN)#(mN) |
| P1_ib2_as | P(mN)#(ibut)(fN)#(mN)(fN)(fN)(fN)(mN)(fN)(mN)(fN)(mN)(fN)(mN)(fN)#(mN) |
| | #(fN)#(mN)#(mN)#(mN)#(fN)#(mN) |
| P1_ib3_as | P(mN)#(fN)#(ibut)(mN)(fN)(fN)(fN)(mN)(fN)(mN)(fN)(mN)(fN)(mN)(fN)#(mN) |
| | #(fN)#(mN)#(mN)#(mN)#(fN)#(mN) |
| P1_ib4_as | P(mN)#(fN)#(mN)(ibut)(fN)(fN)(fN)(mN)(fN)(mN)(fN)(mN)(fN)(mN)(fN)#(mN) |
| | #(fN)#(mN)#(mN)#(mN)#(fN)#(mN) |
| P1_ib5_as | P(mN)#(fN)#(mN)(fN)(ibut)(fN)(fN)(mN)(fN)(mN)(fN)(mN)(fN)(mN)(fN)#(mN) |
| | #(fN)#(mN)#(mN)#(mN)#(fN)#(mN) |
| P1_ib6_as | P(mN)#(fN)#(mN)(fN)(fN)(ibut)(fN)(mN)(fN)(mN)(fN)(mN)(fN)(mN)(fN)#(mN) |
| | #(fN)#(mN)#(mN)#(mN)#(fN)#(mN) |
| P1_ib7_as | P(mN)#(fN)#(mN)(fN)(fN)(fN)(ibut)(mN)(fN)(mN)(fN)(mN)(fN)(mN)(fN)#(mN) |
| | #(fN)#(mN)#(mN)#(mN)#(fN)#(mN) |
| P1_ib8_as | P(mN)#(fN)#(mN)(fN)(fN)(fN)(mN)(ibut)(fN)(mN)(fN)(mN)(fN)(mN)(fN)#(mN) |
| | #(fN)#(mN)#(mN)#(mN)#(fN)#(mN) |
| P1_ib9_as | P(mN)#(fN)#(mN)(fN)(fN)(fN)(mN)(fN)(ibut)(mN)(fN)(mN)(fN)(mN)(fN)#(mN) |
| | #(fN)#(mN)#(mN)#(mN)#(fN)#(mN) |
| P1_ib10_ | P(mN)#(fN)#(mN)(fN)(fN)(fN)(mN)(fN)(mN)(ibut)(fN)(mN)(fN)(mN)(fN)#(mN) |
| as | #(fN)#(mN)#(mN)#(mN)#(fN)#(mN) |
| P1_ib11_ | P(mN)#(fN)#(mN)(fN)(fN)(fN)(mN)(fN)(mN)(fN)(ibut)(mN)(fN)(mN)(fN)#(mN) |
| as | #(fN)#(mN)#(mN)#(mN)#(fN)#(mN) |
| P1_ib12_ | P(mN)#(fN)#(mN)(fN)(fN)(fN)(mN)(fN)(mN)(fN)(mN)(ibut)(fN)(mN)(fN)#(mN) |
| as | #(fN)#(mN)#(mN)#(mN)#(fN)#(mN) |
| P1_ib13_ | P(mN)#(fN)#(mN)(fN)(fN)(fN)(mN)(fN)(mN)(fN)(mN)(fN)(ibut)(mN)(fN)#(mN) |
| as | #(fN)#(mN)#(mN)#(mN)#(fN)#(mN) |
| P1_ib14_ | P(mN)#(fN)#(mN)(fN)(fN)(fN)(mN)(fN)(mN)(fN)(mN)(fN)(mN)(ibut)(fN)#(mN) |
| as | #(fN)#(mN)#(mN)#(mN)#(fN)#(mN) |
| P1_ib15_ | P(mN)#(fN)#(mN)(fN)(fN)(fN)(mN)(fN)(mN)(fN)(mN)(fN)(mN)(fN)#(ibut)(mN) |
| as | #(fN)#(mN)#(mN)#(mN)#(fN)#(mN) |
| P1_ib16_ | P(mN)#(fN)#(mN)(fN)(fN)(fN)(mN)(fN)(mN)(fN)(mN)(fN)(mN)(fN)#(mN)#(ibut) |
| as | (fN)#(mN)#(mN)#(mN)#(fN)#(mN) |
| P1_ib17_ | P(mN)#(fN)#(mN)(fN)(fN)(fN)(mN)(fN)(mN)(fN)(mN)(fN)(mN)(fN)#(mN)#(fN) |
| as | #(ibut)(mN)#(mN)#(mN)#(fN)#(mN) |
| P1_ib18_ | P(mN)#(fN)#(mN)(fN)(fN)(fN)(mN)(fN)(mN)(fN)(mN)(fN)(mN)(fN)#(mN)#(fN) |
| as | #(mN)#(ibut)(mN)#(mN)#(fN)#(mN) |
| P1_ib19_ | P(mN)#(fN)#(mN)(fN)(fN)(fN)(mN)(fN)(mN)(fN)(mN)(fN)(mN)(fN)#(mN)#(fN) |
| as | #(mN)#(mN)#(ibut)(mN)#(fN)#(mN) |
| P1_ib20_ | P(mN)#(fN)#(mN)(fN)(fN)(fN)(mN)(fN)(mN)(fN)(mN)(fN)(mN)(fN)#(mN)#(fN) |
| as | #(mN)#(mN)#(mN)#(ibut)(fN)#(mN) |
| P1_ib21_ | P(mN)#(fN)#(mN)(fN)(fN)(fN)(mN)(fN)(mN)(fN)(mN)(fN)(mN)(fN)#(mN)#(fN) |
| as | #(mN)#(mN)#(mN)#(fN)#(ibut)(mN) |
| P1_ib22_ | P(mN)#(fN)#(mN)(fN)(fN)(fN)(mN)(fN)(mN)(fN)(mN)(fN)(mN)(fN)#(mN)#(fN) |
| as | #(mN)#(mN)#(mN)#(fN)#(mN)(ibut) |
| P2_ib1_as | P(ibut)(mN)#(fN)#(mN)(mN)(mN)(fN)(mN)(mN)(mN)(mN)(mN)(mN)(mN)(fN)# |
| | (mN)#(fN)#(mN)#(mN)#(mN)#(fN)#(mN) |
| P2_ib2_as | P(mN)#(ibut)(fN)#(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib3_as | P(mN)#(fN)#(ibut)(mN)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib4_as | P(mN)#(fN)#(mN)(ibut)(mN)(mN)(fN)(mN)(mN)(mN)(mN)(mN)(mN)(mN)(fN) |
| | #(mN)#(fN)#(mN)#(mN)#(mN)#(fN)#(mN) |
| P2_ib5_as | P(mN)#(fN)#(mN)(mN)(ibut)(mN)(fN)(mN)(mN)(mN)(mN)(mN)(mN)(mN)(fN) |
| | #(mN)#(fN)#(mN)#(mN)#(mN)#(fN)#(mN) |
| P2_ib6_as | P(mN)#(fN)#(mN)(mN)(mN)(ibut)(fN)(mN)(mN)(mN)(mN)(mN)(mN)(mN)(fN) |
| | #(mN)#(fN)#(mN)#(mN)#(mN)#(fN)#(mN) |
| P2_ib7_as | P(mN)#(fN)#(mN)(mN)(mN)(fN)(ibut)(mN)(mN)(mN)(mN)(mN)(mN)(mN)(fN) |
| | #(mN)#(fN)#(mN)#(mN)#(mN)#(fN)#(mN) |
| P2_ib8_as | P(mN)#(fN)#(mN)(mN)(mN)(fN)(mN)(ibut)(mN)(mN)(mN)(mN)(mN)(mN)(fN) |
| | #(mN)#(fN)#(mN)#(mN)#(mN)#(fN)#(mN) |
| P2_ib9_as | P(mN)#(fN)#(mN)(mN)(mN)(fN)(mN)(mN)(ibut)(mN)(mN)(mN)(mN)(mN)(fN) |
| | #(mN)#(fN)#(mN)#(mN)#(mN)#(fN)#(mN) |
| P2_ib10_ | P(mN)#(fN)#(mN)(mN)(mN)(fN)(mN)(mN)(mN)(ibut)(mN)(mN)(mN)(mN)(fN) |
| as | #(mN)#(fN)#(mN)#(mN)#(mN)#(fN)#(mN) |
| P2_ib11_ | P(mN)#(fN)#(mN)(mN)(mN)(fN)(mN)(mN)(mN)(mN)(ibut)(mN)(mN)(mN)(fN) |
| as | #(mN)#(fN)#(mN)#(mN)#(mN)#(fN)#(mN) |
| P2_ib12_ | P(mN)#(fN)#(mN)(mN)(mN)(fN)(mN)(mN)(mN)(mN)(mN)(ibut)(mN)(mN)(fN) |
| as | #(mN)#(fN)#(mN)#(mN)#(mN)#(fN)#(mN) |
| P2_ib13_ | P(mN)#(fN)#(mN)(mN)(mN)(fN)(mN)(mN)(mN)(mN)(mN)(mN)(ibut)(mN)(fN) |
| as | #(mN)#(fN)#(mN)#(mN)#(mN)#(fN)#(mN) |
| P2_ib14_ | P(mN)#(fN)#(mN)(mN)(mN)(fN)(mN)(mN)(mN)(mN)(mN)(mN)(mN)(ibut)(fN) |
| as | #(mN)#(fN)#(mN)#(mN)#(mN)#(fN)#(mN) |
| P2_ib15_ | P(mN)#(fN)#(mN)(mN)(mN)(fN)(mN)(mN)(mN)(mN)(mN)(mN)(mN)(fN)(ibut) |
| as | #(mN)#(fN)#(mN)#(mN)#(mN)#(fN)#(mN) |
| P2_ib16_ | P(mN)#(fN)#(mN)(mN)(mN)(fN)(mN)(mN)(mN)(mN)(mN)(mN)(mN)(fN)#(mN) |
| as | (ibut)#(fN)#(mN)#(mN)#(mN)#(fN)#(mN) |
| P2_ib17_ | P(mN)#(fN)#(mN)(mN)(mN)(fN)(mN)(mN)(mN)(mN)(mN)(mN)(mN)(fN)#(mN) |
| as | #(fN)(ibut)#(mN)#(mN)#(mN)#(fN)#(mN) |
| P2_ib18_ | P(mN)#(fN)#(mN)(mN)(mN)(fN)(mN)(mN)(mN)(mN)(mN)(mN)(mN)(fN)#(mN) |
| as | #(fN)#(mN)(ibut)#(mN)#(mN)#(fN)#(mN) |
| P2_ib19_ | P(mN)#(fN)#(mN)(mN)(mN)(fN)(mN)(mN)(mN)(mN)(mN)(mN)(mN)(fN)#(mN) |
| as | #(fN)#(mN)#(mN)#(ibut)(mN)#(fN)#(mN) |
| P2_ib20_ | P(mN)#(fN)#(mN)(mN)(mN)(fN)(mN)(mN)(mN)(mN)(mN)(mN)(mN)(fN)#(mN) |
| as | #(fN)#(mN)#(mN)#(mN)#(ibut)(fN)#(mN) |
| P2_ib21_ | P(mN)#(fN)#(mN)(mN)(mN)(fN)(mN)(mN)(mN)(mN)(mN)(mN)(mN)(fN)#(mN) |
| as | #(fN)#(mN)#(mN)#(mN)#(fN)#(ibut)(mN) |
| P2_ib22_ | P(mN)#(fN)#(mN)(mN)(mN)(fN)(mN)(mN)(mN)(mN)(mN)(mN)(mN)(fN)#(mN) |
| as | #(fN)#(mN)#(mN)#(mN)#(fN)#(mN)(ibut) |
| as_P5_TW_ | P(mN)#(fN)#(mN)(mN)(mN)(fN)(mN)(mN)(mN)(mN)(mN)(mN)(mN)(fN)#(mN) |
| iBut1 | #(fN)#(mN)#(mN)#(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut2 | #(fN)#(ibut)(mN)#(mN)#(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut3 | #(fN)#(mN)#(ibut)(mN)#(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut4 | #(fN)#(mN)#(mN)#(ibut)(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut5 | #(fN)#(mN)#(mN)#(mN)#(ibut)(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut6 | #(fN)#(mN)#(mN)#(mN)#(mN)#(ibut)(mN) |
| as_P5_TW_ | P(mN)#(fN)#(mN)(mN)(mN)(fN)(mN)(mN)(mN)(mN)(mN)(mN)(mN)(fN)#(mN) |
| iBut7 | #(fN)#(ibut)(mN)#(ibut)(mN)#(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut8 | #(fN)#(mN)#(ibut)(mN)#(ibut)(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut9 | #(fN)#(mN)#(mN)#(ibut)(mN)#(ibut)(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut10 | #(fN)#(mN)#(mN)#(mN)#(ibut)(mN)#(ibut)(mN) |
| as_P5_TW_ | P(mN)#(fN)#(mN)(mN)(mN)(fN)(mN)(mN)(mN)(mN)(mN)(mN)(mN)(fN)#(mN) |
| iBut11 | #(fN)#(ibut)(mN)#(mN)#(ibut)(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut12 | #(fN)#(mN)#(mN)#(ibut)(mN)#(mN)#(ibut)(mN) |
| as_P5_TW_ | P(mN)#(fN)#(mN)(mN)(mN)(fN)(mN)(mN)(mN)(mN)(mN)(mN)(mN)(fN)#(mN) |
| iBut13 | #(fN)#(mN)#(ibut)(mN)#(mN)#(ibut)(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut14 | #(fN)#(ibut)(mN)#(ibut)(mN)#(ibut)(mN)#(mN)#(mN) |
| as_P5_TW_ | P(mN)#(fN)#(mN)(mN)(mN)(fN)(mN)(mN)(mN)(mN)(mN)(mN)(mN)(fN)#(mN) |
| iBut15 | #(fN)#(mN)#(mN)#(ibut)(mN)#(ibut)(mN)#(ibut)(mN) |
| as_P5_TW_ | P(mN)#(fN)#(mN)(mN)(mN)(fN)(mN)(mN)(mN)(mN)(mN)(mN)(mN)(fN)#(mN) |
| iBut16 | #(fN)#(ibut)(mN)#(ibut)(mN)#(ibut)(mN)#(ibut)(mN)#(ibut)(mN) |

35. The dsRNA of claim 15, comprising a sense strand with one of the following chemical modification patterns:

| | |
|---|---|
| P1_b1_s | (but)#(mN)#(mN)(fN)(mN)(fN)(mN)(fN)(mN)(fN)(mN)(mN)(mN)(fN)#(mN)#(mN) |
| P1_b2_s | (mN)#(but)#(mN)(fN)(mN)(fN)(mN)(fN)(mN)(fN)(mN)(mN)(mN)(fN)#(mN)#(mN) |
| P1_b3_s | (mN)#(mN)#(but)(fN)(mN)(fN)(mN)(fN)(mN)(fN)(mN)(mN)(mN)(fN)#(mN)#(mN) |
| P1_b4_s | (mN)#(mN)#(mN)(but)(mN)(fN)(mN)(fN)(mN)(fN)(mN)(mN)(mN)(fN)#(mN)# |
| | (mN) |
| P1_b5_s | (mN)#(mN)#(mN)(fN)(but)(fN)(mN)(fN)(mN)(fN)(mN)(mN)(mN)(fN)#(mN)# |
| | (mN) |
| P1_b6_s | (mN)#(mN)#(mN)(fN)(mN)(but)(mN)(fN)(mN)(fN)(mN)(mN)(mN)(fN)#(mN)# |
| | (mN) |
| P1_b7_s | (mN)#(mN)#(mN)(fN)(mN)(fN)(but)(fN)(mN)(fN)(mN)(mN)(mN)(fN)#(mN)# |
| | (mN) |
| P1_b8_s | (mN)#(mN)#(mN)(fN)(mN)(fN)(mN)(but)(mN)(fN)(mN)(mN)(mN)(fN)#(mN)# |
| | (mN) |
| P1_b9_s | (mN)#(mN)#(mN)(fN)(mN)(fN)(mN)(fN)(but)(fN)(mN)(mN)(mN)(fN)#(mN)# |
| | (mN) |
| P1_b10_ | (mN)#(mN)#(mN)(fN)(mN)(fN)(mN)(fN)(mN)(but)(mN)(mN)(mN)(fN)#(mN)# |
| s | (mN) |
| P1_b11_ | (mN)#(mN)#(mN)(fN)(mN)(fN)(mN)(fN)(mN)(fN)(but)(mN)(mN)(fN)#(mN)# |
| s | (mN) |
| P1_b12_ | (mN)#(mN)#(mN)(fN)(mN)(fN)(mN)(fN)(mN)(fN)(mN)(but)(mN)(fN)#(mN)# |
| s | (mN) |
| P1_b13_ | (mN)#(mN)#(mN)(fN)(mN)(fN)(mN)(fN)(mN)(fN)(mN)(mN)(but)(fN)#(mN)# |
| s | (mN) |
| P1_b14_ | (mN)#(mN)#(mN)(fN)(mN)(fN)(mN)(fN)(mN)(fN)(mN)(mN)(mN)(but)#(mN)# |
| s | (mN) |
| P1_b15_ | (mN)#(mN)#(mN)(fN)(mN)(fN)(mN)(fN)(mN)(fN)(mN)(mN)(mN)(fN)#(but)# |
| s | (mN) |
| P1_b16_ | (mN)#(mN)#(mN)(fN)(mN)(fN)(mN)(fN)(mN)(fN)(mN)(mN)(mN)(fN)#(mN)# |
| s | (but) |
| P2_b1_s | (but)#(mN)#(mN)(mN)(mN)(fN)(fN)(fN)(mN)(fN)(mN)(mN)(mN)(mN)#(mN)# |
| | (mN) |
| P2_b2_s | (mN)#(but)#(mN)(mN)(mN)(fN)(fN)(fN)(mN)(fN)(mN)(mN)(mN)(mN)#(mN)# |
| | (mN) |
| P2_b3_s | (mN)#(mN)#(but)(mN)(mN)(fN)(fN)(fN)(mN)(fN)(mN)(mN)(mN)(mN)#(mN)# |
| | (mN) |
| P2_b4_s | (mN)#(mN)#(mN)(but)(mN)(fN)(fN)(fN)(mN)(fN)(mN)(mN)(mN)(mN)#(mN)# |
| | (mN) |
| P2_b5_s | (mN)#(mN)#(mN)(mN)(but)(fN)(fN)(fN)(mN)(fN)(mN)(mN)(mN)(mN)#(mN)# |
| | (mN) |
| P2_b6_s | (mN)#(mN)#(mN)(mN)(mN)(but)(fN)(fN)(mN)(fN)(mN)(mN)(mN)(mN)#(mN)# |
| | (mN) |
| P2_b7_s | (mN)#(mN)#(mN)(mN)(mN)(fN)(but)(fN)(mN)(fN)(mN)(mN)(mN)(mN)#(mN)# |
| | (mN) |
| P2_b8_s | (mN)#(mN)#(mN)(mN)(mN)(fN)(fN)(but)(mN)(fN)(mN)(mN)(mN)(mN)#(mN)# |
| | (mN) |
| P2_b9_s | (mN)#(mN)#(mN)(mN)(mN)(fN)(fN)(fN)(but)(fN)(mN)(mN)(mN)(mN)#(mN)# |
| | (mN) |
| P2_b10_ | (mN)#(mN)#(mN)(mN)(mN)(fN)(fN)(fN)(mN)(but)(mN)(mN)(mN)(mN)#(mN)# |
| s | (mN) |
| P2_b11_ | (mN)#(mN)#(mN)(mN)(mN)(fN)(fN)(fN)(mN)(fN)(but)(mN)(mN)(mN)#(mN)# |
| s | (mN) |
| P2_b12_ | (mN)#(mN)#(mN)(mN)(mN)(fN)(fN)(fN)(mN)(fN)(mN)(but)(mN)(mN)#(mN)# |
| s | (mN) |
| P2_b13_ | (mN)#(mN)#(mN)(mN)(mN)(fN)(fN)(fN)(mN)(fN)(mN)(mN)(but)(mN)#(mN)# |
| s | (mN) |
| P2_b14_ | (mN)#(mN)#(mN)(mN)(mN)(fN)(fN)(fN)(mN)(fN)(mN)(mN)(mN)(but)#(mN)# |
| s | (mN) |
| P2_b15_ | (mN)#(mN)#(mN)(mN)(mN)(fN)(fN)(fN)(mN)(fN)(mN)(mN)(mN)(mN)#(but)# |
| s | (mN) |
| P2_b16_ | (mN)#(mN)#(mN)(mN)(mN)(fN)(fN)(fN)(mN)(fN)(mN)(mN)(mN)(mN)#(mN)# |
| s | (but) |
| P1_ib1_s | (ibut)(mN)#(mN)#(mN)(fN)(mN)(fN)(mN)(fN)(mN)(fN)(mN)(mN)(mN)(fN)# |
| | (mN)#(mN) |
| P1_ib2_s | (mN)#(ibut)(mN)#(mN)(fN)(mN)(fN)(mN)(fN)(mN)(fN)(mN)(mN)(mN)(fN)# |
| | (mN)#(mN) |
| P1_ib3_s | (mN)#(mN)#(ibut)(mN)(fN)(mN)(fN)(mN)(fN)(mN)(fN)(mN)(mN)(mN)(fN)# |
| | (mN)#(mN) |
| P1_ib4_s | (mN)#(mN)#(mN)(ibut)(fN)(mN)(fN)(mN)(fN)(mN)(fN)(mN)(mN)(mN)(fN)# |
| | (mN)#(mN) |
| P1_ib5_s | (mN)#(mN)#(mN)(fN)(ibut)(mN)(fN)(mN)(fN)(mN)(fN)(mN)(mN)(mN)(fN)# |
| | (mN)#(mN) |
| P1_ib6_s | (mN)#(mN)#(mN)(fN)(mN)(ibut)(fN)(mN)(fN)(mN)(fN)(mN)(mN)(mN)(fN)# |
| | (mN)#(mN) |
| P1_ib7_s | (mN)#(mN)#(mN)(fN)(mN)(fN)(ibut)(mN)(fN)(mN)(fN)(mN)(mN)(mN)(fN)# |
| | (mN)#(mN) |
| P1_ib8_s | (mN)#(mN)#(mN)(fN)(mN)(fN)(mN)(ibut)(fN)(mN)(fN)(mN)(mN)(mN)(fN)# |
| | (mN)#(mN) |
| P1_ib9_s | (mN)#(mN)#(mN)(fN)(mN)(fN)(mN)(fN)(ibut)(mN)(fN)(mN)(mN)(mN)(fN)# |
| | (mN)#(mN) |
| P1_ib10_ | (mN)#(mN)#(mN)(fN)(mN)(fN)(mN)(fN)(mN)(ibut)(fN)(mN)(mN)(mN)(fN)# |
| s | (mN)#(mN) |
| P1_ib11_ | (mN)#(mN)#(mN)(fN)(mN)(fN)(mN)(fN)(mN)(fN)(ibut)(mN)(mN)(mN)(fN)# |
| s | (mN)#(mN) |
| P1_ib12_ | (mN)#(mN)#(mN)(fN)(mN)(fN)(mN)(fN)(mN)(fN)(mN)(ibut)(mN)(mN)(fN)# |
| s | (mN)#(mN) |
| P1_ib13_ | (mN)#(mN)#(mN)(fN)(mN)(fN)(mN)(fN)(mN)(fN)(mN)(mN)(ibut)(mN)(fN)# |
| s | (mN)#(mN) |
| P1_ib14_ | (mN)#(mN)#(mN)(fN)(mN)(fN)(mN)(fN)(mN)(fN)(mN)(mN)(mN)(ibut)(fN)# |
| s | (mN)#(mN) |
| P1_ib15_ | (mN)#(mN)#(mN)(fN)(mN)(fN)(mN)(fN)(mN)(fN)(mN)(mN)(mN)(fN)#(ibut) |
| s | (mN)#(mN) |
| P1_ib16_ | (mN)#(mN)#(mN)(fN)(mN)(fN)(mN)(fN)(mN)(fN)(mN)(mN)(mN)(fN)#(mN)# |
| s | (ibut)(mN) |
| P1_ib17_ | (mN)#(mN)#(mN)(fN)(mN)(fN)(mN)(fN)(mN)(fN)(mN)(mN)(mN)(fN)#(mN)# |
| s | (mN)(ibut) |
| P2_ib1_s | (ibut)(mN)#(mN)#(mN)(mN)(mN)(fN)(fN)(fN)(mN)(fN)(mN)(mN)(mN)(mN)# |
| | (mN)#(mN) |
| P2_ib2_s | (mN)(ibut)#(mN)#(mN)(mN)(mN)(fN)(fN)(fN)(mN)(fN)(mN)(mN)(mN)(mN)# |
| | (mN)#(mN) |
| P2_ib3_s | (mN)#(mN)(ibut)#(mN)(mN)(mN)(fN)(fN)(fN)(mN)(fN)(mN)(mN)(mN)(mN)# |
| | (mN)#(mN) |
| P2_ib4_s | (mN)#(mN)#(mN)(ibut)(mN)(mN)(fN)(fN)(fN)(mN)(fN)(mN)(mN)(mN)(mN)# |
| | (mN)#(mN) |
| P2_ib5_s | (mN)#(mN)#(mN)(mN)(ibut)(mN)(fN)(fN)(fN)(mN)(fN)(mN)(mN)(mN)(mN)# |
| | (mN)#(mN) |
| P2_ib6_s | (mN)#(mN)#(mN)(mN)(mN)(ibut)(fN)(fN)(fN)(mN)(fN)(mN)(mN)(mN)(mN)# |
| | (mN)#(mN) |
| P2_ib7_s | (mN)#(mN)#(mN)(mN)(mN)(fN)(ibut)(fN)(fN)(mN)(fN)(mN)(mN)(mN)(mN)# |
| | (mN)#(mN) |
| P2_ib8_s | (mN)#(mN)#(mN)(mN)(mN)(fN)(fN)(ibut)(fN)(mN)(fN)(mN)(mN)(mN)(mN)# |
| | (mN)#(mN) |
| P2_ib9_s | (mN)#(mN)#(mN)(mN)(mN)(fN)(fN)(fN)(ibut)(mN)(fN)(mN)(mN)(mN)(mN)# |
| | (mN)#(mN) |
| P2_ib10_ | (mN)#(mN)#(mN)(mN)(mN)(fN)(fN)(fN)(mN)(ibut)(fN)(mN)(mN)(mN)(mN)# |
| s | (mN)#(mN) |
| P2_ib11_ | (mN)#(mN)#(mN)(mN)(mN)(fN)(fN)(fN)(mN)(fN)(ibut)(mN)(mN)(mN)(mN)# |
| s | (mN)#(mN) |
| P2_ib12_ | (mN)#(mN)#(mN)(mN)(mN)(fN)(fN)(fN)(mN)(fN)(mN)(ibut)(mN)(mN)(mN)# |
| s | (mN)#(mN) |
| P2_ib13_ | (mN)#(mN)#(mN)(mN)(mN)(fN)(fN)(fN)(mN)(fN)(mN)(mN)(ibut)(mN)(mN)# |
| s | (mN)#(mN) |
| P2_ib14_ | (mN)#(mN)#(mN)(mN)(mN)(fN)(fN)(fN)(mN)(fN)(mN)(mN)(mN)(ibut)(mN)# |
| s | (mN)#(mN) |
| P2_ib15_ | (mN)#(mN)#(mN)(mN)(mN)(fN)(fN)(fN)(mN)(fN)(mN)(mN)(mN)(mN)(ibut)# |
| s | (mN)#(mN) |
| P2_ib16_ | (mN)#(mN)#(mN)(mN)(mN)(fN)(fN)(fN)(mN)(fN)(mN)(mN)(mN)(mN)#(mN)# |
| s | (ibut)(mN) |
| P2_ib17_ | (mN)#(mN)#(mN)(mN)(mN)(fN)(fN)(fN)(mN)(fN)(mN)(mN)(mN)(mN)#(mN)# |
| s | (mN)(ibut) |

36. A double stranded (ds) RNA, comprising an antisense strand and a sense strand, each strand with a 5′ end and a 3′ end, and at least one single stranded nucleotide overhang of 2-5 nucleotides, wherein the single stranded nucleotide overhang comprises at least two nucleotide modifications selected from the group consisting of a 2′-deoxy modification, a 2′-MOE modification, an LNA modification, a UNA modification, and an alkyl modification.

37. The dsRNA of claim 36, wherein:
the single stranded nucleotide overhang comprises 2, 3, 4, or 5 nucleotide modifications selected from the group consisting of a 2′-deoxy modification, a 2′-MOE modification, an LNA modification, a UNA modification, and an alkyl modification, and/or
each nucleotide in the single stranded nucleotide overhang comprises the same nucleotide modification, or the single stranded nucleotide overhang comprises at least two different nucleotide modifications.

38-39. (canceled)

40. A double stranded (ds) RNA, comprising an antisense strand and a sense strand, each strand with a 5′ end and a 3′ end, wherein the antisense strand or sense strand comprises a chemical modification pattern of any one of the chemical modification patterns provided in Tables 1-8.

41. (canceled)

42. A method for reducing expression of a target mRNA in a subject, comprising administering to the subject the RNA molecule of claim 1, thereby reducing the expression of the target mRNA.

43. The method of claim 42, wherein:
the expression of the target mRNA is reduced by at least about 20%, at least about 30%, at least about 40%, or at least about 50% over an expression level prior to administration of the RNA molecule or dsRNA; and/or
the expression of the target mRNA is reduced for at least about 3 months, at least about 4 months, at least about 5 months, at least about 6 months, at least about 7 months, at least about 8 months, at least about 9 months, at least about 10 months, at least about 11 months, or at least about 12 months after administration of the RNA molecule or dsRNA.

44. (canceled)
